SEQUENCE LISTING (1188 sequences)

SEQ ID NO: 1 - protein, 5 aa, synthetic construct
DYYIT

SEQ ID NO: 2 - protein, 17 aa, synthetic construct
WIYPGSGNTKYNEKFKG

SEQ ID NO: 3 - protein, 8 aa, synthetic construct
YGNYWFAY

SEQ ID NO: 4 - protein, 15 aa, synthetic construct
KASQSVDFDGDSYMN

SEQ ID NO: 5 - protein, 7 aa, synthetic construct
AASNLES

SEQ ID NO: 6 - protein, 9 aa, synthetic construct
QQSNEDPWT

SEQ ID NO: 7 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 13 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 14 - protein, 448 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NYGMN

SEQ ID NO: 17 - protein, 17 aa, synthetic construct
WINTYTGEPTYTDDFKG

SEQ ID NO: 18 - protein, 12 aa, synthetic construct
GGFGSSYWYFDV

SEQ ID NO: 19 - protein, 11 aa, synthetic construct
KASQDVSIAVA

SEQ ID NO: 20 - protein, 7 aa, synthetic construct
SASYRYT

SEQ ID NO: 21 - protein, 9 aa, synthetic construct
QQHYITPLT

SEQ ID NO: 22 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 23 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 24 - protein, 5 aa, synthetic construct
TAGMQ

SEQ ID NO: 25 - protein, 17 aa, synthetic construct
WINTHSGVPKYAEDFKG

SEQ ID NO: 26 - protein, 12 aa, synthetic construct
SGFGSSYWYFDV

SEQ ID NO: 27 - protein, 11 aa, synthetic construct
KASQDVSTAVA

SEQ ID NO: 28 - protein, 7 aa, synthetic construct
SASYRYT

SEQ ID NO: 29 - protein, 9 aa, synthetic construct
QQHYITPLT

SEQ ID NO: 30 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 31 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 32 - protein, 5 aa, synthetic construct
SQNIY

SEQ ID NO: 33 - protein, 17 aa, synthetic construct
YIEPYNVVPMYNPKFKG

SEQ ID NO: 34 - protein, 8 aa, synthetic construct
SGSSNFDY

SEQ ID NO: 35 - protein, 12 aa, synthetic construct
SASSSISSHYLH

SEQ ID NO: 36 - protein, 7 aa, synthetic construct
RTSNLAS

SEQ ID NO: 37 - protein, 9 aa, synthetic construct
QQGSSLPLT

SEQ ID NO: 38 - protein, 117 aa, synthetic construct
EIQLVQSGAEVKKPGASVKVSCKASGYAFTSQNIYWVRQAPGQGLEWIGYIEPYNVVPMYNPKFKGRATLTVDKSTSTAYLELSSLRSEDTAVYYCARSGSSNFDYWGQGTLVTVSS

SEQ ID NO: 39 - protein, 108 aa, synthetic construct
DIQLTQSPSSLSASVGDRVTITCSASSSISSHYLHWYQQKPGKSPKLLIYRTSNLASGVPSRFSGSGSGTDYTLTISSLQPEDFATYYCQQGSSLPLTFGQGTKVEIK

SEQ ID NO: 40 - protein, 5 aa, synthetic construct
NYAMH

SEQ ID NO: 41 - protein, 17 aa, synthetic construct
LIWYDGSNKFYGDSVKG

SEQ ID NO: 42 - protein, 6 aa, synthetic construct
EGSGHY

SEQ ID NO: 43 - protein, 11 aa, synthetic construct
RASQGISSALA

SEQ ID NO: 44 - protein, 7 aa, synthetic construct
DASSLES

SEQ ID NO: 45 - protein, 9 aa, synthetic construct
QQFNSYPIT

SEQ ID NO: 46 - protein, 115 aa, synthetic construct
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSS

SEQ ID NO: 47 - protein, 107 aa, synthetic construct
AIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIK

SEQ ID NO: 48 - protein, 5 aa, synthetic construct
NYAMS

SEQ ID NO: 49 - protein, 17 aa, synthetic construct
YISPGGDYIYYADSVKG

SEQ ID NO: 50 - protein, 12 aa, synthetic construct
DRRHYGSYAMDY

SEQ ID NO: 51 - protein, 16 aa, synthetic construct
RSSKSLLHSNLNTYLY

SEQ ID NO: 52 - protein, 7 aa, synthetic construct
RMSNLAS

SEQ ID NO: 53 - protein, 9 aa, synthetic construct
MQHLEYPFT

SEQ ID NO: 54 - protein, 121 aa, synthetic construct
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 55 - protein, 112 aa, synthetic construct
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIK

SEQ ID NO: 56 - protein, 5 aa, synthetic construct
TYAFH

SEQ ID NO: 57 - protein, 17 aa, synthetic construct
GIVPIFGTLKYAQKFQD

SEQ ID NO: 58 - protein, 11 aa, synthetic construct
AIQLEGRPFDH

SEQ ID NO: 59 - protein, 11 aa, synthetic construct
RASQGITSYLA

SEQ ID NO: 60 - protein, 7 aa, synthetic construct
AASALQS

SEQ ID NO: 61 - protein, 10 aa, synthetic construct
QQVNRGAAIT

SEQ ID NO: 62 - protein, 120 aa, synthetic construct
QVQLVQSGAEVKKPGSSVRVSCRASGGSSTTYAFHWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 63 - protein, 108 aa, synthetic construct
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIK

SEQ ID NO: 64 - protein, 5 aa, synthetic construct
TYAFH

SEQ ID NO: 65 - protein, 17 aa, synthetic construct
GIVPIFGTLKYAQKFQD

SEQ ID NO: 66 - protein, 11 aa, synthetic construct
AIQLEGRPFDH

SEQ ID NO: 67 - protein, 11 aa, synthetic construct
RASQGITSYLA

SEQ ID NO: 68 - protein, 7 aa, synthetic construct
AASALQS

SEQ ID NO: 69 - protein, 10 aa, synthetic construct
QQVNRGAAIT

SEQ ID NO: 70 - protein, 120 aa, synthetic construct; identical to SEQ ID NO: 62

SEQ ID NO: 71 - protein, 108 aa, synthetic construct; identical to SEQ ID NO: 63

SEQ ID NO: 72 - protein, 5 aa, synthetic construct
RYTMH

SEQ ID NO: 73 - protein, 17 aa, synthetic construct
VISFDGSNKYYVDSVKG

SEQ ID NO: 74 - protein, 10 aa, synthetic construct
EARGSYAFDI

SEQ ID NO: 75 - protein, 11 aa, synthetic construct
RASQSVSSYLA

SEQ ID NO: 76 - protein, 7 aa, synthetic construct
DASNRAT

SEQ ID NO: 77 - protein, 10 aa, synthetic construct
QQRSNWPPFT

SEQ ID NO: 78 - protein, 119 aa, synthetic construct
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 79 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 80 - protein, 5 aa, synthetic construct
SFWMH

SEQ ID NO: 81 - protein, 17 aa, synthetic construct
YINPRSGYTEYNEIFRD

SEQ ID NO: 82 - protein, 9 aa, synthetic construct
FLGRGAMDY

SEQ ID NO: 83 - protein, 11 aa, synthetic construct
RASQDISNYLA

SEQ ID NO: 84 - protein, 7 aa, synthetic construct
YTSKIHS

SEQ ID NO: 85 - protein, 9 aa, synthetic construct
QQGNTFPYT

SEQ ID NO: 86 - protein, 118 aa, synthetic construct [sequence omitted]

SEQ ID NO: 87 - protein, 107 aa, synthetic construct [sequence omitted]

SEQ ID NO: 88 - protein, 7 aa, synthetic construct
TSSYYWG

SEQ ID NO: 89 - protein, 16 aa, synthetic construct
TIYYNGSTYYSPSLKS

SEQ ID NO: 90 - protein, 11 aa, synthetic construct
QGYDIKINIDV

SEQ ID NO: 91 - protein, 11 aa, synthetic construct
RASQSVSSYLA

SEQ ID NO: 92 - protein, 7 aa, synthetic construct
VASNRAT

SEQ ID NO: 93 - protein, 9 aa, synthetic construct
QQRSNWPLT

SEQ ID NO: 94 - protein, 121 aa, synthetic construct
QLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSS

SEQ ID NO: 95 - protein, 107 aa, synthetic construct
EIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIK

SEQ ID NO: 96 - protein, 5 aa, synthetic construct
SSWMN

SEQ ID NO: 97 - protein, 17 aa, synthetic construct
RIYPGDGNTHYAQKFQG

SEQ ID NO: 98 - protein, 8 aa, synthetic construct
GYLDPMDY

SEQ ID NO: 99 - protein, 11 aa, synthetic construct
QASQGINNYLN

SEQ ID NO: 100 - protein, 7 aa, synthetic construct
YTSGLHA

SEQ ID NO: 101 - protein, 9 aa, synthetic construct
QQYSILPWT

SEQ ID NO: 102 - protein, 117 aa, synthetic construct
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 103 - protein, 107 aa, synthetic construct
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 104 - protein, 5 aa, synthetic construct
SYGMH

SEQ ID NO: 105 - protein, 17 aa, synthetic construct
VISYDGSNKYYADSVKG

SEQ ID NO: 106 - protein, 18 aa, synthetic construct
DMGWGSGWRPYYYYGMDV

SEQ ID NO: 107 - protein, 11 aa, synthetic construct
RTSQSISSYLN

SEQ ID NO: 108 - protein, 7 aa, synthetic construct
WASTRES

SEQ ID NO: 109 - protein, 9 aa, synthetic construct
QQSYDIPYT

SEQ ID NO: 110 - protein, 127 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 111 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 112 - protein, 5 aa, synthetic construct
NYWMS

SEQ ID NO: 113 - protein, 17 aa, synthetic construct
NIKQDGSEKFYADSVKG

SEQ ID NO: 114 - protein, 9 aa, synthetic construct
VGPSWEQDY

SEQ ID NO: 115 - protein, 14 aa, synthetic construct
TGSSSNIGSYYGVH

SEQ ID NO: 116 - protein, 7 aa, synthetic construct
SDTNRPS

SEQ ID NO: 117 - protein, 11 aa, synthetic construct
QSYDKGFGHRV

SEQ ID NO: 118 - protein, 118 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 119 - protein, 111 aa, synthetic construct
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVL

SEQ ID NO: 120 - protein, 5 aa, synthetic construct
SYAIS

SEQ ID NO: 121 - protein, 17 aa, synthetic construct
GIIPIFGTANYAQKFQG

SEQ ID NO: 122 - protein, 6 aa, synthetic construct
GLLWNY

SEQ ID NO: 123 - protein, 11 aa, synthetic construct
RASQSVSSNLA

SEQ ID NO: 124 - protein, 7 aa, synthetic construct
GASTTAS

SEQ ID NO: 125 - protein, 11 aa, synthetic construct
QQYNNWPPAYT

SEQ ID NO: 126 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 127 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 128 - protein, 5 aa, synthetic construct
NYGMN

SEQ ID NO: 129 - protein, 17 aa, synthetic construct
WINTYTGEPTYGEDFKG

SEQ ID NO: 130 - protein, 7 aa, synthetic construct
FGNYVDY

SEQ ID NO: 131 - protein, 16 aa, synthetic construct
RSSKNLLHSNGITYLY

SEQ ID NO: 132 - protein, 7 aa, synthetic construct
QMSNLAS

SEQ ID NO: 133 - protein, 9 aa, synthetic construct
AQNLEIPRT

SEQ ID NO: 134 - protein, 116 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 135 - protein, 112 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 136 - protein, 5 aa, synthetic construct
KYGMN

SEQ ID NO: 137 - protein, 17 aa, synthetic construct
WINTYTEEPTYGDDFKG

SEQ ID NO: 138 - protein, 7 aa, synthetic construct
FGSAVDY

SEQ ID NO: 139 - protein, 16 aa, synthetic construct
RSSKSLLHSNGITYLY

SEQ ID NO: 140 - protein, 7 aa, synthetic construct
QMSNRAS

SEQ ID NO: 141 - protein, 9 aa, synthetic construct
AQNLELPRT

SEQ ID NO: 142 - protein, 116 aa, synthetic construct
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 143 - protein, 113 aa, synthetic construct
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKR

SEQ ID NO: 144 - protein, 5 aa, synthetic construct
DYSMH

SEQ ID NO: 145 - protein, 17 aa, synthetic construct
WINTETGEPTYADDFKG

SEQ ID NO: 146 - protein, 4 aa, synthetic construct
TAVY

SEQ ID NO: 147 - protein, 11 aa, synthetic construct
RASQEISVSLS

SEQ ID NO: 148 - protein, 7 aa, synthetic construct
ATSTLDS

SEQ ID NO: 149 - protein, 9 aa, synthetic construct
LQYASYPWT

SEQ ID NO: 150 - protein, 113 aa, synthetic construct
QVKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 151 - protein, 108 aa, synthetic construct
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKR

SEQ ID NO: 152 - protein, 5 aa, synthetic construct
NYGMN

SEQ ID NO: 153 - protein, 17 aa, synthetic construct
WINTYSGEPRYADDFKG

SEQ ID NO: 154 - protein, 9 aa, synthetic construct
DYGRWYFDV

SEQ ID NO: 155 - protein, 10 aa, synthetic construct
RASSSVSHMH

SEQ ID NO: 156 - protein, 7 aa, synthetic construct
ATSNLAS

SEQ ID NO: 157 - protein, 9 aa, synthetic construct
QQWSSTPRT

SEQ ID NO: 158 - protein, 118 aa, synthetic construct
QIQLVQSGSELKKPGASVKVSCKASGYTFTNYGMNWVRQAPGQDLKWMGWINTYSGEPRYADDFKGRFVFSLDKSVNTAYLQISSLKAEDTAVYYCARDYGRWYFDVWGQGTTVTVSS

SEQ ID NO: 159 - protein, 107 aa, synthetic construct
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIKR

SEQ ID NO: 160 - protein, 5 aa, synthetic construct
RYWMS

SEQ ID NO: 161 - protein, 17 aa, synthetic construct
EINPDSSTINYAPSLKD

SEQ ID NO: 162 - protein, 10 aa, synthetic construct
PDGNYWYFDV

SEQ ID NO: 163 - protein, 11 aa, synthetic construct
KASQDVGIAVA

SEQ ID NO: 164 - protein, 7 aa, synthetic construct
WASTRHT

SEQ ID NO: 165 - protein, 9 aa, synthetic construct
QQYSSYPYT

SEQ ID NO: 166 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 167 - protein, 108 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 168 - protein, 5 aa, synthetic construct
SFAMS

SEQ ID NO: 169 - protein, 17 aa, synthetic construct
AISGSGGGTYYADSVKG

SEQ ID NO: 170 - protein, 13 aa, synthetic construct
DKILWFGEPVFDY

SEQ ID NO: 171 - protein, 11 aa, synthetic construct
RASQSVSSYLA

SEQ ID NO: 172 - protein, 7 aa, synthetic construct
DASNRAT

SEQ ID NO: 173 - protein, 9 aa, synthetic construct
QQRSNWPPT

SEQ ID NO: 174 - protein, 122 aa, synthetic construct [sequence omitted]

SEQ ID NO: 175 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 176 - protein, 5 aa, synthetic construct
SYRMH

SEQ ID NO: 177 - protein, 17 aa, synthetic construct
YINPSTGYTEYNQKFKD

SEQ ID NO: 178 - protein, 7 aa, synthetic construct
GGGVFDY

SEQ ID NO: 179 - protein, 10 aa, synthetic construct
SASSSISYMH

SEQ ID NO: 180 - protein, 7 aa, synthetic construct
TTSNLAS

SEQ ID NO: 181 - protein, 9 aa, synthetic construct
HQRSTYPLT

SEQ ID NO: 182 - protein, 116 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 183 - protein, 106 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 184 - protein, 5 aa, synthetic construct
SYWMH

SEQ ID NO: 185 - protein, 17 aa, synthetic construct
EIIPINGHTNYNEKFKS

SEQ ID NO: 186 - protein, 14 aa, synthetic construct
GGYYYYGSRDYFDY

SEQ ID NO: 187 - protein, 15 aa, synthetic construct
KASQSVDYDGDSYMN

SEQ ID NO: 188 - protein, 7 aa, synthetic construct
AASDLES

SEQ ID NO: 189 - protein, 9 aa, synthetic construct
QQSHEDPFT

SEQ ID NO: 190 - protein, 123 aa, synthetic construct
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 191 - protein, 111 aa, synthetic construct
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 192 - protein, 5 aa, synthetic construct
SYWMH

SEQ ID NO: 193 - protein, 17 aa, synthetic construct
EIIPIFGHTNYNEKFKS

SEQ ID NO: 194 - protein, 14 aa, synthetic construct
GGYYYYPRQGFLDY

SEQ ID NO: 195 - protein, 15 aa, synthetic construct
KASQSVDYSGDSYMN

SEQ ID NO: 196 - protein, 7 aa, synthetic construct
AASDLES

SEQ ID NO: 197 - protein, 9 aa, synthetic construct
QQSHEDPFT

SEQ ID NO: 198 - protein, 123 aa, synthetic construct
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 199 - protein, 111 aa, synthetic construct
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 200 - protein, 4 aa, synthetic construct
YGMN

SEQ ID NO: 201 - protein, 17 aa, synthetic construct
YISSSSSTIYYADSVKG

SEQ ID NO: 202 - protein, 6 aa, synthetic construct
GPGMDV

SEQ ID NO: 203 - protein, 17 aa, synthetic construct
KSSQSVLYSSNNKNYLA

SEQ ID NO: 204 - protein, 7 aa, synthetic construct
WASTRES

SEQ ID NO: 205 - protein, 10 aa, synthetic construct
QQYYSTPQLT

SEQ ID NO: 206 - protein, 114 aa, synthetic construct
QVQLQQSGGGVVQPGRSLGLSCAASGFTFSSYGMNWVRQAPGKGLEWVSYISSSSSTIYYADSVKGRFTISRDNSKNTLYLQMNSLRAEDTAVYYCARGPGMDVWGQGTTVTVS

SEQ ID NO: 207 - protein, 114 aa, synthetic construct
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIK

SEQ ID NO: 208 - protein, 7 aa, synthetic construct
TSGMGVG

SEQ ID NO: 209 - protein, 16 aa, synthetic construct
HIWWDDDKRYNPALKS

SEQ ID NO: 210 - protein, 10 aa, synthetic construct
MELWSYYFDY

SEQ ID NO: 211 - protein, 10 aa, synthetic construct
SASSSVSYMH

SEQ ID NO: 212 - protein, 7 aa, synthetic construct
DTSKLAS

SEQ ID NO: 213 - protein, 9 aa, synthetic construct
FQGSVYPFT

SEQ ID NO: 214 - protein, 120 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 215 - protein, 107 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 216 - protein, 5 aa, synthetic construct
NYGMN

SEQ ID NO: 217 - protein, 17 aa, synthetic construct
WINTYTGEPTYADAFKG

SEQ ID NO: 218 - protein, 9 aa, synthetic construct
DYGDYGMDY

SEQ ID NO: 219 - protein, 15 aa, synthetic construct
RASKSVSTSGYSFMH

SEQ ID NO: 220 - protein, 7 aa, synthetic construct
LASNLES

SEQ ID NO: 221 - protein, 9 aa, synthetic construct
QHSREVPWT

SEQ ID NO: 222 - protein, 118 aa, synthetic construct; identical to SEQ ID NO: 12

SEQ ID NO: 223 - protein, 111 aa, synthetic construct; identical to SEQ ID NO: 13

SEQ ID NO: 224 - protein, 6 aa, synthetic construct
SGYSWH

SEQ ID NO: 225 - protein, 16 aa, synthetic construct
YIHSSGSTNYNPSLKS

SEQ ID NO: 226 - protein, 7 aa, synthetic construct
YDDYFEY

SEQ ID NO: 227 - protein, 11 aa, synthetic construct
KASQNVGFNVA

SEQ ID NO: 228 - protein, 7 aa, synthetic construct
SASYRYS

SEQ ID NO: 229 - protein, 9 aa, synthetic construct
QQYNWYPFT

SEQ ID NO: 230 - protein, 116 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 231 - protein, 107 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 232 - protein, 5 aa, synthetic construct
NYWIE

SEQ ID NO: 233 - protein, 17 aa, synthetic construct
EILPGTGRTIYNEKFKG

SEQ ID NO: 234 - protein, 13 aa, synthetic construct
RDYYGNFYYAMDY

SEQ ID NO: 235 - protein, 11 aa, synthetic construct
SASQGINNYLN

SEQ ID NO: 236 - protein, 7 aa, synthetic construct
YTSTLQS

SEQ ID NO: 237 - protein, 9 aa, synthetic construct
QQYSKLPRT

SEQ ID NO: 238 - protein, 122 aa, synthetic construct
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 239 - protein, 107 aa, synthetic construct
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 240 - protein, 7 aa, synthetic construct
TYGMGVG

SEQ ID NO: 241 - protein, 16 aa, synthetic construct
NIWWSEDKHYSPSLKS

SEQ ID NO: 242 - protein, 11 aa, synthetic construct
IDYGNDYAFTY

SEQ ID NO: 243 - protein, 16 aa, synthetic construct
RSSKSLLHSNGITYLY

SEQ ID NO: 244 - protein, 7 aa, synthetic construct
QMSNLAS

SEQ ID NO: 245 - protein, 9 aa, synthetic construct
AQNLELPYT

SEQ ID NO: 246 - protein, 121 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 247 - protein, 112 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 248 - protein, 5 aa, synthetic construct
RYTMH

SEQ ID NO: 249 - protein, 17 aa, synthetic construct
VISFDGSNKYYVDSVKG

SEQ ID NO: 250 - protein, 10 aa, synthetic construct
EARGSYAFDI

SEQ ID NO: 251 - protein, 11 aa, synthetic construct
RASQSVSSYLA

SEQ ID NO: 252 - protein, 7 aa, synthetic construct
DASNRAT

SEQ ID NO: 253 - protein, 10 aa, synthetic construct
QQRSNWPPFT

SEQ ID NO: 254 - protein, 119 aa, synthetic construct; identical to SEQ ID NO: 78

SEQ ID NO: 255 - protein, 108 aa, synthetic construct; identical to SEQ ID NO: 79

SEQ ID NO: 256 - protein, 5 aa, synthetic construct
SFGMH

SEQ ID NO: 257 - protein, 17 aa, synthetic construct
YISSGSFTIYYADSVKG

SEQ ID NO: 258 - protein, 9 aa, synthetic construct
MRKGYAMDY

SEQ ID NO: 259 - protein, 16 aa, synthetic construct
RSSQIIIHSDGNTYLE

SEQ ID NO: 260 - protein, 7 aa, synthetic construct
KVSNRFS

SEQ ID NO: 261 - protein, 9 aa, synthetic construct
FQGSHVPHT

SEQ ID NO: 262 - protein, 118 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 263 - protein, 112 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 264 - protein, 5 aa, synthetic construct
NYGVN

SEQ ID NO: 265 - protein, 17 aa, synthetic construct
WINPNTGEPTFDDDFKG

SEQ ID NO: 266 - protein, 11 aa, synthetic construct
SRGKNEAWFAY

SEQ ID NO: 267 - protein, 16 aa, synthetic construct
RSSQSLVHRNGNTYLH

SEQ ID NO: 268 - protein, 7 aa, synthetic construct
TVSNRFS

SEQ ID NO: 269 - protein, 9 aa, synthetic construct
SQSSHVPPT

SEQ ID NO: 270 - protein, 120 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 271 - protein, 112 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 272 - protein, 5 aa, synthetic construct
TYWMS

SEQ ID NO: 273 - protein, 17 aa, synthetic construct
EIHPDSSTINYAPSLKD

SEQ ID NO: 274 - protein, 10 aa, synthetic construct
LYFGFPWFAY

SEQ ID NO: 275 - protein, 11 aa, synthetic construct
KASQDVGTSVA

SEQ ID NO: 276 - protein, 7 aa, synthetic construct
WTSTRHT

SEQ ID NO: 277 - protein, 8 aa, synthetic construct
QQYSLYRS

SEQ ID NO: 278 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 279 - protein, 106 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 280 - protein, 5 aa, synthetic construct
YYGMN

SEQ ID NO: 281 - protein, 17 aa, synthetic construct
WIDTTTGEPTYAQKFQG

SEQ ID NO: 282 - protein, 10 aa, synthetic construct
RGPYNWYFDV

SEQ ID NO: 283 - protein, 16 aa, synthetic construct
RSSKSLLHSNGNTYLY

SEQ ID NO: 284 - protein, 7 aa, synthetic construct
RMSNLVS

SEQ ID NO: 285 - protein, 9 aa, synthetic construct
LQHLEYPFT

SEQ ID NO: 286 - protein, 119 aa, synthetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 287 - protein, 112 aa, synthetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 288 - protein, 5 aa, synthetic construct
NYGMN

SEQ ID NO: 289 - protein, 17 aa, synthetic construct
WINTYTGEPTYADDFKG

SEQ ID NO: 290 - protein, 9 aa, synthetic construct
IGDSSPSDY

SEQ ID NO: 291 - protein, 11 aa, synthetic construct
KASQSVSNDVV

SEQ ID NO: 292 - protein, 7 aa, synthetic construct
YASNRYT

SEQ ID NO: 293 - protein, 9 aa, synthetic construct
QQDYTSPWT

SEQ ID NO: 294 - protein, 118 aa, synthetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 295 - protein, 107 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 296 - protein, 5 aa, synthetic construct
SYWIE

SEQ ID NO: 297 - protein, 17 aa, synthetic construct
EILPGSGNTYYNERFKD

SEQ ID NO: 298 - protein, 14 aa, synthetic construct
RAAAYYSNPEWFAY

SEQ ID NO: 299 - protein, 12 aa, synthetic construct
TASSSVNSFYLH

SEQ ID NO: 300 - protein, 7 aa, synthetic construct
STSNLAS

SEQ ID NO: 301 - protein, 9 aa, synthetic construct
HQYHRSPYT

SEQ ID NO: 302 - protein, 123 aa, synthetic construct
QVQLVQSGAEVKKPGSSVKVSCKASGGTFSSYWIEWVRQAPGQGLEWMGEILPGSGNTYYNERFKDRVTITADESTSTAYMELSSLRSEDTAVYYCARRAAAYYSNPEWFAYWGQGTLVTVSS

SEQ ID NO: 303 - protein, 108 aa, synthetic construct
EIVLTQSPATLSLSPGERATLSCTASSSVNSFYLHWYQQKPGLAPRLLIYSTSNLASGIPDRFSGSGSGTDFTLTISRLEPEDFAVYYCHQYHRSPYTFGQGTKLEIK

SEQ ID NO: 304 - protein, 5 aa, synthetic construct
SYWMQ

SEQ ID NO: 305 - protein, 17 aa, synthetic construct
TIYPGDGDTTYTQKFQG

SEQ ID NO: 306 - protein, 10 aa, synthetic construct
YDAPGYAMDY

SEQ ID NO: 307 - protein, 11 aa, synthetic construct
RASQDINNYLA

SEQ ID NO: 308 - protein, 7 aa, synthetic construct
YTSTLHP

SEQ ID NO: 309 - protein, 9 aa, synthetic construct
LQYDNLLYT

SEQ ID NO: 310 - protein, 119 aa, synthetic construct
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 311 - protein, 107 aa, synthetic construct
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 312 - protein, 6 aa, synthetic construct
RDFAWN

SEQ ID NO: 313 - protein, 16 aa, synthetic construct
YISYNGNTRYQPSLKS

SEQ ID NO: 314 - protein, 7 aa, synthetic construct
ASRGFPY

SEQ ID NO: 315 - protein, 11 aa, synthetic construct
HSSQDINSNIG

SEQ ID NO: 316 - protein, 7 aa, synthetic construct
HGTNLDD

SEQ ID NO: 317 - protein, 9 aa, synthetic construct
VQYAQFPWT

SEQ ID NO: 318 - protein, 116 aa, synthetic construct
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 319 - protein, 107 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 320 - protein, 6 aa, synthetic construct
RDFAWN

SEQ ID NO: 321 - protein, 16 aa, synthetic construct
YISYNGNTRYQPSLKS

SEQ ID NO: 322 - protein, 7 aa, synthetic construct
ASRGFPY

SEQ ID NO: 323 - protein, 11 aa, synthetic construct
HSSQDINSNIG

SEQ ID NO: 324 - protein, 7 aa, synthetic construct
HGTNLDD

SEQ ID NO: 325 - protein, 9 aa, synthetic construct
VQYAQFPWT

SEQ ID NO: 326 - protein, 116 aa, synthetic construct; identical to SEQ ID NO: 318

SEQ ID NO: 327 - protein, 107 aa, synthetic construct; identical to SEQ ID NO: 319

SEQ ID NO: 328 - protein, 5 aa, synthetic construct
NYGVH

SEQ ID NO: 329 - protein, 16 aa, synthetic construct
VIWSGGNTDYNTPFTS

SEQ ID NO: 330 - protein, 11 aa, synthetic construct
ALTYYDYEFAY

SEQ ID NO: 331 - protein, 11 aa, synthetic construct
RASQSIGTNIH

SEQ ID NO: 332 - protein, 7 aa, synthetic construct
YASESIS

SEQ ID NO: 333 - protein, 9 aa, synthetic construct
QQNNNWPTT

SEQ ID NO: 334 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 335 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 336 - protein, 5 aa, synthetic construct
GYFMN

SEQ ID NO: 337 - protein, 17 aa, synthetic construct
RIHPYDGDTFYNQKFQG

SEQ ID NO: 338 - protein, 9 aa, synthetic construct
YDGSRAMDY

SEQ ID NO: 339 - protein, 15 aa, synthetic construct
KASQSVSFAGTSLMH

SEQ ID NO: 340 - protein, 7 aa, synthetic construct
RASNLEA

SEQ ID NO: 341 - protein, 9 aa, synthetic construct
QQSREYPYT

SEQ ID NO: 342 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 343 - protein, 111 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 344 - protein, 5 aa, synthetic construct
GYGLS

SEQ ID NO: 345 - protein, 17 aa, synthetic construct
MISSGGSYTYYADSVKG

SEQ ID NO: 346 - protein, 10 aa, synthetic construct
HGDDPAWFAY

SEQ ID NO: 347 - protein, 12 aa, synthetic construct
SVSSSISSNNLH

SEQ ID NO: 348 - protein, 7 aa, synthetic construct
GTSNLAS

SEQ ID NO: 349 - protein, 11 aa, synthetic construct
QQWSSYPYMYT

SEQ ID NO: 350 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 351 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 352 - protein, 5 aa, synthetic construct
NYWMN

SEQ ID NO: 353 - protein, 19 aa, synthetic construct
EIRLKSNNYTTHYAESVKG

SEQ ID NO: 354 - protein, 6 aa, synthetic construct
HYYFDY

SEQ ID NO: 355 - protein, 16 aa, synthetic construct
RSSKSLLHSNGITYFF

SEQ ID NO: 356 - protein, 7 aa, synthetic construct
QMSNLAS

SEQ ID NO: 357 - protein, 9 aa, synthetic construct
AQNLELPPT

SEQ ID NO: 358 - protein, 117 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 359 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 360 - protein, 5 aa, synthetic construct
SYWIG

SEQ ID NO: 361 - protein, 17 aa, synthetic construct
IIDPGDSRTRYSPSFQG

SEQ ID NO: 362 - protein, 11 aa, synthetic construct
GQLYGGTYMDG

SEQ ID NO: 363 - protein, 14 aa, synthetic construct
TGTSSDIGGYNSVS

SEQ ID NO: 364 - protein, 7 aa, synthetic construct
GVNNRPS

SEQ ID NO: 365 - protein, 11 aa, synthetic construct
SSYDIESATPV

SEQ ID NO: 366 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 367 - protein, 111 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 368 - protein, 5 aa, synthetic construct
AYNIH

SEQ ID NO: 369 - protein, 17 aa, synthetic construct
SFDPYDGGSSYNQKFKD

SEQ ID NO: 370 - protein, 7 aa, synthetic construct
GWYYFDY

SEQ ID NO: 371 - protein, 11 aa, synthetic construct
RASKSISKYLA

SEQ ID NO: 372 - protein, 7 aa, synthetic construct
SGSTLQS

SEQ ID NO: 373 - protein, 9 aa, synthetic construct
QQHDESPYT

SEQ ID NO: 374 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 375 - protein, 107 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 376 - protein, 11 aa, synthetic construct
GSIKSGSYYWG

SEQ ID NO: 377 - protein, 16 aa, synthetic construct
NIYYSGSTYYNPSLRS

SEQ ID NO: 378 - protein, 12 aa, synthetic construct
AREGSYPNQFDP

SEQ ID NO: 379 - protein, 11 aa, synthetic construct
RASQSVSSNLA

SEQ ID NO: 380 - protein, 7 aa, synthetic construct
GASTRAT

SEQ ID NO: 381 - protein, 9 aa, synthetic construct
QQYHSFPFT

SEQ ID NO: 382 - protein, 120 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 383 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 384 - protein, 5 aa, synthetic construct
SFGMH

SEQ ID NO: 385 - protein, 17 aa, synthetic construct
YISSDSSAIYYADTVKG

SEQ ID NO: 386 - protein, 13 aa, synthetic construct
GRENIYYGSRLDY

SEQ ID NO: 387 - protein, 11 aa, synthetic construct
KASQNVDTNVA

SEQ ID NO: 388 - protein, 7 aa, synthetic construct
SASYRYS

SEQ ID NO: 389 - protein, 9 aa, synthetic construct
QQYNNYPFT

SEQ ID NO: 390 - protein, 122 aa, synthetic construct
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 391 - protein, 107 aa, synthetic construct
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 392 - protein, 10 aa, synthetic construct
SYWMQWVRQA

SEQ ID NO: 393 - protein, 11 aa, synthetic construct
TIYPGDGDTRY

SEQ ID NO: 394 - protein, 12 aa, synthetic construct
RGIPRLWYFDVM

SEQ ID NO: 395 - protein, 10 aa, synthetic construct
ITCRASQDIS

SEQ ID NO: 396 - protein, 11 aa, synthetic construct
YTSRLHSGVPS

SEQ ID NO: 397 - protein, 12 aa, synthetic construct
QQGNTLPPFTGG

SEQ ID NO: 398 - protein, 122 aa, synthetic construct; identical to SEQ ID NO: 390

SEQ ID NO: 399 - protein, 107 aa, synthetic construct; identical to SEQ ID NO: 391

SEQ ID NO: 400 - protein, 5 aa, synthetic construct
SYGMS

SEQ ID NO: 401 - protein, 17 aa, synthetic construct
TINSGGSNTYYPDSLKG

SEQ ID NO: 402 - protein, 8 aa, synthetic construct
HDGGAMDY

SEQ ID NO: 403 - protein, 11 aa, synthetic construct
RASESIYSYLA

SEQ ID NO: 404 - protein, 7 aa, synthetic construct
NTKTLPE

SEQ ID NO: 405 - protein, 10 aa, synthetic construct
QHHYGTPPWT

SEQ ID NO: 406 - protein, 117 aa, synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 407 - protein, 108 aa, synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 408 - protein, 5 aa, synthetic construct
SFGMH

SEQ ID NO: 409 - protein, 17 aa, synthetic construct
YISSGSGTIYYADTVKG

SEQ ID NO: 410 - protein, 10 aa, synthetic construct
HGYRYEGFDY

SEQ ID NO: 411 - protein, 11 aa, synthetic construct
KASQNVDTNVA

SEQ ID NO: 412 - protein, 7 aa, synthetic construct
SASYRYS

SEQ ID NO: 413 - protein, 9 aa, synthetic construct
QQYNNYPFT

SEQ ID NO: 414 - protein, 119 aa, synthetic construct
EVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 415 - protein, 107 aa, synthetic construct
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIK

SEQ ID NO: 416 - protein, 5 aa, synthetic construct
NYVMH

SEQ ID NO: 417 - protein, 17 aa, synthetic construct
YINPYNDDVKYNEKFKG

SEQ ID NO: 418 - protein, 13 aa, synthetic construct
WGYYGSPLYYFDY

SEQ ID NO: 419 - protein, 10 aa, synthetic construct
RASSRLIYMH

SEQ ID NO: 420 - protein, 7 aa, synthetic construct
ATSNLAS

SEQ ID NO: 421 - protein, 9 aa, synthetic construct
QQWNSNPPT

SEQ ID NO: 422 - protein, 122 aa, synthetic construct
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 423 - protein, 106 aa, synthetic construct
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 424 - protein, 5 aa, synthetic construct
NYVMH

SEQ ID NO: 425 - protein, 17 aa, synthetic construct
YINPYNDDVKYNEKFKG

SEQ ID NO: 426 - protein, 13 aa, synthetic construct
WGYYGSPLYYFDY

SEQ ID NO: 427 - protein, 10 aa, synthetic construct
RASSRLIYMH

SEQ ID NO: 428 - protein, 7 aa, synthetic construct
ATSNLAS

SEQ ID NO: 429 - protein, 9 aa, synthetic construct
QQWNSNPPT

SEQ ID NO: 430 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 431 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 432 - protein, 5 aa, synthetic construct
SYTIH

SEQ ID NO: 433 - protein, 17 aa, synthetic construct
YINPNSRNTDYAQKFQG

SEQ ID NO: 434 - protein, 12 aa, synthetic construct
YSGSTPYWYFDV

SEQ ID NO: 435 - protein, 10 aa, synthetic construct
RASSSVSYMN

SEQ ID NO: 436 - protein, 7 aa, synthetic construct
ATSNLAS

SEQ ID NO: 437 - protein, 9 aa, synthetic construct
QQWSSNPLT

SEQ ID NO: 438 - protein, 121 aa, synthetic construct
EVQLVQSGAEVKKPGSSVKVSCKASGYSFTSYTIHWVRQAPGQGLEWMGYINPNSRNTDYAQKFQGRVTLTADKSTSTAYMELSSLRSEDTAVYYCARYSGSTPYWYFDVWGQGTTVTVSS

SEQ ID NO: 439 - protein, 106 aa, synthetic construct
DIQLTQSPSFLSASVGDRVTITCRASSSVSYMNWYQQKPGKSPKPWIYATSNLASGVPSRFSVSVSGTEHTLTISSLQPEDFATYYCQQWSSNPLTFGQGTKLEIK

SEQ ID NO: 440 - protein, 5 aa, synthetic construct
SYWMH

SEQ ID NO: 441 - protein, 17 aa, synthetic construct
LIHPDSGSTNYNEMFKN

SEQ ID NO: 442 - protein, 7 aa, synthetic construct
GGRLYFD

SEQ ID NO: 443 - protein, 16 aa, synthetic construct
RSSQSLVHSNGDTYLR

SEQ ID NO: 444 - protein, 7 aa, synthetic construct
KVSNRFS

SEQ ID NO: 445 - protein, 9 aa, synthetic construct
SQSTHVPYT

SEQ ID NO: 446 - protein, 117 aa, synthetic construct
EVQLVQSGAEVKKPGSSVKVSCKASGYTFSSYWMHWVRQAPGQGLEWIGLIHPDSGSTNYNEMFKNRATLTVDRSTSTAYVELSSLRSEDTAVYFCAGGGRLYFDYWGQGTTVTVSS

SEQ ID NO: 447 - protein, 112 aa, synthetic construct
DVVMTQSPLSLPVTPGEPASISCRSSQSLVHSNGDTYLRWYLQKPGQSPQLLIYKVSNRFSGVPDRFSGSGSGTDFTLKISRVEAEDVGVYYCSQSTHVPYTFGGGTKVEIK

SEQ ID NO: 448 - protein, 5 aa, synthetic construct
SYWMH

SEQ ID NO: 449 - protein, 17 aa, synthetic construct
LIHPESGSTNYNEMFKN

SEQ ID NO: 450 - protein, 8 aa, synthetic construct
GGRLYFDY

SEQ ID NO: 451 - protein, 16 aa, synthetic construct
RSSQSLVHSNQDTYLR

SEQ ID NO: 452 - protein, 7 aa, synthetic construct
KVSNRFS

SEQ ID NO: 453 - protein, 9 aa, synthetic construct
SQSTHVPYT

SEQ ID NO: 454 - protein, 117 aa, synthetic construct
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 455 - protein, 113 aa, synthetic construct
DIVMTQSPLSLPVTPGEPASISCRSSQSLVHSNQDTYLRWYLQKPGQSPQLLIYKVSNRFSGVPDRFSGSGSGTDFTLKKISRVEAEDVGVYYCSQSTHVPYTFGGGTKVEIK

SEQ ID NO: 456 - protein, 6 aa, synthetic construct
SGYSWH

SEQ ID NO: 457 - protein, 16 aa, synthetic construct
YIHSSGSTNYNPSLKS

SEQ ID NO: 458 - protein, 7 aa, synthetic construct
YDDYFEY

SEQ ID NO: 459 - protein, 11 aa, synthetic construct
KASQNVGFNVA

SEQ ID NO: 460 - protein, 7 aa, synthetic construct
SASYRYS

SEQ ID NO: 461 - protein, 9 aa, synthetic construct
QQYNWYPFT

SEQ ID NO: 462 - protein, 116 aa, synthetic construct; identical to SEQ ID NO: 230

SEQ ID NO: 463 - protein, 107 aa, synthetic construct; identical to SEQ ID NO: 231

SEQ ID NO: 464 - protein, 5 aa, synthetic construct
NYDIN

SEQ ID NO: 465 - protein, 17 aa, synthetic construct
WIFPGDDSTQYNEKFKG

SEQ ID NO: 466 - protein, 9 aa, synthetic construct
QTTGTWFAY

SEQ ID NO: 467 - protein, 11 aa, synthetic construct
RASQSISDYLY

SEQ ID NO: 468 - protein, 7 aa, synthetic construct
YASQSIS

SEQ ID NO: 469 - protein, 9 aa, synthetic construct
QNGHSFPLT

SEQ ID NO: 470 - protein, 118 aa, synthetic construct
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSS

SEQ ID NO: 471 - protein, 107 aa, synthetic construct
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELK

SEQ ID NO: 472 - protein, 119 aa, synthetic construct
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 473 - protein, 109 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 474 - protein, 5 aa, synthetic construct
IYNVH

SEQ ID NO: 475 - protein, 17 aa, synthetic construct
TIFPGNGDTSYNQKFKD

SEQ ID NO: 476 - protein, 10 aa, synthetic construct
WDDGNVGFAH

SEQ ID NO: 477 - protein, 11 aa, synthetic construct
RASENINNYLT

SEQ ID NO: 478 - protein, 7 aa, synthetic construct
HAKTLAE

SEQ ID NO: 479 - protein, 9 aa, synthetic construct
QHHYGTPPT

SEQ ID NO: 480 - protein, 119 aa, synthetic construct
QVQLQQPGAELVKPGASVKMSCKASGYTFTIYNVHWIKQTPGQGLEWMGTIFPGNGDTSYNQKFKDKATLTTDKSSKTAYMQLNSLTSEDSAVYYCARWDDGNVGFAHWGQGTLVTVSA

SEQ ID NO: 481 - protein, 107 aa, synthetic construct
DIQMTQSPASLSASVGETVTITCRASENINNYLTWFQQKQGKSPQLLVYHAKTLAEGVPSRFSGSGSGTQFSLKINSLQPEDFGSYYCQHHYGTPPTFGGGTKLEIK

SEQ ID NO: 482 - protein, 119 aa, synthetic construct
EVQLVQSGAEVKKPGASVKVSCKASGYTFTIYNVHWVRQAPGQGLEWMGTIFPGNGDTSYNQKFKDKVTMTTDTSTSTAYMELSSLRSEDTAVYYCARWDDGNVGFAHWGQGTLVTVSS

SEQ ID NO: 483 - protein, 107 aa, synthetic construct
DIQMTQSPSSLSASVGDRVTITCRASENINNYLTWFQQKQGKSPQLLIYHAKTLAEGVPSRFSGSGSGTDFTLTISSLQPEDFATYYCQHHYGTPPTFGGGTKVEIK

SEQ ID NO: 484 - protein, 119 aa, synthetic construct
EVQLVQSGAEVKKPGASVKVSCKASGYTFTIYNVHWIRQAPGQGLEWMGTIFPGNGDTSYNQKFKDRATLTTDKSTKTAYMELRSLRSDDTAVYYCARWDDGNVGFAHWGQGTLVTVSS

SEQ ID NO: 485 - protein, 107 aa, synthetic construct
DIQMTQSPSSLSASVGDRVTITCRASENINNYLTWFQQKPGKAPKLLVYHAKTLAEGVPSRFSGSGSGTQFTLTISSLQPEDFATYYCQHHYGTPPTFGQGTKLEIK

SEQ ID NO: 486 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 488 - protein, 445 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 489 - protein, 217 aa, synthetic construct
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 490 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 491 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 449 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 7 aa, synthetic construct
TSNMGVG

SEQ ID NO: 495 - protein, 16 aa, synthetic construct
HIWWDDDKYYSPSLKS

SEQ ID NO: 496 - protein, 10 aa, synthetic construct
SNYGYAWFAY

SEQ ID NO: 497 - protein, 11 aa, synthetic construct
KASQDIYPYLN

SEQ ID NO: 498 - protein, 7 aa, synthetic construct
RTNRLLD

SEQ ID NO: 499 - protein, 9 aa, synthetic construct
LQYDEFPLT

SEQ ID NO: 500 - protein, 120 aa, synthetic construct
QITLKESGPTLVKPTQTLTLTCTFSGFSLSTSNMGVGWIRQPPGKALEWLAHIWWDDDKYYSPSLKSRLTITKDTSKNQVVLTMTNMDPVDTATYYCVRSNYGYAWFAYWGQGTLVTVSS

SEQ ID NO: 501 - protein, 107 aa, synthetic construct
DIQMTQSPSSLSASVGDRVTITCKASQDIYPYLNWFQQKPGKAPKTLIYRTNRLLDGVPSRFSGSGSGTDFTFTISSLQPEDIATYYCLQYDEFPLTFGAGTKLEIK

SEQ ID NO: 502 - protein, 5 aa, synthetic construct
DYAVH

SEQ ID NO: 503 - protein, 17 aa, synthetic construct
VISTYNDYTYNNQDFKG

SEQ ID NO: 504 - protein, 10 aa, synthetic construct
GNSYFYALDY

SEQ ID NO: 505 - protein, 15 aa, synthetic construct
RASESVDSYGKSFMH

SEQ ID NO: 506 - protein, 7 aa, synthetic construct
RASNLES

SEQ ID NO: 507 - protein, 9 aa, synthetic construct
QQSNEDPWT

SEQ ID NO: 508 - protein, 119 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 509 - protein, 111 aa, synthetic construct
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 510 - protein, 5 aa, synthetic construct
RYWMS

SEQ ID NO: 511 - protein, 17 aa, synthetic construct
DLNPDSSAINYVDSVKG

SEQ ID NO: 512 - protein, 11 aa, synthetic construct
ITTLVPYTMDF

SEQ ID NO: 513 - protein, 11 aa, synthetic construct
ITNTDIDDDMN

SEQ ID NO: 514 - protein, 7 aa, synthetic construct
EGNGLRP

SEQ ID NO: 515 - protein, 9 aa, synthetic construct
LQSDNLPLT

SEQ ID NO: 516 - protein, 120 aa, synthetic construct
EVQLVESGGGLVQPGGSLRLSCAASGFDFSRYWMSWVRQAPGKGLEWIGDLNPDSSAINYVDSVKGRFTISRDNAKNSLYLQMNSLRAEDTAVYYCTLITTLVPYTMDFWGQGTSVTVSS

SEQ ID NO: 517 - protein, 107 aa, synthetic construct
ETTLTQSPAFMSATPGDKVNISCITNTDIDDDMNWYQQKPGEAAILLISEGNGLRPGIPPRFSGSGYGTDFTLTINNIESEDAAYYFCLQSDNLPLTFGSGTKLEIK

SEQ ID NO: 518 - protein, 5 aa, synthetic construct
DYYMH

SEQ ID NO: 519 - protein, 17 aa, synthetic construct
WIDPENGDTEYGPKFQG

SEQ ID NO: 520 - protein, 11 aa, synthetic construct
HNAHYGTWFAY

SEQ ID NO: 521 - protein, 16 aa, synthetic construct
RSSQSLLHSSGNTYLE

SEQ ID NO: 522 - protein, 7 aa, synthetic construct
KISTRFS

SEQ ID NO: 523 - protein, 9 aa, synthetic construct
FQGSHVPYT

SEQ ID NO: 524 - protein, 120 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 525 - protein, 112 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 526 - protein, 5 aa, synthetic construct
DYNVN

SEQ ID NO: 527 - protein, 17 aa, synthetic construct
VINPKYGTTRYNQKFKG

SEQ ID NO: 528 - protein, 7 aa, synthetic construct
GLNAWDY

SEQ ID NO: 529 - protein, 11 aa, synthetic construct
GASENIYGALN

SEQ ID NO: 530 - protein, 7 aa, synthetic construct
GATNLED

SEQ ID NO: 531 - protein, 9 aa, synthetic construct
QNVLTTPYT

SEQ ID NO: 532 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 533 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 534 - protein, 5 aa, synthetic construct
GYFMN

SEQ ID NO: 535 - protein, 17 aa, synthetic construct
LINPYNGDSFYNQKFKG

SEQ ID NO: 536 - protein, 8 aa, synthetic construct
GLRRDFDY

SEQ ID NO: 537 - protein, 16 aa, synthetic construct
KSSQSLLDSDGKTYLN

SEQ ID NO: 538 - protein, 7 aa, synthetic construct
LVSELDS

SEQ ID NO: 539 - protein, 9 aa, synthetic construct
WQGTHFPRT

SEQ ID NO: 540 - protein, 117 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 541 - protein, 112 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 542 - protein, 5 aa, synthetic construct
DFGMN

SEQ ID NO: 543 - protein, 17 aa, synthetic construct
WINTFTGEPSYGNVFKG

SEQ ID NO: 544 - protein, 10 aa, synthetic construct
RHGNGNVFDS

SEQ ID NO: 545 - protein, 11 aa, synthetic construct
RASQSIGSNIH

SEQ ID NO: 546 - protein, 7 aa, synthetic construct
YTSESIS

SEQ ID NO: 547 - protein, 9 aa, synthetic construct
QQSNSWPLT

SEQ ID NO: 548 - protein, 119 aa, synthetic construct
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 549 - protein, 108 aa, synthetic construct
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKR

SEQ ID NO: 550 - protein, 5 aa, synthetic construct
TAAIS

SEQ ID NO: 551 - protein, 17 aa, synthetic construct
GIIPIFGKAHYAQKFQG

SEQ ID NO: 552 - protein, 14 aa, synthetic construct
KFHFVSGSPFGMDV

SEQ ID NO: 553 - protein, 11 aa, synthetic construct
RASQSVSSYLA

SEQ ID NO: 554 - protein, 7 aa, synthetic construct
DASNRAT

SEQ ID NO: 555 - protein, 8 aa, synthetic construct
QQRSNWPT

SEQ ID NO: 556 - protein, 123 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 557 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 558 - protein, 5 aa, synthetic construct
SYAIS

SEQ ID NO: 559 - protein, 17 aa, synthetic construct
GIIPIFGTANYAQKFQG

SEQ ID NO: 560 - protein, 21 aa, synthetic construct
APLRFLEWSTQDHYYYYYMDV

SEQ ID NO: 561 - protein, 11 aa, synthetic construct
QGDSLRSYYAT

SEQ ID NO: 562 - protein, 7 aa, synthetic construct
GENKRPS

SEQ ID NO: 563 - protein, 11 aa, synthetic construct
KSRDGSGQHLV

SEQ ID NO: 564 - protein, 130 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 565 - protein, 108 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 566 - protein, 5 aa, synthetic construct
SYWIN

SEQ ID NO: 567 - protein, 17 aa, synthetic construct
NIYPSDSYTNYNQKFKD

SEQ ID NO: 568 - protein, 9 aa, synthetic construct
SWRGNSFDY

SEQ ID NO: 569 - protein, 17 aa, synthetic construct
KSSQSLLNSGNQKNYLT

SEQ ID NO: 570 - protein, 7 aa, synthetic construct
WASTRES

SEQ ID NO: 571 - protein, 9 aa, synthetic construct
QNDYSYPFT

SEQ ID NO: 572 - protein, 118 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 573 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 574 - protein, 5 aa, synthetic construct
NYGMN

SEQ ID NO: 575 - protein, 17 aa, synthetic construct
WINTNTGEPTYAEEFKG

SEQ ID NO: 576 - protein, 9 aa, synthetic construct
LGFGNAMDY

SEQ ID NO: 577 - protein, 17 aa, synthetic construct
KSSQSLLNSGNQKNYLT

SEQ ID NO: 578 - protein, 7 aa, synthetic construct
WASTRES

SEQ ID NO: 579 - protein, 9 aa, synthetic construct
QNDYSYPLT

SEQ ID NO: 580 - protein, 118 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 581 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 582 - protein, 5 aa, synthetic construct
SYNMN

SEQ ID NO: 583 - protein, 17 aa, synthetic construct
YISSSSSTIYYADSVKG

SEQ ID NO: 584 - protein, 8 aa, synthetic construct
AYYYGMDV

SEQ ID NO: 585 - protein, 11 aa, synthetic construct
RASQGISGWLA

SEQ ID NO: 586 - protein, 7 aa, synthetic construct
AASTLQS

SEQ ID NO: 587 - protein, 9 aa, synthetic construct
QQANSFPPT

SEQ ID NO: 588 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 589 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 590 - protein, 5 aa, synthetic construct
SYGMH

SEQ ID NO: 591 - protein, 17 aa, synthetic construct
VIWYDGSNQYYADSVKG

SEQ ID NO: 592 - protein, 11 aa, synthetic construct
GLTSGRYGMDV

SEQ ID NO: 593 - protein, 16 aa, synthetic construct
RSSQSLLLSHGFNYLD

SEQ ID NO: 594 - protein, 7 aa, synthetic construct
LGSSRAS

SEQ ID NO: 595 - protein, 9 aa, synthetic construct
MQPLQIPWT

SEQ ID NO: 596 - protein, 120 aa, synthetic construct
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 597 - protein, 112 aa, synthetic construct
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 598 - protein, 5 aa, synthetic construct
SGYWN

SEQ ID NO: 599 - protein, 16 aa, synthetic construct
YISDSGITYYNPSLKS

SEQ ID NO: 600 - protein, 11 aa, synthetic construct
RTLATYYAMDY

SEQ ID NO: 601 - protein, 16 aa, synthetic construct
RASQSLVHSDGNTYLH

SEQ ID NO: 602 - protein, 7 aa, synthetic construct
RVSNRFS

SEQ ID NO: 603 - protein, 9 aa, synthetic construct
SQSTHVPPT

SEQ ID NO: 604 - protein, 119 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 605 - protein, 113 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKR

SEQ ID NO: 606 - protein, 5 aa, synthetic construct
NYAMS

SEQ ID NO: 607 - protein, 17 aa, synthetic construct
SISGSGDYTYYTDSVKG

SEQ ID NO: 608 - protein, 9 aa, synthetic construct
SPWGYYLDS

SEQ ID NO: 609 - protein, 11 aa, synthetic construct
RASQGISSRLA

SEQ ID NO: 610 - protein, 7 aa, synthetic construct
AASSLQS

SEQ ID NO: 611 - protein, 9 aa, synthetic construct
QQYNSYPYT

SEQ ID NO: 612 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 613 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 614 - protein, 5 aa, synthetic construct
DHAIH

SEQ ID NO: 615 - protein, 17 aa, synthetic construct
YFSPGNDDIKYNEKFRG

SEQ ID NO: 616 - protein, 6 aa, synthetic construct
SLSTPY

SEQ ID NO: 617 - protein, 17 aa, synthetic construct
KSSQSLLNRGNHKNYLT

SEQ ID NO: 618 - protein, 7 aa, synthetic construct
WASTRES

SEQ ID NO: 619 - protein, 9 aa, synthetic construct
QNDYTYPYT

SEQ ID NO: 620 - protein, 115 aa, synthetic construct
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 621 - protein, 113 aa, synthetic construct
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 622 - protein, 5 aa, synthetic construct
SYNMH

SEQ ID NO: 623 - protein, 17 aa, synthetic construct
AIYPGNGDTSYNQKFKG

SEQ ID NO: 624 - protein, 12 aa, synthetic construct
STYYGGDWYFNV

SEQ ID NO: 625 - protein, 10 aa, synthetic construct
RASSSVSYIH

SEQ ID NO: 626 - protein, 7 aa, synthetic construct
ATSNLAS

SEQ ID NO: 627 - protein, 9 aa, synthetic construct
QQWTSNPPT

SEQ ID NO: 628 - protein, 121 aa, synthetic construct [sequence omitted]

SEQ ID NO: 629 - protein, 106 aa, synthetic construct [sequence omitted]

SEQ ID NO: 630 - protein, 5 aa, synthetic construct
DTYIH

SEQ ID NO: 631 - protein, 17 aa, synthetic construct
RIYPTNGYTRYADSVKG

SEQ ID NO: 632 - protein, 11 aa, synthetic construct
WGGDGFYAMDY

SEQ ID NO: 633 - protein, 11 aa, synthetic construct
RASQDVNTAVA

SEQ ID NO: 634 - protein, 7 aa, synthetic construct
SASFLYS

SEQ ID NO: 635 - protein, 9 aa, synthetic construct
QQHYTTPPT

SEQ ID NO: 636 - protein, 120 aa, synthetic construct [sequence omitted]

SEQ ID NO: 637 - protein, 107 aa, synthetic construct [sequence omitted]

SEQ ID NO: 638 - protein, 5 aa, synthetic construct
SYWIE

SEQ ID NO: 639 - protein, 17 aa, synthetic construct
EILPGGGDTNYNEIFKG

SEQ ID NO: 640 - protein, 8 aa, synthetic construct
RVPIRLDY

SEQ ID NO: 641 - protein, 15 aa, synthetic construct
KASQSVDYEGDSFLN

SEQ ID NO: 642 - protein, 7 aa, synthetic construct
AASNLES

SEQ ID NO: 643 - protein, 9 aa, synthetic construct
QQSNEDPLT

SEQ ID NO: 644 - protein, 117 aa, synthetic construct [sequence omitted]

SEQ ID NO: 645 - protein, 111 aa, synthetic construct [sequence omitted]

SEQ ID NO: 646 - protein, 5 aa, synthetic construct
DFAMS

SEQ ID NO: 647 - protein, 17 aa, synthetic construct
TIGRVAFHTYYPDSMKG

SEQ ID NO: 648 - protein, 11 aa, synthetic construct
HRGFDVGHFDF

SEQ ID NO: 649 - protein, 16 aa, synthetic construct
RSSETLVHSSGNTYLE

SEQ ID NO: 650 - protein, 7 aa, synthetic construct
RVSNRFS

SEQ ID NO: 651 - protein, 9 aa, synthetic construct
FQGSFNPLT

SEQ ID NO: 652 - protein, 120 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 653 - protein, 112 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 654 - protein, 6 aa, synthetic construct
NDYAWN

SEQ ID NO: 655 - protein, 16 aa, synthetic construct
YISYSGYTTYNPSLKS

SEQ ID NO: 656 - protein, 7 aa, synthetic construct
WTSGLDY

SEQ ID NO: 657 - protein, 11 aa, synthetic construct
KASDLIHNWLA

SEQ ID NO: 658 - protein, 7 aa, synthetic construct
GATSLET

SEQ ID NO: 659 - protein, 9 aa, synthetic construct
QQYWTTPFT

SEQ ID NO: 660 - protein, 116 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 661 - protein, 107 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 662 - protein, 6 aa, synthetic construct
SDYAWN

SEQ ID NO: 663 - protein, 16 aa, synthetic construct
YISNSGSTSYNPSLKS

SEQ ID NO: 664 - protein, 15 aa, synthetic construct
ERNYDYDDYYYAMDY

SEQ ID NO: 665 - protein, 17 aa, synthetic construct
KSSQSLLYRSNQKNYLA

SEQ ID NO: 666 - protein, 7 aa, synthetic construct
WASTRES

SEQ ID NO: 667 - protein, 9 aa, synthetic construct
QQYYNYPRT

SEQ ID NO: 668 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 669 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 670 - protein, 5 aa, synthetic construct
NYWMH

SEQ ID NO: 671 - protein, 17 aa, synthetic construct
ATYRGHSDTYYNQKFKG

SEQ ID NO: 672 - protein, 12 aa, synthetic construct
GAIYDGYDVLDN

SEQ ID NO: 673 - protein, 11 aa, synthetic construct
SASQDISNYLN

SEQ ID NO: 674 - protein, 7 aa, synthetic construct
YTSNLHS

SEQ ID NO: 675 - protein, 9 aa, synthetic construct
QQYRKLPWT

SEQ ID NO: 676 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 677 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 678 - protein, 5 aa, synthetic construct
AYTMH

SEQ ID NO: 679 - protein, 17 aa, synthetic construct
WIKPNNGLANYAQKFQG

SEQ ID NO: 680 - protein, 9 aa, synthetic construct
SEITTEFDY

SEQ ID NO: 681 - protein, 15 aa, synthetic construct
KSSESVDSYANSFLH

SEQ ID NO: 682 - protein, 7 aa, synthetic construct
RASTRES

SEQ ID NO: 683 - protein, 9 aa, synthetic construct
QQSKEDPLT

SEQ ID NO: 684 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 685 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 686 - protein, 6 aa, synthetic construct
SDFAWN

SEQ ID NO: 687 - protein, 16 aa, synthetic construct
YISYSGNTRYQPSLKS

SEQ ID NO: 688 - protein, 7 aa, synthetic construct
AGRGFPY

SEQ ID NO: 689 - protein, 11 aa, synthetic construct
HSSQDINSNIG

SEQ ID NO: 690 - protein, 7 aa, synthetic construct
HGTNLDD

SEQ ID NO: 691 - protein, 9 aa, synthetic construct
VQYAQFPWT

SEQ ID NO: 692 - protein, 116 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 693 - protein, 107 aa, synthetic construct; identical to SEQ ID NO: 319

SEQ ID NO: 694 - protein, 5 aa, synthetic construct
DYYMA

SEQ ID NO: 695 - protein, 17 aa, synthetic construct
SINYDGSSTYYVDSVKG

SEQ ID NO: 696 - protein, 8 aa, synthetic construct
DRGYYFDY

SEQ ID NO: 697 - protein, 16 aa, synthetic construct
RSSQSLVHSNGNTYLH

SEQ ID NO: 698 - protein, 7 aa, synthetic construct
KVSNRFS

SEQ ID NO: 699 - protein, 10 aa, synthetic construct
SQSTHVPPFT

SEQ ID NO: 700 - protein, 117 aa, synthetic construct
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 701 - protein, 113 aa, synthetic construct
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 702 - protein, 5 aa, synthetic construct
SYGMH

SEQ ID NO: 703 - protein, 17 aa, synthetic construct
VIWYDGSNQYYADSVKG

SEQ ID NO: 704 - protein, 11 aa, synthetic construct
GLTSGRYGMDV

SEQ ID NO: 705 - protein, 16 aa, synthetic construct
RSSQSLLLSHGFNYLD

SEQ ID NO: 706 - protein, 7 aa, synthetic construct
LGSSRAS

SEQ ID NO: 707 - protein, 9 aa, synthetic construct
MQPLQIPWT

SEQ ID NO: 708 - protein, 120 aa, synthetic construct; identical to SEQ ID NO: 596

SEQ ID NO: 709 - protein, 112 aa, synthetic construct; identical to SEQ ID NO: 597

SEQ ID NO: 710 - protein, 5 aa, synthetic construct
NAWMS

SEQ ID NO: 711 - protein, 17 aa, synthetic construct
YISSSGSTIYYADSVKG

SEQ ID NO: 712 - protein, 8 aa, synthetic construct
EGLWAFDY

SEQ ID NO: 713 - protein, 14 aa, synthetic construct
TGSSSNIGAGYVVH

SEQ ID NO: 714 - protein, 7 aa, synthetic construct
DNNKRPS

SEQ ID NO: 715 - protein, 11 aa, synthetic construct
AAWDDRLNGPV

SEQ ID NO: 716 - protein, 117 aa, synthetic construct
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 717 - protein, 111 aa, synthetic construct
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 718 - protein, 5 aa, synthetic construct
GYYWS

SEQ ID NO: 719 - protein, 16 aa, synthetic construct
EINHRGNTNDNPSLKS

SEQ ID NO: 720 - protein, 11 aa, synthetic construct
ERGYTYGNFDH

SEQ ID NO: 721 - protein, 11 aa, synthetic construct
RASQSVSRNLA

SEQ ID NO: 722 - protein, 7 aa, synthetic construct
GASTRAT

SEQ ID NO: 723 - protein, 9 aa, synthetic construct
QQYKTWPRT

SEQ ID NO: 724 - protein, 119 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 725 - protein, 107 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 726 - protein, 5 aa, synthetic construct
SYAMN

SEQ ID NO: 727 - protein, 17 aa, synthetic construct
TTSGSGASTYYADSVKG

SEQ ID NO: 728 - protein, 7 aa, synthetic construct
IWIAFDI

SEQ ID NO: 729 - protein, 12 aa, synthetic construct
RASQSVSSSYLA

SEQ ID NO: 730 - protein, 7 aa, synthetic construct
GASSRAT

SEQ ID NO: 731 - protein, 9 aa, synthetic construct
QQYGSSPYT

SEQ ID NO: 732 - protein, 116 aa, synthetic construct
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 733 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 734 - protein, 7 aa, synthetic construct
SFNYYWS

SEQ ID NO: 735 - protein, 16 aa, synthetic construct
YIYYSGSTYSNPSLKS

SEQ ID NO: 736 - protein, 9 aa, synthetic construct
GYNWNYFDY

SEQ ID NO: 737 - protein, 11 aa, synthetic construct
RASQSVDNNLV

SEQ ID NO: 738 - protein, 7 aa, synthetic construct
GASTRAT

SEQ ID NO: 739 - protein, 10 aa, synthetic construct [sequence omitted]

SEQ ID NO: 740 - protein, 119 aa, synthetic construct [sequence omitted]

SEQ ID NO: 741 - protein, 108 aa, synthetic construct [sequence omitted]

SEQ ID NO: 742 - protein, 5 aa, synthetic construct
TYWMH

SEQ ID NO: 743 - protein, 17 aa, synthetic construct
EIDPSDSYSNYNQKFKD

SEQ ID NO: 744 - protein, 11 aa, synthetic construct
NGGLGPAWFSY

SEQ ID NO: 745 - protein, 11 aa, synthetic construct
KASQYVGTAVA

SEQ ID NO: 746 - protein, 7 aa, synthetic construct
SASNRYT

SEQ ID NO: 747 - protein, 9 aa, synthetic construct
QQYSSYPWT

SEQ ID NO: 748 - protein, 120 aa, synthetic construct
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 749 - protein, 107 aa, synthetic construct
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 750 - protein, 5 aa, synthetic construct
SYAMS

SEQ ID NO: 751 - protein, 17 aa, synthetic construct
AISGSGTSTYYADSVKG

SEQ ID NO: 752 - protein, 13 aa, synthetic construct
VRYNWNHGDWFDP

SEQ ID NO: 753 - protein, 13 aa, synthetic construct
SGSSSNIGNNYVS

SEQ ID NO: 754 - protein, 7 aa, synthetic construct
ENYNRPA

SEQ ID NO: 755 - protein, 11 aa, synthetic construct
SSWDDSLNYWV

SEQ ID NO: 756 - protein, 122 aa, synthetic construct
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 757 - protein, 110 aa, synthetic construct
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 758 - protein, 5 aa, synthetic construct
SYGMS

SEQ ID NO: 759 - protein, 17 aa, synthetic construct
TISSGGSYKYYVDSVKG

SEQ ID NO: 760 - protein, 11 aa, synthetic construct
HPDYDGVWFAY

SEQ ID NO: 761 - protein, 10 aa, synthetic construct
SVSSSVFYVH

SEQ ID NO: 762 - protein, 7 aa, synthetic construct
DTSKLAS

SEQ ID NO: 763 - protein, 9 aa, synthetic construct
QQWNSNPPT

SEQ ID NO: 764 - protein, 120 aa, synthetic construct
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 765 - protein, 107 aa, synthetic construct
DIQMTQSPSSLSASVGDRVTITCSVSSSVFYVHWYQQKPGKAPKLLIYDTSKLASSGVPSRFSGSGSGTDFTFTISSLQPEDIATYYCQQWNSNPPTFGGGTKVEIK

SEQ ID NO: 766 - protein, 5 aa, synthetic construct
SYGMS

SEQ ID NO: 767 - protein, 17 aa, synthetic construct
TISSGGSYTYYPDSVKG

SEQ ID NO: 768 - protein, 11 aa, synthetic construct
HPDYDGVWFAY

SEQ ID NO: 769 - protein, 10 aa, synthetic construct
SVSSSVFYVH

SEQ ID NO: 770 - protein, 7 aa, synthetic construct
DTSKLAS

SEQ ID NO: 771 - protein, 9 aa, synthetic construct
QQWNSNPPT

SEQ ID NO: 772 - protein, 120 aa, synthetic construct
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 773 - protein, 105 aa, synthetic construct
QIVLTQSPAIMASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 774 - protein, 5 aa, synthetic construct
SYYMH

SEQ ID NO: 775 - protein, 17 aa, synthetic construct
IINPSGGSTSYAQKFQG

SEQ ID NO: 776 - protein, 16 aa, synthetic construct
DGVLRYFDWLLDYYYY

SEQ ID NO: 777 - protein, 11 aa, synthetic construct
RASQSVGSYLA

SEQ ID NO: 778 - protein, 7 aa, synthetic construct
DASNRAT

SEQ ID NO: 779 - protein, 8 aa, synthetic construct
QQRANVFT

SEQ ID NO: 780 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 781 - protein, 106 aa, synthetic construct
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 782 - protein, 5 aa, synthetic construct
SYYMH

SEQ ID NO: 783 - protein, 17 aa, synthetic construct
IINPSGGSTSYAQKFQG

SEQ ID NO: 784 - protein, 19 aa, synthetic construct
DAELRHFDHLLDYHYYMDV

SEQ ID NO: 785 - protein, 11 aa, synthetic construct
RASQSVGSYLA

SEQ ID NO: 786 - protein, 7 aa, synthetic construct
DASNRAT

SEQ ID NO: 787 - protein, 8 aa, synthetic construct
QQRAQEFT

SEQ ID NO: 788 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 789 - protein, 106 aa, synthetic construct
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 790 - protein, 120 aa, synthetic construct
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 791 - protein, 107 aa, synthetic construct
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 792 - protein, 120 aa, synthetic construct
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 793 - protein, 107 aa, synthetic construct; identical to SEQ ID NO: 791

SEQ ID NO: 794 - protein, 5 aa, synthetic construct
NYYMA

SEQ ID NO: 795 - protein, 17 aa, synthetic construct
SITKGGGNTYYRDSVKG

SEQ ID NO: 796 - protein, 14 aa, synthetic construct
QVTIAAVSTSYFDS

SEQ ID NO: 797 - protein, 15 aa, synthetic construct
KTNQKVDYYGNSYVY

SEQ ID NO: 798 - protein, 7 aa, synthetic construct
LASNLAS

SEQ ID NO: 799 - protein, 9 aa, synthetic construct
QQSRNLPYT

SEQ ID NO: 800 - protein, 123 aa, synthetic construct
EVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 801 - protein, 110 aa, synthetic construct
DIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELK

SEQ ID NO: 802 - protein, 5 aa, synthetic construct
DYYMK

SEQ ID NO: 803 - protein, 17 aa, synthetic construct
DIIPSNGATFYNQKFKG

SEQ ID NO: 804 - protein, 11 aa, synthetic construct
SHLLRASWFAY

SEQ ID NO: 805 - protein, 17 aa, synthetic construct
KSSQSLLNSGNQKNYLT

SEQ ID NO: 806 - protein, 7 aa, synthetic construct
WASTRES

SEQ ID NO: 807 - protein, 9 aa, synthetic construct
QNDYSYPYT

SEQ ID NO: 808 - protein, 120 aa, synthetic construct
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 809 - protein, 113 aa, synthetic construct
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 810 - protein, 5 aa, synthetic construct
DYEMH

SEQ ID NO: 811 - protein, 17 aa, synthetic construct
GIDPETGGTAYNQKFKG

SEQ ID NO: 812 - protein, 6 aa, synthetic construct
YYSFAY

SEQ ID NO: 813 - protein, 16 aa, synthetic construct
RSSQSIVHSNANTYLQ

SEQ ID NO: 814 - protein, 7 aa, synthetic construct
KVSNRFS

SEQ ID NO: 815 - protein, 9 aa, synthetic construct
FQVSHVPYT

SEQ ID NO: 816 - protein, 115 aa, synthetic construct
EVQLVQSGAEVKKPGATVKISCKVSGYTFTDYEMHWVQQAPGKGLEWMGGIDPETGGTAYNQKFKGRVTLTADKSTDTAYMELSSLRSEDTAVYYCGRYYSFAYWGQGTLVTVSS

SEQ ID NO: 817 - protein, 112 aa, synthetic construct
DVVMTQSPLSLPVTLGQPASISCRSSQSIVHSNANTYLQWFQQRPGQSPRLLIYKVSNRFSGVPDRFSGSGSGTDFTLKISRVEAEDVGVYYCFQVSHVPYTFGQGTKLEIK

SEQ ID NO: 818-833 - skipped (16 entries)

SEQ ID NO: 834 - protein, 5 aa, synthetic construct
TAAIS

SEQ ID NO: 835 - protein, 17 aa, synthetic construct
GIIPIFGKAHYAQKFQG

SEQ ID NO: 836 - protein, 14 aa, synthetic construct
KFHFVSGSPFGMDV

SEQ ID NO: 837 - protein, 11 aa, synthetic construct
RASQSVSSYLA

SEQ ID NO: 838 - protein, 7 aa, synthetic construct
DASNRAT

SEQ ID NO: 839 - protein, 8 aa, synthetic construct
QQRSNWPT

SEQ ID NO: 840 - protein, 123 aa, synthetic construct; identical to SEQ ID NO: 556

SEQ ID NO: 841 - protein, 106 aa, synthetic construct; identical to SEQ ID NO: 557

SEQ ID NO: 842 - protein, 9 aa, synthetic construct
GTFSSYAIS

SEQ ID NO: 843 - protein, 17 aa, synthetic construct
SIIPIFGTANYAQKFQG

SEQ ID NO: 844 - protein, 16 aa, synthetic construct
GPSEVGAILGYVWFDP

SEQ ID NO: 845 - protein, 16 aa, synthetic construct
RSSQSLLHSNGYNYLD

SEQ ID NO: 846 - protein, 7 aa, synthetic construct
LGSNRAS

SEQ ID NO: 847 - protein, 9 aa, synthetic construct
MQARRIPIT

SEQ ID NO: 848 - protein, 125 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 849 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 850 - protein, 12 aa, synthetic construct
GYTFTDHAIHWV

SEQ ID NO: 851 - protein, 10 aa, synthetic construct
FSPGNDDIKY

SEQ ID NO: 852 - protein, 8 aa, synthetic construct
KRSLSTPY

SEQ ID NO: 853 - protein, 12 aa, synthetic construct
QSLLNRGNHKNY

SEQ ID NO: 854 - protein, 7 aa, synthetic construct
WASTRES

SEQ ID NO: 855 - protein, 9 aa, synthetic construct
QNDYTYPYT

SEQ ID NO: 856 - protein, 115 aa, synthetic construct; identical to SEQ ID NO: 620

SEQ ID NO: 857 - protein, 113 aa, synthetic construct; identical to SEQ ID NO: 621

SEQ ID NO: 858 - protein, 5 aa, synthetic construct
NYDIN

SEQ ID NO: 859 - protein, 17 aa, synthetic construct
WIYPGDGSTKYNEKFKA

SEQ ID NO: 860 - protein, 8 aa, synthetic construct
GYEDAMDY

SEQ ID NO: 861 - protein, 11 aa, synthetic construct
KASQDINSYLS

SEQ ID NO: 862 - protein, 7 aa, synthetic construct
RANRLVD

SEQ ID NO: 863 - protein, 9 aa, synthetic construct
LQYDEFPLT

SEQ ID NO: 864 - protein, 117 aa, synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 865 - protein, 107 aa, synthetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 866 - protein, 5 aa, synthetic construct
NYGMN

SEQ ID NO: 867 - protein, 17 aa, synthetic construct
WINTYSGEPRYADDFKG

SEQ ID NO: 868 - protein, 9 aa, synthetic construct
DYGRWYFDV

SEQ ID NO: 869 - protein, 10 aa, synthetic construct
RASSSVSHMH

SEQ ID NO: 870 - protein, 7 aa, synthetic construct
ATSNLAS

SEQ ID NO: 871 - protein, 9 aa, synthetic construct
QQWSSTPRT

SEQ ID NO: 872 - protein, 118 aa, synthetic construct; identical to SEQ ID NO: 158

SEQ ID NO: 873 - protein, 106 aa, synthetic construct
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIK

SEQ ID NO: 874 - protein, 5 aa, synthetic construct
DYYIH

SEQ ID NO: 875 - protein, 17 aa, synthetic construct
YINPNSGYTNYAQKFQG

SEQ ID NO: 876 - protein, 12 aa, synthetic construct
YMWERVTGFFDF

SEQ ID NO: 877 - protein, 11 aa, synthetic construct
LASEDISDDLA

SEQ ID NO: 878 - protein, 7 aa, synthetic construct
TTSSLQS

SEQ ID NO: 879 - protein, 9 aa, synthetic construct
QQTYKFPPT

SEQ ID NO: 880 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 881 - protein, 107 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 882 - protein, 8 aa, synthetic construct
GFTFSNYA

SEQ ID NO: 883 - protein, 8 aa, synthetic construct
ISGSGDYT

SEQ ID NO: 884 - protein, 11 aa, synthetic construct
ARSPWGYYLDS

SEQ ID NO: 885 - protein, 6 aa, synthetic construct
QGISSR

SEQ ID NO: 886 - skipped

SEQ ID NO: 887 - protein, 9 aa, synthetic construct
QQYNSYPYT

SEQ ID NO: 888 - protein, 118 aa, synthetic construct; identical to SEQ ID NO: 612

SEQ ID NO: 889 - protein, 107 aa, synthetic construct; identical to SEQ ID NO: 613

SEQ ID NO: 890 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 891 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 892 - protein, 107 aa, synthetic construct
GQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 893 - protein, 107 aa, synthetic construct
RTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 894 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 895 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 896 - protein, 447 aa, synthetic construct
QVQLVQSGAEVKKPGASVKVSCKASGYTFTDYYITWVRQAPGQGLEWMGWIYPGSGNTKYNEKFKGRVTMTRDTSISTAYMELSRLRSDDTAVYYCANYGNYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 897 - protein, 218 aa, synthetic construct
DIVMTQSPDSLAVSLGERATINCKASQSVDFDGDSYMNWYQQKPGQPPKLLIYAASNLESGVPDRFSGSGSGTDFTLTISSLQAEDVAVYYCQQSNEDPWTFGQGTKVEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 898 - protein, 117 aa, synthetic construct
QVQLVQSGAEVKKPGASVKVSCKASGYTFTDYYITWVRQAPGQGLEWMGWIYPGSGNTKYNEKFKGRVTMTRDTSISTAYMELSRLRSDDTAVYYCANYGNYWFAYWGQGTLVTVSS

SEQ ID NO: 899 - protein, 111 aa, synthetic construct
DIVMTQSPDSLAVSLGERATINCKASQSVDFDGDSYMNWYQQKPGQPPKLLIYAASNLESGVPDRFSGSGSGTDFTLTISSLQAEDVAVYYCQQSNEDPWTFGQGTKVEIK

SEQ ID NO: 900 - protein, 5 aa, synthetic construct
HYMMA

SEQ ID NO: 901 - protein, 17 aa, synthetic construct
RIGPSGGPTHYADSVKG

SEQ ID NO: 902 - protein, 19 aa, synthetic construct
YDSGYDYVAVAGPAEYFQH

SEQ ID NO: 903 - protein, 11 aa, synthetic construct
RASQSISTWLA

SEQ ID NO: 904 - protein, 7 aa, synthetic construct
KASNLHT

SEQ ID NO: 905 - protein, 9 aa, synthetic construct
QQYNSYSRT

SEQ ID NO: 906 - protein, 128 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 907 - protein, 107 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 908 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 909 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 910 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 911 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 912 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 913 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 914 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 915 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 916 - protein, 214 aa, synthetic construct; identical to SEQ ID NO: 913

SEQ ID NO: 917 - protein, 5 aa, synthetic construct
SGYWN

SEQ ID NO: 918 - protein, 16 aa, synthetic construct
YISDSGITYYNPSLKS

SEQ ID NO: 919 - protein, 11 aa, synthetic construct
RTLATYYAMDY

SEQ ID NO: 920 - protein, 16 aa, synthetic construct
RASQSLVHSDGNTYLH

SEQ ID NO: 921 - protein, 7 aa, synthetic construct
RVSNRFS

SEQ ID NO: 922 - protein, 9 aa, synthetic construct
SQSTHVPPT

SEQ ID NO: 923 - protein, 119 aa, synthetic construct; identical to SEQ ID NO: 604

SEQ ID NO: 924 - protein, 112 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIK

SEQ ID NO: 925 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 926 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 927 - protein, 219 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 928 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 929 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 930 - protein, 219 aa, synthetic construct; identical to SEQ ID NO: 927

SEQ ID NO: 931 - protein, 5 aa, synthetic construct
DYNVN

SEQ ID NO: 932 - protein, 17 aa, synthetic construct
VINPKYGTTRYNQKFKG

SEQ ID NO: 933 - protein, 7 aa, synthetic construct
GLNAWDY

SEQ ID NO: 934 - protein, 11 aa, synthetic construct
GASENIYGALN

SEQ ID NO: 935 - protein, 7 aa, synthetic construct
GATNLED

SEQ ID NO: 936 - protein, 9 aa, synthetic construct
QNVLTTPYT

SEQ ID NO: 937 - protein, 116 aa, synthetic construct; identical to SEQ ID NO: 532

SEQ ID NO: 938 - protein, 107 aa, synthetic construct; identical to SEQ ID NO: 533

SEQ ID NO: 939 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 940 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 941 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 942 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 943 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 944 - protein, 214 aa, synthetic construct; identical to SEQ ID NO: 941

SEQ ID NO: 945 - protein, 7 aa, synthetic construct
SGSYYWG

SEQ ID NO: 946 - protein, 16 aa, synthetic construct
NIYYSGSTYYNPSLRS

SEQ ID NO: 947 - protein, 10 aa, synthetic construct
EGSYPNQFDP

SEQ ID NO: 948 - protein, 11 aa, synthetic construct
RASQSVSSNLA

SEQ ID NO: 949 - protein, 7 aa, synthetic construct
GASTRAT

SEQ ID NO: 950 - protein, 9 aa, synthetic construct
QQYHSFPFT

SEQ ID NO: 951 - protein, 120 aa, synthetic construct; identical to SEQ ID NO: 382

SEQ ID NO: 952 - protein, 107 aa, synthetic construct; identical to SEQ ID NO: 383

SEQ ID NO: 953 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 954 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 955 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 956 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 957 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 958 - protein, 214 aa, synthetic construct; identical to SEQ ID NO: 955

SEQ ID NO: 959 - protein, 11 aa, synthetic construct
GSISSSSYYWG

SEQ ID NO: 960 - protein, 16 aa, synthetic construct
NIYYSGSTYYNPSLKS

SEQ ID NO: 961 - protein, 12 aa, synthetic construct
AREGSYPNWFDP

SEQ ID NO: 962 - protein, 11 aa, synthetic construct
RASQSVSSNLA

SEQ ID NO: 963 - protein, 7 aa, synthetic construct
GASTRAT

SEQ ID NO: 964 - protein, 9 aa, synthetic construct
QQYHSFPFT

SEQ ID NO: 965 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSS

SEQ ID NO: 966 - protein, 107 aa, synthetic construct; identical to SEQ ID NO: 383

SEQ ID NO: 967 - protein, 11 aa, synthetic construct
GSIKSGSHYWG

SEQ ID NO: 968 - protein, 16 aa, synthetic construct
NIYYSGSTYYNPSLRS

SEQ ID NO: 969 - protein, 12 aa, synthetic construct
AREGSYPNWFDP

SEQ ID NO: 970 - protein, 11 aa, synthetic construct
RASQSVSSNLA

SEQ ID NO: 971 - protein, 7 aa, synthetic construct
GASTRAT

SEQ ID NO: 972 - protein, 9 aa, synthetic construct
QQYHSFPFT

SEQ ID NO: 973 - protein, 120 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSS

SEQ ID NO: 974 - protein, 107 aa, synthetic construct; identical to SEQ ID NO: 383

SEQ ID NO: 975 - protein, 11 aa, synthetic construct
GSIKSGSHYWG

SEQ ID NO: 976 - protein, 16 aa, synthetic construct
NIYYSGSTYYNPSLKS

SEQ ID NO: 977 - protein, 12 aa, synthetic construct
AREGSYPNWLDP

SEQ ID NO: 978 - protein, 11 aa, synthetic construct
RASQSVSSNLA

SEQ ID NO: 979 - protein, 7 aa, synthetic construct
GASTRAT

SEQ ID NO: 980 - protein, 9 aa, synthetic construct
QQYHSFPFT

SEQ ID NO: 981 - protein, 120 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSS

SEQ ID NO: 982 - protein, 107 aa, synthetic construct; identical to SEQ ID NO: 383

SEQ ID NO: 983 - protein, 11 aa, synthetic construct
GSIKSGSYYWG

SEQ ID NO: 984 - protein, 16 aa, synthetic construct
NIYYSGSTYYNPSLKS

SEQ ID NO: 985 - protein, 12 aa, synthetic construct
AREGSYPNQFDP

SEQ ID NO: 986 - protein, 11 aa, synthetic construct
RASQSVSSNLA

SEQ ID NO: 987 - protein, 7 aa, synthetic construct
GASTRAT

SEQ ID NO: 988 - protein, 9 aa, synthetic construct
QQYHSFPFT

SEQ ID NO: 989 - protein, 120 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSS

SEQ ID NO: 990 - protein, 107 aa, synthetic construct; identical to SEQ ID NO: 383

SEQ ID NO: 991 - protein, 11 aa, synthetic construct
GSIKSGSHYWG

SEQ ID NO: 992 - protein, 16 aa, synthetic construct
NIYYSGSTYYNPSLKS

SEQ ID NO: 993 - protein, 12 aa, synthetic construct
AREGSYPNWFDP

SEQ ID NO: 994 - protein, 11 aa, synthetic construct
RASQSVSTNLA

SEQ ID NO: 995 - protein, 7 aa, synthetic construct
DASARVT

SEQ ID NO: 996 - protein, 9 aa, synthetic construct
QQYHSFPFT

SEQ ID NO: 997 - protein, 120 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSS

SEQ ID NO: 998 - protein, 107 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIK

SEQ ID NO: 999 - protein, 11 aa, synthetic construct
GSISSSSYYWG

SEQ ID NO: 1000 - protein, 16 aa, synthetic construct
NIYYSGSTYYNPSLKS

SEQ ID NO: 1001 - protein, 12 aa, synthetic construct
AREGSYTTVLNV

SEQ ID NO: 1002 - protein, 12 aa, synthetic construct
RASQSVSSSYLA

SEQ ID NO: 1003 - protein, 7 aa, synthetic construct
GASSRAT

SEQ ID NO: 1004 - protein, 9 aa, synthetic construct
QQAASYPLT

SEQ ID NO: 1005 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 1006 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIK

SEQ ID NO: 1007 - protein, 11 aa, synthetic construct
GSIGRGSYYWG

SEQ ID NO: 1008 - protein, 16 aa, synthetic construct
NIYYSGSTYYNPSLKS

SEQ ID NO: 1009 - protein, 12 aa, synthetic construct
AREGSYTTVLNV

SEQ ID NO: 1010 - protein, 12 aa, synthetic construct
RASQSVASSHLA

SEQ ID NO: 1011 - protein, 7 aa, synthetic construct
DAVSRAT

SEQ ID NO: 1012 - protein, 9 aa, synthetic construct
QQAASYPLT

SEQ ID NO: 1013 - protein, 120 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 1014 - protein, 108 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIK

SEQ ID NO: 1015 - protein, 11 aa, synthetic construct
GSISSGGYYWS

SEQ ID NO: 1016 - protein, 16 aa, synthetic construct
NIYYSGSTYYNPSLKS

SEQ ID NO: 1017 - protein, 13 aa, synthetic construct
ARESSTISADFDL

SEQ ID NO: 1018 - protein, 11 aa, synthetic construct
RASQGISRWLA

SEQ ID NO: 1019 - protein, 7 aa, synthetic construct
AASSLQS

SEQ ID NO: 1020 - protein, 9 aa, synthetic construct
QQAHTFPYT

SEQ ID NO: 1021 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSS

SEQ ID NO: 1022 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIK

SEQ ID NO: 1023 - protein, 11 aa, synthetic construct
GSISHGGYYWS

SEQ ID NO: 1024 - protein, 16 aa, synthetic construct
NIYYSGSTYYNPSLKS

SEQ ID NO: 1025 - protein, 13 aa, synthetic construct
ARESSTISADFDL

SEQ ID NO: 1026 - protein, 11 aa, synthetic construct
RASQGISRWLA

SEQ ID NO: 1027 - protein, 7 aa, synthetic construct
AASSLQS

SEQ ID NO: 1028 - protein, 9 aa, synthetic construct
QQAHTFPYT

SEQ ID NO: 1029 - protein, 121 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSS

SEQ ID NO: 1030 - protein, 107 aa, synthetic construct; identical to SEQ ID NO: 1022

SEQ ID NO: 1031 - protein, 11 aa, synthetic construct
GSISSGGYYWS

SEQ ID NO: 1032 - protein, 16 aa, synthetic construct
NIYYSGSTYYNPSLKS

SEQ ID NO: 1033 - protein, 13 aa, synthetic construct
ARGLSTIDEAFDP

SEQ ID NO: 1034 - protein, 11 aa, synthetic construct
RASQSISSWLA

SEQ ID NO: 1035 - protein, 7 aa, synthetic construct
KASSLES

SEQ ID NO: 1036 - protein, 9 aa, synthetic construct
QQDNSYPYT

SEQ ID NO: 1037 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSS

SEQ ID NO: 1038 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIK

SEQ ID NO: 1039 - protein, 11 aa, synthetic construct
GSISDGSYYWS

SEQ ID NO: 1040 - protein, 16 aa, synthetic construct
NIYYSGSTYYNPSLRS

SEQ ID NO: 1041 - protein, 13 aa, synthetic construct
ARGLSTIDEAFDP

SEQ ID NO: 1042 - protein, 11 aa, synthetic construct
RASQSISSWLA

SEQ ID NO: 1043 - protein, 7 aa, synthetic construct
KASSLES

SEQ ID NO: 1044 - protein, 9 aa, synthetic construct
QQDNSYPYT

SEQ ID NO: 1045 - protein, 121 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 1046 - protein, 107 aa, synthetic construct; identical to SEQ ID NO: 1038

SEQ ID NO: 1047 - protein, 11 aa, synthetic construct
GSISDGSYYWS

SEQ ID NO: 1048 - protein, 16 aa, synthetic construct
NIYYSGSTYYNPSLRS

SEQ ID NO: 1049 - protein, 13 aa, synthetic construct
ARGLSTIDEAFDP

SEQ ID NO: 1050 - protein, 11 aa, synthetic construct
RASKSISSWLA

SEQ ID NO: 1051 - protein, 7 aa, synthetic construct
EASSLHS

SEQ ID NO: 1052 - protein, 9 aa, synthetic construct
QQDNSYPYT

SEQ ID NO: 1053 - protein, 121 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 1054 - protein, 107 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIK

SEQ ID NO: 1055 - protein, 9 aa, synthetic construct
GSISSYYWS

SEQ ID NO: 1056 - protein, 16 aa, synthetic construct
YIYSSGSTNYNPSLKS

SEQ ID NO: 1057 - protein, 15 aa, synthetic construct
ARGSGQYAAPDYGMD

SEQ ID NO: 1058 - protein, 11 aa, synthetic construct
RASQSISSWLA

SEQ ID NO: 1059 - protein, 7 aa, synthetic construct
KASSLES

SEQ ID NO: 1060 - protein, 9 aa, synthetic construct
QQDNSFPFT

SEQ ID NO: 1061 - protein, 122 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSS

SEQ ID NO: 1062 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIK

SEQ ID NO: 1063 - protein, 9 aa, synthetic construct
GSIISYYWG

SEQ ID NO: 1064 - protein, 16 aa, synthetic construct
YIYSSGSTSYNPSLKS

SEQ ID NO: 1065 - protein, 16 aa, synthetic construct
ARGSGLYAAPDYGLDV

SEQ ID NO: 1066 - protein, 11 aa, synthetic construct
RASQSISSWLA

SEQ ID NO: 1067 - protein, 7 aa, synthetic construct
KASSLES

SEQ ID NO: 1068 - protein, 9 aa, synthetic construct
QQDNSFPFT

SEQ ID NO: 1069 - protein, 122 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSS

SEQ ID NO: 1070 - protein, 107 aa, synthetic construct; identical to SEQ ID NO: 1062

SEQ ID NO: 1071 - protein, 9 aa, synthetic construct
FTFSSYAMS

SEQ ID NO: 1072 - protein, 17 aa, synthetic construct
TISGSGGSTYYADSVKG

SEQ ID NO: 1073 - protein, 13 aa, synthetic construct
ARGAGHYDLVGRY

SEQ ID NO: 1074 - protein, 11 aa, synthetic construct
RASQSISSYLN

SEQ ID NO: 1075 - protein, 7 aa, synthetic construct
AASSLQS

SEQ ID NO: 1076 - protein, 9 aa, synthetic construct
QQLYSLPPT

SEQ ID NO: 1077 - protein, 120 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSS

SEQ ID NO: 1078 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIK

SEQ ID NO: 1079 - protein, 9 aa, synthetic construct
FTFSSYAMS

SEQ ID NO: 1080 - protein, 17 aa, synthetic construct
AISGSGGSTYYADSVKG

SEQ ID NO: 1081 - protein, 10 aa, synthetic construct
ARVGFRALNY

SEQ ID NO: 1082 - protein, 11 aa, synthetic construct
RASQDISSWLA

SEQ ID NO: 1083 - protein, 7 aa, synthetic construct
AASSLQS

SEQ ID NO: 1084 - protein, 10 aa, synthetic construct
QQATSYPPWT

SEQ ID NO: 1085 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSS

SEQ ID NO: 1086 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIK

SEQ ID NO: 1087 - protein, 9 aa, synthetic construct
GTFSSYAIS

SEQ ID NO: 1088 - protein, 17 aa, synthetic construct
GIIPIFGTASYAQKFQG

SEQ ID NO: 1089 - protein, 13 aa, synthetic construct
ARQQYDGRRYFGL

SEQ ID NO: 1090 - protein, 11 aa, synthetic construct
RASQSVSSNLA

SEQ ID NO: 1091 - protein, 7 aa, synthetic construct
SASTRAT

SEQ ID NO: 1092 - protein, 9 aa, synthetic construct
QQVNVWPPT

SEQ ID NO: 1093 - protein, 120 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSS

SEQ ID NO: 1094 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIK

SEQ ID NO: 1095 - protein, 9 aa, synthetic construct
GTFSSYAIS

SEQ ID NO: 1096 - protein, 17 aa, synthetic construct
GIIPIFGTANYAQKFQG

SEQ ID NO: 1097 - protein, 9 aa, synthetic construct
ARGGPWFDP

SEQ ID NO: 1098 - protein, 11 aa, synthetic construct
RASQSISSWLA

SEQ ID NO: 1099 - protein, 7 aa, synthetic construct
KASSLES

SEQ ID NO: 1100 - protein, 10 aa, synthetic construct
QQYNSYPPFT

SEQ ID NO: 1101 - protein, 116 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSS

SEQ ID NO: 1102 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIK

SEQ ID NO: 1103 - protein, 9 aa, synthetic construct
FTFSSYAMS

SEQ ID NO: 1104 - protein, 17 aa, synthetic construct
AISGSGGSTSYADSVKG

SEQ ID NO: 1105 - protein, 13 aa, synthetic construct
AKPSLATMLAFDI

SEQ ID NO: 1106 - protein, 11 aa, synthetic construct
RASQSISSWLA

SEQ ID NO: 1107 - protein, 7 aa, synthetic construct
DASSLES

SEQ ID NO: 1108 - protein, 9 aa, synthetic construct
QQSKSYPRT

SEQ ID NO: 1109 - protein, 120 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSS

SEQ ID NO: 1110 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIK

SEQ ID NO: 1111 - protein, 11 aa, synthetic construct
GSISSSVYYWS

SEQ ID NO: 1112 - protein, 16 aa, synthetic construct
SILVSGSTYYNPSLKS

SEQ ID NO: 1113 - protein, 9 aa, synthetic construct
ARAVSFLDV

SEQ ID NO: 1114 - protein, 11 aa, synthetic construct
RASQSISSYLN

SEQ ID NO: 1115 - protein, 7 aa, synthetic construct
GASSLQS

SEQ ID NO: 1116 - protein, 9 aa, synthetic construct
QQSYDPPWT

SEQ ID NO: 1117 - protein, 117 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSS

SEQ ID NO: 1118 - protein, 107 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIK

SEQ ID NO: 1119 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1120 - protein, 214 aa, synthetic construct; identical to SEQ ID NO: 955

SEQ ID NO: 1121 - protein, 450 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1122 - protein, 214 aa, synthetic construct; identical to SEQ ID NO: 955

SEQ ID NO: 1123 - protein, 450 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1124 - protein, 214 aa, synthetic construct; identical to SEQ ID NO: 955

SEQ ID NO: 1125 - protein, 450 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1126 - protein, 214 aa, synthetic construct; identical to SEQ ID NO: 955

SEQ ID NO: 1127 - protein, 450 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1128 - protein, 214 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1129 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1130 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1131 - protein, 450 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1132 - protein, 215 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1133 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1134 - protein, 214 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1135 - protein, 451 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1136 - protein, 214 aa, synthetic construct; identical to SEQ ID NO: 1134

SEQ ID NO: 1137 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1138 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1139 - protein, 451 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1140 - protein, 214 aa, synthetic construct; identical to SEQ ID NO: 1138

SEQ ID NO: 1141 - protein, 451 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1142 - protein, 214 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1143 - protein, 452 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1144 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1145 - protein, 452 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1146 - protein, 214 aa, synthetic construct; identical to SEQ ID NO: 1144

SEQ ID NO: 1147 - protein, 450 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1148 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1149 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1150 - protein, 215 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1151 - protein, 450 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1152 - protein, 214 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1153 - protein, 446 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1154 - protein, 215 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1155 - protein, 450 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1156 - protein, 214 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1157 - protein, 447 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1158 - protein, 214 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1159 - protein, 5 aa, synthetic construct
NYGMN

SEQ ID NO: 1160 - protein, 17 aa, synthetic construct
WINTYTGEPTYADAFKG

SEQ ID NO: 1161 - protein, 9 aa, synthetic construct
DYGDYGMDY

SEQ ID NO: 1162 - protein, 15 aa, synthetic construct
RASKSVSTSGYSFMH

SEQ ID NO: 1163 - protein, 7 aa, synthetic construct
LASNLES

SEQ ID NO: 1164 - protein, 9 aa, synthetic construct
QHSREVPWT

SEQ ID NO: 1165 - protein, 450 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1166 - protein, 213 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1167 - protein, 449 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1168 - protein, 219 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1169 - protein, 449 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1170 - protein, 215 aa, synthetic construct
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1171 - protein, 755 aa, synthetic construct
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 1172 - protein, 8 aa, synthetic construct
KGAHRPEH

SEQ ID NO: 1173 - protein, 4 aa, synthetic construct
GGGG

SEQ ID NO: 1174 - protein, 4 aa, synthetic construct
GVKG

SEQ ID NO: 1175 - protein, 4 aa, synthetic construct
VKGG

SEQ ID NO: 1176 - protein, 4 aa, synthetic construct
GGFG

SEQ ID NO: 1177 - protein, 5 aa, synthetic construct
GGFGG

SEQ ID NO: 1178 - protein, 5 aa, synthetic construct
DYYIH

SEQ ID NO: 1179 - protein, 17 aa, synthetic construct
RVNPDHGDSYYNQKFKD

SEQ ID NO: 1180 - protein, 6 aa, synthetic construct
NYLFDH

SEQ ID NO: 1181 - protein, 11 aa, synthetic construct
KASQDVGTAVA

SEQ ID NO: 1182 - protein, 7 aa, synthetic construct
WASIRHT

SEQ ID NO: 1183 - protein, 7 aa, synthetic construct
HQFATYT

SEQ ID NO: 1184 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 1185 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 1186 - protein, 445 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1187 - protein, 444 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1188 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0209383 A1
(43) Pub. Date: Jul. 23, 2026

(54) SITE-SPECIFIC ENGINEERED CYSTEINE ANTIBODY DRUG CONJUGATES

(71) Applicant: SEAGEN INC., Bothell, WA (US)

(72) Inventors: Jamie Apperson MITCHELL; Robert Patrick LYON, Sammamish, WA (US)

(21) Appl. No.: 19/137,946

(22) PCT Filed: Dec. 12, 2023
(86) PCT No.: PCT/US2023/083664

§ 371 Date: Jun. 11, 2025

Related U.S. Application Data
(60) Provisional application No. 63/387,246, filed on Dec. 13, 2022.

(51) Int. Cl. C07K 16/30; A61K 38/07; A61K 38/08; A61K 47/68; A61P 35/02; C12N 15/63

(52) U.S. Cl. CPC C07K 16/30; A61K 38/07; A61K 38/08; A61K 47/6851; A61P 35/02; C12N 15/63; C07K 2317/21; C07K 2317/24; C07K 2317/31; C07K 2317/565

(57) ABSTRACT

The present application provides compositions and methods relating to antibodies that comprise cysteine engineered residues for conjugating drug moieties, thereby forming antibody drug conjugates (ADCs). The present disclosure also provides methods for decreasing ADC toxicity. The antibodies and ADCs (e.g., cysteine engineered antibodies and cysteine engineered ADCs) are useful for treatment of various cancers.

### CROSS REFERENCE TO RELATED APPLICATIONS

This patent application claims priority benefits of U.S. Provisional Application No. 63/387,246 filed on Dec. 13, 2022, the content of which is incorporated herein by reference in its entirety.

### REFERENCE TO AN ELECTRONIC SEQUENCE LISTING

The content of the electronic sequence listing (761682009740SEQLIST.xml; Size: 1,083,619 bytes; and Date of Creation: Dec. 8, 2023) is herein incorporated by reference in its entirety.

### FIELD OF THE INVENTION

The present invention is in some aspects related to cysteine engineered antibodies, and more particularly compositions comprising cysteine engineered antibodies conjugated to one or more drug moieties (e.g., cysteine engineered antibody drug conjugates (ADCs)) conjugates, and compositions comprising the same. In other aspects, the present invention is related to methods of reducing toxicity of ADCs and methods of treating cancer employing said cysteine engineered antibodies and/or cysteine engineered ADCs.

### BACKGROUND OF THE INVENTION

Antibodies in which selected amino acids have been mutated to cysteine (e.g., cysteine engineered antibodies) are particularly suitable for use in cysteine engineered conjugates (e.g., cysteine engineered antibody drug conjugates (ADCs)) because the cysteine engineered conjugates derived from said cysteine engineered antibodies may have favorable properties including homogeneity, improved pharmacokinetics, stability, and solubility, compared to conjugates that do not comprise amino acids mutated to cysteine. The current repertoire of cysteine engineered antibodies used in cysteine engineered ADCs exhibit these properties. However, these favorable properties also prolong patient exposure to cysteine engineered ADC exposure, which can cause toxicity and/or side effects such as ocular toxicity, bone marrow toxicity, and lung (pulmonary) toxicity. Therefore, clinical applications of cysteine engineered ADCs built from existing engineered cysteine antibodies are limited. The present disclosure addresses this and other needs.

### BRIEF SUMMARY OF THE INVENTION

The present disclosure provides compositions and methods related to cysteine engineered antibodies and cysteine engineered antibody drug conjugates (ADCs) comprising cysteine amino acid substitutions. Importantly, cysteine engineered ADCs of the present invention permit slower antibody clearance and decreased exposure to the conjugated drug moiety(s) (e.g., have reduced stability), thereby reducing toxicity of the cysteine engineered ADC compared to a second cysteine engineered ADC that comprises a drug moiety conjugated to a cysteine amino acid at one or more other positions, when administered to a patient. Such cysteine engineered ADCs are advantageous in clinical settings for use in the treatment of cancer.

Thus, in some aspects, provided herein is a cysteine engineered antibody comprising cysteine amino acid substitutions, wherein: i) the cysteine engineered antibody comprises a heavy chain and a light chain; and, ii) the cysteine amino acid substitutions are selected from the group consisting of: a) position 121 of the light chain and position 375 of the heavy chain; b) position 121 of the light chain and position 400 of the heavy chain; and, c) position 114 of the light chain and position 400 of the heavy chain, wherein the amino acid position numbering in the light chain is according to Kabat numbering and the amino acid position numbering in the heavy chain is according to EU numbering.

In some embodiments, the cysteine engineered antibody is a full-length antibody, a Fab, a Fab′, a (Fab′)₂, an Fv, or a single chain Fv (scFv).

In some embodiments, the cysteine engineered antibody comprises two heavy chain and two light chains, wherein the cysteine amino acid substitutions are selected from the group consisting of: a) position 121 of at least one the light chains and position 375 of at least one of the heavy chains; b) position 121 of at least one of the light chains and position 400 of at least one of the heavy chains; and, c) position 114 of at least one of the light chains and position 400 of each of at least one of the heavy chains, wherein the amino acid position numbering in the light chains are according to Kabat numbering and the amino acid position numbering in the heavy chains are according to EU numbering. In some embodiments, the cysteine amino acid substitutions are selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

In some embodiments, the cysteine amino acid substitutions are of free cysteines.

In some embodiments, the cysteine engineered antibody is chimeric, human, or humanized. In some embodiments, the cysteine engineered antibody is a full-length antibody. In some embodiments, the cysteine engineered antibody is a full-length antibody selected from the group consisting of IgA, IgD, IgE, IgG, and IgM. In some embodiments, the cysteine engineered antibody is monospecific. In some embodiments, the cysteine engineered antibody is multispecific.

In some embodiments, the cysteine engineered antibody specifically binds to B7-H4, ITGB6, CD30, CD40, EpCAM, CEACAM5, and LIV1. In some embodiments, the cysteine engineered antibody is selected from the group consisting of an anti-B7-H4 antibody, an anti-ITGB6 antibody, an anti-CD30 antibody, an anti-CD40 antibody, an anti-EpCAM antibody, an anti-CEACAM5 antibody, and an anti-LIV1 antibody. In some embodiments, the cysteine engineered antibody comprises a heavy chain variable domain (VH) comprising a heavy chain complementarity determining region (CDRH1) comprising the amino acid sequence of any one of SEQ ID NOs: 1, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, 360, 368, 376, 384, 392, 400, 408, 416, 424, 432, 440, 448, 456, 464, 474, 494, 502, 510, 518, 526, 534, 542, 550, 558, 566, 574, 582, 590, 598, 606, 614, 622, 630, 638, 646, 654, 662, 670, 678, 686, 694, 702, 710, 718, 726, 734, 742, 750, 758, 766, 774, 782, 794, 802, 810, 834, 842, 850, 858, 866, 874, and 882, a CDRH2 comprising the amino acid sequence of any one of SEQ ID NOs: 2, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, 337, 345, 353, 361, 369, 377, 385, 393, 401, 409, 417, 425, 433, 441, 449, 457, 465, 475, 495, 503, 511, 519, 527, 535, 543, 551, 559, 567, 575, 583, 591, 599, 607, 615, 623, 631, 639, 647, 655, 663, 671, 679, 687, 695, 703, 711, 719, 727, 735, 743, 751, 759, 767, 775, 783, 795, 803, 811, 835, 843, 851, 859, 867, 875, and 883, a CDRH3 comprising the amino acid sequence DMG or any one of SEQ ID NOs: 3, 18, 26, 34, 42, 50, 58, 66, 74, 82, 90, 98, 114, 122, 130, 138, 146, 154, 162, 170, 178, 186, 194, 202, 210, 218, 226, 234, 242, 250, 258, 266, 274, 282, 290, 298, 306, 314, 322, 330, 338, 346, 354, 362, 370, 378, 386, 394, 402, 410, 418, 426, 434, 442, 450, 458, 466, 476, 496, 504, 512, 520, 528, 536, 544, 552, 560, 568, 576, 584, 592, 600, 608, 616, 624, 632, 640, 648, 656, 664, 672, 680, 688, 696, 704, 712, 720, 728, 736, 744, 752, 760, 768, 776, 784, 796, 804, 812, 836, 844, 852, 860, 868, 876, and 884; and a light chain variable domain (VL) comprising a light chain complementarity determining region (CDRL1) comprising the amino acid sequence of any one of SEQ ID NOs: 4, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, 307, 315, 323, 331, 339, 347, 355, 363, 371, 379, 387, 395, 403, 411, 419, 427, 435, 443, 451, 459, 467, 477, 497, 505, 513, 521, 529, 537, 545, 553, 561, 569, 577, 585, 593, 601, 609, 617, 625, 633, 641, 649, 657, 665, 673, 681, 689, 697, 705, 713, 721, 729, 737, 745, 753, 761, 769, 777, 785, 797, 805, 813, 837, 845, 853, 861, 869, 877, and 885, a CDRL2 comprising the amino acid sequence of AAS or any one of SEQ ID NOs: 5, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, 308, 316, 324, 332, 340, 348, 356, 364, 372, 380, 388, 396, 404, 412, 420, 428, 436, 444, 452, 460, 468, 478, 498, 506, 514, 522, 530, 538, 546, 554, 562, 570, 578, 586, 594, 602, 610, 618, 626, 634, 642, 650, 658, 666, 674, 682, 690, 698, 706, 714, 722, 730, 738, 746, 754, 762, 770, 778, 786, 798, 806, 814, 838, 846, 854, 862, 870, and 878, and a CDRL3 comprising the amino acid sequence of any one of SEQ ID NOs: 6, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333, 341, 349, 357, 365, 373, 381, 389, 397, 405, 413, 421, 429, 437, 445, 453, 461, 469, 479, 499, 507, 515, 523, 531, 539, 547, 555, 563, 571, 579, 587, 595, 603, 611, 619, 627, 635, 643, 651, 659, 667, 675, 683, 691, 699, 707, 715, 723, 731, 739, 747, 755, 763, 771, 779, 787, 799, 807, 815, 839, 847, 855, 863, 871, 879, and 887. In some embodiments, the cysteine engineered antibody comprises a VH comprising an amino acid sequence of any one of SEQ ID NOs: 7, 12, 22, 30, 38, 46, 54, 66, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, 310, 318, 326, 334, 342, 350, 358, 366, 374, 382, 390, 398, 406, 414, 422, 430, 438, 446, 454, 462, 470, 472, 480, 482, 484, 500, 508, 516, 524, 532, 540, 548, 556, 564, 572, 580, 588, 596, 604, 612, 620, 628, 636, 644, 652, 660, 668, 676, 684, 692, 700, 708, 716, 724, 732, 740, 748, 756, 764, 772, 780, 788, 800, 808, 816, 840, 848, 856, 864, 872, 880, 888, and 898, and a VL comprising the amino acid sequence of any one of SEQ ID NOs: 8, 13, 23, 31, 39, 47, 55, 67, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, 359, 367, 375, 383, 391, 399, 407, 415, 423, 431, 439, 447, 455, 463, 471, 473, 481, 483, 485, 501, 509, 517, 525, 533, 541, 549, 557, 565, 573, 581, 589, 597, 605, 613, 621, 629, 637, 645, 653, 661, 669, 677, 685, 693, 701, 709, 717, 725, 733, 741, 749, 757, 765, 773, 781, 789, 801, 809, 817, 841, 849, 857, 865, 873, 881, 889, and 899.

In other aspects, provided herein is a cysteine engineered antibody drug conjugate (ADC) comprising the cysteine engineered antibody of any one of the preceding embodiments conjugated to a drug moiety. In some embodiments, the drug moiety is selected from the group consisting of a cytotoxic agent, growth inhibitory agent, and a chemotherapy agent. In some embodiments, the cysteine engineered antibody is conjugated to a cytotoxic agent.

In some embodiments, the cysteine engineered antibody is conjugated to the drug moiety via a linker moiety attached to a cysteine amino acid substitution. In some embodiments, the cysteine engineered ADC comprises a drug moiety each conjugated to each individual cysteine amino acid substitution. In some embodiments, each of the drug moieties are the same drug moiety. In some embodiments, the drug moiety is a microtubule inhibitor.

In some embodiments, the drug moiety is a microtubule inhibitor, optionally wherein the microtubule inhibitor is an auristatin or a tubulysin. In some embodiments, the drug moiety is selected from the group consisting of auristatin T, tubulysin M, tubulysin OEt, MMAE (monomethyl auristatin E), and MMAF (monomethyl auristatin F). In some embodiments, the drug moiety is MMAE.

In some embodiments, the cysteine engineered ADC has a drug antibody ratio (DAR) of about 3.5 to about 4.

In other aspects, provided herein is a nucleic acid encoding the cysteine engineered antibody of any one of the preceding embodiments.

In other aspects, provided herein is a vector comprising the nucleic acid of the preceding embodiment.

In other aspects, provided herein is a host cell comprising the nucleic acid of the preceding embodiment or the vector of the preceding embodiment.

In other aspects, provided herein is a pharmaceutical composition comprising the cysteine engineered antibody of any one of the preceding embodiments or the cysteine engineered ADC of any one of the preceding embodiments, and a pharmaceutically acceptable carrier.

In other aspects, provided herein is a method of reducing toxicity of an ADC comprising an antibody moiety conjugated to a drug moiety, the method comprising: generating a cysteine engineered ADC comprising cysteine amino acid substitutions, wherein the cysteine engineered ADC comprises a cysteine engineered antibody comprising a heavy chain and a light chain, and wherein the cysteine amino acid substitutions are selected from the group consisting of: a) position 121 of the light chain and position 375 of the heavy chain; b) position 121 of the light chain and position 400 of the heavy chain; and, c) position 114 of the light chain and position 400 of the heavy chain, wherein the amino acid position numbering in the light chain is according to Kabat numbering and the amino acid position numbering in the heavy chain is according to EU numbering, wherein the drug moiety is conjugated to each of the cysteine amino acid substitutions, thereby reducing toxicity of the cysteine engineered ADC compared to a second cysteine engineered ADC that comprises a drug moiety conjugated to a cysteine amino acid at one or more other positions, when administered to a patient.

In some embodiments, the cysteine engineered antibody moiety is a full-length antibody, a Fab, a Fab′, a (Fab′)₂, an Fv, or a single chain Fv (scFv).

In some embodiments, the cysteine engineered antibody moiety comprises two heavy chain and two light chains, wherein the cysteine amino acid substitutions are selected from the group consisting of: a) position 121 of at least one the light chains and position 375 of at least one of the heavy chains; b) position 121 of at least one of the light chains and position 400 of at least one of the heavy chains; and, c) position 114 of at least one of the light chains and position 400 of each of at least one of the heavy chains, wherein the amino acid position numbering in the light chains are according to Kabat numbering and the amino acid position numbering in the heavy chains are according to EU numbering. In some embodiments, the cysteine amino acid substitutions are selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

In some embodiments, the cysteine amino acid substitutions are selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

In some embodiments, the generating comprises substituting an amino acid in the cysteine engineered antibody with a cysteine amino acid in or inserting a cysteine amino acid into the cysteine engineered antibody.

In some embodiments, ocular toxicity of the cysteine engineered ADC is reduced compared to the second cysteine engineered ADC. In some embodiments, corneal toxicity of the cysteine engineered ADC is reduced compared to the second cysteine engineered ADC. In some embodiments, bone marrow toxicity of the cysteine engineered ADC is reduced compared to the second cysteine engineered ADC. In some embodiments, lung toxicity of the cysteine engineered ADC is reduced compared to the second cysteine engineered ADC.

In some embodiments, the toxicity of cysteine engineered ADC is reduced compared to the second cysteine engineered ADC at about 7 days after the administration.

In some embodiments, the percentage of conjugated drug moiety is reduced for the cysteine engineered ADC compared to a percentage of conjugated drug moiety of the second cysteine engineered ADC at about 7 days after the administration. In some embodiments, the percentage of conjugated drug moiety is reduced at least about 10%, at least about 15%, at least about 20%, at least about 25%, at least about 30%, at least about 35%, at least about 40%, at least about 45%, at least about 50%, at least about 60%, at least about 70%, or at least about 80% for the cysteine engineered ADC compared to the percentage of conjugated drug moiety of the second cysteine engineered ADC.

In some embodiments, the patient has reduced exposure to the drug moiety for the cysteine engineered ADC compared to the exposure produced by the second cysteine engineered ADC.

In other aspects, provided herein is a method of reducing toxicity of an cysteine engineered ADC when administered to a patient, wherein the cysteine engineered ADC comprises a cysteine engineered antibody and a drug moiety, the method comprising:

conjugating the drug moiety to a cysteine amino acid of the cysteine engineered antibody via a linker moiety, wherein the cysteine amino acid is at a position that reduces the stability of the cysteine engineered ADC compared to a second cysteine engineered ADC that comprises a drug moiety conjugated to a cysteine amino acid of a cysteine engineered antibody at one or more other positions.

In some embodiments, the cysteine amino acid is in a heavy chain of the antibody moiety. In some embodiments, the cysteine amino acid is in a light chain of the antibody moiety. In some embodiments, the drug moiety is conjugated to each cysteine amino acid substitution of the cysteine engineered antibody. In some embodiments, i) the cysteine engineered antibody comprises a heavy chain and a light chain; and, ii) the cysteine amino acid substitutions are selected from the group consisting of: a) position 121 of the light chain and position 375 of the heavy chain; b) position 121 of the light chain and position 400 of the heavy chain; and, c) position 114 of the light chain and position 400 of the heavy chain, wherein the amino acid position numbering in the light chain is according to Kabat numbering and the amino acid position numbering in the heavy chain is according to EU numbering. In some embodiments, the cysteine engineered antibody is a full-length antibody, a Fab, a Fab′, a (Fab′)₂, an Fv, or a single chain Fv (scFv). In some embodiments, the cysteine engineered antibody comprises two heavy chain and two light chains, wherein the cysteine amino acid substitutions are selected from the group consisting of: a) position 121 of at least one the light chains and position 375 of at least one of the heavy chains; b) position 121 of at least one of the light chains and position 400 of at least one of the heavy chains; and, c) position 114 of at least one of the light chains and position 400 of each of at least one of the heavy chains, wherein the amino acid position numbering in the light chains are according to Kabat numbering and the amino acid position numbering in the heavy chains are according to EU numbering. In some embodiments, the cysteine amino acid substitutions are selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

In other aspects, provided herein is a method of treating cancer in a patient, comprising administering an effective amount of the cysteine engineered ADC of any one of the preceding embodiments or an effective amount of the pharmaceutical composition of any one of the preceding embodiments to the patient.

In some embodiments, the cancer is a solid tumor, leukemia, or hematological cancer.

In some embodiments, one or more side effects in the patient is improved after administration of the cysteine engineered ADC as compared to administration of the second ADC. In some embodiments, the one or more side effects comprise ocular toxicity, corneal toxicity, lung toxicity, and/or bone marrow toxicity. In some embodiments, i) the ocular and/or corneal toxicity is determined based on the number mitotic and/or apoptotic figures in the eye; ii) the lung toxicity is determined based on alveolar macrophage aggregation in the lung; and/or, iii) the bone marrow toxicity is determined based on suppression of blood cell production in the bone marrow.

In other aspects, provided herein is a kit comprising: i) the cysteine engineered antibody of any one of the preceding claims or the cysteine engineered ADC of any one of the preceding claims; and, ii) instructions for using the cysteine engineered antibody or the cysteine engineered ADC.

In some embodiments, the cysteine engineered antibody comprises a heavy chain comprising the amino acid sequence set forth in SEQ ID NO: 896 and a light chain comprising the amino acid sequence set forth in SEQ ID NO:897; a heavy chain comprising the amino acid sequence set forth in SEQ ID NO: 894 and a light chain comprising the amino acid sequence set forth in SEQ ID NO:895; or a heavy chain comprising the amino acid sequence set forth in SEQ ID NO: 890 and a light chain comprising the amino acid sequence set forth in SEQ ID NO:891.

All references disclosed here are incorporated by reference in their entireties.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows in vitro plasma stability profiles of EC4-based antibodies conjugated with maleimidocaproyl-valine-citrulline-p-aminobenzoyloxycarbonyl-monomethyl auristatin E (mc-vc-MMAE) (i.e., 1006) compared to conventional 4-load antibodies. H00 indicates a non-targeting antibody. Numbers in parenthesis indicate the drug-antibody ratio (DAR).

FIG. 2A shows in vivo ADC stability over time of selected stable EC4 combinations. ADCs were intravenously injected into Sprague Dawley rats and blood draws were taken at 5 minutes, 1 hour, 6 hours, 1 day, 2 days, 4 day, 7 days, 14 days, 21 days, 28 days. In vivo DAR was calculated by the ratio between the μM concentrations of antibody-conjugated drug and total antibody concentration (as measured by ELISA). Arrows indicate the day 8 and day 28 timepoints.

FIG. 2B shows in vivo ADC stability over time of selected unstable EC4 combinations. ADCs were intravenously injected into Sprague Dawley rats and blood draws were taken at 5 minutes, 1 hour, 6 hours, 1 day, 2 days, 4 day, 7 days, 14 days, 21 days, 28 days. In vivo DAR was calculated by the ratio between the μM concentrations of antibody-conjugated drug and total antibody concentration (as measured by ELISA). Arrows indicate the day 8 and day 28 timepoints.

FIG. 3A shows the formation of high molecular weight species in plasma over time for non-targeting (h00) EC4-mc-vc-MMAE conjugates and conventional (non-substituted) mc-vc-MMAE ADCs.

FIG. 3B shows the formation of high molecular weight species in plasma over time for anti-CD30 (hAC10) EC4-mc-vc-MMAE conjugates.

FIG. 4A shows the mean tumor volume over time after being treated with antibody-monomethyl auristatin E (MMAE) conjugates (i.e., 1006) in xenograft mouse models implanted with the BxPC-3 cell line. H00 represents untargeted antibody; h2A2 is an anti-ITGB6 antibody. Dose and schedule for all ADCs tested were 1 mg/kg every seven days for 3 times (q7dx3).

FIG. 4B shows the mean tumor volume over time after being treated with various antibody-monomethyl auristatin E (MMAE) conjugates in xenograft mouse models implanted with the HPAFII cell line. Dose and schedule for all ADCs tested were 3 mg/kg every seven days for 3 times (q7dx3).

FIG. 5A shows absolute reticulocyte counts of various EC4-mc-vc-MMAE ADCs compared to conventional conjugate. ADCs were administered to rats intravenously at 15 mg/kg, and blood was analyzed at day 4 post administration.

FIG. 5B shows absolute reticulocyte counts of various EC4-mc-vc-MMAE ADCs compared to conventional conjugate. ADCs were administered to rats intravenously at 15 mg/kg, and blood was analyzed at day 4 post administration.

FIG. 6 shows images of bone marrow tissue stained with H&E highlighting the significant depletion of mononuclear cells in rats treated with native cysteine mc-vc-MMAE conjugates.

FIG. 7 shows a chart depicting the correlation between the stability of EC4-ADC (as assayed by percent drug conjugated to various sites at day 7) and ocular toxicity (as quantified by the number of mitotic and apoptotic figures). Percent conjugated drug was quantified on Day 7, and average corneal counts was identified on day 8 post injection.

FIG. 8A shows the percent of remaining BxPC-3 cells after being treated with different concentrations (ng/mL) of non-targeting (h00) S400C S114C-mc-vc-MMAE ADCs or anti-ITGB6 (h2A2) S400C S114C-mc-vc-MMAE ADCs, compared to conventional (non-substituted) mc-vc-MMAE ADCs. Numbers in parenthesis indicate the drug-antibody ratio (DAR). The horizontal dashed line represents 50% cell viability.

FIG. 8B shows the percent of remaining HPAFII cells after being treated with different concentrations (ng/ml) of non-targeting (h00) S400C S114C-mc-vc-MMAE ADCs or anti-ITGB6 (h2A2) S400C S114C-mc-vc-MMAE ADCs, compared to conventional (non-substituted) mc-vc-MMAE ADCs. Numbers in parenthesis indicate the drug-antibody ratio (DAR). The horizontal dashed line represents 50% cell viability.

FIG. 9 shows in vivo ADC stability over time of selected non-targeting (h00) EC4-mc-vc-MMAE ADCs. Four copies of the mc-vc-MMAE were conjugated to each non-targeting antibody. 15 mg/kg of ADCs were intravenously injected into Sprague Dawley rats and blood draws were taken across seven days for analyses. In vivo DAR was calculated by the ratio between the μM concentrations of antibody-conjugated drug and total antibody concentration (as measured by ELISA). Numbers in parenthesis indicate the drug-antibody ratio (DAR).

FIG. 10 shows absolute reticulocyte counts of selected non-targeting (h00) EC4-mc-vc-MMAE ADCs. ADCs were administered to rats intravenously at 15 mg/kg, and blood was analyzed at Day 5 post-administration. Numbers in parenthesis indicate the drug-antibody ratio (DAR).

FIG. 11A shows absolute corneal counts (mitotic and apoptotic figures) from corneal tissue of Sprague Dawley rats intravenously injected with 15 mg/kg conventional (non-substituted) mc-vc-MMAE ADC, low stability non-targeting (h00) EC4-mc-vc-MMAE ADCs (h00 S375C S121C-mc-vc-MMAE, h00 S400C S121C-mc-vc-MMAE, and h00 $400C S114C-mc-vc-MMAE), or non-targeting (h00) EC4-mc-vc-MMAE ADCs from Table E4. The dashed vertical line separates the low stability non-targeting (h00) EC4-mc-vc-MMAE ADCs and the non-targeting (h00) EC4-mc-vc-MMAE ADCs from Table E4.

Corneal tissue was analyzed at Day 8 post-administration.

FIG. 11B shows a chart depicting the correlation between the stability of EC4-ADCs (as assayed by percent drug conjugated to various sites) and ocular toxicity (as quantified by the number of mitotic and apoptotic figures). Percent conjugated drug was quantified on Day 7, and average corneal counts was identified on Day 8 post-injection. The correlation between the stability of EC4-ADCs and ocular toxicity was calculated for conventional (non-substituted) mc-vc-MMAE ADCs, low stability non-targeting (h00) EC4-mc-vc-MMAE ADCs, and non-targeting (h00) EC4-mc-vc-MMAE ADCs from Table E4.

FIG. 12 shows a graph of the change in percentage of drug-antibody ratio over 7 days in unbuffered rat plasma. A non-targeting antibody (h00) with or without engineered cysteines were conjugated to different drug moieties (mc-MMAF or mc-GlucQ-TubM) with a DAR of about 4. The engineered cysteines included S400C/S114C and S239C/V205C. The ADCs were assessed for maleimide stability in unbuffered Sprague Dawley rat plasma. Numbers in parenthesis indicate the drug-antibody ratio (DAR).

FIG. 13 shows absolute corneal counts (mitotic and apoptotic figures) from corneal tissue of Sprague Dawley rats intravenously injected with 10 mg/kg or 15 mg/kg h00 S400C S114C conjugated with MC-GlucQ-TubM or h00 S239C V205C conjugated with MC-GlucQ-TubM. Corneal tissue was analyzed at Day 8 post-administration.

FIG. 14A shows a graph of the percent change of drug to antibody ratio (DAR) over 7 days in unbuffered rat plasma. Various S400C/S114C antibodies were conjugated with mc-vc-MMAE with a DAR of about 4. h00 is an untargeted antibody; h2A2 is an anti-ITGB6 antibody; cAC10 is a CD30 antibody; Ab4 is a CEACAM5 antibody; and Ab5 is a B7-H4 antibody. Numbers in parenthesis indicate the drug-antibody ratio (DAR).

FIG. 14B shows a graph of the percent change of hydrolyzed maleimide over 7 days in unbuffered rat plasma. Various S400C/S114C antibodies were conjugated with mc-vc-MMAE with a DAR of about 4. h00 is an untargeted antibody; h2A2 is an anti-ITGB6 antibody; cAC10 is a CD30 antibody; Ab4 is a CEACAM5 antibody; and Ab5 is a B7-H4 antibody. Numbers in parenthesis indicate the drug-antibody ratio (DAR).

### DETAILED DESCRIPTION OF THE INVENTION

The present application provides cysteine engineered antibodies comprising cysteine amino acid substitutions, such as four cysteine amino acid substitutions, at particular positions in the light chain and the heavy chain of the antibody. Also provided herein are methods related to the use of the cysteine engineered antibody in the form of a cysteine engineered antibody drug conjugate (ADC). A drug moiety, such as a cytotoxic agent, conjugated to the cysteine engineered antibody exerts a cytotoxic effect on cells that express a target antigen, and may be used to treat various diseases, such as immunological disorders or cancers.

Existing cysteine engineered antibodies typically confer increased stability to a cysteine engineered ADC comprising a drug moiety conjugated to the cysteine engineered antibody at the sites of the cysteine amino acid substitutions, compared to a second cysteine engineered ADC that comprises a drug moiety conjugated to cysteine amino acids at one or more other positions. Efforts to continue optimize cysteine engineered antibodies and cysteine engineered ADC generated therefrom generally focus on faster in vivo tissue uptake and increasing clearance of the drug moiety (e.g., optimizing pharmacokinetic profiles), a rapid clinical response and/or complete regression of a disease state (e.g., a cancer), and/or increasing cysteine engineered ADC stability (e.g., increasing the stability of the conjugation between the drug moiety and the cysteine engineered antibody of the cysteine engineered ADC and/or the overall stability of the cysteine engineered ADC in serum). These cysteine engineered ADCs have potential for use in targeted treatment of immune related diseases or cancers, but also may induce adverse reactions when administered to patients. In many cases, although the clinical efficacy of the cysteine engineered ADC increases as the dosage increases, the degree of side effects such as ocular toxicity and bone marrow toxicity may likewise rise, thereby inducing blindness and/or suppression of the immune system in the patient. Therefore, there is a large unmet need for improved cysteine engineered ADCs.

The present disclosure is based at least in part on the inventor's finding that combinations of particular cysteine amino acid substitutions on the light chain of an antibody and the heavy chain of an antibody confer improved properties when used as sites for conjugation of the cysteine engineered antibody to a drug moiety. The resulting cysteine engineered ADCs retain clinically effectiveness and have decreased toxicity when administered to a patient, but are less stable in plasma compared to other cysteine engineered ADCs comprising drug moieties conjugated to cysteine amino acids at one or more other positions (e.g., other cysteine engineered ADCs having a drug-antibody ratio of about 4). Therefore, in contrast with the previous understanding in the art, the presently claimed cysteine engineered ADCs have decreased stability which results in a corresponding decrease in toxicity.

In particular, the present invention provides a cysteine engineered antibody comprising two heavy chains and two light chains, wherein the cysteine amino acid substitutions are at position 121 (e.g., S121C) or position 114 (e.g., S114C) of each of the light chains, according to the Kabat numbering convention for antibodies, and the cysteine amino acid substitutions are at position 375 (e.g., S375C) or position 400 (e.g., S400C) of each of the heavy chains, according to the EU numbering convention for antibodies. In some embodiments, the cysteine amino acid substitutions are selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

A cysteine engineered ADC according to the present disclosure may comprise a cysteine engineered antibody moiety, such as any one of the cysteine engineered antibody moieties comprising cysteine amino acid substitutions provided herein, that binds to a target antigen, that is conjugated to one or more drug moieties. These target antigens may include highly expressed markers on cancer cell types such as solid tumors, leukemias, hematological cancer cells, and other types of malignancies. The cysteine engineered antibody moiety may recognize the target antigen, and the target cell subsequently uptakes the cysteine engineered ADC. Within the cell, the cysteine engineered ADC linkage to the drug moiety is cleaved and the drug moiety or payload is released within the target cell, thereby treating a disease in a patient. In some embodiments, the drug moiety is a microtubule inhibitor. In some embodiments, the microtubule inhibitor is an auristatin or a tubulysin. In some embodiments, the drug moiety is selected from the group consisting of auristatin T, tubulysin M, tubulysin OEt, MMAE (monomethyl auristatin E), and MMAF (monomethyl auristatin F).

The present application also provides a method of reducing toxicity of an ADC when administered to a patient, wherein the ADC comprises an antibody moiety (e.g., a cysteine engineered antibody, such as any of the cysteine engineered antibodies described herein) conjugated to a drug moiety. In further embodiments, provided herein is a method of treating cancer in a patient, comprising administering an effective amount of a cysteine engineered ADC, such as any of the cysteine engineered ADCs described herein, or an effective amount of a pharmaceutical composition comprising any of the cysteine engineered ADCs described herein, to the patient.

### I. Definitions

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a drug moiety, such as a cytotoxic agent. Typically, ADC bind to a target antigen on a cell surface, followed by internalization of the ADC into the cell and subsequent release of the drug into the cell. In some embodiments, an ADC is a cysteine engineered ADC. In some embodiments, the target antigen is ITGB6, B7-H4, CD30, CD40, EpCAM, CEACAM5, or LIV1.

The term “drug-antibody ratio” or “DAR” used herein is the average number of drug moieties conjugated to each individual cysteine engineered antibody.

The “stability” of a cysteine engineered antibody, cysteine engineered ADC, and/or drug moiety of a cysteine engineered ADC provided herein refers to the respective molecule's ability to remain intact over time. Various measures for stability are described throughout the present disclosure.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by sequence identification number (i.e., SEQ ID NO) includes post-translational modifications of such residues.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′) 2, a Fv fragment, a diabody, a single-chain antibody, an scFv fragment, or an scFv-Fc. Genetically, engineered intact antibodies and fragments such as chimeric antibodies, humanized antibodies, fully human antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen. The antibodies provided herein may be “half antibodies”, comprising a single light chain and a single heavy chain.

The antibodies of the present disclosure include “asymmetric antibodies”. The term “asymmetric antibodies” as used herein refers to an antibody comprising two heavy chain-light chain pairs, wherein a first heavy chain-light chain pair comprises different features compared to the second heavy chain-light chain pair. In the context of cysteine engineered antibodies and cysteine engineered ADCs generated therefrom, different features may include different cysteine amino acid substitutions in a first heavy chain-light chain pair compared to a second heavy chain-light chain pair.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a drug moiety, such as a cytotoxic agent. In some embodiments, the cytotoxic agent can be a cytostatic agent.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “human” antibody or antigen-binding fragment thereof means an antibody or antigen-binding fragment thereof having an amino acid sequence derived from a human immunoglobulin gene locus, where such antibody or antigen-binding fragment is made using techniques known in the art. This definition of a human antibody or antigen-binding fragment thereof includes intact or full-length antibodies and fragments thereof.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12:131-140, 1999; Nguyen et al., EMBO J. 19:921-930, 2000) or from VH domains of other species to produce single-domain antibodies (“dAbs,” see Ward et al., Nature 341:544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present disclosure, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytotoxic agent, and the like, and may be a monomeric or multimeric protein. Examples of molecules comprising an antigen-binding site of an antibody are known in the art and include, for example, Fv, single-chain Fv (scFv), Fab, Fab′, F(ab′) 2, F (ab) c, diabodies, minibodies, nanobodies, Fab-scFv fusions, bispecific (scFv) 4-IgG, and bispecific (scFv) 2-Fab. (See, e.g., Hu et al, Cancer Res. 56:3055-3061, 1996; Atwell et al., Molecular Immunology 33:1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1:11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, MD, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320:415-428, 2002; Iwahashi et al., Mol. Immunol. 36:1079-1091, 1999; Tamura et al, Journal of Immunology, 164:1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein. In some embodiments, a cytotoxic agent is a cytostatic agent.

A “cytostatic agent” refers to a compound that has a cytostatic effect on a cell, thereby mediating inhibition of growth and/or expansion of a specific cell type and/or subset of cells. A “cytostatic effect” refers to the inhibition of cell proliferation.

As used herein, the terms “individual” and “patient” refer to organisms to be treated by the methods of the present disclosure. Such organisms preferably include, but are not limited to, mammals (e.g., murines, simians, equines, bovines, porcines, canines, felines, and the like), and more preferably i OF THE INVENTION ncludes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

“Tumor” as it applies to a subject diagnosed with, or suspected of having, cancer (e.g., solid cancer or breast cancer), refers to a malignant or potentially malignant neoplasm or tissue mass of any size.

As used herein, the term “effective amount” refers to the amount of a compound (e.g., a cysteine engineered antibody or antigen-binding fragment thereof or antibody-drug conjugate) sufficient to effect beneficial or desired results. An effective amount of an antibody or antigen-binding fragment thereof or antibody-drug conjugate (e.g., a cysteine engineered ADC) can be administered in one or more administrations, applications or dosages and is not intended to be limited to a particular formulation or administration route.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which a cysteine engineered antibody is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

### II. Cysteine Engineered Constructs

Provided herein are cysteine engineered constructs, including cysteine engineered antibodies and cysteine engineered antibody drug conjugates (ADCs) generated therefrom.

In some embodiments, the cysteine engineered construct is a cysteine engineered antibody comprising cysteine amino acid substitutions. In some embodiments, the cysteine engineered antibody comprises a heavy chain and a light chain, and comprises a cysteine amino acid substitution on the light chain and a cysteine amino acid substitution on the heavy chain. In some embodiments, the cysteine engineered antibody comprises a cysteine amino acid substitution at position 121 of the light chain, according to Kabat numbering. In some embodiments, the cysteine engineered antibody comprises a cysteine amino acid substitution at position 114 the light chain, according to Kabat numbering. In some embodiments, the cysteine engineered antibody comprises a cysteine amino acid substitution at position 400 of the heavy chain, according to EU numbering. In some embodiments, the cysteine engineered antibody comprises a cysteine amino acid substitution at position 375 of the heavy chain, according to EU numbering. In some embodiments, the cysteine amino acid substitutions are at position 121 of the light chain, according to Kabat numbering, and position 375 of the heavy chain, according to EU numbering. In some embodiments, the cysteine amino acid substitutions are at position 121 of the light chain, according to Kabat numbering, and position 400 of the heavy chain, according to EU numbering. In some embodiments, the cysteine amino acid substitutions are at position 114 of the light chain, according to Kabat numbering, and position 400 of the heavy chain, according to EU numbering. In some embodiments, cysteine engineered antibody is a full-length antibody, a Fab, a Fab′, a (Fab′)₂, an Fv, or a single chain Fv (scFv). In some embodiments, the cysteine engineered antibody comprises two heavy chains and two light chains, and comprises a cysteine amino acid substitution on each of the light chains and a cysteine amino acid substitution on each of the heavy chains. In some embodiments, the cysteine engineered antibody comprises a cysteine amino acid substitution at a different position on each of the light chains and/or a cysteine amino acid substitution at a different position on each of the heavy chains. In some embodiments, the cysteine engineered antibody comprises the same cysteine amino acid substitution on each of the light chains and/or the same cysteine amino acid substitution on each of the heavy chains. In some embodiments, the cysteine engineered antibody comprises a cysteine amino acid substitution at position 121 of each of the light chains, according to Kabat numbering. In some embodiments, the cysteine engineered antibody comprises a cysteine amino acid substitution at position 114 of each of the light chain, according to Kabat numbering. In some embodiments, the cysteine engineered antibody comprises a cysteine amino acid substitution at position 400 of the each of the heavy chains, according to EU numbering. In some embodiments, the cysteine engineered antibody comprises a cysteine amino acid substitution at position 375 of each of the heavy chains, according to EU numbering. In some embodiments, the cysteine engineered antibody comprises the same cysteine amino acid substitution on each of the light chains and the same cysteine amino acid substitution on each of the heavy chains. In some embodiments, the cysteine amino acid substitutions are at position 121 of each of the light chains, according to Kabat numbering, and position 375 of each of the heavy chains, according to EU numbering. In some embodiments, the cysteine amino acid substitutions are at position 121 of each of the light chains, according to Kabat numbering, and position 400 of each of the heavy chains, according to EU numbering. In some embodiments, the cysteine amino acid substitutions are at position 114 of each of the light chains, according to Kabat numbering, and position 400 of each of the heavy chains, according to EU numbering.

In some embodiments, the cysteine engineered construct is a cysteine engineered ADC comprising a cysteine engineered antibody, such as any of the cysteine engineered antibodies described herein, and a drug moiety. In some embodiments, the cysteine engineered antibody comprises a heavy chain and a light chain, and comprises a cysteine amino acid substitution on the light chain and a cysteine amino acid substitution on the heavy chain. In some embodiments, the cysteine engineered antibody comprises a cysteine amino acid substitution at position 121 of the light chain, according to Kabat numbering. In some embodiments, the cysteine engineered antibody comprises a cysteine amino acid substitution at position 114 the light chain, according to Kabat numbering. In some embodiments, the cysteine engineered antibody comprises a cysteine amino acid substitution at position 400 of the heavy chain, according to EU numbering. In some embodiments, the cysteine engineered antibody comprises a cysteine amino acid substitution at position 375 of the heavy chain, according to EU numbering. In some embodiments, the cysteine amino acid substitutions are at position 121 of the light chain, according to Kabat numbering, and position 375 of the heavy chain, according to EU numbering. In some embodiments, the cysteine amino acid substitutions are at position 121 of the light chain, according to Kabat numbering, and position 400 of the heavy chain, according to EU numbering. In some embodiments, the cysteine amino acid substitutions are at position 114 of the light chain, according to Kabat numbering, and position 400 of the heavy chain, according to EU numbering. In some embodiments, cysteine engineered antibody is a full-length antibody, a Fab, a Fab′, a (Fab′)₂, an Fv, or a single chain Fv (scFv). In some embodiments, the cysteine engineered antibody comprises two heavy chains and two light chains, and comprises a cysteine amino acid substitution on each of the light chains and a cysteine amino acid substitution on each of the heavy chains. In some embodiments, the cysteine engineered antibody comprises a cysteine amino acid substitution at a different position on each of the light chains and/or a cysteine amino acid substitution at a different position on each of the heavy chains. In some embodiments, the cysteine engineered antibody comprises the same cysteine amino acid substitution on each of the light chains and/or the same cysteine amino acid substitution on each of the heavy chains. In some embodiments, the cysteine engineered antibody comprises a cysteine amino acid substitution at position 121 of each of the light chains, according to Kabat numbering. In some embodiments, the cysteine engineered antibody comprises a cysteine amino acid substitution at position 114 of each of the light chain, according to Kabat numbering. In some embodiments, the cysteine engineered antibody comprises a cysteine amino acid substitution at position 400 of the each of the heavy chains, according to EU numbering. In some embodiments, the cysteine engineered antibody comprises a cysteine amino acid substitution at position 375 of each of the heavy chains, according to EU numbering. In some embodiments, the cysteine engineered antibody comprises the same cysteine amino acid substitution on each of the light chains and the same cysteine amino acid substitution on each of the heavy chains. In some embodiments, the cysteine amino acid substitutions are at position 121 of each of the light chains, according to Kabat numbering, and position 375 of each of the heavy chains, according to EU numbering. In some embodiments, the cysteine amino acid substitutions are at position 121 of each of the light chains, according to Kabat numbering, and position 400 of each of the heavy chains, according to EU numbering. In some embodiments, the cysteine amino acid substitutions are at position 114 of each of the light chains, according to Kabat numbering, and position 400 of each of the heavy chains, according to EU numbering. In some embodiments, the drug moiety is a cytotoxic agent, such as a microtubule inhibitor. In some embodiments, the microtubule inhibitor is an auristatin or a tubulysin. In some embodiments, the drug moiety is selected from the group consisting of auristatin T, tubulysin M, tubulysin OEt, MMAE (monomethyl auristatin E), and MMAF (monomethyl auristatin F). In some embodiments, the cysteine engineered ADC has a drug antibody ratio (DAR) of about 3.5 to about 4. In some embodiments, the toxicity of the cysteine engineered ADC is reduced compared to a second cysteine engineered ADC that comprises a drug moiety conjugated to a cysteine amino acid at one or more other positions, when administered to a patient. In some embodiments, the stability of the cysteine engineered ADC is reduced compared to a second cysteine engineered ADC that comprises a drug moiety conjugated to a cysteine amino acid at one or more other positions, when administered to a patient.

### A. Cysteine Engineered Antibody

### 1. Antigens and Antibodies

In some aspects, provided herein is a cysteine engineered antibody. A number of suitable antibodies can be employed in cysteine engineered antibodies and cysteine engineered ADCs used in the compositions and methods of the present invention. Antibodies (e.g., cysteine engineered antibodies) of the present invention are useful for a number of applications, including in vitro or in vivo diagnosis, in vivo imaging, and therapy for diseases and conditions associated with various target antigens. Five human antibody classes (IgG, IgA, IgM, IgD and IgE), as well as various subclasses (e.g., IgG1, IgG2, IgG3, IgG4, IgA1 and IgA2) within these classes, are recognized on the basis of structural differences, such as the number of immunoglobulin units in a single antibody molecule, the disulfide bridge structure of the individual units, and differences in chain length and sequence. The class and subclass of an antibody is referred to as the antibody's isotype.

The cysteine engineered antibody can be an intact antibody or an antigen-binding antibody fragment, provided that the antibody fragment contains at least one inter-chain disulfide bond. In some embodiments, the cysteine engineered antibody is a full-length antibody. In some embodiments, the cysteine engineered antibody is a full length antibody selected from the group consisting of IgA, IgD, IgE, IgG, and IgM. In some embodiments, the cysteine engineered antibody is an IgG. In some embodiments, the cysteine engineered antibody is IgG1. In some embodiments, the light chain is a human kappa light chain. In some embodiments, the cysteine engineered antibody is monoclonal.

Typically, the cysteine engineered antibodies are human, rodent (e.g., mouse and rat), donkey, sheep, rabbit, goat, guinea pig, camelid, horse, or chicken. The cysteine engineered antibody can be, for example, a murine, a chimeric, humanized, or fully human antibody produced by techniques well-known to one of skill in the art. In some embodiments, the cysteine engineered antibody is chimeric, human or humanized. In some embodiments, the cysteine engineered antibody is human. In some embodiments, the cysteine engineered antibody is humanized.

Recombinant cysteine engineered antibodies, such as chimeric and humanized monoclonal antibodies, comprising both human and non-human portions which can be made using standard recombinant DNA techniques, may be used as the cysteine engineered antibodies of the present disclosure. A chimeric cysteine engineered antibody is a molecule in which different portions are derived from different animal species, such as those having a variable region derived from a murine monoclonal and human immunoglobulin constant regions. (See, e.g., Cabilly et al., U.S. Pat. No. 4,816,567; and Boss et al., U.S. Pat. No. 4,816,397, which are incorporated herein by reference in their entirety.) Humanized cysteine engineered antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized cysteine engineered monoclonal antibodies can be produced by recombinant DNA techniques known in the art. As used herein, “human” cysteine engineered antibodies include cysteine engineered antibodies having the amino acid sequence of a human immunoglobulin and include antibodies isolated from human immunoglobulin libraries, from human B cells, or from animals transgenic for one or more human immunoglobulin, as described for example in U.S. Pat. Nos. 5,939,598 and 6,111,166, in addition to four free cysteine substitutions.

The antibodies may be monospecific, bispecific, trispecific, or of greater multispecificity. In some embodiments, the cysteine engineered antibody is monospecific. In some embodiments, the cysteine engineered antibody is bispecific. In some embodiments, the cysteine engineered antibody is multispecific.

In some embodiments, the cysteine engineered antibody is directed to a target antigen. The cysteine engineered antibody may be directed against any target antigen of interest, such as of medical and/or therapeutic interest. For example, the antigen can be one associated with pathogens (such as but not limited to viruses, bacteria, fungi, and protozoa), parasites, tumor cells, or particular medical conditions. In the case of a tumor-associated antigen (TAA), the cancer may be of the immune system, lung, colon, rectum, breast, ovary, prostate gland, head, neck, bone, or any other anatomical location. Antigens of interest include, but are not limited to, B7-H4, ITGB6, CD30, CD40, EpCAM, CEACAM5, and LIV1.

Useful antibodies include polyclonal antibodies, which are heterogeneous populations of antibody molecules derived from the sera of immunized animals. Other useful antibodies are monoclonal antibodies, which are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to targeted cells (e.g., cancer cell antigens, viral antigens, or microbial antigens) or other antibodies bound to tumor cells or matrix. In this regard, “functionally active” means that the fragment, derivative or analog is able to immunospecifically binds to target cells. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md; Kabat E et al., 1980, J. Immunology 125 (3): 961-969).

Other useful antibodies include fragments of antibodies such as, but not limited to, F(ab′) 2 fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety). Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety). Such chimeric and humanized monoclonal antibodies can be produced by recombinant DNA techniques known in the art, for example using methods, each of which is specifically incorporated herein by reference, as described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., Science (1988) 240:1041-1043; Liu et al., Proc. Natl. Acad. Sci. (USA) (1987) 84:3439-3443; Liu et al., J. Immunol. (1987) 139:3521-3526; Sun et al. Proc. Natl. Acad. Sci. (USA) (1987) 84:214-218; Nishimura et al. Cancer. Res. (1987) 47:999-1005; Wood et al., Nature (1985) 314:446-449; Shaw et al., J. Natl. Cancer Inst. (1988) 80:1553-1559; Morrison, Science (1985) 229:1202-1207; Oi et al. BioTechniques (1986) 4:214; U.S. Pat. No. 5,225,539; Jones et al., Nature (1986) 321:552-525; Verhoeyan et al., Science (1988) 239:1534; and Beidler et al., J. Immunol. (1988) 141:4053-4060.

Completely human antibodies are particularly preferred and can be produced using transgenic mice that are incapable of expressing endogenous immunoglobulin heavy and light chains genes, but which can express human heavy and light chain genes.

Antibodies include analogs and derivatives that are either modified, i.e., by the covalent attachment of any type of molecule if such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivitization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

In specific embodiments, known antibodies for the treatment of cancer are used. In some embodiments, the antibody will selectively bind to a cancer antigen of a hematological malignancy.

In some embodiments, cysteine engineered antibody is a full-length antibody, a Fab, a Fab′, a (Fab′)₂, an Fv, or a single chain Fv (scFv). In some embodiments, cysteine engineered antibody is a half antibody. In some embodiments, cysteine engineered antibody is a full-length antibody. In some embodiments, cysteine engineered antibody is an asymmetric antibody. In some embodiments, the cysteine engineered antibody comprises at least one heavy chain variable domain (VH, also referred to herein as a “heavy chain”) and at least one light chain variable domain (VL, also referred to herein as a “light chain”). In some embodiments, the heavy chain comprises three heavy chain complementarity determining regions (CDRHs). In some embodiments, the light chain comprises three light chain complementarity determining regions (CDRLs).

Exemplary antigens for the cysteine engineered antibodies are provided below. Exemplary antibodies that bind the indicated antigen are shown in parentheses.

In some embodiments, the antigen (e.g., the antigen for the cysteine engineered antibody) is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab tiuxetan), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SIT1, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAMI, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, Gp100, GPA33, gpNMB (exemplary antibodies include glembatumumab), ICAM1, LICAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLRI (exemplary antibodies include farletuzumab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAM9, TMPRSS11D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab tiuxetan), CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD112, CD155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

In some embodiments, the antigen is CD30. In some embodiments, the antibody (e.g., cysteine engineered antibody) is an antibody or antigen-binding fragment that binds to CD30, such as described in International Patent Publication No. WO 02/43661. In some embodiments, the anti-CD30 antibody is cAC10, which is described in International Patent Publication No. WO 02/43661. cAC10 is also known as brentuximab. In some embodiments, the anti-CD30 antibody comprises the CDRs of cAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9, SEQ ID NO: 10, or SEQ ID NO: 894 and a light chain comprising the amino acid sequence of SEQ ID NO: 11 or SEQ ID NO: 895. In some embodiments, the anti-CD30 antibody is hAC10, In some embodiments, the anti-CD30 antibody comprises the CDRs of hAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 898 and a light chain variable region comprising an amino acid sequence that is at least 95% at least 96%, at least 97%, at last 98%, at least 99%, or 100% identical to the amino acid sequence of SEQ ID NO: 899. In some embodiments, the anti-CD30 antibody comprises a heavy chain comprising the amino acid sequence of SEQ ID NO: 896 and a light chain comprising the amino acid sequence of SEQ ID NO: 897. In some embodiments, the anti-CD-30 antibody comprises a heavy chain comprising the amino acid sequence of SEQ ID NO: 894 and a light chain comprising the amino acid sequence of SEQ ID NO: 895. In some embodiments, the antigen is CD40. In some embodiments, the antibody (e.g., cysteine engineered antibody) is an antibody or antigen-binding fragment that binds to CD40. In some embodiments, the anti-CD40 antibody is hS2C6, which is described in International Patent Publication No. WO 16/069919. In some embodiments, the anti-CD40 antibody comprises the CDRs of hS2C6. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the antigen is CD70. In some embodiments, the antibody (e.g., cysteine engineered antibody) is an antibody or antigen-binding fragment that binds to CD70, such as described in International Patent Publication No. WO 2006/113909. In some embodiments, the antibody is a h1F6 anti-CD70 antibody, which is described in International Patent Publication No. WO 2006/113909. h1F6 is also known as vorsetuzumab. In some embodiments, the anti-CD70 antibody comprises a heavy chain variable region comprising the three CDRs of SEQ ID NO:12 and a light chain variable region comprising the three CDRs of SEQ ID NO:13. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD70 antibody comprises a heavy chain variable region comprising an amino acid sequence that is at least 95%, at least 96%, at least 97%, at last 98%, at least 99%, or 100% identical to the amino acid sequence of SEQ ID NO: 12 and a light chain variable region comprising an amino acid sequence that is at least 95% at least 96%, at least 97%, at last 98%, at least 99%, or 100% identical to the amino acid sequence of SEQ ID NO: 13. In some embodiments, the anti-CD70 antibody comprises a heavy chain comprising the amino acid sequence of SEQ ID NO: 14 and a light chain comprising the amino acid sequence of SEQ ID NO: 15.

In some embodiments, the antigen is interleukin-1 receptor accessory protein (IL1RAP). IL1RAP is a co-receptor of the IL1 receptor (ILIR1) and is required for interleukin-1 (IL1) signaling. IL1 has been implicated in the resistance to certain chemotherapy regimens. IL1RAP is overexpressed in various solid tumors, both on cancer cells and in the tumor microenvironment, but has low expression on normal cells. IL1RAP is also overexpressed in hematopoietic stem and progenitor cells, making it a candidate to target for chronic myeloid leukemia (CML). IL1RAP has also been shown to be overexpressed in acute myeloid leukemia (AML). Antibody binding to IL1RAP could block signal transduction from IL-1 and IL-33 into cells and allow NK-cells to recognize tumor cells and subsequent killing by antibody dependent cellular cytotoxicity (ADCC).

In some embodiments, the antigen is ASCT2. ASCT2 is also known as SLC1A5. ASCT2 is a ubiquitously expressed, broad-specificity, sodium-dependent neutral amino acid exchanger. ASCT2 is involved in glutamine transport. ASCT2 is overexpressed in different cancers and is closely related to poor prognosis, Downregulating ASCT2 has been shown to suppress intracellular glutamine levels and downstream glutamine metabolism, including glutathione production. Due to its high expression in many cancers, ASCT2 is a potential therapeutic target. These effects attenuated growth and proliferation, increased apoptosis and autophagy, and increased oxidative stress and mTORC1 pathway suppression in head and neck squamous cell carcinoma (HNSCC). Additionally, silencing ASCT2 improved the response to cetuximab in HNSCC. In some embodiments, a cysteine engineered antibody provided herein binds to TROP2. In some embodiments, the cysteine engineered antibody (e.g., a cysteine engineered antibody of the cysteine engineered ADC) comprises CDR-H1, CDR-H2, CDR-H3, CDR-L1, CDR-L2, and CDR-L3 comprising the amino acid sequences of SEQ ID NOs: 16, 17, 18, 19, 20, and 21, respectively. In some embodiments, the cysteine engineered antibody comprises a heavy chain variable region comprising the amino acid sequence of SEQ ID NO: 22 and a light chain variable region comprising the amino acid sequence of SEQ ID NO: 23. In some embodiments, the the cysteine engineered antibody is sacituzumab. In some embodiments, the cysteine engineered antibody comprises CDR-H1, CDR-H2, CDR-H3, CDR-L1, CDR-L2, and CDR-L3 comprising the amino acid sequences of SEQ ID NOs: 24, 25, 26, 27, 28, and 29, respectively. In some embodiments, the cysteine engineered antibody comprises a heavy chain variable region comprising the amino acid sequence of SEQ ID NO: 30 and a light chain variable region comprising the amino acid sequence of SEQ ID NO: 31. In some embodiments, the cysteine engineered antibody is datopotamab.

In some embodiments, a cysteine engineered antibody (e.g., the cysteine engineered antibody of the cysteine engineered ADC) provided herein binds to MICA. In some embodiments, the cysteine engineered antibody comprises CDR-H1, CDR-H2, CDR-H3, CDR-L1, CDR-L2, and CDR-L3 comprising the amino acid sequences of SEQ ID NOs: 32, 33, 34, 35, 36, and 37, respectively. In some embodiments, the cysteine engineered antibody comprises a heavy chain variable region comprising the amino acid sequence of SEQ ID NO: 38 and a light chain variable region comprising the amino acid sequence of SEQ ID NO: 39. In some embodiments, the cysteine engineered antibody is h1D5v11 hIgGIK. In some embodiments, the cysteine engineered antibody comprises CDR-H1, CDR-H2, CDR-H3, CDR-L1, CDR-L2, and CDR-L3 comprising the amino acid sequences of SEQ ID NOs: 40, 41, 42, 43, 44, and 45, respectively. In some embodiments, the cysteine engineered antibody comprises a heavy chain variable region comprising the amino acid sequence of SEQ ID NO: 46 and a light chain variable region comprising the amino acid sequence of SEQ ID NO: 47. In some embodiments, the cysteine engineered antibody is MICA.36 hlgG1K G236A. In some embodiments, the cysteine engineered antibody comprises CDR-H1, CDR-H2, CDR-H3, CDR-L1, CDR-L2, and CDR-L3 comprising the amino acid sequences of SEQ ID NOs: 48, 49, 50, 51, 52, and 53, respectively. In some embodiments, the cysteine engineered antibody comprises a heavy chain variable region comprising the amino acid sequence of SEQ ID NO: 54 and a light chain variable region comprising the amino acid sequence of SEQ ID NO: 55. In some embodiments, the cysteine engineered antibody is h3F9 H1L3 hlgG1K. In some embodiments, the cysteine engineered antibody comprises CDR-H1, CDR-H2, CDR-H3, CDR-L1, CDR-L2, and CDR-L3 comprising the amino acid sequences of SEQ ID NOs: 56, 57, 58, 59, 60, and 61, respectively. In some embodiments, the cysteine engineered antibody comprises a heavy chain variable region comprising the amino acid sequence of SEQ ID NO: 62 and a light chain variable region comprising the amino acid sequence of SEQ ID NO: 63. In some embodiments, the cysteine engineered antibody is CM33322 Ab28 hlgG1K.

In some embodiments, an antibody-drug conjugate provided herein binds to CD24. In some embodiments, the cysteine engineered antibody comprises CDR-H1, CDR-H2, CDR-H3, CDR-L1, CDR-L2, and CDR-L3 comprising the amino acid sequences of SEQ ID NOs: 64, 65, 66, 67, 68, and 69, respectively. In some embodiments, the cysteine engineered antibody comprises a heavy chain variable region comprising the amino acid sequence of SEQ ID NO: 70 and a light chain variable region comprising the amino acid sequence of SEQ ID NO: 71. In some embodiments, the cysteine engineered antibody is SWA11.

In some embodiments, a cysteine engineered antibody (e.g., a cysteine engineered antibody of a cysteine engineered ADC) provided herein binds to ITGav. In some embodiments, the cysteine engineered antibody comprises CDR-H1, CDR-H2, CDR-H3, CDR-L1, CDR-L2, and CDR-L3 comprising the amino acid sequences of SEQ ID NOs: 72, 73, 74, 75, 76, and 77, respectively. In some embodiments, the cysteine engineered antibody comprises a heavy chain variable region comprising the amino acid sequence of SEQ ID NO: 78 and a light chain variable region comprising the amino acid sequence of SEQ ID NO: 79. In some embodiments, the cysteine engineered antibody is intetumumab. In some embodiments, the cysteine engineered antibody comprises CDR-H1, CDR-H2, CDR-H3, CDR-L1, CDR-L2, and CDR-L3 comprising the amino acid sequences of SEQ ID NOs: 80, 81, 82, 83, 84, and 85, respectively. In some embodiments, the cysteine engineered antibody comprises a heavy chain variable region comprising the amino acid sequence of SEQ ID NO: 86 and a light chain variable region comprising the amino acid sequence of SEQ ID NO: 87. In some embodiments, the cysteine engineered antibody is abituzumab.

In some embodiments, a cysteine engineered antibody (e.g., a cysteine engineered antibody of a cysteine engineered ADC) provided herein binds to gpA33. In some embodiments, the cysteine engineered antibody comprises CDR-H1, CDR-H2, CDR-H3, CDR-L1, CDR-L2, and CDR-L3 comprising the amino acid sequences of SEQ ID NOs: 88, 89, 90, 91, 92, and 93, respectively. In some embodiments, the cysteine engineered antibody comprises a heavy chain variable region comprising the amino acid sequence of SEQ ID NO: 94 and a light chain variable region comprising the amino acid sequence of SEQ ID NO: 95.

In some embodiments, a cysteine engineered antibody (e.g., a cysteine engineered antibody of a cysteine engineered ADC) provided herein binds to IL1Rap. In some embodiments, the cysteine engineered antibody comprises CDR-H1, CDR-H2, CDR-H3, CDR-L1, CDR-L2, and CDR-L3 comprising the amino acid sequences of SEQ ID NOs: 96, 97, 98, 99, 100, and 101, respectively. In some embodiments, the cysteine engineered antibody comprises a heavy chain variable region comprising the amino acid sequence of SEQ ID NO: 102 and a light chain variable region comprising the amino acid sequence of SEQ ID NO: 103. In some embodiments, the cysteine engineered antibody is nidanilimab.

In some embodiments, a cysteine engineered antibody (e.g., a cysteine engineered antibody of a cysteine engineered ADC) provided herein binds to EpCAM. In some embodiments, the cysteine engineered antibody comprises CDR-H1, CDR-H2, CDR-H3, CDR-L1, CDR-L2, and CDR-L3 comprising the amino acid sequence of DMG or any of SEQ ID NOs: 104, 105, 107, 108, and 109, respectively. In some embodiments, the cysteine engineered antibody comprises a heavy chain variable region comprising the amino acid sequence of SEQ ID NO: 110 and a light chain variable region comprising the amino acid sequence of SEQ ID NO: 111. In some embodiments, the cysteine engineered antibody is adecatumumab. In some embodiments, the cysteine engineered antibody comprises CDR-H1, CDR-H2, CDR-H3, CDR-L1, CDR-L2, and CDR-L3 comprising the amino acid sequences of SEQ ID NOs: 112, 113, 114, 115, 116, and 117, respectively. In some embodiments, the cysteine engineered antibody comprises a heavy chain variable region comprising the amino acid sequence of SEQ ID NO: 118 and a light chain variable region comprising the amino acid sequence of SEQ ID NO: 119. In some embodiments, the cysteine engineered antibody is Ep157305. In some embodiments, the cysteine engineered antibody comprises CDR-H1, CDR-H2, CDR-H3, CDR-L1, CDR-L2, and CDR-L3 comprising the amino acid sequences of SEQ ID NOs: 120, 121, 122, 123, 124, and 125, respectively. In some embodiments, the cysteine engineered antibody comprises a heavy chain variable region comprising the amino acid sequence of SEQ ID NO: 126 and a light chain variable region comprising the amino acid sequence of SEQ ID NO: 127. In some embodiments, the cysteine engineered antibody is Ep3-171. In some embodiments, the cysteine engineered antibody comprises CDR-H1, CDR-H2, CDR-H3, CDR-L1, CDR-L2, and CDR-L3 comprising the amino acid sequences of SEQ ID NOs: 128, 129, 130, 131, 132, and 133, respectively. In some embodiments, the cysteine engineered antibody comprises a heavy chain variable region comprising the amino acid sequence of SEQ ID NO: 134 and a light chain variable region comprising the amino acid sequence of SEQ ID NO: 135. In some embodiments, the cysteine engineered antibody is Ep3622w94. In some embodiments, the cysteine engineered antibody comprises CDR-H1, CDR-H2, CDR-H3, CDR-L1, CDR-L2, and CDR-L3 comprising the amino acid sequences of SEQ ID NOs: 136, 137, 138, 139, 140, and 141, respectively. In some embodiments, the cysteine engineered antibody comprises a heavy chain variable region comprising the amino acid sequence of SEQ ID NO: 142 and a light chain variable region comprising the amino acid sequence of SEQ ID NO: 143. In some embodiments, the cysteine engineered antibody is EpING1. In some embodiments, the cysteine engineered antibody comprises CDR-H1, CDR-H2, CDR-H3, CDR-L1, CDR-L2, and CDR-L3 comprising the amino acid sequences of SEQ ID NOs: 144, 145, 146, 147, 148, and 149, respectively. In some embodiments, the cysteine engineered antibody comprises a heavy chain variable region comprising the amino acid sequence of SEQ ID NO: 150 and a light chain variable region comprising the amino acid sequence of SEQ ID NO: 151. In some embodiments, the cysteine engineered antibody is EpAb2-6.

In some embodiments, a cysteine engineered antibody (e.g., a cysteine engineered antibody of a cysteine engineered ADC) provided herein binds to CD352. In some embodiments, the cysteine engineered antibody comprises CDR-H1, CDR-H2, CDR-H3, CDR-L1, CDR-L2, and CDR-L3 comprising the amino acid sequences of SEQ ID NOs: 152, 153, 154, 155, 156, and 157, respectively. In some embodiments, the cysteine engineered antibody comprises a heavy chain variable region comprising the amino acid sequence of SEQ ID NO: 158 and a light chain variable region comprising the amino acid sequence of SEQ ID NO: 159. In some embodiments, the cysteine engineered antibody is h20F3.

In some embodiments, a cysteine engineered antibody (e.g., a cysteine engineered antibody of a cysteine engineered ADC) provided herein binds to CS1. In some embodiments, the cysteine engineered antibody comprises CDR-H1, CDR-H2, CDR-H3, CDR-L1, CDR-L2, and CDR-L3 comprising the amino acid sequences of SEQ ID NOs: 160, 161, 162, 163, 164, and 165, respectively. In some embodiments, the cysteine engineered antibody comprises a heavy chain variable region comprising the amino acid sequence of SEQ ID NO: 166 and a light chain variable region comprising the amino acid sequence of SEQ ID NO: 167. In some embodiments, the cysteine engineered antibody is elotuzumab.

In some embodiments, a cysteine engineered antibody (e.g., a cysteine engineered antibody of a cysteine engineered ADC) provided herein binds to CD38. In some embodiments, the cysteine engineered antibody comprises CDR-H1, CDR-H2, CDR-H3, CDR-L1, CDR-L2, and CDR-L3 comprising the amino acid sequences of SEQ ID NOs: 168, 169, 170, 171, 172, and 173, respectively. In some embodiments, the cysteine engineered antibody comprises a heavy chain variable region comprising the amino acid sequence of SEQ ID NO: 174 and a light chain variable region comprising the amino acid sequence of SEQ ID NO: 175. In some embodiments, the cysteine engineered antibody is daratumumab.

In some embodiments, a cysteine engineered antibody (e.g., a cysteine engineered antibody of a cysteine engineered ADC) provided herein binds to CD25. In some embodiments, the cysteine engineered antibody comprises CDR-H1, CDR-H2, CDR-H3, CDR-L1, CDR-L2, and CDR-L3 comprising the amino acid sequences of SEQ ID NOs: 176, 177, 178, 179, 180, and 181, respectively. In some embodiments, the cysteine engineered antibody comprises a heavy chain variable region comprising the amino acid sequence of SEQ ID NO: 182 and a light chain variable region comprising the amino acid sequence of SEQ ID NO: 183. In some embodiments, the cysteine engineered antibody is daclizumab.

In some embodiments, a cysteine engineered antibody (e.g., a cysteine engineered antibody of a cysteine engineered ADC) provided herein binds to ADAM9. In some embodiments, the cysteine engineered antibody comprises CDR-H1, CDR-H2, CDR-H3, CDR-L1, CDR-L2, and CDR-L3 comprising the amino acid sequences of SEQ ID NOs: 184, 185, 186, 187, 188, and 189, respectively. In some embodiments, the cysteine engineered antibody comprises a heavy chain variable region comprising the amino acid sequence of SEQ ID NO: 190 and a light chain variable region comprising the amino acid sequence of SEQ ID NO: 191. In some embodiments, the cysteine engineered antibody is chMAbA9-A. In some embodiments, the cysteine engineered antibody comprises CDR-H1, CDR-H2, CDR-H3, CDR-L1, CDR-L2, and CDR-L3 comprising the amino acid sequences of SEQ ID NOs: 192, 193, 194, 195, 196, and 197, respectively. In some embodiments, the cysteine engineered antibody comprises a heavy chain variable region comprising the amino acid sequence of SEQ ID NO: 198 and a light chain variable region comprising the amino acid sequence of SEQ ID NO: 199. In some embodiments, the cysteine engineered antibody is hMAbA9-A.

In some embodiments, a cysteine engineered antibody (e.g., a cysteine engineered antibody of a cysteine engineered ADC) provided herein binds to CD59. In some embodiments, the cysteine engineered antibody comprises CDR-H1, CDR-H2, CDR-H3, CDR-L1, CDR-L2, and CDR-L3 comprising the amino acid sequences of SEQ ID NOs: 200, 201, 202, 203, 204, and 205, respectively. In some embodiments, the cysteine engineered antibody comprises a heavy chain variable region comprising the amino acid sequence of SEQ ID NO: 206 and a light chain variable region comprising the amino acid sequence of SEQ ID NO: 207.

In some embodiments, a cysteine engineered antibody (e.g., a cysteine engineered antibody of a cysteine engineered ADC) provided herein binds to CD25. In some embodiments, the cysteine engineered antibody is Clone123.

In some embodiments, a cysteine engineered antibody (e.g., a cysteine engineered antibody of a cysteine engineered ADC) provided herein binds to CD229. In some embodiments, the cysteine engineered antibody is h8A10.

In some embodiments, a cysteine engineered antibody (e.g., a cysteine engineered antibody of a cysteine engineered ADC) provided herein binds to CD19. In some embodiments, the cysteine engineered antibody comprises CDR-H1, CDR-H2, CDR-H3, CDR-L1, CDR-L2, and CDR-L3 comprising the amino acid sequences of SEQ ID NOs: 208, 209, 210, 211, 212, and 213, respectively. In some embodiments, the cysteine engineered antibody comprises a heavy chain variable region comprising the amino acid sequence of SEQ ID NO: 214 and a light chain variable region comprising the amino acid sequence of SEQ ID NO: 215. In some embodiments, the cysteine engineered antibody comprises a heavy chain comprising the amino acid sequence of SEQ ID NO: 1165 and a light chain comprising the amino acid sequence of SEQ ID NO: 1166. In some embodiments, the cysteine engineered antibody comprises a heavy chain comprising the amino acid sequence of SEQ ID NO: 1165, wherein position 375 or position 400 is substituted with a cysteine. In some embodiments the cysteine engineered antibody comprises a light chain the amino acid sequence of SEQ ID NO: 1166, wherein position 114 or position 121 is substituted with a cysteine. In some embodiments, the cysteine engineered antibody is denintuzumab, which is also known as hBU12. See WO2009052431.

In some embodiments, a cysteine engineered antibody (e.g., a cysteine engineered antibody of a cysteine engineered ADC) provided herein binds to CD70. In some embodiments, the cysteine engineered antibody comprises CDR-H1, CDR-H2, CDR-H3, CDR-L1, CDR-L2, and CDR-L3 comprising the amino acid sequences of SEQ ID NOs: 216, 217, 218, 219, 220, and 221, respectively. In some embodiments, the cysteine engineered antibody comprises a heavy chain variable region comprising the amino acid sequence of SEQ ID NO: 222 and a light chain variable region comprising the amino acid sequence of SEQ ID NO: 223. In some embodiments, the cysteine engineered antibody is vorsetuzumab.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 224, 225, 226, 227, 228, and 229, respectively. In some embodiments, the cysteine engineered antibody comprises a heavy chain variable region comprising the amino acid sequence of SEQ ID NO: 230 and a light chain variable region comprising the amino acid sequence of SEQ ID NO: 231. In some embodiments, the cysteine engineered antibody is mirzotamab.

In some embodiments, a cysteine engineered antibody (e.g., a cysteine engineered antibody of a cysteine engineered ADC) provided herein binds to CD138. In some embodiments, the cysteine engineered antibody comprises CDR-H1, CDR-H2, CDR-H3, CDR-L1, CDR-L2, and CDR-L3 comprising the amino acid sequences of SEQ ID NOs: 232, 233, 234, 235, 236, and 237, respectively. In some embodiments, the cysteine engineered antibody comprises a heavy chain variable region comprising the amino acid sequence of SEQ ID NO: 238 and a light chain variable region comprising the amino acid sequence of SEQ ID NO: 239. In some embodiments, the cysteine engineered antibody is indatuxumab.

In some embodiments, a cysteine engineered antibody (e.g., a cysteine engineered antibody of a cysteine engineered ADC) provided herein binds to CD166. In some embodiments, the cysteine engineered antibody comprises CDR-H1, CDR-H2, CDR-H3, CDR-L1, CDR-L2, and CDR-L3 comprising the amino acid sequences of SEQ ID NOs: 240, 241, 242, 243, 244, and 245, respectively. In some embodiments, the cysteine engineered antibody comprises a heavy chain variable region comprising the amino acid sequence of SEQ ID NO: 246 and a light chain variable region comprising the amino acid sequence of SEQ ID NO: 247. In some embodiments, the cysteine engineered antibody is praluzatamab.

In some embodiments, a cysteine engineered antibody (e.g., a cysteine engineered antibody of a cysteine engineered ADC) provided herein binds to CD51. In some embodiments, the cysteine engineered antibody comprises CDR-H1, CDR-H2, CDR-H3, CDR-L1, CDR-L2, and CDR-L3 comprising the amino acid sequences of SEQ ID NOs: 248, 249, 250, 251, 252, and 253, respectively. In some embodiments, the cysteine engineered antibody comprises a heavy chain variable region comprising the amino acid sequence of SEQ ID NO: 254 and a light chain variable region comprising the amino acid sequence of SEQ ID NO: 255. In some embodiments, the cysteine engineered antibody is intetumumab.

In some embodiments, a cysteine engineered antibody (e.g., a cysteine engineered antibody of a cysteine engineered ADC) provided herein binds to CD56. In some embodiments, the cysteine engineered antibody comprises CDR-H1, CDR-H2, CDR-H3, CDR-L1, CDR-L2, and CDR-L3 comprising the amino acid sequences of SEQ ID NOs: 256, 257, 258, 259, 260, and 261, respectively. In some embodiments, the cysteine engineered antibody comprises a heavy chain variable region comprising the amino acid sequence of SEQ ID NO: 262 and a light chain variable region comprising the amino acid sequence of SEQ ID NO: 263. In some embodiments, the cysteine engineered antibody is lorvotuzumab.

In some embodiments, a cysteine engineered antibody (e.g., a cysteine engineered antibody of a cysteine engineered ADC) provided herein binds to CD74. In some embodiments, the cysteine engineered antibody comprises CDR-H1, CDR-H2, CDR-H3, CDR-L1, CDR-L2, and CDR-L3 comprising the amino acid sequences of SEQ ID NOs: 264, 265, 266, 267, 268, and 269, respectively. In some embodiments, the cysteine engineered antibody comprises a heavy chain variable region comprising the amino acid sequence of SEQ ID NO: 270 and a light chain variable region comprising the amino acid sequence of SEQ ID NO: 271. In some embodiments, the cysteine engineered antibody is milatuzumab.

In some embodiments, a cysteine engineered antibody (e.g., a cysteine engineered antibody of a cysteine engineered ADC) provided herein binds to CEACAM5. In some embodiments, the cysteine engineered antibody comprises CDR-H1, CDR-H2, CDR-H3, CDR-L1, CDR-L2, and CDR-L3 comprising the amino acid sequences of SEQ ID NOs: 272, 273 274, 275, 276, and 277, respectively. In some embodiments, the cysteine engineered antibody comprises a heavy chain variable region comprising the amino acid sequence of SEQ ID NO: 278 and a light chain variable region comprising the amino acid sequence of SEQ ID NO: 279. In some embodiments, the cysteine engineered antibody is labetuzumab.

In some embodiments, a cysteine engineered antibody (e.g., a cysteine engineered antibody of a cysteine engineered ADC) provided herein binds to CanAg. In some embodiments, the cysteine engineered antibody comprises CDR-H1, CDR-H2, CDR-H3, CDR-L1, CDR-L2, and CDR-L3 comprising the amino acid sequences of SEQ ID NOs: 280, 281, 282, 283, 284, and 285, respectively. In some embodiments, the cysteine engineered antibody comprises a heavy chain variable region comprising the amino acid sequence of SEQ ID NO: 286 and a light chain variable region comprising the amino acid sequence of SEQ ID NO: 287. In some embodiments, the cysteine engineered antibody is cantuzumab.

In some embodiments, a cysteine engineered antibody (e.g., a cysteine engineered antibody of a cysteine engineered ADC) provided herein binds to DLL-3. In some embodiments, the cysteine engineered antibody comprises CDR-H1, CDR-H2, CDR-H3, CDR-L1, CDR-L2, and CDR-L3 comprising the amino acid sequences of SEQ ID NOs: 288, 289, 290, 291, 292, and 293, respectively. In some embodiments, the cysteine engineered antibody comprises a heavy chain variable region comprising the amino acid sequence of SEQ ID NO: 294 and a light chain variable region comprising the amino acid sequence of SEQ ID NO: 295. In some embodiments, the cysteine engineered antibody is rovalpituzumab.

In some embodiments, a cysteine engineered antibody (e.g., a cysteine engineered antibody of a cysteine engineered ADC) provided herein binds to DPEP-3. In some embodiments, the cysteine engineered antibody comprises CDR-H1, CDR-H2, CDR-H3, CDR-L1, CDR-L2, and CDR-L3 comprising the amino acid sequences of SEQ ID NOs: 296, 297, 298, 299, 300, and 301, respectively. In some embodiments, the cysteine engineered antibody comprises a heavy chain variable region comprising the amino acid sequence of SEQ ID NO: 302 and a light chain variable region comprising the amino acid sequence of SEQ ID NO: 303. In some embodiments, the cysteine engineered antibody is tamrintamab.

In some embodiments, a cysteine engineered antibody (e.g., a cysteine engineered antibody of a cysteine engineered ADC) provided herein binds to EGFR. In some embodiments, the cysteine engineered antibody comprises CDR-H1, CDR-H2, CDR-H3, CDR-L1, CDR-L2, and CDR-L3 comprising the amino acid sequences of SEQ ID NOs: 304, 305, 306, 307, 308, and 309, respectively. In some embodiments, the cysteine engineered antibody comprises a heavy chain variable region comprising the amino acid sequence of SEQ ID NO: 310 and a light chain variable region comprising the amino acid sequence of SEQ ID NO: 311. In some embodiments, the cysteine engineered antibody is laprituximab. In some embodiments, the cysteine engineered antibody comprises CDR-H1, CDR-H2, CDR-H3, CDR-L1, CDR-L2, and CDR-L3 comprising the amino acid sequences of SEQ ID NOs: 312, 313, 314, 315, 316, and 317, respectively. In some embodiments, the cysteine engineered antibody comprises a heavy chain variable region comprising the amino acid sequence of SEQ ID NO: 318 and a light chain variable region comprising the amino acid sequence of SEQ ID NO: 319. In some embodiments, the cysteine engineered antibody is losatuxizumab. In some embodiments, the cysteine engineered antibody comprises CDR-H1, CDR-H2, CDR-H3, CDR-L1, CDR-L2, and CDR-L3 comprising the amino acid sequences of SEQ ID NOs: 320, 321, 322, 323, 324, and 325, respectively. In some embodiments, the cysteine engineered antibody comprises a heavy chain variable region comprising the amino acid sequence of SEQ ID NO: 326 and a light chain variable region comprising the amino acid sequence of SEQ ID NO: 327. In some embodiments, the cysteine engineered antibody is serclutamab. In some embodiments, the cysteine engineered antibody comprises CDR-H1, CDR-H2, CDR-H3, CDR-L1, CDR-L2, and CDR-L3 comprising the amino acid sequences of SEQ ID NOs: 328, 329, 330, 331, 332, and 333, respectively. In some embodiments, the cysteine engineered antibody comprises a heavy chain variable region comprising the amino acid sequence of SEQ ID NO: 334 and a light chain variable region comprising the amino acid sequence of SEQ ID NO: 335. In some embodiments, the cysteine engineered antibody is cetuximab.

In some embodiments, a cysteine engineered antibody (e.g., a cysteine engineered antibody of a cysteine engineered ADC) provided herein binds to FRa. In some embodiments, the cysteine engineered antibody comprises CDR-H1, CDR-H2, CDR-H3, CDR-L1, CDR-L2, and CDR-L3 comprising the amino acid sequences of SEQ ID NOs: 336, 337, 338, 339, 340, and 341, respectively. In some embodiments, the cysteine engineered antibody comprises a heavy chain variable region comprising the amino acid sequence of SEQ ID NO: 342 and a light chain variable region comprising the amino acid sequence of SEQ ID NO: 343. In some embodiments, the cysteine engineered antibody is mirvetuximab. In some embodiments, the cysteine engineered antibody comprises CDR-H1, CDR-H2, CDR-H3, CDR-L1, CDR-L2, and CDR-L3 comprising the amino acid sequences of SEQ ID NOs: 344, 345, 346, 347, 348, and 349, respectively. In some embodiments, the cysteine engineered antibody comprises a heavy chain variable region comprising the amino acid sequence of SEQ ID NO: 350 and a light chain variable region comprising the amino acid sequence of SEQ ID NO: 351. In some embodiments, the cysteine engineered antibody is farletuzumab.

In some embodiments, a cysteine engineered antibody (e.g., a cysteine engineered antibody of a cysteine engineered ADC) provided herein binds to MUC-1. In some embodiments, the cysteine engineered antibody comprises CDR-H1, CDR-H2, CDR-H3, CDR-L1, CDR-L2, and CDR-L3 comprising the amino acid sequences of SEQ ID NOs: 352, 353, 354, 355, 356, and 357, respectively. In some embodiments, the cysteine engineered antibody comprises a heavy chain variable region comprising the amino acid sequence of SEQ ID NO: 358 and a light chain variable region comprising the amino acid sequence of SEQ ID NO: 359. In some embodiments, the cysteine engineered antibody is gatipotuzumab.

In some embodiments, a cysteine engineered antibody (e.g., a cysteine engineered antibody of a cysteine engineered ADC) provided herein binds to mesothelin. In some embodiments, the cysteine engineered antibody comprises CDR-H1, CDR-H2, CDR-H3, CDR-L1, CDR-L2, and CDR-L3 comprising the amino acid sequences of SEQ ID NOs: 360, 361, 362, 363, 364, and 365, respectively. In some embodiments, the cysteine engineered antibody comprises a heavy chain variable region comprising the amino acid sequence of SEQ ID NO: 366 and a light chain variable region comprising the amino acid sequence of SEQ ID NO: 367. In some embodiments, the cysteine engineered antibody is anetumab.

In some embodiments, a cysteine engineered antibody (e.g., a cysteine engineered antibody of a cysteine engineered ADC) provided herein binds to ROR-1. In some embodiments, the cysteine engineered antibody comprises CDR-H1, CDR-H2, CDR-H3, CDR-L1, CDR-L2, and CDR-L3 comprising the amino acid sequences of SEQ ID NOs: 368, 369, 370, 371, 372, and 373, respectively. In some embodiments, the cysteine engineered antibody comprises a heavy chain variable region comprising the amino acid sequence of SEQ ID NO: 374 and a light chain variable region comprising the amino acid sequence of SEQ ID NO: 375. In some embodiments, the cysteine engineered antibody is zilovertamab.

In some embodiments, a cysteine engineered antibody (e.g., a cysteine engineered antibody of a cysteine engineered ADC) provided herein binds to ASCT2. In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 376, 377, 378, 379, 380, and 381, respectively. In some embodiments, the cysteine engineered antibody comprises a heavy chain variable region comprising the amino acid sequence of SEQ ID NO: 382 and a light chain variable region comprising the amino acid sequence of SEQ ID NO: 383. In some embodiments, the cysteine engineered antibody is 20502. See WO2019040780.

In some embodiments, a cysteine engineered antibody (e.g., a cysteine engineered antibody of a cysteine engineered ADC) provided herein binds to B7-H3. In some embodiments, the cysteine engineered antibody comprises CDR-H1, CDR-H2, CDR-H3, CDR-L1, CDR-L2, and CDR-L3 comprising the amino acid sequences of SEQ ID NOs: 384, 385, 386, 387, 388, and 389, respectively. In some embodiments, the cysteine engineered antibody comprises a heavy chain variable region comprising the amino acid sequence of SEQ ID NO: 390 and a light chain variable region comprising the amino acid sequence of SEQ ID NO: 391. In some embodiments, the cysteine engineered antibody is chAb-A (BRCA84D). In some embodiments, the cysteine engineered antibody comprises CDR-H1, CDR-H2, CDR-H3, CDR-L1, CDR-L2, and CDR-L3 comprising the amino acid sequences of SEQ ID NOs: 392, 393, 394, 395, 396, and 397, respectively. In some embodiments, the cysteine engineered antibody comprises a heavy chain variable region comprising the amino acid sequence of SEQ ID NO: 398 and a light chain variable region comprising the amino acid sequence of SEQ ID NO: 399. In some embodiments, the cysteine engineered antibody is hAb-B. In some embodiments, the cysteine engineered antibody comprises CDR-H1, CDR-H2, CDR-H3, CDR-L1, CDR-L2, and CDR-L3 comprising the amino acid sequences of SEQ ID NOs: 400, 401, 402, 403, 404, and 405, respectively. In some embodiments, the cysteine engineered antibody comprises a heavy chain variable region comprising the amino acid sequence of SEQ ID NO: 406 and a light chain variable region comprising the amino acid sequence of SEQ ID NO: 407. In some embodiments, the cysteine engineered antibody is hAb-C. In some embodiments, the cysteine engineered antibody comprises CDR-H1, CDR-H2, CDR-H3, CDR-L1, CDR-L2, and CDR-L3 comprising the amino acid sequences of SEQ ID NOs: 408, 409, 410, 411, 412, and 413, respectively. In some embodiments, the cysteine engineered antibody comprises a heavy chain variable region comprising the amino acid sequence of SEQ ID NO: 414 and a light chain variable region comprising the amino acid sequence of SEQ ID NO: 415. In some embodiments, the cysteine engineered antibody is hAb-D. In some embodiments, the cysteine engineered antibody comprises CDR-H1, CDR-H2, CDR-H3, CDR-L1, CDR-L2, and CDR-L3 comprising the amino acid sequences of SEQ ID NOs: 416, 417, 418, 419, 420, and 421, respectively. In some embodiments, the cysteine engineered antibody comprises a heavy chain variable region comprising the amino acid sequence of SEQ ID NO: 422 and a light chain variable region comprising the amino acid sequence of SEQ ID NO: 423. In some embodiments, the cysteine engineered antibody is chM30. In some embodiments, the cysteine engineered antibody comprises CDR-H1, CDR-H2, CDR-H3, CDR-L1, CDR-L2, and CDR-L3 comprising the amino acid sequences of SEQ ID NOs: 424, 425, 426, 427, 428, and 429, respectively. In some embodiments, the cysteine engineered antibody comprises a heavy chain variable region comprising the amino acid sequence of SEQ ID NO: 430 and a light chain variable region comprising the amino acid sequence of SEQ ID NO: 431. In some embodiments, the cysteine engineered antibody is hM30-H1-L4. In some embodiments, the cysteine engineered antibody comprises CDR-H1, CDR-H2, CDR-H3, CDR-L1, CDR-L2, and CDR-L3 comprising the amino acid sequences of SEQ ID NOs: 432, 433, 434, 435, 436, and 437, respectively. In some embodiments, the cysteine engineered antibody comprises a heavy chain variable region comprising the amino acid sequence of SEQ ID NO: 438 and a light chain variable region comprising the amino acid sequence of SEQ ID NO: 439. In some embodiments, the cysteine engineered antibody is AbV_huAb18-v4. In some embodiments, the cysteine engineered antibody comprises CDR-H1, CDR-H2, CDR-H3, CDR-L1, CDR-L2, and CDR-L3 comprising the amino acid sequences of SEQ ID NOs: 440, 441, 442, 443, 444, and 445, respectively. In some embodiments, the cysteine engineered antibody comprises a heavy chain variable region comprising the amino acid sequence of SEQ ID NO: 446 and a light chain variable region comprising the amino acid sequence of SEQ ID NO: 447. In some embodiments, the cysteine engineered antibody is AbV_huAb3-v6. In some embodiments, the cysteine engineered antibody comprises CDR-H1, CDR-H2, CDR-H3, CDR-L1, CDR-L2, and CDR-L3 comprising the amino acid sequences of SEQ ID NOs: 448, 449, 450, 451, 452, and 453, respectively. In some embodiments, the cysteine engineered antibody comprises a heavy chain variable region comprising the amino acid sequence of SEQ ID NO: 454 and a light chain variable region comprising the amino acid sequence of SEQ ID NO: 455. In some embodiments, the cysteine engineered antibody is AbV_huAb3-v2.6. In some embodiments, the cysteine engineered antibody comprises CDR-H1, CDR-H2, CDR-H3, CDR-L1, CDR-L2, and CDR-L3 comprising the amino acid sequences of SEQ ID NOs: 456, 457, 458, 459, 460, and 461, respectively. In some embodiments, the cysteine engineered antibody comprises a heavy chain variable region comprising the amino acid sequence of SEQ ID NO: 462 and a light chain variable region comprising the amino acid sequence of SEQ ID NO: 463. In some embodiments, the cysteine engineered antibody is AbV_huAb13-v1-CR. In some embodiments, the cysteine engineered antibody comprises CDR-H1, CDR-H2, CDR-H3, CDR-L1, CDR-L2, and CDR-L3 comprising the amino acid sequences of SEQ ID NOs: 464, 465, 466, 467, 468, and 469, respectively. In some embodiments, the cysteine engineered antibody comprises a heavy chain variable region comprising the amino acid sequence of SEQ ID NO: 470 and a light chain variable region comprising the amino acid sequence of SEQ ID NO: 471. In some embodiments, the cysteine engineered antibody is 8H9-6m. In some embodiments, the cysteine engineered antibody comprises a heavy chain variable region comprising the amino acid sequence of SEQ ID NO: 472 and a light chain variable region comprising the amino acid sequence of SEQ ID NO: 473. In some embodiments, the cysteine engineered antibody is m8517. In some embodiments, the cysteine engineered antibody comprises CDR-H1, CDR-H2, CDR-H3, CDR-L1, CDR-L2, and CDR-L3 comprising the amino acid sequences of SEQ ID NOs: 474, 475, 476, 477, 478, and 479, respectively. In some embodiments, the cysteine engineered antibody comprises a heavy chain variable region comprising the amino acid sequence of SEQ ID NO: 480 and a light chain variable region comprising the amino acid sequence of SEQ ID NO: 481. In some embodiments, the cysteine engineered antibody is TPP-5706. In some embodiments, the cysteine engineered antibody comprises a heavy chain variable region comprising the amino acid sequence of SEQ ID NO: 482 and a light chain variable region comprising the amino acid sequence of SEQ ID NO: 483. In some embodiments, the cysteine engineered antibody is TPP-6642. In some embodiments, the cysteine engineered antibody comprises a heavy chain variable region comprising the amino acid sequence of SEQ ID NO: 484 and a light chain variable region comprising the amino acid sequence of SEQ ID NO: 485. In some embodiments, the cysteine engineered antibody is TPP-6850.

In some embodiments, a cysteine engineered antibody (e.g., a cysteine engineered antibody of a cysteine engineered ADC) provided herein binds to CDCP1. In some embodiments, the cysteine engineered antibody is 10D7.

In some embodiments, a cysteine engineered antibody (e.g., a cysteine engineered antibody of a cysteine engineered ADC) provided herein binds to HER3. In some embodiments, the cysteine engineered antibody comprises a heavy chain comprising the amino acid sequence of SEQ ID NO: 486 and a light chain comprising the amino acid sequence of SEQ ID NO: 487. In some embodiments, the cysteine engineered antibody is patritumab. In some embodiments, the cysteine engineered antibody comprises a heavy chain comprising the amino acid sequence of SEQ ID NO: 488 and a light chain comprising the amino acid sequence of SEQ ID NO: 489. In some embodiments, the cysteine engineered antibody is seribantumab. In some embodiments, the cysteine engineered antibody comprises a heavy chain comprising the amino acid sequence of SEQ ID NO: 490 and a light chain comprising the amino acid sequence of SEQ ID NO: 491. In some embodiments, the cysteine engineered antibody is elgemtumab. In some embodiments, the cysteine engineered antibody comprises a heavy chain the amino acid sequence of SEQ ID NO: 492 and a light chain comprising the amino acid sequence of SEQ ID NO: 493. In some embodiments, the cysteine engineered antibody is lumretuzumab.

In some embodiments, a cysteine engineered antibody (e.g., a cysteine engineered antibody of a cysteine engineered ADC) provided herein binds to RON. In some embodiments, the cysteine engineered antibody is Zt/g4.

In some embodiments, a cysteine engineered antibody (e.g., a cysteine engineered antibody of a cysteine engineered ADC) provided herein binds to claudin-2.

In some embodiments, a cysteine engineered antibody (e.g., a cysteine engineered antibody of a cysteine engineered ADC) provided herein binds to HLA-G.

In some embodiments, a cysteine engineered antibody (e.g., a cysteine engineered antibody of a cysteine engineered ADC) provided herein binds to PTK7. In some embodiments, the cysteine engineered antibody comprises CDR-H1, CDR-H2, CDR-H3, CDR-L1, CDR-L2, and CDR-L3 comprising the amino acid sequences of SEQ ID NOs: 494, 495, 496, 497, 498, and 499, respectively. In some embodiments, the cysteine engineered antibody comprises a heavy chain variable region comprising the amino acid sequence of SEQ ID NO: 500 and a light chain variable region comprising the amino acid sequence of SEQ ID NO: 501. In some embodiments, the cysteine engineered antibody is PTK7 mab 1. In some embodiments, the cysteine engineered antibody comprises CDR-H1, CDR-H2, CDR-H3, CDR-L1, CDR-L2, and CDR-L3 comprising the amino acid sequences of SEQ ID NOs: 502, 503, 504, 505, 506, and 507, respectively. In some embodiments, the cysteine engineered antibody comprises a heavy chain variable region comprising the amino acid sequence of SEQ ID NO: 508 and a light chain variable region comprising the amino acid sequence of SEQ ID NO: 509. In some embodiments, the cysteine engineered antibody is PTK7 mab 2. In some embodiments, the cysteine engineered antibody comprises CDR-H1, CDR-H2, CDR-H3, CDR-L1, CDR-L2, and CDR-L3 comprising the amino acid sequences of SEQ ID NOs: 510, 511, 512, 513, 514, and 515, respectively. In some embodiments, the cysteine engineered antibody comprises a heavy chain variable region comprising the amino acid sequence of SEQ ID NO: 516 and a light chain variable region comprising the amino acid sequence of SEQ ID NO: 517. In some embodiments, the cysteine engineered antibody is PTK7 mab 3.

In some embodiments, a cysteine engineered antibody (e.g., a cysteine engineered antibody of a cysteine engineered ADC) provided herein binds to LIV1. In some embodiments, the cysteine engineered antibody comprises CDR-H1, CDR-H2, CDR-H3, CDR-L1, CDR-L2, and CDR-L3 comprising the amino acid sequences of SEQ ID NOs: 518, 519, 520, 521, 522, and 523, respectively. In some embodiments, the cysteine engineered antibody comprises a heavy chain variable region comprising the amino acid sequence of SEQ ID NO: 524 and a light chain variable region comprising the amino acid sequence of SEQ ID NO: 525. In some embodiments, the cysteine engineered antibody comprises a heavy chain comprising the amino acid sequence of SEQ ID NO: 1167 and a light chain comprising the amino acid sequence of SEQ ID NO: 1168. In some embodiments, the cysteine engineered antibody comprises a heavy chain comprising the amino acid sequence of SEQ ID NO: 1167, wherein position 375 or position 400 is substituted with a cysteine. In some embodiments the cysteine engineered antibody comprises a light chain the amino acid sequence of SEQ ID NO: 1168, wherein position 114 or position 121 is substituted with a cysteine. In some embodiments, the cysteine engineered antibody is ladiratuzumab, which is also known as hLIV22 and hglg. See WO2012078668.

In some embodiments, a cysteine engineered antibody (e.g., a cysteine engineered antibody of a cysteine engineered ADC) provided herein binds to avb6. In some embodiments, the cysteine engineered antibody is BA6. In some embodiments, the cysteine engineered antibody comprises CDR-H1, CDR-H2, CDR-H3, CDR-L1, CDR-L2, and CDR-L3 comprising the amino acid sequences of SEQ ID NOs: 526, 527, 528, 529, 530, and 531, respectively. In some embodiments, the cysteine engineered antibody comprises a heavy chain variable region comprising the amino acid sequence of SEQ ID NO: 532 and a light chain variable region comprising the amino acid sequence of SEQ ID NO: 533. In some embodiments, the cysteine engineered antibody is h2A2. See WO2021113697. In some embodiments, the cysteine engineered antibody comprises CDR-H1, CDR-H2, CDR-H3, CDR-L1, CDR-L2, and CDR-L3 comprising the amino acid sequences of SEQ ID NOs: 534, 535, 536, 537, 538, and 539, respectively. In some embodiments, the cysteine engineered antibody comprises a heavy chain variable region comprising the amino acid sequence of SEQ ID NO: 540 and a light chain variable region comprising the amino acid sequence of SEQ ID NO: 541. In some embodiments, the cysteine engineered antibody is h15H3 or 15 h3. See WO 2013/123152. In some embodiments, the cysteine engineered antibody comprises a heavy chain variable region comprising the amino acid sequence of SEQ ID NO: 890 and a light chain variable region comprising the amino acid sequence of SEQ ID NO: 891.

In some embodiments, a cysteine engineered antibody (e.g., a cysteine engineered antibody of a cysteine engineered ADC) provided herein binds to CD48. In some embodiments, the cysteine engineered antibody comprises CDR-H1, CDR-H2, CDR-H3, CDR-L1, CDR-L2, and CDR-L3 comprising the amino acid sequences of SEQ ID NOs: 542, 543, 544, 545, 546, and 547, respectively. In some embodiments, the cysteine engineered antibody comprises a heavy chain variable region comprising the amino acid sequence of SEQ ID NO: 548 and a light chain variable region comprising the amino acid sequence of SEQ ID NO: 549. In some embodiments, the cysteine engineered antibody is hMEM102. See WO2016149535.

In some embodiments, a cysteine engineered antibody (e.g., a cysteine engineered antibody of a cysteine engineered ADC) provided herein binds to PD-L1. In some embodiments, the cysteine engineered antibody comprises CDR-H1, CDR-H2, CDR-H3, CDR-L1, CDR-L2, and CDR-L3 comprising the amino acid sequences of SEQ ID NOs: 550, 551, 552, 553, 554, and 555, respectively. In some embodiments, the cysteine engineered antibody comprises a heavy chain variable region comprising the amino acid sequence of SEQ ID NO: 556 and a light chain variable region comprising the amino acid sequence of SEQ ID NO: 557. In some embodiments, the cysteine engineered antibody is SG-559-01 LALA mAb.

In some embodiments, a cysteine engineered antibody (e.g., a cysteine engineered antibody of a cysteine engineered ADC) provided herein binds to IGF-1R. In some embodiments, the cysteine engineered antibody comprises CDR-H1, CDR-H2, CDR-H3, CDR-L1, CDR-L2, and CDR-L3 comprising the amino acid sequences of SEQ ID NOs: 558, 559, 560, 561, 562, and 563, respectively. In some embodiments, the cysteine engineered antibody comprises a heavy chain variable region comprising the amino acid sequence of SEQ ID NO: 564 and a light chain variable region comprising the amino acid sequence of SEQ ID NO: 565. In some embodiments, the cysteine engineered antibody is cixutumumab.

In some embodiments, a cysteine engineered antibody (e.g., a cysteine engineered antibody of a cysteine engineered ADC) provided herein binds to claudin-18.2. In some embodiments, the cysteine engineered antibody comprises CDR-H1, CDR-H2, CDR-H3, CDR-L1, CDR-L2, and CDR-L3 comprising the amino acid sequences of SEQ ID NOs: 566, 567, 568, 569, 570, and 571, respectively. In some embodiments, the cysteine engineered antibody comprises a heavy chain variable region comprising the amino acid sequence of SEQ ID NO: 572 and a light chain variable region comprising the amino acid sequence of SEQ ID NO: 573. In some embodiments, the cysteine engineered antibody is zolbetuximab (175D10). In some embodiments, the cysteine engineered antibody comprises CDR-H1, CDR-H2, CDR-H3, CDR-L1, CDR-L2, and CDR-L3 comprising the amino acid sequences of SEQ ID NOs: 574, 575, 576, 577, 578, and 579, respectively. In some embodiments, the cysteine engineered antibody comprises a heavy chain variable region comprising the amino acid sequence of SEQ ID NO: 580 and a light chain variable region comprising the amino acid sequence of SEQ ID NO: 581. In some embodiments, the cysteine engineered antibody is 163E12.

In some embodiments, a cysteine engineered antibody (e.g., a cysteine engineered antibody of a cysteine engineered ADC) provided herein binds to Nectin-4. In some embodiments, the cysteine engineered antibody comprises CDR-H1, CDR-H2, CDR-H3, CDR-L1, CDR-L2, and CDR-L3 comprising the amino acid sequences of SEQ ID NOs: 582, 583, 584, 585, 586, and 587, respectively. In some embodiments, the cysteine engineered antibody comprises a heavy chain variable region comprising the amino acid sequence of SEQ ID NO: 588 and a light chain variable region comprising the amino acid sequence of SEQ ID NO: 589. In some embodiments, the cysteine engineered antibody is enfortumab. See WO 2012047724.

In some embodiments, a cysteine engineered antibody (e.g., a cysteine engineered antibody of a cysteine engineered ADC) provided herein binds to SLTRK6. In some embodiments, the cysteine engineered antibody comprises CDR-H1, CDR-H2, CDR-H3, CDR-L1, CDR-L2, and CDR-L3 comprising the amino acid sequences of SEQ ID NOs: 590, 591, 592, 593, 594, and 595, respectively. In some embodiments, the cysteine engineered antibody comprises a heavy chain variable region comprising the amino acid sequence of SEQ ID NO: 596 and a light chain variable region comprising the amino acid sequence of SEQ ID NO: 597. In some embodiments, the cysteine engineered antibody is sirtratumab.

In some embodiments, a cysteine engineered antibody (e.g., a cysteine engineered antibody of a cysteine engineered ADC) provided herein binds to CD228. In some embodiments, the cysteine engineered antibody comprises CDR-H1, CDR-H2, CDR-H3, CDR-L1, CDR-L2, and CDR-L3 comprising the amino acid sequences of SEQ ID NOs: 598, 599, 600, 601, 602, and 603, respectively. In some embodiments, the cysteine engineered antibody comprises a heavy chain variable region comprising the amino acid sequence of SEQ ID NO: 604 and a light chain variable region comprising the amino acid sequence of SEQ ID NO: 605. In some embodiments, the cysteine engineered antibody is hL49. See WO 2020/163225.

In some embodiments, a cysteine engineered antibody (e.g., a cysteine engineered antibody of a cysteine engineered ADC) provided herein binds to CD142 (tissue factor; TF). In some embodiments, the cysteine engineered antibody comprises CDR-H1, CDR-H2, CDR-H3, CDR-L1, CDR-L2, and CDR-L3 comprising the amino acid sequences of SEQ ID NOs: 606, 607, 608, 609, 610, and 611, respectively. In some embodiments, the cysteine engineered antibody comprises a heavy chain variable region comprising the amino acid sequence of SEQ ID NO: 612 and a light chain variable region comprising the amino acid sequence of SEQ ID NO: 613. In some embodiments, the cysteine engineered antibody is tisotumab. See WO 2010/066803.

In some embodiments, a cysteine engineered antibody (e.g., a cysteine engineered antibody of a cysteine engineered ADC) provided herein binds to STn. In some embodiments, the cysteine engineered antibody comprises CDR-H1, CDR-H2, CDR-H3, CDR-L1, CDR-L2, and CDR-L3 comprising the amino acid sequences of SEQ ID NOs: 614, 615, 616, 617, 618, and 619, respectively. In some embodiments, the cysteine engineered antibody comprises a heavy chain variable region comprising the amino acid sequence of SEQ ID NO: 620 and a light chain variable region comprising the amino acid sequence of SEQ ID NO: 621. In some embodiments, the cysteine engineered antibody is h2G12.

In some embodiments, a cysteine engineered antibody (e.g., a cysteine engineered antibody of a cysteine engineered ADC) provided herein binds to CD20. In some embodiments, the cysteine engineered antibody comprises CDR-H1, CDR-H2, CDR-H3, CDR-L1, CDR-L2, and CDR-L3 comprising the amino acid sequences of SEQ ID NOs: 622, 623, 624, 625, 626, and 627, respectively. In some embodiments, the cysteine engineered antibody comprises a heavy chain variable region comprising the amino acid sequence of SEQ ID NO: 628 and a light chain variable region comprising the amino acid sequence of SEQ ID NO: 629. In some embodiments, the cysteine engineered antibody is rituximab.

In some embodiments, a cysteine engineered antibody (e.g., a cysteine engineered antibody of a cysteine engineered ADC) provided herein binds to HER2. In some embodiments, the cysteine engineered antibody comprises CDR-H1, CDR-H2, CDR-H3, CDR-L1, CDR-L2, and CDR-L3 comprising the amino acid sequences of SEQ ID NOs: 630, 631, 632, 633, 634, and 635, respectively. In some embodiments, the cysteine engineered antibody comprises a heavy chain variable region comprising the amino acid sequence of SEQ ID NO: 636 and a light chain variable region comprising the amino acid sequence of SEQ ID NO: 637. In some embodiments, the cysteine engineered antibody is trastuzumab.

In some embodiments, a cysteine engineered antibody (e.g., a cysteine engineered antibody of a cysteine engineered ADC) provided herein binds to FLT3.

In some embodiments, a cysteine engineered antibody (e.g., a cysteine engineered antibody of a cysteine engineered ADC) provided herein binds to CD46.

In some embodiments, a cysteine engineered antibody (e.g., a cysteine engineered antibody of a cysteine engineered ADC) provided herein binds to GloboH.

In some embodiments, a cysteine engineered antibody (e.g., a cysteine engineered antibody of a cysteine engineered ADC) provided herein binds to AG7.

In some embodiments, a cysteine engineered antibody (e.g., a cysteine engineered antibody of a cysteine engineered ADC) provided herein binds to mesothelin.

In some embodiments, a cysteine engineered antibody (e.g., a cysteine engineered antibody of a cysteine engineered ADC) provided herein binds to FCRH5.

In some embodiments, a cysteine engineered antibody (e.g., a cysteine engineered antibody of a cysteine engineered ADC) provided herein binds to ETBR.

In some embodiments, a cysteine engineered antibody (e.g., a cysteine engineered antibody of a cysteine engineered ADC) provided herein binds to Tim-1.

In some embodiments, a cysteine engineered antibody (e.g., a cysteine engineered antibody of a cysteine engineered ADC) provided herein binds to SLC44A4.

In some embodiments, a cysteine engineered antibody (e.g., a cysteine engineered antibody of a cysteine engineered ADC) provided herein binds to ENPP3.

In some embodiments, a cysteine engineered antibody (e.g., a cysteine engineered antibody of a cysteine engineered ADC) provided herein binds to CD37.

In some embodiments, a cysteine engineered antibody (e.g., a cysteine engineered antibody of a cysteine engineered ADC) provided herein binds to CA9.

In some embodiments, a cysteine engineered antibody (e.g., a cysteine engineered antibody of a cysteine engineered ADC) provided herein binds to Notch3.

In some embodiments, a cysteine engineered antibody (e.g., a cysteine engineered antibody of a cysteine engineered ADC) provided herein binds to EphA2.

In some embodiments, a cysteine engineered antibody (e.g., a cysteine engineered antibody of a cysteine engineered ADC) provided herein binds to TRFC.

In some embodiments, a cysteine engineered antibody (e.g., a cysteine engineered antibody of a cysteine engineered ADC) provided herein binds to PSMA.

In some embodiments, a cysteine engineered antibody (e.g., a cysteine engineered antibody of a cysteine engineered ADC) provided herein binds to LRRC15.

In some embodiments, a cysteine engineered antibody (e.g., a cysteine engineered antibody of a cysteine engineered ADC) provided herein binds to 5T4.

In some embodiments, a cysteine engineered antibody (e.g., a cysteine engineered antibody of a cysteine engineered ADC) provided herein binds to CD79b. In some embodiments, the cysteine engineered antibody comprises CDR-H1, CDR-H2, CDR-H3, CDR-L1, CDR-L2, and CDR-L3 comprising the amino acid sequences of SEQ ID NOs: 638, 639, 640, 641, 642, and 643, respectively. In some embodiments, the cysteine engineered antibody comprises a heavy chain variable region comprising the amino acid sequence of SEQ ID NO: 644 and a light chain variable region comprising the amino acid sequence of SEQ ID NO: 645. In some embodiments, the cysteine engineered antibody is polatuzumab.

In some embodiments, a cysteine engineered antibody (e.g., a cysteine engineered antibody of a cysteine engineered ADC) provided herein binds to NaPi2B. In some embodiments, the cysteine engineered antibody comprises CDR-H1, CDR-H2, CDR-H3, CDR-L1, CDR-L2, and CDR-L3 comprising the amino acid sequences of SEQ ID NOs: 646, 647, 648, 649, 650, and 651, respectively. In some embodiments, the cysteine engineered antibody comprises a heavy chain variable region comprising the amino acid sequence of SEQ ID NO: 652 and a light chain variable region comprising the amino acid sequence of SEQ ID NO: 653. In some embodiments, the cysteine engineered antibody is lifastuzumab.

In some embodiments, a cysteine engineered antibody (e.g., a cysteine engineered antibody of a cysteine engineered ADC) provided herein binds to Muc16. In some embodiments, the cysteine engineered antibody comprises CDR-H1, CDR-H2, CDR-H3, CDR-L1, CDR-L2, and CDR-L3 comprising the amino acid sequences of SEQ ID NOs: 654, 655, 656, 657, 658, and 659, respectively. In some embodiments, the cysteine engineered antibody comprises a heavy chain variable region comprising the amino acid sequence of SEQ ID NO: 660 and a light chain variable region comprising the amino acid sequence of SEQ ID NO: 661. In some embodiments, the cysteine engineered antibody is sofituzumab.

In some embodiments, a cysteine engineered antibody (e.g., a cysteine engineered antibody of a cysteine engineered ADC) provided herein binds to STEAP1. In some embodiments, the cysteine engineered antibody comprises CDR-H1, CDR-H2, CDR-H3, CDR-L1, CDR-L2, and CDR-L3 comprising the amino acid sequences of SEQ ID NOs: 662, 663, 664, 665, 666, and 667, respectively. In some embodiments, the cysteine engineered antibody comprises a heavy chain variable region comprising the amino acid sequence of SEQ ID NO: 668 and a light chain variable region comprising the amino acid sequence of SEQ ID NO: 669. In some embodiments, the cysteine engineered antibody is vandortuzumab.

In some embodiments, a cysteine engineered antibody (e.g., a cysteine engineered antibody of a cysteine engineered ADC) provided herein binds to BCMA. In some embodiments, the cysteine engineered antibody comprises CDR-H1, CDR-H2, CDR-H3, CDR-L1, CDR-L2, and CDR-L3 comprising the amino acid sequences of SEQ ID NOs: 670, 671, 672, 673, 674, and 675, respectively. In some embodiments, the cysteine engineered antibody comprises a heavy chain variable region comprising the amino acid sequence of SEQ ID NO: 676 and a light chain variable region comprising the amino acid sequence of SEQ ID NO: 677. In some embodiments, the cysteine engineered antibody is belantamab.

In some embodiments, a cysteine engineered antibody (e.g., a cysteine engineered antibody of a cysteine engineered ADC) provided herein binds to c-Met. In some embodiments, the cysteine engineered antibody comprises CDR-H1, CDR-H2, CDR-H3, CDR-L1, CDR-L2, and CDR-L3 comprising the amino acid sequences of SEQ ID NOs: 678, 679, 680, 681, 682, and 683, respectively. In some embodiments, the cysteine engineered antibody comprises a heavy chain variable region comprising the amino acid sequence of SEQ ID NO: 684 and a light chain variable region comprising the amino acid sequence of SEQ ID NO: 685. In some embodiments, the cysteine engineered antibody is telisotuzumab.

In some embodiments, a cysteine engineered antibody (e.g., a cysteine engineered antibody of a cysteine engineered ADC) provided herein binds to EGFR. In some embodiments, the cysteine engineered antibody comprises CDR-H1, CDR-H2, CDR-H3, CDR-L1, CDR-L2, and CDR-L3 comprising the amino acid sequences of SEQ ID NOs: 686, 687, 688, 689, 690, and 691, respectively. In some embodiments, the cysteine engineered antibody comprises a heavy chain variable region comprising the amino acid sequence of SEQ ID NO: 692 and a light chain variable region comprising the amino acid sequence of SEQ ID NO: 693. In some embodiments, the cysteine engineered antibody is depatuxizumab.

In some embodiments, a cysteine engineered antibody (e.g., a cysteine engineered antibody of a cysteine engineered ADC) provided herein binds to SLAMF7. In some embodiments, the cysteine engineered antibody comprises CDR-H1, CDR-H2, CDR-H3, CDR-L1, CDR-L2, and CDR-LV3 comprising the amino acid sequences of SEQ ID NOS: 694, 695, 696, 697, 698, and 699, respectively. In some embodiments, the cysteine engineered antibody comprises a heavy chain variable region comprising the amino acid sequence of SEQ ID NO: 700 and a light chain variable region comprising the amino acid sequence of SEQ ID NO: 701. In some embodiments, the cysteine engineered antibody is azintuxizumab.

In some embodiments, a cysteine engineered antibody (e.g., a cysteine engineered antibody of a cysteine engineered ADC) provided herein binds to SLITRK6. In some embodiments, the cysteine engineered antibody comprises CDR-H1, CDR-H2, CDR-H3, CDR-L1, CDR-L2, and CDR-L3 comprising the amino acid sequences of SEQ ID NOs: 702, 703, 704, 705, 706, and 707, respectively. In some embodiments, the cysteine engineered antibody comprises a heavy chain variable region comprising the amino acid sequence of SEQ ID NO: 708 and a light chain variable region comprising the amino acid sequence of SEQ ID NO: 709. In some embodiments, the cysteine engineered antibody is sirtratumab.

In some embodiments, a cysteine engineered antibody (e.g., a cysteine engineered antibody of a cysteine engineered ADC) provided herein binds to C4.4a. In some embodiments, the cysteine engineered antibody comprises CDR-H1, CDR-H2, CDR-H3, CDR-L1, CDR-L2, and CDR-L3 comprising the amino acid sequences of SEQ ID NOs: 710, 711, 712, 713, 714, and 715, respectively. In some embodiments, the cysteine engineered antibody comprises a heavy chain variable region comprising the amino acid sequence of SEQ ID NO: 716 and a light chain variable region comprising the amino acid sequence of SEQ ID NO: 717. In some embodiments, the cysteine engineered antibody is lupartumab.

In some embodiments, a cysteine engineered antibody (e.g., a cysteine engineered antibody of a cysteine engineered ADC) provided herein binds to GCC. In some embodiments, the cysteine engineered antibody comprises CDR-H1, CDR-H2, CDR-H3, CDR-L1, CDR-L2, and CDR-L3 comprising the amino acid sequences of SEQ ID NOs: 718, 719, 720, 721, 722, and 723, respectively. In some embodiments, the cysteine engineered antibody comprises a heavy chain variable region comprising the amino acid sequence of SEQ ID NO: 724 and a light chain variable region comprising the amino acid sequence of SEQ ID NO: 725. In some embodiments, the cysteine engineered antibody is indusatumab.

In some embodiments, a cysteine engineered antibody (e.g., a cysteine engineered antibody of a cysteine engineered ADC) provided herein binds to Axl. In some embodiments, the cysteine engineered antibody comprises CDR-H1, CDR-H2, CDR-H3, CDR-L1, CDR-L2, and CDR-L3 comprising the amino acid sequences of SEQ ID NOs: 726, 727, 728, 729, 730, and 731, respectively. In some embodiments, the cysteine engineered antibody comprises a heavy chain variable region comprising the amino acid sequence of SEQ ID NO: 732 and a light chain variable region comprising the amino acid sequence of SEQ ID NO: 733. In some embodiments, the cysteine engineered antibody is enapotamab.

In some embodiments, a cysteine engineered antibody (e.g., a cysteine engineered antibody of a cysteine engineered ADC) provided herein binds to gpNMB. In some embodiments, the cysteine engineered antibody comprises CDR-H1, CDR-H2, CDR-H3, CDR-L1, CDR-L2, and CDR-L3 comprising the amino acid sequences of SEQ ID NOs: 734, 735, 736, 737, 738, and 739, respectively. In some embodiments, the cysteine engineered antibody comprises a heavy chain variable region comprising the amino acid sequence of SEQ ID NO: 740 and a light chain variable region comprising the amino acid sequence of SEQ ID NO: 741. In some embodiments, the cysteine engineered antibody comprises a heavy chain comprising the amino acid sequence of SEQ ID NO: 1169 and a light chain comprising the amino acid sequence of SEQ ID NO: 1170. In some embodiments, the cysteine engineered antibody comprises a heavy chain comprising the amino acid sequence of SEQ ID NO: 1169, wherein position 375 or position 400 is substituted with a cysteine. In some embodiments the cysteine engineered antibody comprises a light chain the amino acid sequence of SEQ ID NO: 1170, wherein position 114 or position 121 is substituted with a cysteine. In some embodiments, the cysteine engineered antibody is glembatumumab.

In some embodiments, a cysteine engineered antibody (e.g., a cysteine engineered antibody of a cysteine engineered ADC) provided herein binds to Prolactin receptor. In some embodiments, the cysteine engineered antibody comprises CDR-H1, CDR-H2, CDR-H3, CDR-L1, CDR-L2, and CDR-L3 comprising the amino acid sequences of SEQ ID NOs: 742, 743, 744, 745, 746, and 747, respectively. In some embodiments, the cysteine engineered antibody comprises a heavy chain variable region comprising the amino acid sequence of SEQ ID NO: 748 and a light chain variable region comprising the amino acid sequence of SEQ ID NO: 749. In some embodiments, the cysteine engineered antibody is rolinsatamab.

In some embodiments, a cysteine engineered antibody (e.g., a cysteine engineered antibody of a cysteine engineered ADC) provided herein binds to FGFR2. In some embodiments, the cysteine engineered antibody comprises CDR-H1, CDR-H2, CDR-H3, CDR-L1, CDR-L2, and CDR-L3 comprising the amino acid sequences of SEQ ID NOs: 750, 751, 752, 753, 754, and 755, respectively. In some embodiments, the cysteine engineered antibody comprises a heavy chain variable region comprising the amino acid sequence of SEQ ID NO: 756 and a light chain variable region comprising the amino acid sequence of SEQ ID NO: 757. In some embodiments, the cysteine engineered antibody is aprutumab.

In some embodiments, a cysteine engineered antibody (e.g., a cysteine engineered antibody of a cysteine engineered ADC) provided herein binds to CDCP1. In some embodiments, the cysteine engineered antibody comprises CDR-H1, CDR-H2, CDR-H3, CDR-L1, CDR-L2, and CDR-L3 comprising the amino acid sequences of SEQ ID NOs: 758, 759, 760, 761, 762, and 763, respectively. In some embodiments, the cysteine engineered antibody comprises a heavy chain variable region comprising the amino acid sequence of SEQ ID NO: 764 and a light chain variable region comprising the amino acid sequence of SEQ ID NO: 765. In some embodiments, the cysteine engineered antibody is Humanized CUB4 #135 HC4-H. In some embodiments, the cysteine engineered antibody comprises CDR-H1, CDR-H2, CDR-H3, CDR-L1, CDR-L2, and CDR-L3 comprising the amino acid sequences of SEQ ID NOs: 766, 767, 768, 769, 770, and 771, respectively. In some embodiments, the cysteine engineered antibody comprises a heavy chain variable region comprising the amino acid sequence of SEQ ID NO: 772 and a light chain variable region comprising the amino acid sequence of SEQ ID NO: 773. In some embodiments, the cysteine engineered antibody is CUB4. In some embodiments, the cysteine engineered antibody comprises CDR-H1, CDR-H2, CDR-H3, CDR-L1, CDR-L2, and CDR-L3 comprising the amino acid sequences of SEQ ID NOs: 774, 775, 776, 777, 778, 779, respectively. In some embodiments, the cysteine engineered antibody comprises a heavy chain variable region comprising the amino acid sequence of SEQ ID NO: 780 and a light chain variable region comprising the amino acid sequence of SEQ ID NO: 781. In some embodiments, the cysteine engineered antibody is CP13E10-WT. In some embodiments, the cysteine engineered antibody comprises CDR-H1, CDR-H2, CDR-H3, CDR-L1, CDR-L2, and CDR-L3 comprising the amino acid sequences of SEQ ID NOs: 782, 783, 784, 785, 786, and 787, respectively. In some embodiments, the cysteine engineered antibody comprises a heavy chain variable region comprising the amino acid sequence of SEQ ID NO: 788 and a light chain variable region comprising the amino acid sequence of SEQ ID NO: 789. In some embodiments, the cysteine engineered antibody is CP13E10-54HCv13-89LCv1.

In some embodiments, a cysteine engineered antibody (e.g., a cysteine engineered antibody of a cysteine engineered ADC) provided herein binds to ASCT2. In some embodiments, the cysteine engineered antibody comprises a heavy chain variable region comprising the amino acid sequence of SEQ ID NO: 790 and a light chain variable region comprising the amino acid sequence of SEQ ID NO: 791. In some embodiments, the cysteine engineered antibody is KM8094a. In some embodiments, the cysteine engineered antibody comprises a heavy chain variable region comprising the amino acid sequence of SEQ ID NO: 792 and a light chain variable region comprising the amino acid sequence of SEQ ID NO: 793. In some embodiments, the cysteine engineered antibody is KM8094b. In some embodiments, the cysteine engineered antibody comprises CDR-H1, CDR-H2, CDR-H3, CDR-L1, CDR-L2, and CDR-L3 comprising the amino acid sequences of SEQ ID NOs: 794, 795, 796, 797, 798, and 799, respectively. In some embodiments, the cysteine engineered antibody comprises a heavy chain variable region comprising the amino acid sequence of SEQ ID NO: 800 and a light chain variable region comprising the amino acid sequence of SEQ ID NO: 801. In some embodiments, the cysteine engineered antibody is KM4018.

In some embodiments, a cysteine engineered antibody (e.g., a cysteine engineered antibody of a cysteine engineered ADC) provided herein binds to CD123. In some embodiments, the cysteine engineered antibody comprises CDR-H1, CDR-H2, CDR-H3, CDR-L1, CDR-L2, and CDR-L3 comprising the amino acid sequences of SEQ ID NOs: 802, 803, 804, 805, 806, and 807, respectively. In some embodiments, the cysteine engineered antibody comprises a heavy chain variable region comprising the amino acid sequence of SEQ ID NO: 808 and a light chain variable region comprising the amino acid sequence of SEQ ID NO: 809. In some embodiments, the cysteine engineered antibody is h7G3. See WO 2016201065.

In some embodiments, a cysteine engineered antibody (e.g., a cysteine engineered antibody of a cysteine engineered ADC) provided herein binds to GPC3. In some embodiments, the cysteine engineered antibody comprises CDR-H1, CDR-H2, CDR-H3, CDR-L1, CDR-L2, and CDR-L3 comprising the amino acid sequences of SEQ ID NOs: 810, 811, 812, 813, 814, and 815, respectively. In some embodiments, the cysteine engineered antibody comprises a heavy chain variable region comprising the amino acid sequence of SEQ ID NO: 816 and a light chain variable region comprising the amino acid sequence of SEQ ID NO: 817. In some embodiments, the cysteine engineered antibody is hGPC3-1. See WO 2019161174.

In some embodiments, a cysteine engineered antibody (e.g., a cysteine engineered antibody of a cysteine engineered ADC) provided herein binds to PD-L1. In some embodiments, the cysteine engineered antibody comprises CDR-H1, CDR-H2, CDR-H3, CDR-L1, CDR-L2, and CDR-L3 comprising the amino acid sequences of SEQ ID NOs: 834, 835, 836, 837, 838, and 839, respectively. In some embodiments, the cysteine engineered antibody comprises a heavy chain variable region comprising the amino acid sequence of SEQ ID NO: 840 and a light chain variable region comprising the amino acid sequence of SEQ ID NO: 841. In some embodiments, the cysteine engineered antibody is SG-559-01. See PCT/US2020/054037.

In some embodiments, a cysteine engineered antibody (e.g., a cysteine engineered antibody of a cysteine engineered ADC) provided herein binds to TIGIT. In some embodiments, the cysteine engineered antibody comprises CDR-H1, CDR-H2, CDR-H3, CDR-L1, CDR-L2, and CDR-L3 comprising the amino acid sequences of SEQ ID NOs: 842, 843, 844, 845, 846, and 847, respectively. In some embodiments, the cysteine engineered antibody comprises a heavy chain variable region comprising the amino acid sequence of SEQ ID NO: 848 and a light chain variable region comprising the amino acid sequence of SEQ ID NO: 849. In some embodiments, the cysteine engineered antibody is Clone 13 (also known as ADI-23674 or mAb13). See WO 2020041541.

In some embodiments, a cysteine engineered antibody (e.g., a cysteine engineered antibody of a cysteine engineered ADC) provided herein binds to STN. In some embodiments, the cysteine engineered antibody comprises CDR-H1, CDR-H2, CDR-H3, CDR-L1, CDR-L2, and CDR-L3 comprising the amino acid sequences of SEQ ID NOs: 850, 851, 852, 853, 854, and 855, respectively. In some embodiments, the cysteine engineered antibody comprises a heavy chain variable region comprising the amino acid sequence of SEQ ID NO: 856 and a light chain variable region comprising the amino acid sequence of SEQ ID NO: 857. In some embodiments, the cysteine engineered antibody is 2G12-2B2. See WO 2017083582.

In some embodiments, a cysteine engineered antibody (e.g., a cysteine engineered antibody of a cysteine engineered ADC) provided herein binds to CD33. In some embodiments, the cysteine engineered antibody comprises CDR-H1, CDR-H2, CDR-H3, CDR-L1, CDR-L2, and CDR-L3 comprising the amino acid sequences of SEQ ID NOs: 858, 859, 860, 861, 862, and 863, respectively. In some embodiments, the cysteine engineered antibody comprises a heavy chain variable region comprising the amino acid sequence of SEQ ID NO: 864 and a light chain variable region comprising the amino acid sequence of SEQ ID NO: 865. In some embodiments, the cysteine engineered antibody is h2H12. See WO2013173496.

In some embodiments, a cysteine engineered antibody (e.g., a cysteine engineered antibody of a cysteine engineered ADC) provided herein binds to NTBA (also known as CD352). In some embodiments, the cysteine engineered antibody comprises CDR-H1, CDR-H2, CDR-H3, CDR-L1, CDR-L2, and CDR-L3 comprising the amino acid sequences of SEQ ID NOs: 866, 867, 868, 869, 870, and 871, respectively. In some embodiments, the cysteine engineered antibody comprises a heavy chain variable region comprising the amino acid sequence of SEQ ID NO: 872 and a light chain variable region comprising the amino acid sequence of SEQ ID NO: 873. In some embodiments, the cysteine engineered antibody is h20F3 HDLD. See WO 2017004330.

In some embodiments, a cysteine engineered antibody (e.g., a cysteine engineered antibody of a cysteine engineered ADC) provided herein binds to BCMA. In some embodiments, the cysteine engineered antibody comprises CDR-H1, CDR-H2, CDR-H3, CDR-L1, CDR-L2, and CDR-L3 comprising the amino acid sequences of SEQ ID NOs: 874, 875, 876, 877, 878, and 879, respectively. In some embodiments, the cysteine engineered antibody comprises a heavy chain variable region comprising the amino acid sequence of SEQ ID NO: 880 and a light chain variable region comprising the amino acid sequence of SEQ ID NO: 881. In some embodiments, the cysteine engineered antibody is SEA-BCMA (also known as hSG16.17). See WO 2017/143069.

In some embodiments, a cysteine engineered antibody (e.g., a cysteine engineered antibody of a cysteine engineered ADC) provided herein binds to Tissue Factor (also known as TF). In some embodiments, the cysteine engineered antibody comprises CDR-H1, CDR-H2, CDR-H3, CDR-L1, CDR-L2, and CDR-L3 comprising the amino acid sequence AAS or any of one SEQ ID NOs: 882, 883, 884, 885, and 887, respectively. In some embodiments, the cysteine engineered antibody comprises a heavy chain variable region comprising the amino acid sequence of SEQ ID NO: 888 and a light chain variable region comprising the amino acid sequence of SEQ ID NO: 889. In some embodiments, the cysteine engineered antibody is tisotumab. See WO 2010/066803 and U.S. Pat. No. 9,150,658.

In some embodiments, a cysteine engineered antibody (e.g., a cysteine engineered antibody of a cysteine engineered ADC) provided herein binds to EphA2. In some embodiments, the cysteine engineered antibody comprises CDR-H1, CDR-H2, CDR-H3, CDR-L1, CDR-L2, and CDR-L3 comprising the amino acid sequences of SEQ ID NOs: 900, 901, 902, 903, 904, and 905, respectively. In some embodiments, the anti-EphA2 antibody comprises a heavy chain variable region comprising an amino acid sequence that is at least 95%, at least 96%, at least 97%, at last 98%, at least 99%, or 100% identical to the amino acid sequence of SEQ ID NO: 906 and a light chain variable region comprising an amino acid sequence that is at least 95% at least 96%, at least 97%, at last 98%, at least 99%, or 100% identical to the amino acid sequence of SEQ ID NO: 907. In some embodiments, the anti-EphA2 antibody comprises a heavy chain comprising the amino acid sequence of SEQ ID NO: 908 and a light chain comprising the amino acid sequence of SEQ ID NO: 910. In some embodiments, the anti-EphA2 antibody comprises a heavy chain comprising the amino acid sequence of SEQ ID NO: 909 and a light chain comprising the amino acid sequence of SEQ ID NO: 910. In some embodiments, the anti-EphA2 antibody comprises a heavy chain comprising the amino acid sequence of SEQ ID NO: 911 and a light chain comprising the amino acid sequence of SEQ ID NO: 913. In some embodiments, the anti-EphA2 antibody comprises a heavy chain comprising the amino acid sequence of SEQ ID NO: 912 and a light chain comprising the amino acid sequence of SEQ ID NO: 913. In some embodiments, the anti-EphA2 antibody comprises a heavy chain comprising the amino acid sequence of SEQ ID NO: 914 and a light chain comprising the amino acid sequence of SEQ ID NO: 916. In some embodiments, the anti-EphA2 antibody comprises a heavy chain comprising the amino acid sequence of SEQ ID NO: 915 and a light chain comprising the amino acid sequence of SEQ ID NO: 916. In some embodiments, the cysteine engineered antibody comprises a heavy chain comprising the amino acid sequence of any of SEQ ID NOs: 908, 909, 911, 912, or 914, wherein position 375 or position 400 is substituted with a cysteine. In some embodiments the cysteine engineered antibody comprises a light chain comprising the amino acid sequence of any of SEQ ID NOs: 910, 913, or 916, wherein position 114 or position 121 is substituted with a cysteine. In some embodiments, the antibody of the antibody drug conjugate is h1C1.

In some embodiments, a cysteine engineered antibody (e.g., a cysteine engineered antibody of a cysteine engineered ADC) provided herein binds to CD228. In some embodiments, the cysteine engineered antibody comprises CDR-H1, CDR-H2, CDR-H3, CDR-L1, CDR-L2, and CDR-L3 comprising the amino acid sequences of SEQ ID NOs: 917, 918, 919, 920, 921, and 922, respectively. In some embodiments, the anti-CD228 antibody comprises a heavy chain variable region comprising an amino acid sequence that is at least 95%, at least 96%, at least 97%, at last 98%, at least 99%, or 100% identical to the amino acid sequence of SEQ ID NO: 923 and a light chain variable region comprising an amino acid sequence that is at least 95% at least 96%, at least 97%, at last 98%, at least 99%, or 100% identical to the amino acid sequence of SEQ ID NO: 924. In some embodiments, the anti-CD228 antibody comprises a heavy chain comprising the amino acid sequence of SEQ ID NO: 925 and a light chain comprising the amino acid sequence of SEQ ID NO: 927. In some embodiments, the anti-CD228 antibody comprises a heavy chain comprising the amino acid sequence of SEQ ID NO: 926 and a light chain comprising the amino acid sequence of SEQ ID NO: 927. In some embodiments, the anti-CD228 antibody comprises a heavy chain comprising the amino acid sequence of SEQ ID NO: 928 and a light chain comprising the amino acid sequence of SEQ ID NO: 930. In some embodiments, the anti-CD228 antibody comprises a heavy chain comprising the amino acid sequence of SEQ ID NO: 929 and a light chain comprising the amino acid sequence of SEQ ID NO: 930. In some embodiments, the cysteine engineered antibody comprises a heavy chain comprising the amino acid sequence of any of SEQ ID NOs: 925, 926, 928, or 929, wherein position 375 or position 400 is substituted with a cysteine. In some embodiments the cysteine engineered antibody comprises a light chain comprising the amino acid sequence of any of SEQ ID NOs: 927 or 930, wherein position 114 or position 121 is substituted with a cysteine. In some embodiments, the antibody of the antibody drug conjugate is hL49.

In some embodiments, a cysteine engineered antibody (e.g., a cysteine engineered antibody of a cysteine engineered ADC) provided herein binds to integrin avB6 (or integrin beta 6/ITGB6). In some embodiments, the cysteine engineered antibody comprises CDR-H1, CDR-H2, CDR-H3, CDR-L1, CDR-L2, and CDR-L3 comprising the amino acid sequences of SEQ ID NOs: 931, 932, 933, 934, 935, and 936, respectively. In some embodiments, the anti-integrin avB6 antibody comprises a heavy chain variable region comprising an amino acid sequence that is at least 95%, at least 96%, at least 97%, at last 98%, at least 99%, or 100% identical to the amino acid sequence of SEQ ID NO: 937 and a light chain variable region comprising an amino acid sequence that is at least 95% at least 96%, at least 97%, at last 98%, at least 99%, or 100% identical to the amino acid sequence of SEQ ID NO: 938. In some embodiments, the anti-integrin avB6 antibody comprises a heavy chain comprising the amino acid sequence of SEQ ID NO: 939 and a light chain comprising the amino acid sequence of SEQ ID NO: 941. In some embodiments, the anti-integrin avB6 antibody comprises a heavy chain comprising the amino acid sequence of SEQ ID NO: 940 and a light chain comprising the amino acid sequence of SEQ ID NO: 941. In some embodiments, the anti-integrin avB6 antibody comprises a heavy chain comprising the amino acid sequence of SEQ ID NO: 942 and a light chain comprising the amino acid sequence of SEQ ID NO: 944. In some embodiments, the anti-integrin avB6 antibody comprises a heavy chain comprising the amino acid sequence of SEQ ID NO: 943 and a light chain comprising the amino acid sequence of SEQ ID NO: 944. In some embodiments, the cysteine engineered antibody comprises a heavy chain comprising the amino acid sequence of any of SEQ ID NOs: 939, 940, 942, or 943, wherein position 375 or position 400 is substituted with a cysteine. In some embodiments the cysteine engineered antibody comprises a light chain comprising the amino acid sequence of any of SEQ ID NOs: 941 or 944, wherein position 114 or position 121 is substituted with a cysteine. In some embodiments, the antibody of the antibody drug conjugate is h2A2.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 945, 946, 947, 948, 949, and 950, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 951 and a light chain variable region comprising an amino acid sequence that is at least 95% at least 96%, at least 97%, at last 98%, at least 99%, or 100% identical to the amino acid sequence of SEQ ID NO: 952. In some embodiments, the cysteine engineered antibody comprises a heavy chain comprising the amino acid sequence of SEQ ID NO: 953 and a light chain comprising the amino acid sequence of SEQ ID NO: 955. In some embodiments, the cysteine engineered antibody comprises a heavy chain comprising the amino acid sequence of SEQ ID NO: 954 and a light chain comprising the amino acid sequence of SEQ ID NO: 955. In some embodiments, the cysteine engineered antibody comprises a heavy chain comprising the amino acid sequence of SEQ ID NO: 956 and a light chain comprising the amino acid sequence of SEQ ID NO: 958. In some embodiments, the cysteine engineered antibody comprises a heavy chain comprising the amino acid sequence of SEQ ID NO: 957 and a light chain comprising the amino acid sequence of SEQ ID NO: 958. In some embodiments, the cysteine engineered antibody comprises a heavy chain comprising the amino acid sequence of any of SEQ ID NOs: 953, 954, 956, or 957, wherein position 375 or position 400 is substituted with a cysteine. In some embodiments the cysteine engineered antibody comprises a light chain comprising the amino acid sequence of any of SEQ ID NOs: 955 or 958, wherein position 114 or position 121 is substituted with a cysteine. In some embodiments, the antibody of the antibody drug conjugate is B7H41001.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 959, 960, 961, 962, 963, and 964, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 965 and a light chain variable region comprising an amino acid sequence that is at least 95% at least 96%, at least 97%, at last 98%, at least 99%, or 100% identical to the amino acid sequence of SEQ ID NO: 966. In some embodiments, the cysteine engineered antibody comprises a heavy chain comprising the amino acid sequence of SEQ ID NO: 1119 and a light chain comprising the amino acid sequence of SEQ ID NO: 1120. In some embodiments, the cysteine engineered antibody comprises a heavy chain comprising the amino acid sequence of SEQ ID NO: 1119, wherein position 375 or position 400 is substituted with a cysteine. In some embodiments the cysteine engineered antibody comprises a light chain comprising the amino acid sequence of SEQ ID NO: 1120, wherein position 114 or position 121 is substituted with a cysteine. In some embodiments, the antibody of the antibody drug conjugate is B7H4-15461.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 967, 968, 969, 970, 971, and 972, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 973 and a light chain variable region comprising an amino acid sequence that is at least 95% at least 96%, at least 97%, at last 98%, at least 99%, or 100% identical to the amino acid sequence of SEQ ID NO: 974. In some embodiments, the cysteine engineered antibody comprises a heavy chain comprising the amino acid sequence of SEQ ID NO: 1121 and a light chain comprising the amino acid sequence of SEQ ID NO: 1122. In some embodiments, the cysteine engineered antibody comprises a heavy chain comprising the amino acid sequence of SEQ ID NO: 1121, wherein position 375 or position 400 is substituted with a cysteine. In some embodiments the cysteine engineered antibody comprises a light chain the amino acid sequence of SEQ ID NO: 1122, wherein position 114 or position 121 is substituted with a cysteine. In some embodiments, the antibody of the antibody drug conjugate is B7H4-20500.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 975, 976, 977, 978, 979, and 980, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 981 and a light chain variable region comprising an amino acid sequence that is at least 95% at least 96%, at least 97%, at last 98%, at least 99%, or 100% identical to the amino acid sequence of SEQ ID NO: 982. In some embodiments, the cysteine engineered antibody comprises a heavy chain comprising the amino acid sequence of SEQ ID NO: 1123 and a light chain comprising the amino acid sequence of SEQ ID NO: 1124. In some embodiments, the cysteine engineered antibody comprises a heavy chain comprising the amino acid sequence of SEQ ID NO: 1123, wherein position 375 or position 400 is substituted with a cysteine. In some embodiments the cysteine engineered antibody comprises a light chain the amino acid sequence of SEQ ID NO: 1124, wherein position 114 or position 121 is substituted with a cysteine. In some embodiments, the antibody of the antibody drug conjugate is B7H4-20501.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 983, 984, 985, 986, 987, and 988, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 989 and a light chain variable region comprising an amino acid sequence that is at least 95% at least 96%, at least 97%, at last 98%, at least 99%, or 100% identical to the amino acid sequence of SEQ ID NO: 990. In some embodiments, the cysteine engineered antibody comprises a heavy chain comprising the amino acid sequence of SEQ ID NO: 1125 and a light chain comprising the amino acid sequence of SEQ ID NO: 1126. In some embodiments, the cysteine engineered antibody comprises a heavy chain comprising the amino acid sequence of SEQ ID NO: 1125, wherein position 375 or position 400 is substituted with a cysteine. In some embodiments the cysteine engineered antibody comprises a light chain the amino acid sequence of SEQ ID NO: 1126, wherein position 114 or position 121 is substituted with a cysteine. In some embodiments, the antibody of the antibody drug conjugate is B7H4-20502.1.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 991, 992, 993, 994, 995, and 996, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 997 and a light chain variable region comprising an amino acid sequence that is at least 95% at least 96%, at least 97%, at last 98%, at least 99%, or 100% identical to the amino acid sequence of SEQ ID NO: 998. In some embodiments, the cysteine engineered antibody comprises a heavy chain comprising the amino acid sequence of SEQ ID NO: 1127 and a light chain comprising the amino acid sequence of SEQ ID NO: 1128. In some embodiments, the cysteine engineered antibody comprises a heavy chain comprising the amino acid sequence of SEQ ID NO: 1127, wherein position 375 or position 400 is substituted with a cysteine. In some embodiments the cysteine engineered antibody comprises a light chain the amino acid sequence of SEQ ID NO: 1128, wherein position 114 or position 121 is substituted with a cysteine. In some embodiments, the antibody of the antibody drug conjugate is B7H4-22208.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 999, 1000, 1001, 1002, 1003, and 1004, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1005 and a light chain variable region comprising an amino acid sequence that is at least 95% at least 96%, at least 97%, at last 98%, at least 99%, or 100% identical to the amino acid sequence of SEQ ID NO: 1006. In some embodiments, the cysteine engineered antibody comprises a heavy chain comprising the amino acid sequence of SEQ ID NO: 1129 and a light chain comprising the amino acid sequence of SEQ ID NO: 1130. In some embodiments, the cysteine engineered antibody comprises a heavy chain comprising the amino acid sequence of SEQ ID NO: 1129, wherein position 375 or position 400 is substituted with a cysteine. In some embodiments the cysteine engineered antibody comprises a light chain the amino acid sequence of SEQ ID NO: 1130, wherein position 114 or position 121 is substituted with a cysteine. In some embodiments, the antibody of the antibody drug conjugate is B7H4-15462.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1007, 1008, 1009, 1010, 1011, and 1012, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1013 and a light chain variable region comprising an amino acid sequence that is at least 95% at least 96%, at least 97%, at last 98%, at least 99%, or 100% identical to the amino acid sequence of SEQ ID NO: 1014. In some embodiments, the cysteine engineered antibody comprises a heavy chain comprising the amino acid sequence of SEQ ID NO: 1131 and a light chain comprising the amino acid sequence of SEQ ID NO: 1132. In some embodiments, the cysteine engineered antibody comprises a heavy chain comprising the amino acid sequence of SEQ ID NO: 1131, wherein position 375 or position 400 is substituted with a cysteine. In some embodiments the cysteine engineered antibody comprises a light chain the amino acid sequence of SEQ ID NO: 1132, wherein position 114 or position 121 is substituted with a cysteine. In some embodiments, the antibody of the antibody drug conjugate is B7H4-22213.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1015, 1016, 1017, 1018, 1019, and 1020, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1021 and a light chain variable region comprising an amino acid sequence that is at least 95% at least 96%, at least 97%, at last 98%, at least 99%, or 100% identical to the amino acid sequence of SEQ ID NO: 1022. In some embodiments, the cysteine engineered antibody comprises a heavy chain comprising the amino acid sequence of SEQ ID NO: 1133 and a light chain comprising the amino acid sequence of SEQ ID NO: 1134. In some embodiments, the cysteine engineered antibody comprises a heavy chain comprising the amino acid sequence of SEQ ID NO: 1133, wherein position 375 or position 400 is substituted with a cysteine. In some embodiments the cysteine engineered antibody comprises a light chain the amino acid sequence of SEQ ID NO: 1134, wherein position 114 or position 121 is substituted with a cysteine. In some embodiments, the antibody of the antibody drug conjugate is B7H4-15465.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1023, 1024, 1025, 1026, 1027, and 1028, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1029 and a light chain variable region comprising an amino acid sequence that is at least 95% at least 96%, at least 97%, at last 98%, at least 99%, or 100% identical to the amino acid sequence of SEQ ID NO: 1030. In some embodiments, the cysteine engineered antibody comprises a heavy chain comprising the amino acid sequence of SEQ ID NO: 1135 and a light chain comprising the amino acid sequence of SEQ ID NO: 1136. In some embodiments, the cysteine engineered antibody comprises a heavy chain comprising the amino acid sequence of SEQ ID NO: 1135, wherein position 375 or position 400 is substituted with a cysteine. In some embodiments the cysteine engineered antibody comprises a light chain the amino acid sequence of SEQ ID NO: 1136, wherein position 114 or position 121 is substituted with a cysteine. In some embodiments, the antibody of the antibody drug conjugate is B7H4-20506.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOS: 1031, 1032, 1033, 1034, 1035, and 1036, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1037 and a light chain variable region comprising an amino acid sequence that is at least 95% at least 96%, at least 97%, at last 98%, at least 99%, or 100% identical to the amino acid sequence of SEQ ID NO: 1038. In some embodiments, the cysteine engineered antibody comprises a heavy chain comprising the amino acid sequence of SEQ ID NO: 1137 and a light chain comprising the amino acid sequence of SEQ ID NO: 1138. In some embodiments, the cysteine engineered antibody comprises a heavy chain comprising the amino acid sequence of SEQ ID NO: 1137, wherein position 375 or position 400 is substituted with a cysteine. In some embodiments the cysteine engineered antibody comprises a light chain the amino acid sequence of SEQ ID NO: 1138, wherein position 114 or position 121 is substituted with a cysteine. In some embodiments, the antibody of the antibody drug conjugate is B7H4-15483.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1039, 1040, 1041, 1042, 1043, and 1044, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1045 and a light chain variable region comprising an amino acid sequence that is at least 95% at least 96%, at least 97%, at last 98%, at least 99%, or 100% identical to the amino acid sequence of SEQ ID NO: 1046. In some embodiments, the cysteine engineered antibody comprises a heavy chain comprising the amino acid sequence of SEQ ID NO: 1139 and a light chain comprising the amino acid sequence of SEQ ID NO: 1140. In some embodiments, the cysteine engineered antibody comprises a heavy chain comprising the amino acid sequence of SEQ ID NO: 1139, wherein position 375 or position 400 is substituted with a cysteine. In some embodiments the cysteine engineered antibody comprises a light chain the amino acid sequence of SEQ ID NO: 1140, wherein position 114 or position 121 is substituted with a cysteine. In some embodiments, the antibody of the antibody drug conjugate is B7H4-20513.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1047, 1048, 1049, 1050, 1051, and 1052, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1053 and a light chain variable region comprising an amino acid sequence that is at least 95% at least 96%, at least 97%, at last 98%, at least 99%, or 100% identical to the amino acid sequence of SEQ ID NO: 1054. In some embodiments, the cysteine engineered antibody comprises a heavy chain comprising the amino acid sequence of SEQ ID NO: 1141 and a light chain comprising the amino acid sequence of SEQ ID NO: 1142. In some embodiments, the cysteine engineered antibody comprises a heavy chain comprising the amino acid sequence of SEQ ID NO: 1141, wherein position 375 or position 400 is substituted with a cysteine. In some embodiments the cysteine engineered antibody comprises a light chain the amino acid sequence of SEQ ID NO: 1142, wherein position 114 or position 121 is substituted with a cysteine. In some embodiments, the antibody of the antibody drug conjugate is B7H4-22216.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1055, 1056, 1057, 1058, 1059, and 1060, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1061 and a light chain variable region comprising an amino acid sequence that is at least 95% at least 96%, at least 97%, at last 98%, at least 99%, or 100% identical to the amino acid sequence of SEQ ID NO: 1062. In some embodiments, the cysteine engineered antibody comprises a heavy chain comprising the amino acid sequence of SEQ ID NO: 1143 and a light chain comprising the amino acid sequence of SEQ ID NO: 1144. In some embodiments, the cysteine engineered antibody comprises a heavy chain comprising the amino acid sequence of SEQ ID NO: 1143, wherein position 375 or position 400 is substituted with a cysteine. In some embodiments the cysteine engineered antibody comprises a light chain the amino acid sequence of SEQ ID NO: 1144, wherein position 114 or position 121 is substituted with a cysteine. In some embodiments, the antibody of the antibody drug conjugate is B7H4-15489.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOS: 1063, 1064, 1065, 1066, 1067, and 1068, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1069 and a light chain variable region comprising an amino acid sequence that is at least 95% at least 96%, at least 97%, at last 98%, at least 99%, or 100% identical to the amino acid sequence of SEQ ID NO: 1070. In some embodiments, the cysteine engineered antibody comprises a heavy chain comprising the amino acid sequence of SEQ ID NO: 1145 and a light chain comprising the amino acid sequence of SEQ ID NO: 1146. In some embodiments, the cysteine engineered antibody comprises a heavy chain comprising the amino acid sequence of SEQ ID NO: 1145, wherein position 375 or position 400 is substituted with a cysteine. In some embodiments the cysteine engineered antibody comprises a light chain the amino acid sequence of SEQ ID NO: 1146, wherein position 114 or position 121 is substituted with a cysteine. In some embodiments, the antibody of the antibody drug conjugate is B7H4-20516.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1071, 1072, 1073, 1074, 1075, and 1076, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1077 and a light chain variable region comprising an amino acid sequence that is at least 95% at least 96%, at least 97%, at last 98%, at least 99%, or 100% identical to the amino acid sequence of SEQ ID NO: 1078. In some embodiments, the cysteine engineered antibody comprises a heavy chain comprising the amino acid sequence of SEQ ID NO: 1147 and a light chain comprising the amino acid sequence of SEQ ID NO: 1148. In some embodiments, the cysteine engineered antibody comprises a heavy chain comprising the amino acid sequence of SEQ ID NO: 1147, wherein position 375 or position 400 is substituted with a cysteine. In some embodiments the cysteine engineered antibody comprises a light chain the amino acid sequence of SEQ ID NO: 1148, wherein position 114 or position 121 is substituted with a cysteine. In some embodiments, the antibody of the antibody drug conjugate is B7H4-15472.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1079, 1080, 1081, 1082, 1083, and 1084, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1085 and a light chain variable region comprising an amino acid sequence that is at least 95% at least 96%, at least 97%, at last 98%, at least 99%, or 100% identical to the amino acid sequence of SEQ ID NO: 1086. In some embodiments, the cysteine engineered antibody comprises a heavy chain comprising the amino acid sequence of SEQ ID NO: 1149 and a light chain comprising the amino acid sequence of SEQ ID NO: 1150. In some embodiments, the cysteine engineered antibody comprises a heavy chain comprising the amino acid sequence of SEQ ID NO: 1149, wherein position 375 or position 400 is substituted with a cysteine. In some embodiments the cysteine engineered antibody comprises a light chain the amino acid sequence of SEQ ID NO: 1150, wherein position 114 or position 121 is substituted with a cysteine. In some embodiments, the antibody of the antibody drug conjugate is B7H4-15503.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1087, 1088, 1089, 1090, 1091, and 1092, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1093 and a light chain variable region comprising an amino acid sequence that is at least 95% at least 96%, at least 97%, at last 98%, at least 99%, or 100% identical to the amino acid sequence of SEQ ID NO: 1094. In some embodiments, the cysteine engineered antibody comprises a heavy chain comprising the amino acid sequence of SEQ ID NO: 1151 and a light chain comprising the amino acid sequence of SEQ ID NO: 1152. In some embodiments, the cysteine engineered antibody comprises a heavy chain comprising the amino acid sequence of SEQ ID NO: 1151, wherein position 375 or position 400 is substituted with a cysteine. In some embodiments the cysteine engineered antibody comprises a light chain the amino acid sequence of SEQ ID NO: 1152, wherein position 114 or position 121 is substituted with a cysteine. In some embodiments, the antibody of the antibody drug conjugate is B7H4-15495.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1095, 1096, 1097, 1098, 1099, and 1100, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1101 and a light chain variable region comprising an amino acid sequence that is at least 95% at least 96%, at least 97%, at last 98%, at least 99%, or 100% identical to the amino acid sequence of SEQ ID NO: 1102. In some embodiments, the cysteine engineered antibody comprises a heavy chain comprising the amino acid sequence of SEQ ID NO: 1153 and a light chain comprising the amino acid sequence of SEQ ID NO: 1154. In some embodiments, the cysteine engineered antibody comprises a heavy chain comprising the amino acid sequence of SEQ ID NO: 1153, wherein position 375 or position 400 is substituted with a cysteine. In some embodiments the cysteine engineered antibody comprises a light chain the amino acid sequence of SEQ ID NO: 1154, wherein position 114 or position 121 is substituted with a cysteine. In some embodiments, the antibody of the antibody drug conjugate is B7H4-15478.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOs: 1103, 1104, 1105, 1106, 1107, and 1108, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1109 and a light chain variable region comprising an amino acid sequence that is at least 95% at least 96%, at least 97%, at last 98%, at least 99%, or 100% identical to the amino acid sequence of SEQ ID NO: 1110. In some embodiments, the cysteine engineered antibody comprises a heavy chain comprising the amino acid sequence of SEQ ID NO: 1155 and a light chain comprising the amino acid sequence of SEQ ID NO: 1156. In some embodiments, the cysteine engineered antibody comprises a heavy chain comprising the amino acid sequence of SEQ ID NO: 1155, wherein position 375 or position 400 is substituted with a cysteine. In some embodiments the cysteine engineered antibody comprises a light chain the amino acid sequence of SEQ ID NO: 1156, wherein position 114 or position 121 is substituted with a cysteine. In some embodiments, the antibody of the antibody drug conjugate is B7H4-15441.

In some embodiments, a cysteine engineered antibody (e.g., a cysteine engineered antibody of a cysteine engineered ADC) provided herein binds to B7H4. In some embodiments, the cysteine engineered antibody comprises CDR-H1, CDR-H2, CDR-H3, CDR-L1, CDR-L2, and CDR-L3 comprising the amino acid sequences of SEQ ID NOS: 1111, 1112, 1113, 1114, 1115, and 1116, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1117 and a light chain variable region comprising an amino acid sequence that is at least 95% at least 96%, at least 97%, at last 98%, at least 99%, or 100% identical to the amino acid sequence of SEQ ID NO: 1118. In some embodiments, the cysteine engineered antibody comprises a heavy chain comprising the amino acid sequence of SEQ ID NO: 1157 and a light chain comprising the amino acid sequence of SEQ ID NO: 1158. In some embodiments, the cysteine engineered antibody comprises a heavy chain comprising the amino acid sequence of SEQ ID NO: 1157, wherein position 375 or position 400 is substituted with a cysteine. In some embodiments the cysteine engineered antibody comprises a light chain the amino acid sequence of SEQ ID NO: 1158, wherein position 114 or position 121 is substituted with a cysteine. In some embodiments, the antibody of the antibody drug conjugate is B7H4-20496.

In some embodiments, a cysteine engineered antibody (e.g., a cysteine engineered antibody of a cysteine engineered ADC) provided herein binds to CD70. In some embodiments, the cysteine engineered antibody comprises CDR-H1, CDR-H2, CDR-H3, CDR-L1, CDR-L2, and CDR-L3 comprising the amino acid sequences of SEQ ID NOS: 1159, 1160, 1161, 1162, 1163, and 1164, respectively. In some embodiments, the antibody of the antibody drug conjugate is a h1F6 anti-CD70 antibody, which is described in International Patent Publication No. WO 2006/113909. h1F6 is also known as vorsetuzumab.

In some embodiments, the cysteine engineered antibody comprises CDR-H1, CDR-H2, CDR-H3, CDR-L1, CDR-L2, and CDR-L3 comprising the amino acid sequences of SEQ ID NOs: 1178, 1179, 1180, 1131, 1182, and 1183, respectively. In some embodiments, the cysteine engineered antibody comprises a heavy chain variable region comprising an amino acid sequence that is at least 95%, at least 96%, at least 97%, at last 98%, at least 99%, or 100% identical to the amino acid sequence of SEQ ID NO: 1184 and a light chain variable region comprising an amino acid sequence that is at least 95% at least 96%, at least 97%, at last 98%, at least 99%, or 100% identical to the amino acid sequence of SEQ ID NO: 1185. In some embodiments, the cysteine engineered antibody comprises a heavy chain comprising the amino acid sequence of SEQ ID NO: 1186 and a light chain comprising the amino acid sequence of SEQ ID NO: 1188. In some embodiments, the cysteine engineered antibody comprises a heavy chain comprising the amino acid sequence of SEQ ID NO: 1187 and a light chain comprising the amino acid sequence of SEQ ID NO: 1188. In some embodiments, the cysteine engineered antibody comprises a heavy chain comprising the amino acid sequence of any of SEQ ID NOs: 1186 or 1187, wherein position 375 or position 400 is substituted with a cysteine. In some embodiments the cysteine engineered antibody comprises a light chain the amino acid sequence of SEQ ID NO: 1188, wherein position 114 or position 121 is substituted with a cysteine. In some embodiments, the antibody of the antibody drug conjugate is disitamab/RC-48.

### 2. Cysteine Amino Acid Substitutions

In some embodiments, a cysteine engineered antibody comprises one or more cysteine amino acid substitutions. In some embodiments, the cysteine amino acid substitutions are of free cysteine amino acids. In some embodiments, the cysteine engineered antibody comprises at least one, two, three, four or more, cysteine amino acid substitutions. In some embodiments, the cysteine engineered antibody comprises less than four, three, two or fewer, cysteine amino acid substitutions. In some embodiments, the cysteine engineered antibody comprises one, two, three, or four cysteine amino acid substitutions. In some embodiments, the cysteine engineered antibody comprises two cysteine amino acid substitutions. In some embodiments, the cysteine engineered antibody comprises four cysteine amino acid substitutions.

In some embodiments, the cysteine engineered antibody is a half antibody. In some embodiments, the cysteine engineered antibody comprises one heavy chain. In some embodiments, the cysteine engineered antibody comprises one light chain. In some embodiments, the cysteine engineered antibody comprises one heavy chain and one light chain. In some embodiments, cysteine engineered antibody comprises two cysteine amino acid substitutions. In some embodiments, the heavy chain comprises the amino acid sequence of SEQ ID NO: 892 and the light chain comprises the amino acid sequence of SEQ ID NO: 893.

In some embodiments, the cysteine engineered antibody is a full-length antibody. In some embodiments, the cysteine engineered antibody comprises two heavy chains. In some embodiments, the two heavy chains are identical in amino acid sequence. In some embodiments, the two heavy chains have a different amino acid sequence. In some embodiments, the two heavy chains differ by about one, two, three, four, or more, amino acid residues. In some embodiments, the two heavy chains have different cysteine amino acid substitutions. In some embodiments, the cysteine engineered antibody comprises two light chains. In some embodiments, the two light chains are identical in amino acid sequence. In some embodiments, the two light chains have a different amino acid sequence. In some embodiments, the two light chains differ by about one, two, three, four, or more, amino acid residues. In some embodiments, the two light chains have different cysteine amino acid substitutions. In some embodiments, the cysteine engineered antibody comprises two heavy chains and two light chains, such as two heavy chain-light chain pairs (e.g., a first heavy chain-light chain pair and a second heavy chain-light chain pair). In some embodiments, a first heavy chain-light chain pair is identical in amino acid sequence to a second heavy chain-light chain pair. In some embodiments, the first heavy chain-light chain pair is different in amino acid sequence compared to the second heavy chain-light chain pair. In some embodiments, the first heavy chain-light chain pair has different cysteine amino acid substitutions compared to the second heavy chain-light chain pair. For example, in some embodiments, the cysteine engineered antibody is an asymmetric antibody. In some embodiments, the cysteine engineered antibody comprises four cysteine amino acid substitutions.

In some embodiments, a cysteine amino acid substitution is present at a position on a light chain of the cysteine engineered antibody. In some embodiments, the cysteine engineered antibody comprises a single light chain, and the cysteine amino acid substitution is present at a position on the single light chain. In some embodiments, the cysteine amino acid substitution is at position 121 of the light chain of the cysteine engineered antibody, according to Kabat numbering. In some embodiments, the cysteine amino acid substitution is at position 114 of the light chain of the cysteine engineered antibody, according to Kabat numbering. In some embodiments, the cysteine engineered antibody comprises one cysteine amino acid substitution.

In some embodiments, the cysteine engineered antibody comprises two light chains, and the cysteine amino acid substitution is present at a position on one of the light chains of the cysteine engineered antibody. In some embodiments, the cysteine amino acid substitution is present at a position on each of the light chains of the cysteine engineered antibody. In some embodiments, the cysteine amino acid substitution is at the same position on each of the light chains of the cysteine engineered antibody. In some embodiments, the cysteine amino acid substitution is at a different position on each of the light chains of the cysteine engineered antibody. In some embodiments, the cysteine amino acid substitution is at position 121 of a light chain of the cysteine engineered antibody, according to Kabat numbering. In some embodiments, the cysteine amino acid substitution is at position 121 of each of the light chains of the cysteine engineered antibody, according to Kabat numbering. In some embodiments, the cysteine amino acid substitution is at position 114 of the light chain of the cysteine engineered antibody, according to Kabat numbering. In some embodiments, the cysteine amino acid substitution is at position 114 of each of the light chains of the cysteine engineered antibody, according to Kabat numbering. In some embodiments, the cysteine amino acid substitution is at position 114 on a first light chain of the cysteine engineered antibody and at position 121 of a second light chain of the cysteine engineered antibody, according to Kabat numbering. In some embodiments, the cysteine engineered antibody comprises two cysteine amino acid substitutions. In some embodiments, the cysteine engineered antibody comprises SEQ ID NO. 893.

In some embodiments, a cysteine amino acid substitution is present at a position on a heavy chain of the cysteine engineered antibody. In some embodiments, the cysteine engineered antibody comprises a single heavy chain, and the cysteine amino acid substitution is present at a position on the single heavy chain. In some embodiments, the cysteine amino acid substitution is at position 375 of the heavy chain of the cysteine engineered antibody, according to EU numbering. In some embodiments, the cysteine amino acid substitution is at position 400 of the heavy chain of the cysteine engineered antibody, according to EU numbering. In some embodiments, the cysteine engineered antibody comprises one cysteine amino acid substitution. In some embodiments, the cysteine engineered antibody comprises SEQ ID NO. 892.

In some embodiments, a cysteine amino acid substitution is present at a position on one of the heavy chains of the cysteine engineered antibody. In some embodiments, the cysteine amino acid substitution is present at a position on each of the heavy chains of the cysteine engineered antibody. In some embodiments, the cysteine amino acid substitution is the same on each of the heavy chains of the cysteine engineered antibody. In some embodiments, the cysteine amino acid substitution is different on each of the heavy chains of the cysteine engineered antibody. In some embodiments, the cysteine amino acid substitution is at position 375 of the heavy chain of the cysteine engineered antibody, according to EU numbering. In some embodiments, the cysteine amino acid substitution is at position 375 of each of the heavy chains of the cysteine engineered antibody, according to EU numbering. In some embodiments, the cysteine amino acid substitution is at position 400 of the heavy chain of the cysteine engineered antibody, according to EU numbering. In some embodiments, the cysteine amino acid substitution is at position 400 of each of the heavy chains of the cysteine engineered antibody, according to EU numbering. In some embodiments, the cysteine amino acid substitution is at position 400 of a first heavy chain of the cysteine engineered antibody and at position 375 of a second heavy chain of the cysteine engineered antibody, according to EU numbering. In some embodiments, the cysteine engineered antibody comprises two cysteine amino acid substitutions. In some embodiments, the cysteine engineered antibody comprises SEQ ID NO. 892.

In some embodiments, the cysteine engineered antibody comprises at least one cysteine amino acid substitution on a light chain of the cysteine engineered antibody and at least one cysteine amino acid substitution on a heavy chain of the cysteine engineered antibody. In some embodiments, the cysteine engineered antibody comprises two cysteine amino acid substitutions. In some embodiments, the cysteine amino acid substitutions are at position 121 the light chain, according to Kabat numbering, and position 375 of the heavy chain, according to EU numbering. In some embodiments, the cysteine amino acid substitutions are at position 121 of the light chain, according to Kabat numbering, and position 400 of the heavy chain, according to EU numbering. In some embodiments, the cysteine amino acid substitutions are at position 114 of the light chain, according to Kabat numbering, and position 400 of the heavy chain, according to EU numbering. In some embodiments, the cysteine engineered antibody is a half antibody, and comprises a cysteine amino acid substitution at position 121 the light chain, according to Kabat numbering, and position 375 of the heavy chain, according to EU numbering. In some embodiments, the cysteine engineered antibody is a half antibody, and comprises a cysteine amino acid substitution at position 114 and position 121 of the light chain, according to Kabat numbering. In some embodiments, the cysteine engineered antibody is a half antibody, and comprises a cysteine amino acid substitution at position 121 of the light chain, according to Kabat numbering, and position 400 of the heavy chain, according to EU numbering. In some embodiments, the cysteine engineered antibody is a half antibody, and comprises a cysteine amino acid substitution at position 114 of the light chain, according to Kabat numbering, and position 400 of the heavy chain, according to EU numbering. In some embodiments, the heavy chain comprises the amino acid sequence of SEQ ID NO: 892 and the light chain comprises the amino acid sequence of SEQ ID NO: 893.

In some embodiments, the cysteine engineered antibody comprises four cysteine amino acid substitutions. In some embodiments, the cysteine engineered antibody comprises two heavy chains and two light chains, and comprises a cysteine amino acid substitution on each of the light chains and a cysteine amino acid substitution on each of the heavy chains. In some embodiments, the cysteine engineered antibody comprises the same cysteine amino acid substitution on each of the light chains. In some embodiments, the cysteine engineered antibody comprises a cysteine amino acid substitution at position 121 of each of the light chains, according to Kabat numbering. In some embodiments, the cysteine engineered antibody comprises a cysteine amino acid substitution at position 114 of each of the light chains, according to Kabat numbering. In some embodiments, the cysteine engineered antibody comprises the same cysteine amino acid substitution on each of the heavy chains. In some embodiments, the cysteine engineered antibody comprises a cysteine amino acid substitution at position 400 of each of the heavy chains, according to EU numbering. In some embodiments, the cysteine engineered antibody comprises a cysteine amino acid substitution at position 375 of each of the heavy chains, according to EU numbering. In some embodiments, the cysteine engineered antibody comprises a cysteine amino acid substitution at position 375 and a cysteine amino acid substitution at position 400 of each of the heavy chains, according to EU numbering. In some embodiments, the cysteine engineered antibody comprises the same cysteine amino acid substitution on each of the light chains and the same cysteine amino acid substitution on each of the heavy chains. In some embodiments, the cysteine amino acid substitutions are at position 121 of each of the light chains, according to Kabat numbering, and position 375 of each of the heavy chains, according to EU numbering. In some embodiments, the cysteine amino acid substitutions are at position 121 of each of the light chains, according to Kabat numbering, and position 400 of each of the heavy chains, according to EU numbering. In some embodiments, the cysteine amino acid substitutions are at position 114 of each of the light chains, according to Kabat numbering, and position 400 of each of the heavy chains, according to EU numbering. In some embodiments, the cysteine engineered antibody comprises the heavy chain comprising the amino acid sequence of SEQ ID NO: 892 and the light chain comprising the amino acid sequence of SEQ ID NO: 893.

In some embodiments, the cysteine engineered antibody comprises a different cysteine amino acid substitution on each of the light chains or each of the heavy chains. In some embodiments, the cysteine engineered antibody comprises a different cysteine amino acid substitution on each of the light chains. In some embodiments, the cysteine engineered antibody comprises a different cysteine amino acid substitution on each of the heavy chains. In some embodiments, the cysteine engineered antibody comprises a different cysteine amino acid substitution on each of the light chains and the same cysteine amino acid substitution on each of the heavy chains. In some embodiments, the cysteine engineered antibody comprises a different cysteine amino acid substitution on each of the heavy chains and the same cysteine amino acid substitution on each of the light chains. In some embodiments, the cysteine engineered antibody comprises a cysteine amino acid substitution at position 121 or position 114 of a first light chain, according to Kabat numbering, and position 375 or position 400 of one of a first heavy chain, according to EU numbering. In some embodiments, the cysteine engineered antibody comprises a cysteine amino acid substitution at position 121 of a first the light chain, according to Kabat numbering, and position 375 of a first heavy chain, according to EU numbering. In some embodiments, the cysteine engineered antibody comprises a cysteine amino acid substitution at position 121 of a first light chain, according to Kabat numbering, and position 400 of a first heavy chain, according to EU numbering. In some embodiments, the cysteine engineered antibody comprises a cysteine amino acid substitution at position 114 of a first light chain, according to Kabat numbering, and position 400 of a first heavy chain, according to EU numbering. The first light chain and the first heavy chain (e.g., half antibody) of the cysteine engineered antibodies provided herein may be combined with any other suitable half antibody comprising a second light chain and a second heavy chain. For example, in some embodiments, the second half antibody does not comprise cysteine amino acid substitutions in either the second light chain or the second heavy chain. In some embodiments, the cysteine engineered antibody comprises the heavy chain comprising the amino acid sequence of SEQ ID NO: 892 and the light chain comprising the amino acid sequence of SEQ ID NO: 893.

Alternatively, the second half antibody may comprise any cysteine amino acid substitutions suitable for generating cysteine engineered antibodies, known in the art. Cysteine amino acid substitutions suitable for use in cysteine engineered antibody and cysteine engineered ADCs generated therefrom are described, for example, in U.S. Pat. Nos. 7,521,541, 9,845,355, 11,147,852, and US 2020/0338207, the contents of each of which are herein incorporated by reference in their entirety. In some embodiments, the second half antibody comprises a cysteine amino acid substitution at any of position 15, 110, 114, 121, 127, 168, 205, or a combination thereof, of the second light chain, according to Kabat numbering, and a cysteine amino acid substitution at position 5, 23, 84, 112, 118, 120, 282, 375 and 400, or a combination thereof, of the second heavy chain, according to EU numbering. In some embodiments, the second half antibody comprises a cysteine amino acid substitution at any of position 107, 108, 109, 114, 129, 142, 143, 145, 152, 154, 156, 159, 161, 165, 168, 169, 170, 182, 183, 197, 199, and 203, or a combination thereof, of the second light chain, and a cysteine amino acid substitution at position 121, 124, 152, 171, 258, 292, 333, 334, 360, 375, 392 and 400, or a combination thereof, of the second heavy chain, both according to EU numbering. In some embodiments, the second half antibody comprises a cysteine amino acid substitution at position 142 of the second light chain and position 375 of the second heavy chain. In some embodiments, the second half antibody comprises a cysteine amino acid substitution at position 165 of the second light chain and position 258 of the second heavy chain. In some embodiments, the second half antibody comprises a cysteine amino acid substitution at position 142 of the second light chain and position 171 of the second heavy chain. In some embodiments, the second half antibody comprises a cysteine amino acid substitution at position 205 of the second light chain and position 149 of the second heavy chain. In some embodiments, the second half antibody comprises a cysteine amino acid substitution at position 205 of the second light chain and position 118 of the second heavy chain. In some embodiments, the second half antibody comprises a cysteine amino acid substitution at position 205 of the second light chain and position 140 of the second heavy chain. In some embodiments, the second half antibody comprises a cysteine amino acid substitution at position 142 of the second light chain and position 118 of the second heavy chain. In some embodiments, the second half antibody comprises a cysteine amino acid substitution at position 205 of the second light chain and position 375 of the second heavy chain. In some embodiments, the second half antibody comprises a cysteine amino acid substitution at position 183 of the second light chain and position 290 of the second heavy chain. In some embodiments, the second half antibody comprises a cysteine amino acid substitution at position 392 of the heavy chain and position 334 of the second heavy chain. In some embodiments, the second half antibody comprises a cysteine amino acid substitution at position 205 of the second light chain and position 239 of the second heavy chain. In some embodiments, the cysteine engineered antibody comprises a cysteine amino acid substitution at position 121 of the first light chain and position 114 of second light chain, according to Kabat numbering, and position 375 of the first heavy chain and position 400 of the second heavy chain, according to EU numbering.

The cysteine engineered antibodies that are useful in the present methods can be produced by any method known in the art for the synthesis of proteins, typically, e.g., by recombinant expression techniques. Recombinant expression of a cysteine engineered antibody or derivative thereof requires construction of an expression vector containing a nucleic acid that encodes the antibody or derivative thereof. Once a nucleic acid encoding such a protein has been obtained, the vector for the production of the protein molecule may be produced by recombinant DNA technology using techniques well known in the art. Standard techniques such as, for example, those described in Sambrook and Russell, Molecular Cloning: A Laboratory Manual (Cold Spring Harbor Laboratory Press, Cold Spring Harbor, N.Y., 3rd ed., 2001); Sambrook et al, Molecular Cloning: A Laboratory Manual (Cold Spring Harbor Laboratory Press, Cold Spring Harbor, N.Y., 2nd ed., 1989); Short Protocols in Molecular Biology (Ausubel et al, John Wiley & Sons, New York, 4th ed., 1999); and Glick & Pasternak, Molecular Biotechnology: Principles and Applications of Recombinant DNA (ASM Press, Washington, D.C., 2nd ed., 1998) can be used for recombinant nucleic acid methods, nucleic acid synthesis, cell culture, transgene incorporation, and recombinant protein expression.

For example, for recombinant expression of a cysteine engineered antibody, an expression vector may encode a heavy or light chain thereof, or a heavy or light chain variable domain, operably linked to a promoter. An expression vector may include, for example, the nucleotide sequence encoding the constant region of the antibody molecule (see, e.g., PCT Publication WO 86/05807; PCT Publication WO 89/01036; and U.S. Pat. No. 5,122,464), and the variable domain of the antibody may be cloned into such a vector for expression of the entire heavy or light chain. The expression vector is transferred to a host cell by conventional techniques, and the transfected cells are then cultured by conventional techniques to produce the anti-CD70 antibody. In typical embodiments for the expression of double-chained antibodies, vectors encoding both the heavy and light chains can be co-expressed in the host cell for expression of the entire immunoglobulin molecule.

A variety of prokaryotic and eukaryotic host-expression vector systems can be utilized to express a cysteine engineered antibody or derivative thereof. Typically, eukaryotic cells, particularly for whole recombinant cysteine engineered antibody molecules, are used for the expression of the recombinant protein. For example, mammalian cells such as Chinese hamster ovary cells (CHO), in conjunction with a vector such as the major intermediate early gene promoter element from human cytomegaloviras, is an effective expression system for the production of anti-CD70 antiobies and derivatives thereof (see, e.g., Foecking et al., 1986, Gene 45:101; Cockett et al, 1990, Bio/Technology 8:2).

Other host-expression systems include, for example, plasmid-based expression systems in bacterial cells (see, e.g., Ruther et al, 1983, EMBO 1,2:1791; Inouye & Inouye, 1985, Nucleic Acids Res. 13:3101-3109; Van Heeke & Schuster, 1989, J. Biol. Chem. 24:5503-5509); insect systems such as, e.g., the use of Autographa californica nuclear polyhedrosis virus (AcNPV) expression vector in Spodoptera frugiperda cells; and viral-based expression systems in mammalian cells, such as, e.g., adenoviral-based systems (see, e.g., Logan & Shenk, 1984, Proc. Natl. Acad. Sci. USA 81:355-359; Bittner et al, 1987, Methods in Enzymol. 153:51-544).

In addition, a host cell strain can be chosen that modulates the expression of the inserted sequences, or modifies and processes the gene product in the specific fashion desired. Appropriate cell lines or host systems can be chosen to ensure the correct modification and processing (e.g., glycosylation, phosphorylation, and cleavage) of the foreign protein expressed. To this end, eukaryotic host cells which possess the cellular machinery for proper processing of the primary transcript and gene product can be used. Such mammalian host cells include, for example, CHO, VERO, BHK, HeLa, COS, MDCK, 293, 3T3, and W138.

A stable expression system is typically used for long-term, high-yield production of recombinant cysteine engineered antibody or derivative thereof. For example, cell lines that stably express the cysteine engineered antibody or derivative thereof can be engineered by transformation of host cells with DNA controlled by appropriate expression control elements (e.g., promoter, enhancer, sequences, transcription terminators, polyadenylation sites) and a selectable marker, followed by growth of the transformed cells in a selective media. The selectable marker confers resistance to the selection and allows cells to stably integrate the DNA into their chromosomes and grow to form foci which in turn can be cloned and expanded into cell lines. A number of selection systems can be used, including, for example, the hepes simplex virus thymidine kinase, hypoxanthineguanine phosphoribosyltransferase, and adenine phosphoribosyltransferase genes, which can be employed in tk “, hgprt” or aprf cells, respectively. Also, antimetabolite resistance can be used as the basis of selection for the following genes: dhfr, which confers resistance to methotrexate; gpt, which confers resistance to mycophenolic acid; neo, which confers resistance to the aminoglycoside G-418; and hygro, which confers resistance to hygromycin. Methods commonly known in the art of recombinant DNA technology can be routinely applied to select the desired recombinant clone, and such methods are described, for example, in Current Protocols in Molecular Biology (Ausubel et al. eds., John Wiley & Sons, N.Y., 1993); Kriegler, Gene Transfer and Expression, A Laboratory Manual (Stockton Press, N.Y., 1990); Current Protocols in Human Genetics (Dracopoli et al. eds., John Wiley & Sons, N.Y., 1994, Chapters 12 and 13); and Colberre-Garapin et al, 1981, J. Mol. Biol. 150:1.

The expression levels of an antibody or derivative can be increased by vector amplification. (See generally, e.g., Bebbington & Hentschel, The Use of Vectors Based on Gene Amplification for the Expression of Cloned Genes in Mammalian Cells in DNACloning, Vol. 3 (Academic Press, New York, 1987).) When a marker in the vector system expressing a cysteine engineered antibody or derivative thereof is amplifiable, an increase in the level of inhibitor present in host cell culture media will select host cells that have increased copy number of a marker gene conferring resistance to the inhibitor. The copy number of an associated antibody gene will also be increased, thereby increasing expression of the antibody or derivative thereof (see Crouse et al, 1983, Mol. Cell. Biol 3:257).

Where the cysteine engineered antibody comprises both a heavy and a light chain or derivatives thereof, the host cell may be co-transfected with two expression vectors, the first vector encoding the heavy chain protein and the second vector encoding the light chain protein. The two vectors may contain identical selectable markers which enable equal expression of heavy and light chain proteins. Alternatively, a single vector may be used which encodes, and is capable of expressing, both heavy and light chain proteins. In such situations, the light chain is typically placed before the heavy chain to avoid an excess of toxic free heavy chain (see Proudfoot, 1986, Nature 322:52; Kohler, 1980, Proc. Natl. Acad. Sci. USA 77:2197). The coding sequences for the heavy and light chains may comprise cDNA or genomic DNA.

Once a cysteine engineered antibody or derivative thereof has been produced (e.g., by an animal, chemical synthesis, or recombinant expression), it can be purified by any suitable method for purification of proteins, including, for example, by chromatography (e.g., ion exchange or affinity chromatography (such as, for example, Protein A chromatography for purification of antibodies having an intact Fc region)), centrifugation, differential solubility, or by any other standard technique for the purification of proteins. A cysteine engineered antibody or derivative thereof can, for example, be fused to a marker sequence, such as a peptide, to facilitate purification by affinity chromatography. Suitable marker amino acid sequences include, e.g., a hexa-histidine peptide, such as the tag provided in a pQE vector (QIAGEN, Inc., 9259 Eton Avenue, Chatsworth, CA, 91311), and the “HA” tag, which corresponds to an epitope derived from the influenza hemagglutinin protein (Wilson et al, 1984, Cell 37:767), and the “flag” tag.

Once a cysteine engineered antibody is produced, its ability to exert a cytotoxic effect on a target antigen cancer cell (when conjugated to a cytotoxic agent, e.g., where the cysteine engineered antibody is comprised in a cysteine engineered ADC, such as any of the cysteine engineered ADCs described herein) is determined by the methods described herein or as known in the art.

### B. Cysteine Engineered Antibody Drug Conjugate (ADC)

In some aspects, provided herein is a cysteine engineered ADC comprising a cysteine engineered antibody, such as any of the cysteine engineered antibodies described herein (e.g., those described in Section II.A), and a drug moiety. Cysteine amino acid substitutions (e.g., cysteine amino acid substitutions of free cysteines) on a cysteine engineered antibody may serve as useful handles for installation of a variety of functional groups, including imaging agents (such as chromophores and fluorophores), diagnostic agents (such as MRI contrast reagents and radioisotopes), stability agents (such as polyetheylene glycol polymers), and cytotoxic agents. Thus, in some aspects, the drug moiety may be attached to one or more cysteine amino acid substitutions of the cysteine engineered antibody via a linker moiety to form the cysteine engineered ADCs described herein.

In some embodiments, a cysteine engineered ADC is of the following formula:

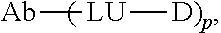

wherein:
Ab is a cysteine engineered antibody,
LU is a linker moiety,
D is a drug moiety, and
the subscript p is a value from 1 to 8.

In the formula above, the linker, LU, is conjugated to the cysteine engineered antibody via one or more cysteine amino acid substitutions. The value of the subscript p is dependent on the number of cysteine amino acid substitutions (e.g., free cysteines) available for conjugation. For example, for a cysteine engineered antibody having two free cysteines, (e.g., one position on a heavy chain and one position on a light chain), the value of p can be two. Similarly, for a cysteine engineered antibody having four cysteine amino acid substitutions (e.g., one position on each heavy chain and one position on each light chain), the value of p can be four. In some embodiments, p is a value from about 1 to about 4. In some embodiments, p is about 4 (e.g., a drug-antibody ratio (DAR) of 4).

### 1. Drug Moiety Loading

The drug-antibody ratio (DAR) (or average number of drug-linker molecules, also referred to herein as “drug moiety-linker molecules”, per cysteine engineered antibody in a cysteine engineered ADC) is an important characteristic of an ADC, as it is a primary determinant of the amount of drug moiety that may be delivered to a target cell. An average DAR of a cysteine engineered ADC includes drug moieties conjugated to free cysteines (e.g., cysteine amino acid substitutions of the cysteine engineered antibodies described herein), as well as drug moeties conjugated to positions other than the intended free cysteines, and the amount of unconjugated cysteine engineered antibodies in the composition. Sites for conjugation of the drug moieties, such as any of the drug moieties described herein, can be generated by generating cysteine amino acid substitutions (e.g., at one position on each heavy chain of the cysteine engineered antibody and at one position on each light chain of the cysteine engineered antibody). In some embodiments, the cysteine engineered ADC has an average DAR of about three and a half to about four, wherein the cysteine engineered ADC comprises four drug moieties per cysteine engineered antibody.

In some embodiments, the cysteine engineered ADC comprises a drug moiety conjugated to each individual cysteine amino acid substitution. In some embodiments, each of the drug moieties is the same drug moiety. In some embodiments, the drug moieties can be different. In some embodiments, each of the drug moieties are different. Typically, cysteine engineered ADCs prepared with cysteine engineered antibodies comprising four cysteine amino acid substitutions have an average drug moiety-loading of from about 3.4 to about 4.5 drug moieties per cysteine engineered antibody. The average number of drug moieties per cysteine engineered antibody can be, for example, any of about 3.3, 3.4, 3.5, 3.6, 3.7, 3.8, 3.9, or 4.0. In some embodiments, the average drug moiety-loading for a cysteine engineered ADC comprising four cysteine amino acid substitutions is from about 3.6 to about 4.2 drug moieties per cysteine engineered antibody, or from about 3.8 to about 4 drug moieties per cysteine engineered antibody. In some embodiments, the cysteine engineered ADC comprises four drug moieties, each conjugated to one of the four cysteine amino acid substitutions. In some embodiments, the four drug moieties are the same drug moiety. In some embodiments, the drug moieties may be different from one another. In some embodiments, each of the four drug moieties are different.

Various analytical methods can be used to determine the yields and isomeric mixtures of the cysteine engineered ADCs. Following conjugation of the drug moiety to the cysteine engineered antibody, the cysteine engineered ADC may be selected (e.g., separated) from a mixture. In some embodiments, the cysteine engineered ADC (e.g., cysteine engineered antibody conjugated to one or more drug moieties, such as via a linker moiety) may be separated from a mixture based on the characteristics of the cysteine engineered antibody, the drug moiety, and/or the cysteine engineered ADC itself. Other techniques useful for analysis of cysteine engineered ADC include, but are not limited to, reversed-phase chromatography, capillary electrophoresis, and mass spectrometry. Cysteine engineered ADCs can be analyzed, for example, by LC/MS coupled with proteolytic digestion to determine the location of a drug moiety in a cysteine engineered ADC.

### 2. Cysteine Amino Acid Substitutions

The site of a cysteine amino acid substitution within a cysteine engineered antibody of a cysteine engineered ADC can have an impact on the properties of the cysteine engineered ADC. For instance, cysteine amino acid substitutions entirely buried within the structure of the cysteine engineered antibody can be difficult to conjugate with a drug moiety because of poor access to the solvent, while cysteine amino acid substitutions on the exterior surface of the cysteine engineered antibody may result in cysteine engineered ADCs that have lower stability because of prolonged exposure to plasma when administered to a patient. Also, cysteine engineered ADCs prepared from cysteine engineered antibodies with highly surface exposed cysteine amino acid substitutions may be sensitive to the hydrophobicity of the drug moiety, while cysteine amino acid substitutions in more protected locations may be less sensitive to the properties of the drug moiety. The location of cysteine amino acid substitutions can also be used to modulate effector function as desired for a particular cysteine engineered ADC. For example, conjugation of a drug moiety-linker to a cysteine amino acid substitution in an effector function binding domain can be used to block binding to effector function-mediating receptors.

In some embodiments, a cysteine engineered ADC comprises a cysteine engineered antibody comprising one or more cysteine amino acid substitutions. In some embodiments, the cysteine amino acid substitutions are of free cysteines. As described herein, the cysteine amino acid substitutions (e.g., free cysteines) on the cysteine engineered antibody of the cysteine engineered ADC may be conjugatable. By conjugatable it is meant that the cysteine amino acid substitution is capable of being conjugated to a functional agent (e.g., a drug moiety) without first denaturing the cysteine engineered antibody.

In some embodiments, the cysteine amino acid substitution is located at a position in the Fc region of the cysteine engineered antibody. In some embodiments, the cysteine amino acid substitution is located at a position in a heavy chain of the cysteine engineered antibody. In some embodiments, the cysteine amino acid substitution is located at a position in the constant region of the heavy chain. In some embodiments, the cysteine amino acid substitution is located at a position in a light chain of the cysteine engineered antibody. In some embodiments, the cysteine amino acid substitution is located at a position in the constant region of the light chain. In some embodiments, the cysteine engineered antibody of the cysteine engineered ADC comprises one heavy chain. In some embodiments, the cysteine engineered antibody of the cysteine engineered ADC comprises one light chain. In some embodiments, the cysteine engineered antibody of the cysteine engineered ADC comprises one heavy chain and one light chain, e.g., the cysteine engineered antibody of the cysteine engineered ADC is a half antibody. In some embodiments, the cysteine engineered antibody of the cysteine engineered ADC comprises two heavy chains. In some embodiments, the cysteine engineered antibody of the cysteine engineered ADC comprises two light chains. In some embodiments, the cysteine engineered antibody of the cysteine engineered ADC comprises two heavy chains and two light chains. In some embodiments, cysteine engineered antibody is a half antibody. In some embodiments, cysteine engineered antibody is a full-length antibody. In some embodiments, cysteine engineered antibody is an asymmetric antibody.

In some embodiments, a cysteine engineered antibody of the cysteine engineered ADC comprises one or more cysteine amino acid substitutions. In some embodiments, the cysteine amino acid substitutions are of free cysteine amino acids. In some embodiments, the cysteine engineered antibody comprises at least one, two, three, four or more, cysteine amino acid substitutions. In some embodiments, the cysteine engineered antibody comprises less than four, three, two or fewer, cysteine amino acid substitutions. In some embodiments, the cysteine engineered antibody comprises one, two, three, or four cysteine amino acid substitutions. In some embodiments, the cysteine engineered antibody comprises two cysteine amino acid substitutions. In some embodiments, the cysteine engineered antibody comprises four cysteine amino acid substitutions.

In some embodiments, the cysteine engineered antibody of the cysteine engineered ADC is a half antibody. In some embodiments, the cysteine engineered antibody comprises one heavy chain. In some embodiments, the cysteine engineered antibody comprises one light chain. In some embodiments, the cysteine engineered antibody comprises one heavy chain and one light chain. In some embodiments, cysteine engineered antibody comprises two cysteine amino acid substitutions.

In some embodiments, the cysteine engineered antibody of the cysteine engineered ADC is a full-length antibody. In some embodiments, the cysteine engineered antibody comprises two heavy chains. In some embodiments, the two heavy chains are identical in amino acid sequence. In some embodiments, the two heavy chains have a different amino acid sequence. In some embodiments, the two heavy chains differ by about one, two, three, four, or more, amino acid residues. In some embodiments, the two heavy chains have different cysteine amino acid substitutions. In some embodiments, the cysteine engineered antibody comprises two light chains. In some embodiments, the two light chains are identical in amino acid sequence. In some embodiments, the two light chains have a different amino acid sequence. In some embodiments, the two light chains differ by about one, two, three, four, or more, amino acid residues. In some embodiments, the two light chains have different cysteine amino acid substitutions. In some embodiments, the cysteine engineered antibody comprises two heavy chains and two light chains, such as two heavy chain-light chain pairs (e.g., a first heavy chain-light chain pair and a second heavy chain-light chain pair). In some embodiments, a first heavy chain-light chain pair is identical in amino acid sequence to a second heavy chain-light chain pair. In some embodiments, the first heavy chain-light chain pair is different in amino acid sequence compared to the second heavy chain-light chain pair. In some embodiments, the first heavy chain-light chain pair has different cysteine amino acid substitutions compared to the second heavy chain-light chain pair. For example, in some embodiments, the cysteine engineered antibody is an asymmetric antibody. In some embodiments, the cysteine engineered antibody comprises four cysteine amino acid substitutions.

In some embodiments, a cysteine amino acid substitution is present at a position on a light chain of the cysteine engineered antibody of the cysteine engineered ADC. In some embodiments, the cysteine engineered antibody comprises a single light chain, and the cysteine amino acid substitution is present at a position on the single light chain. In some embodiments, the cysteine amino acid substitution is at position 121 of the light chain of the cysteine engineered antibody, according to Kabat numbering. In some embodiments, the cysteine amino acid substitution is at position 114 of the light chain of the cysteine engineered antibody, according to Kabat numbering. In some embodiments, the cysteine engineered antibody comprises one cysteine amino acid substitution.

In some embodiments, the cysteine engineered antibody of the cysteine engineered ADC comprises two light chains, and the cysteine amino acid substitution is present at a position on one of the light chains of the cysteine engineered antibody. In some embodiments, the cysteine amino acid substitution is present at a position on each of the light chains of the cysteine engineered antibody. In some embodiments, the cysteine amino acid substitution is at the same position on each of the light chains of the cysteine engineered antibody. In some embodiments, the cysteine amino acid substitution is at a different position on each of the light chains of the cysteine engineered antibody. In some embodiments, the cysteine amino acid substitution is at position 121 of a light chain of the cysteine engineered antibody, according to Kabat numbering. In some embodiments, the cysteine amino acid substitution is at position 121 of each of the light chains of the cysteine engineered antibody, according to Kabat numbering. In some embodiments, the cysteine amino acid substitution is at position 114 of the light chain of the cysteine engineered antibody, according to Kabat numbering. In some embodiments, the cysteine amino acid substitution is at position 114 of each of the light chains of the cysteine engineered antibody, according to Kabat numbering. In some embodiments, the cysteine amino acid substitution is at position 114 on a first light chain of the cysteine engineered antibody and at position 121 of a second light chain of the cysteine engineered antibody, according to Kabat numbering. In some embodiments, the cysteine engineered antibody comprises two cysteine amino acid substitutions.

In some embodiments, a cysteine amino acid substitution is present at a position on a heavy chain of the cysteine engineered antibody of the cysteine engineered ADC. In some embodiments, the cysteine engineered antibody comprises a single heavy chain, and the cysteine amino acid substitution is present at a position on the single heavy chain. In some embodiments, the cysteine amino acid substitution is at position 375 of the heavy chain of the cysteine engineered antibody, according to EU numbering. In some embodiments, the cysteine amino acid substitution is at position 400 of the heavy chain of the cysteine engineered antibody, according to EU numbering. In some embodiments, the cysteine engineered antibody comprises one cysteine amino acid substitution.

In some embodiments, a cysteine amino acid substitution is present at a position on one of the heavy chains of the cysteine engineered antibody of the cysteine engineered ADC. In some embodiments, the cysteine amino acid substitution is present at a position on each of the heavy chains of the cysteine engineered antibody. In some embodiments, the cysteine amino acid substitution is the same on each of the heavy chains of the cysteine engineered antibody. In some embodiments, the cysteine amino acid substitution is different on each of the heavy chains of the cysteine engineered antibody. In some embodiments, the cysteine amino acid substitution is at position 375 of the heavy chain of the cysteine engineered antibody, according to EU numbering. In some embodiments, the cysteine amino acid substitution is at position 375 of each of the heavy chains of the cysteine engineered antibody, according to EU numbering. In some embodiments, the cysteine amino acid substitution is at position 400 of the heavy chain of the cysteine engineered antibody, according to EU numbering. In some embodiments, the cysteine amino acid substitution is at position 400 of each of the heavy chains of the cysteine engineered antibody, according to EU numbering. In some embodiments, the cysteine amino acid substitution is at position 400 of a first heavy chain of the cysteine engineered antibody and at position 375 of a second heavy chain of the cysteine engineered antibody, according to EU numbering. In some embodiments, the cysteine engineered antibody comprises two cysteine amino acid substitutions.

In some embodiments, the cysteine engineered antibody of the cysteine engineered ADC comprises at least one cysteine amino acid substitution on a light chain of the cysteine engineered antibody and at least one cysteine amino acid substitution on a heavy chain of the cysteine engineered antibody. In some embodiments, the cysteine engineered antibody comprises two cysteine amino acid substitutions. In some embodiments, the cysteine amino acid substitutions are at position 121 the light chain, according to Kabat numbering, and position 375 of the heavy chain, according to EU numbering. In some embodiments, the cysteine amino acid substitutions are at position 121 of the light chain, according to Kabat numbering, and position 400 of the heavy chain, according to EU numbering. In some embodiments, the cysteine amino acid substitutions are at position 114 of the light chain, according to Kabat numbering, and position 400 of the heavy chain, according to EU numbering. In some embodiments, the cysteine engineered antibody is a half antibody, and comprises a cysteine amino acid substitution at position 121 the light chain, according to Kabat numbering, and position 375 of the heavy chain, according to EU numbering. In some embodiments, the cysteine engineered antibody is a half antibody, and comprises a cysteine amino acid substitution at position 121 of the light chain, according to Kabat numbering, and position 400 of the heavy chain, according to EU numbering. In some embodiments, the cysteine engineered antibody is a half antibody, and comprises a cysteine amino acid substitution at position 114 of the light chain, according to Kabat numbering, and position 400 of the heavy chain, according to EU numbering.

In some embodiments, the cysteine engineered antibody of the cysteine engineered ADC comprises four cysteine amino acid substitutions. In some embodiments, the cysteine engineered antibody comprises two heavy chains and two light chains, and comprises a cysteine amino acid substitution on each of the light chains and a cysteine amino acid substitution on each of the heavy chains. In some embodiments, the cysteine engineered antibody comprises the same cysteine amino acid substitution on each of the light chains. In some embodiments, the cysteine engineered antibody comprises a cysteine amino acid substitution at position 121 of each of the light chains, according to Kabat numbering. In some embodiments, the cysteine engineered antibody comprises a cysteine amino acid substitution at position 114 of each of the light chains, according to Kabat numbering. In some embodiments, the cysteine engineered antibody comprises the same cysteine amino acid substitution on each of the heavy chains. In some embodiments, the cysteine engineered antibody comprises a cysteine amino acid substitution at position 400 of each of the heavy chains, according to EU numbering. In some embodiments, the cysteine engineered antibody comprises a cysteine amino acid substitution at position 375 of each of the heavy chains, according to EU numbering. In some embodiments, the cysteine engineered antibody comprises the same cysteine amino acid substitution on each of the light chains and the same cysteine amino acid substitution on each of the heavy chains. In some embodiments, the cysteine amino acid substitutions are at position 121 of each of the light chains, according to Kabat numbering, and position 375 of each of the heavy chains, according to EU numbering. In some embodiments, the cysteine amino acid substitutions are at position 121 of each of the light chains, according to Kabat numbering, and position 400 of each of the heavy chains, according to EU numbering. In some embodiments, the cysteine amino acid substitutions are at position 114 of each of the light chains, according to Kabat numbering, and position 400 of each of the heavy chains, according to EU numbering.

In some embodiments, the cysteine engineered antibody of the cysteine engineered ADC comprises a different cysteine amino acid substitution on each of the light chains or each of the heavy chains. In some embodiments, the cysteine engineered antibody comprises a different cysteine amino acid substitution on each of the light chains. In some embodiments, the cysteine engineered antibody comprises a different cysteine amino acid substitution on each of the heavy chains. In some embodiments, the cysteine engineered antibody comprises a different cysteine amino acid substitution on each of the light chains and the same cysteine amino acid substitution on each of the heavy chains. In some embodiments, the cysteine engineered antibody comprises a different cysteine amino acid substitution on each of the heavy chains and the same cysteine amino acid substitution on each of the light chains. In some embodiments, the cysteine engineered antibody comprises a cysteine amino acid substitution at position 121 or position 114 of a first light chain, according to Kabat numbering, and position 375 or position 400 of one of a first heavy chain, according to EU numbering. In some embodiments, the cysteine engineered antibody comprises a cysteine amino acid substitution at position 121 of a first the light chain, according to Kabat numbering, and position 375 of a first heavy chain, according to EU numbering. In some embodiments, the cysteine engineered antibody comprises a cysteine amino acid substitution at position 121 of a first light chain, according to Kabat numbering, and position 400 of a first heavy chain, according to EU numbering. In some embodiments, the cysteine engineered antibody comprises a cysteine amino acid substitution at position 114 of a first light chain, according to Kabat numbering, and position 400 of a first heavy chain, according to EU numbering. The first light chain and the first heavy chain (e.g., half antibody) of the cysteine engineered antibodies provided herein may be combined with any other suitable half antibody comprising a second light chain and a second heavy chain. For example, in some embodiments, the second half antibody does not comprise cysteine amino acid substitutions in either the second light chain or the second heavy chain.

Alternatively, the second half antibody may comprise any cysteine amino acid substitutions suitable for generating cysteine engineered antibodies and cysteine engineered ADCs generated therefrom, known in the art. Cysteine amino acid substitutions suitable for use in cysteine engineered antibody and cysteine engineered ADCs generated therefrom are described, for example, in U.S. Pat. Nos. 7,521,541, 9,845,355, 11,147,852, and US 2020/0338207, the contents of each of which are herein incorporated by reference in their entirety. In some embodiments, the second half antibody comprises a cysteine amino acid substitution at any of position 15, 110, 114, 121, 127, 168, 205, or a combination thereof, of the second light chain, according to Kabat numbering, and a cysteine amino acid substitution at position 5, 23, 84, 112, 118, 120, 282, 375 and 400, or a combination thereof, of the second heavy chain, according to EU numbering. In some embodiments, the second half antibody comprises a cysteine amino acid substitution at any of position 107, 108, 109, 114, 129, 142, 143, 145, 152, 154, 156, 159, 161, 165, 168, 169, 170, 182, 183, 197, 199, and 203, or a combination thereof, of the second light chain, and a cysteine amino acid substitution at position 121, 124, 152, 171, 258, 292, 333, 334, 360, 375, 392 and 400, or a combination thereof, of the second heavy chain, both according to EU numbering. In some embodiments, the second half antibody comprises a cysteine amino acid substitution at position 142 of the second light chain and position 375 of the second heavy chain. In some embodiments, the second half antibody comprises a cysteine amino acid substitution at position 165 of the second light chain and position 258 of the second heavy chain. In some embodiments, the second half antibody comprises a cysteine amino acid substitution at position 142 of the second light chain and position 171 of the second heavy chain. In some embodiments, the second half antibody comprises a cysteine amino acid substitution at position 205 of the second light chain and position 149 of the second heavy chain. In some embodiments, the second half antibody comprises a cysteine amino acid substitution at position 205 of the second light chain and position 118 of the second heavy chain. In some embodiments, the second half antibody comprises a cysteine amino acid substitution at position 205 of the second light chain and position 140 of the second heavy chain. In some embodiments, the second half antibody comprises a cysteine amino acid substitution at position 142 of the second light chain and position 118 of the second heavy chain. In some embodiments, the second half antibody comprises a cysteine amino acid substitution at position 205 of the second light chain and position 375 of the second heavy chain. In some embodiments, the second half antibody comprises a cysteine amino acid substitution at position 183 of the second light chain and position 290 of the second heavy chain. In some embodiments, the second half antibody comprises a cysteine amino acid substitution at position 392 of the second heavy chain and position 334 of the second heavy chain. In some embodiments, the cysteine engineered antibody comprises a cysteine amino acid substitution at position 121 of the first light chain and position 114 of second light chain, according to Kabat numbering, and position 375 of the first heavy chain and position 400 of the second heavy chain, according to EU numbering.

### 3. Linker

In some embodiments, the cysteine engineered ADC comprises a cysteine engineered antibody that is conjugated to a drug moiety via a cysteine amino acid (e.g., free cysteine). The drug moiety can be conjugated to the cysteine engineered antibody in a manner that reduces its activity unless it is detached from the cysteine engineered antibody (e.g., by hydrolysis, by antibody degradation, or by a cleaving agent). In some embodiments, the drug moiety can be attached to the cysteine engineered antibody at a cysteine amino acid substitution via a linker moiety. A drug moiety conjugated to a linker moiety is also referred to herein as a drug moiety-linker. The nature of the linker moiety can vary widely. The components that make up the linker moiety are chosen on the basis of their characteristics, which may be dictated in part, by the conditions at the location to which the conjugate is delivered within a patient. In some embodiments, the linker moiety LU has a formula selected from the group consisting of:

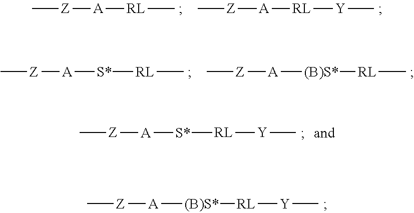

wherein Z is a Stretcher Unit; A is a bond or a Connecter Unit; B is a Parallel Connector Unit; S* is a Partitioning Agent; RL is Releasable Linker; and Y is a Spacer Unit; and wherein D is covalently attached to LU via any suitable attachment site on D, optionally wherein a hydrogen atom of a hydroxyl, thiol, primary amine, or secondary amine of D is replaced with a bond to LU or a tertiary amine of D is quaternized to form a bond to LU.

In other embodiments, the linker moiety LU has a formula selected from the group consisting of:

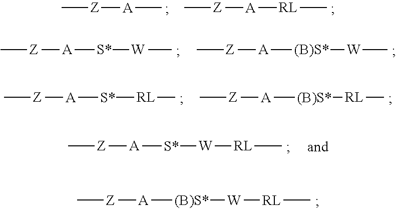

wherein Z is a Stretcher Unit, A is a bond or a Connecter Unit; B is a Parallel Connector Unit; S* is a Partitioning Agent; RL is a Releasable Linker other than a Glycoside (e.g., Glucuronide) Unit; and W is an Amino Acid Unit; and wherein D is covalently attached to LU via any suitable attachment site on D, optionally wherein a hydrogen atom of a hydroxyl, thiol, primary amine, or secondary amine of D is replaced with a bond to LU or a tertiary amine of D is quaternized to form a bond to LU.

In one group of embodiments, LU has a formula selected from the group consisting of:—Z-A-S*-RL- and —Z-A-S*-RL-Y—.

In another group of embodiments, LU has a formula selected from the group consisting of —Z-A-B(S*)—RL- and —Z-A-B(S*)—RL-Y—.

In still another group of embodiments, LU has a formula selected from the group consisting of —Z-A-RL- and —Z-A-RL-Y—.

### Stretcher Unit (Z) or (Z′):

A Stretcher Unit (Z) is a component of a cysteine engineered ADC or a cysteine engineered-linker compound or other intermediate that acts to connect the linker moiety to the remainder of the cysteine engineered ADC. In that regard a Stretcher Unit, prior to attachment to a cysteine engineered antibody (i.e. a Stretcher Unit precursor, Z′), has a functional group that can form a bond with a functional group of a targeting ligand.

In some embodiments, a Stretcher Unit precursor (Z′) has an electrophilic group that is capable of interacting with a reactive nucleophilic group present on a cysteine engineered antibody to provide a covalent bond between a cysteine engineered antibody and the Stretcher Unit of a linker moiety. Nucleophilic groups on an antibody having that capability include but are not limited to, sulfhydryl, hydroxyl, and amino functional groups. The heteroatom of the nucleophilic group of an antibody can be reactive to an electrophilic group on a Stretcher Unit precursor and can provide a covalent bond between the cysteine engineered antibody and Stretcher Unit of a linker moiety or drug-linker moiety. Useful electrophilic groups for that purpose include, but are not limited to, maleimide, haloacetamide groups, and NHS esters. The electrophilic group provides a convenient site for cysteine engineered antibody attachment to form a cysteine engineered ADC or a cysteine engineered antibody-linker moiety intermediate.

In other embodiments, a Stretcher Unit precursor has a reactive site which has a nucleophilic group that is reactive to an electrophilic group present on a cysteine engineered antibody. Useful electrophilic groups on an antibody for that purpose include, but are not limited to, aldehyde and ketone carbonyl groups. The heteroatom of a nucleophilic group of a Stretcher Unit precursor can react with an electrophilic group on an antibody and form a covalent bond to the antibody. Useful nucleophilic groups on a Stretcher Unit precursor for that purpose include, but are not limited to, hydrazide, hydroxylamine, amino, hydrazine, thiosemicarbazone, hydrazine carboxylate, and arylhydrazide. The electrophilic group on an antibody provides a convenient site for cysteine engineered antibody attachment to form a cysteine engineered ADC or a cysteine engineered antibody-linker moiety intermediate.

In some embodiments, a sulfur atom of a cysteine engineered antibody is bound to a succinimide ring system of a Stretcher Unit formed by reaction of a thiol functional group of a targeting ligand with a maleimide moiety of the corresponding Stretcher Unit precursor. In other embodiments, a thiol functional group of a cysteine engineered antibody reacts with an alpha haloacetamide moiety to provide a sulfur-bonded Stretcher Unit by nucleophilic displacement of its halogen substituent.

Representative Stretcher Units of such embodiments include those having the structures of:

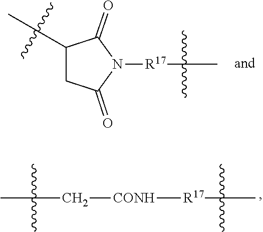

wherein the wavy line adjacent to R¹⁷ indicates attachment to the Parallel Connector Unit (B) or Connector Unit (A) if B is absent, or a Partitioning Agent (S*), if B is absent, the other wavy line indicates covalent attachment to a sulfur atom of a Ligand Unit, and R¹⁷ is —C₁-C₁₀ alkylene-, —CH₂—CH₂—(OCH₂CH₂)ₖ—, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein k is an integer ranging from 1 to 36. In some embodiments, R¹⁷ is-C₁-C₁₀ alkylene-. In some embodiments, R¹⁷ is-CH₂—CH₂—(OCH₂CH₂)ₖ—, wherein k is an integer ranging from 1 to 36.

In some embodiments, the R¹⁷ group is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein subscript x is an integer of from 1-4 and each R^{a} is independently selected from the group consisting of C₁₋₆ alkyl and C₁₋₆ haloalkyl, or two Ra groups are combined with the nitrogen to which they are attached to form an azetidinyl, pyrrolidinyl or piperidinyl group.

An illustrative Stretcher Unit is that of Formula Za or Za-BU in which R¹⁷ is —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, or −(C₃-C₈ heterocyclo)-C₁-C₁₀ alkylene-C(═O)—.

Accordingly, some preferred embodiments are represented by formula Za and Za-BU:

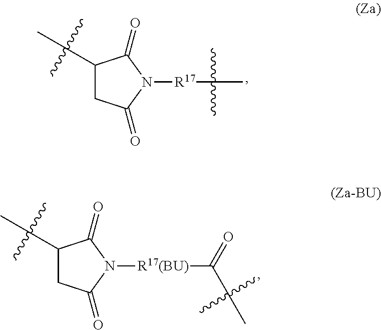

wherein the wavy line adjacent the carbonyl carbon atom indicates attachment to LP, B, A, or S*, in the formulae above, depending on the presence or absence of A and/or B, and the other wavy line indicates covalent bonding of the succinimide ring carbon atom to a sulfur atom of a cysteine engineered antibody. During synthesis, the basic amino functional group of the Basic Unit (BU) can be protected by a protecting group.

More preferred embodiments of Stretcher Units of formula Za and Za-BU are as follows:

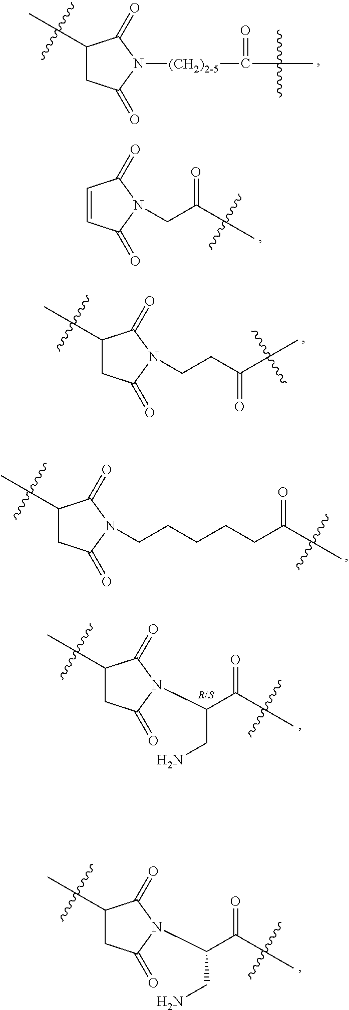

wherein the wavy line adjacent the carbonyl carbon atom indicates attachment to B, A, or S*, in the formulae above, depending on the presence or absence of A and/or B, and the other wavy line indicates covalent bonding of the succinimide ring carbon atom to a sulfur atom of a cysteine engineered antibody.

It will be understood that a cysteine engineered antibody-substituted succinimide may exist in hydrolyzed form(s). Those forms are exemplified below for hydrolysis of Za or Za-BU, wherein the structures representing the regioisomers from that hydrolysis have formula Zb and Zc or Zb-BU and Zc-BU.

Accordingly, in other preferred embodiments a Stretcher unit (Z) is comprised of a succinic acid-amide moiety represented by the following:

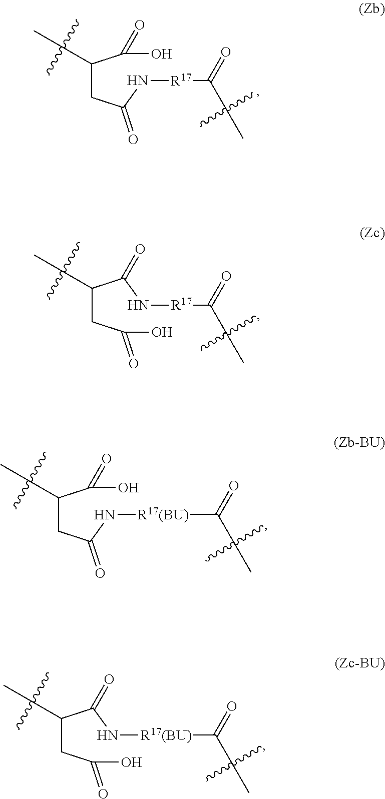

wherein the wavy line adjacent to the carbonyl carbon atom bonded to R¹⁷ and the wavy line adjacent to the carbon atom of the acid-amide moiety is as defined for Za or Za-BU, depending on the presence or absence of A and/or B; and R¹⁷ is —C₁-C₅ alkylene-, wherein in Zb-BU and Zc-BU the alkylene is substituted by a Basic Unit (BU), wherein BU is —(CH₂) xNH2, —(CH₂)ₓNHR^{a}, or —(CH₂)ₓN(R^{a})₂, wherein subscript x is an integer of from 1-4 and each R^{a} is independently selected from the group consisting of C₁₋₆ alkyl and C₁₋₆ haloalkyl, or both R^{a} together with the nitrogen to which they are attached define an azetidinyl, pyrrolidinyl or piperidinyl group.

In more preferred embodiment, —Z-A-comprises a moiety derived from a maleimido-alkanoic acid moiety or an mDPR moiety. See, for example, see WO 2013/173337. In one group of embodiments, Z-A- is derived from a maleimido-propionyl moiety.

Accordingly in some of those more preferred embodiments, a Stretcher unit (Z) is comprised of an succinic acid-amide moiety represented by the structure of formula Zb′, Zc′, (R/S)-Zb′-BU, (S)-Zb′-BU, (R/S)-Zc′-BU or(S)-Zc′-BU as follows:

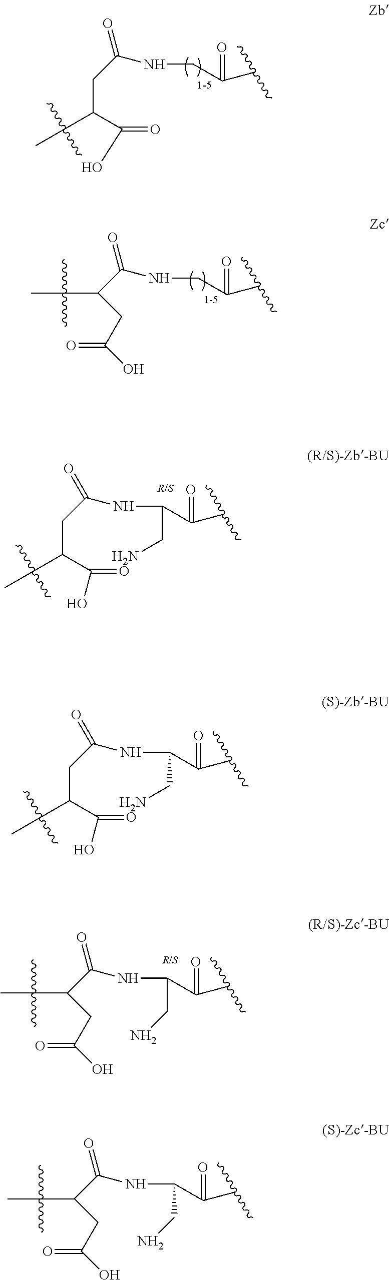

wherein the wavy lines are as defined for Za or Za-BU.

In particularly preferred embodiments, a Stretcher unit (Z) is comprised of a succinimide moiety represented by the structure of

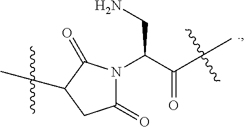 [SMILES: *C(=O)[C@H](CN)N1C(=O)CC(*)C1=O]

which may be generated from a maleimido-amino-propionyl (mDPR) analog (a 3-amino-2-(2,5-dioxo-2,5-dihydro-1H-pyrrol-1-yl) propanoic acid derivative), or is comprised of a succinic acid-amide moiety represented by the structure of:

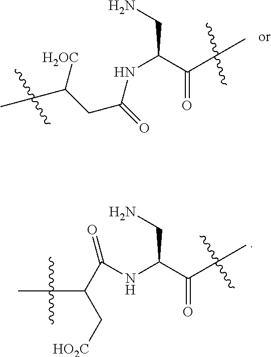

Illustrative Stretcher Units bonded to a Connector Unit (A) which are comprised of Za′, Zb′ or Zc′, in which —R¹⁷— of Za, Zb or Zc is —CH₂— or —CH₂CH₂—, or are comprised of Za′-BU, Zb′-BU or Zc′-BU in which-R¹⁷ (BU)- of Za-BU, Zb-BU or Zc-BU is-CH(CH₂NH₂)—, have the following structures:

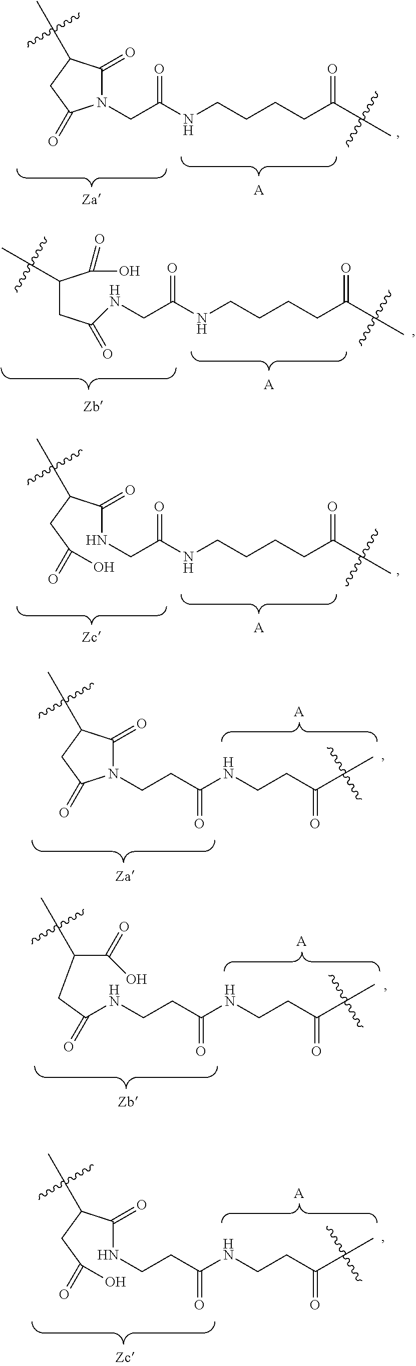

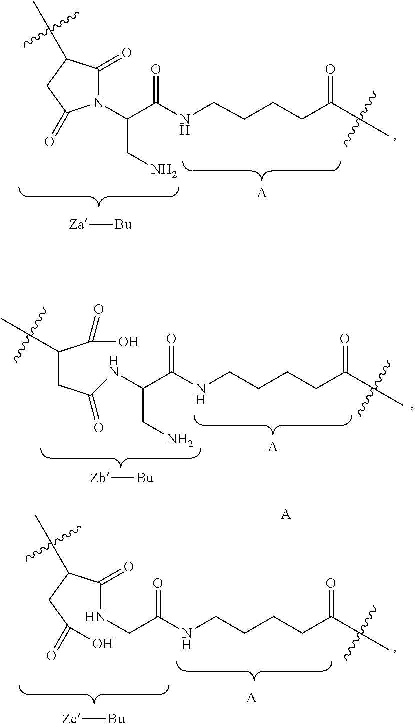

wherein the wavy lines are as defined for Za or Za-BU.

Other Stretcher Units bonded to a Ligand Unit (L) and a Connector Unit (A) have the structures above wherein A in any one of the above -Za-A-,-Za (BU)-A-, -Za′-A-, -Za′ (BU)-A-, -Zb-A-, -Zb (BU)-A-, -Zb′-A-, -Zb′ (BU)-, -Zc′-A- and Zc′ (BU)-A-structures is replaced by a Parallel Connector Unit having the structure of:

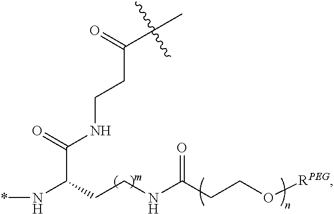

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to a Stretcher Unit corresponding in structure to formula Za, Za′, Zb′ or Zc′ and the wavy line indicates covalent attachment to the Releasable Linker (RL).

Illustrative Stretcher Units prior to conjugation to the cysteine engineered antibody (i.e., Stretcher Unit precursors) are comprised of a maleimide moiety and are represented by structures including that of formula Z′a

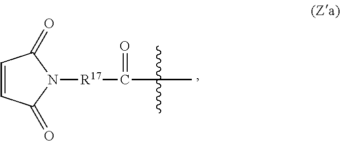

wherein the wavy line adjacent the carbonyl carbon atom indicates attachment to B, A, or S*, in the formulae above, depending on the presence or absence of A and/or B, R¹⁷ is —(CH₂)₁₋₅—, optionally substituted with a Basic Unit, such as an optionally substituted aminoalkyl, e.g., —(CH₂)ₓNH₂, —(CH₂)ₓNHR^{a}, and —(CH₂)ₓN(R^{a})₂, wherein subscript x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

Other illustrative Stretcher Units prior to conjugation to the cysteine engineered antibody (i.e., Stretcher Unit precursors) are comprised of a maleimide moiety and are represented by structures including that of formula Z′a-BU.

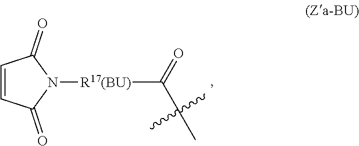

wherein the wavy line adjacent the carbonyl carbon atom indicates attachment to B, A, or S*, in the formulae above, depending on the presence or absence of A and/or B, R¹⁷ is —(CH₂)₁₋₅—, substituted with a Basic Unit, such as an optionally substituted aminoalkyl, e.g., —(CH₂)ₓNH₂, —(CH₂)ₓNHR^{a}, and —(CH₂)ₓN(R^{a})₂, wherein subscript x is an integer of from 1-4, preferably R¹⁷ is —CH₂— or —CH₂CH₂— and subscript x is 1 or 2, and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

In some preferred embodiments of formula Z′a, a Stretcher Unit precursor (Z′) is represented by one of the following structures:

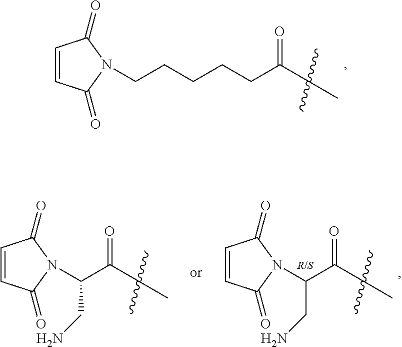 [SMILES: *C(=O)CCCCCN1C(=O)C=CC1=O ; *C(=O)[C@H](CN)N1C(=O)C=CC1=O ; *C(=O)C(CN)N1C(=O)C=CC1=O]

wherein the wavy line adjacent to the carbonyl is as defined for Z′a or Z′a-BU.

In more preferred embodiments the Stretcher unit precursor (Z′) is comprised of a maleimide moiety and is represented by the structure of:

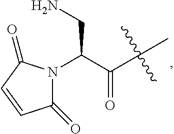 [SMILES: *C(=O)[C@H](CN)N1C(=O)C=CC1=O]

wherein the wavy line adjacent to the carbonyl is as defined for Za′ and the amino group is optional protonated or protected by an amino protecting group.

In Stretcher Units having a BU moiety, it will be understood that the amino functional group of that moiety is typically protected by an amino protecting group during synthesis, e.g., an acid labile protecting group (e.g., BOC).

Illustrative Stretcher Unit precursors covalently attached to a Connector Unit that are comprised of the structure of Z′a or Z′a-BU in which —R¹⁷— or —R¹⁷ (BU)- is —CH₂—, —CH₂CH₂—, or —CH(CH₂NH₂)— have the following structures:

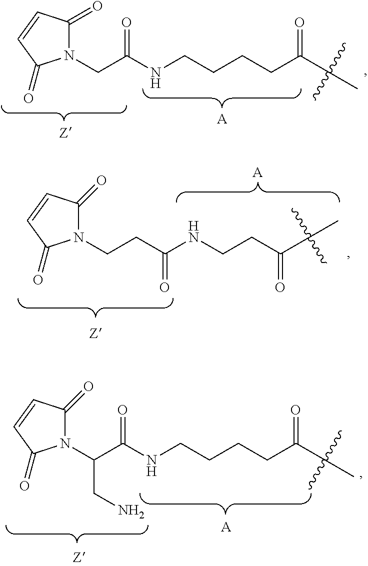

wherein the wavy line adjacent to the carbonyl is as defined for Z′a or Z′a-BU.

Other Stretcher Unit precursors bonded a Connector Unit (A) have the structures above wherein A in any one of the above Z′-A- and Z′ (BU)-A-structures is replaced by a Parallel Connector Unit and Partitioning Agent (—B(S*)—) having the structure of

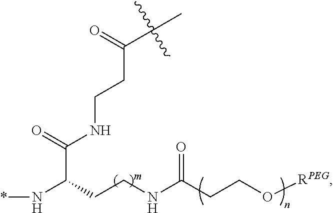

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to the Stretcher Unit precursor corresponding in structure to formula Za or Za′ and the wavy line indicates covalent attachment to RL. In instances such as those shown here, the shown PEG group is meant to be exemplary of a variety of Partitioning Agents including PEG groups of different lengths and other Partitioning Agents that can be directly attached or modified for attachment to the Parallel Connector Unit.

In another embodiment, the Stretcher Unit is attached to the cysteine engineered antibody via a disulfide bond between a sulfur atom of the cysteine engineered antibody and a sulfur atom of the Stretcher unit. A representative Stretcher Unit of this embodiment is depicted within the square brackets of Formula Zb:

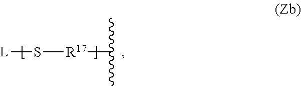

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, —CH₂—CH₂—(OCH₂CH₂)ₖ—, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein k is an integer ranging from 1 to 36. In some embodiments, R¹⁷ is —CH₂—CH₂—(OCH₂CH₂)ₖ—, wherein k is an integer ranging from 1 to 36.

In yet another embodiment, the reactive group of a Stretcher Unit precursor contains a reactive site that can form a bond with a primary or secondary amino group of a Ligand Unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher Units of this embodiment are depicted within the square brackets of Formulas Zci, Zcii and Zciii:

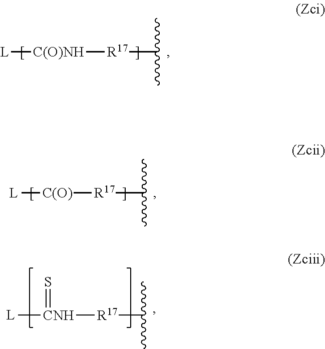

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, —CH₂—CH₂—(OCH₂CH₂)ₖ—, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein k is an integer ranging from 1 to 36.

In still other embodiments, the reactive group of the Stretcher Unit precursor contains a reactive nucleophile that is capable of reacting with an electrophile present on, or introduced to, a Ligand Unit. For example, a carbohydrate moiety on a targeting ligand can be mildly oxidized using a reagent such as sodium periodate and the resulting electrophilic functional group (—CHO) of the oxidized carbohydrate can be condensed with a Stretcher Unit precursor that contains a reactive nucleophile such as a hydrazide, an oxime, a primary or secondary amine, a hydrazine, a thiosemicarbazone, a hydrazine carboxylate, or an arylhydrazide such as those described by Kaneko, T. et al. (1991) Bioconjugate Chem. 2:133-41. Representative Stretcher Units of this embodiment are depicted within the square brackets of Formulas Zdi, Zdii, and Zdiii:

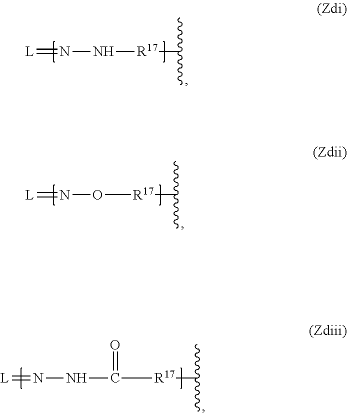

wherein the wavy line indicates attachment to the Parallel Connector Unit (B) or Connector Unit (A), or a Partitioning Agent (S*), if A and B are absent and R¹⁷ is —C₁-C₁₀ alkylene-, —CH₂—CH₂—(OCH₂CH₂)ₖ—, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein k is an integer ranging from 1 to 36. In some embodiments, R¹⁷ is —CH₂—CH₂—(OCH₂CH₂)ₖ—, wherein k is an integer ranging from 1 to 36.

In some aspects of the prevent invention the Stretcher Unit has a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

### Connector Unit (A)

In some embodiments, a Connector Unit (A), is included in a cysteine engineered ADC or cysteine engineered antibody-linker compound in instances where it is desirable to add additional distance between the Stretcher Unit (Z) or precursor thereof (Z′) and the Releasable Linker. In some embodiments, the extra distance will aid with activation within RL. Accordingly, the Connector Unit (A), when present, extends the framework of the Linker Unit. In that regard, a Connector Unit (A) is covalently bonded with the Stretcher Unit (or its precursor) at one terminus and is covalently bonded to the optional Parallel Connector Unit or the Partitioning Agent (S*) at its other terminus.

The skilled artisan will appreciate that the Connector Unit can be any group that serves to provide for attachment of the Releasable Linker to the remainder of the Linker Unit (Q). The Connector Unit can be, for example, comprised of one or more (e.g., 1-10, preferably, 1, 2, 3, or 4) proteinogenic or non-proteinogenic amino acid, amino alcohol, amino aldehyde, diamino residues. In some embodiments, the Connector Unit is a single proteinogenic or non-proteinogenic amino acid, amino alcohol, amino aldehyde, or diamino residue. An exemplary amino acid capable of acting as Connector units is β-alanine.

In some of those embodiments, the Connector Unit has the formula denoted below:

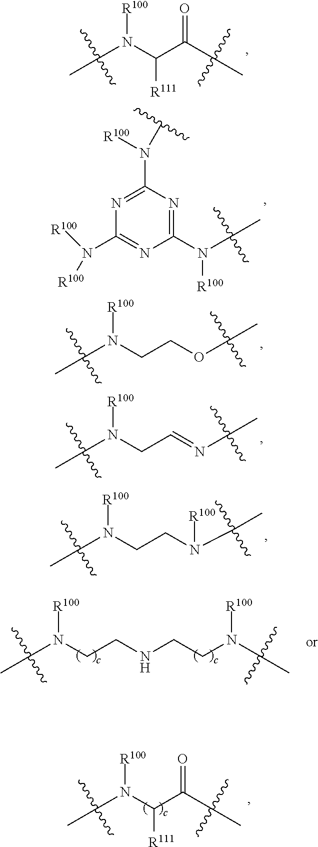

wherein the wavy lines indicate attachment of the Connector Unit within the cysteine engineered ADC or cysteine engineered antibody-linker compound; and wherein R¹¹¹ is independently selected from the group consisting of hydrogen, p-hydroxybenzyl, methyl, isopropyl, isobutyl, sec-butyl, —CH₂OH, —CH(OH) CH₃, —CH₂CH₂SCH₃, —CH₂CONH₂, —CH₂COOH, —CH₂CH₂CONH₂, —CH₂CH₂COOH, —(CH₂)₃NHC(═NH) NH₂, —(CH₂)₃NH₂, —(CH₂)₃NHCOCH₃, —(CH₂)₃NHCHO, —(CH₂)₄NHC(═NH) NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH) CH₂NH₂, 2-pyridylmethyl-, 3-pyridylmethyl-, 4-pyridylmethyl-,

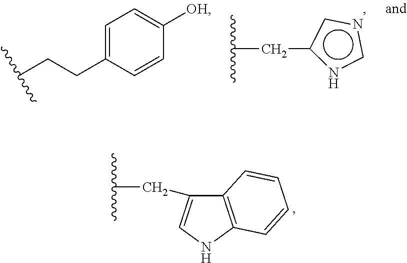

and each R¹⁰⁰ is independently selected from hydrogen or —C₁-C₃ alkyl, preferably hydrogen or CH₃; and subscript c is an independently selected integer from 1 to 10, preferably 1 to 3.

A representative Connector Unit having a carbonyl group for attachment to the Partitioning Agent (S*) or to —B(S*)— is as follows:

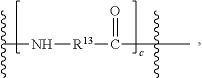

wherein in each instance R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and the subscript c is an integer ranging from 1 to 4. In some embodiments R¹³ is —C₁-C₆ alkylene and c is 1.

Another representative Connector Unit having a carbonyl group for attachment to Partitioning Agent (S*) or to —B(S*)— is as follows:

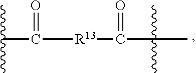

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, or —(C₃-C₈ heterocyclo)-C₁-C₁₀ alkylene-. In some embodiments R¹³ is —C₁-C₆ alkylene.

A representative Connector Unit having a NH moiety that attaches to Partitioning Agent (S*) or to —B(S*)— is as follows:

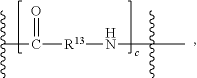

wherein in each instance, R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and subscript c is from 1 to 14. In some embodiments R¹³ is-C₁-C₆ alkylene and subscript c is 1.

Another representative Connector Unit having a NH moiety that attaches to Partitioning Agent (S*) or to —B(S*)— is as follows:

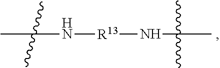

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, —(C₃-C₈ heterocyclo)-C₁-C₁₀ alkylene-, C(═O) C₁-C₆ alkylene-or —C₁-C₆ alkylene-C(═O)—C₁-C₆ alkylene.

Selected embodiments of Connector Units include those having the following structure of:

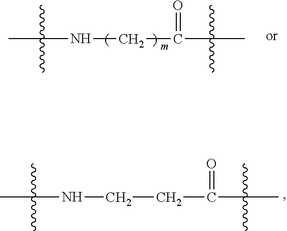

wherein the wavy line adjacent to the nitrogen indicates covalent attachment a Stretcher Unit (Z) (or its precursor Z′), and the wavy line adjacent to the carbonyl indicates covalent attachment to Partitioning Agent (S*) or to —B(S*)—; and m is an integer ranging from 1 to 6, preferably 2 to 6, more preferably 2 to 4.

### Releasable Linker (RL)

The Releasable Linker (RL) is capable of linking to the Spacer Unit (Y) or the drug moiety (D). RL comprises a cleavable bond (i.e., a reactive site) that upon action by an enzyme present within a hyper-proliferating cell or hyper-activated immune cells or characteristic of the immediate environment of these abnormal or unwanted cells, or upon non-enzymatic action due to conditions more likely experienced by hyper-proliferating cells in comparison to normal cells, releases free drug. Alternatively, RL comprises a cleavable bond that is more likely acted upon intracellularly in a hyper-proliferating cell or hyper-activated immune cell due to preferential entry into such cells in comparison to normal cells.

### Peptide Releasable Linkers

In some embodiments, the Releasable Linker is a Peptide Releasable Linker. In some embodiments, the Peptide Releasable Linker (RL) will comprise one or more contiguous or non-contiguous sequences of amino acids (e.g., so that RL has 1 to no more than 12 amino acids). The Peptide Releasable Linker can comprise or consist of, for example, an amino acid, a dipeptide, tripeptide, tetrapeptide, pentapeptide, hexapeptide, heptapeptide, octapeptide, nonapeptide, decapeptide, undecapeptide or dodecapeptide unit. In some aspects, in the presence of an enzyme (e.g., a tumor-associated protease), an amide linkage between the amino acids is cleaved, which ultimately leads to release of free drug.

Each amino acid can be proteinogenic or non-proteinogenic and/or a D-or L-isomer provided that RL comprises a cleavable bond that, when cleaved, initiates release of the drug. In some embodiments, the Peptide Releasable Linker will comprise only proteinogenic amino acids. In some aspects, the Peptide Releasable Linker will have from 1 to no more than 12 amino acids in contiguous sequence.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine. In some embodiments, each amino acid is selected from the proteinogenic or the non-proteinogenic amino acids.

In another embodiment, each amino acid is independently selected from the group consisting of the following L-(proteinogenic) amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In another embodiment, each amino acid is independently selected from the group consisting of the following D-isomers of these proteinogenic amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In certain embodiments, the Peptide Releasable Linker is comprised only of proteinogenic amino acids. In other embodiments, the Peptide Releasable Linker is comprised only of non-proteinogenic amino acids. In some embodiments, the Peptide Releasable Linker is comprised of a proteinogenic amino acid attached to a non-proteinogenic amino acid. In some embodiments, Peptide Releasable Linker is comprised of a proteinogenic amino acid attached to a D-isomer of a proteinogenic amino acid.

In another embodiment, each amino acid is independently selected from the group consisting of β-alanine, N-methylglycine, glycine, lysine, valine, and phenylalanine.

Exemplary Peptide Releasable Linkers include dipeptides or tripeptides with -Val-Lys-Gly-, -Val-Cit-, -Phe-Lys- or -Val-Ala-.

Useful Peptide Releasable Linkers can be designed and optimized in their selectivity for enzymatic cleavage by a particular enzyme, for example, a tumor-associated protease. In some embodiments, cleavage of a linkage is catalyzed by cathepsin B, C or D, or a plasmin protease.

In some embodiments, the Peptide Releasable Linker (RL) will be represented by -(-AA-)₁₋₁₂-, or (-AA-AA-)₁₋₆ wherein AA is at each occurrence independently selected from proteinogenic or non-proteinogenic amino acids. In one aspect, AA is at each occurrence independently selected from proteinogenic amino acids. In another aspect, RL is a tripeptide having the formula: AA₁-AA₂-AA₃, wherein AA₁, AA₂ and AA₃ are each independently an amino acid and wherein AA₁ attaches to —NH— and AA₃ attaches to S*. In yet another aspect, AA₃ is gly or β-ala.

In some embodiments, the Peptide Releasable Linker has the formula denoted below in the square brackets, the subscript w is an integer ranging from 1 to 12; or w is 1, 2, 3, 4, 5, 6, 7, 8, 9, 0, 11, or 12; or w is 2, 3, or 4; or w is 3; or w is 4:

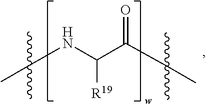

wherein R¹⁹ is, in each instance, independently selected from the group consisting of hydrogen, methyl, isopropyl, isobutyl, sec-butyl, benzyl, p-hydroxybenzyl, —CH₂OH, —CH(OH) CH₃, —CH₂CH₂SCH₃, —CH₂CONH₂, —CH₂COOH, —CH₂CH₂CONH₂, —CH₂CH₂COOH, —(CH₂)₃NHC(═NH) NH₂, —(CH₂)₃NH₂, —(CH₂)₃NHCOCH₃, —(CH₂)₃NHCHO, —(CH₂)₄NHC(═NH) NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

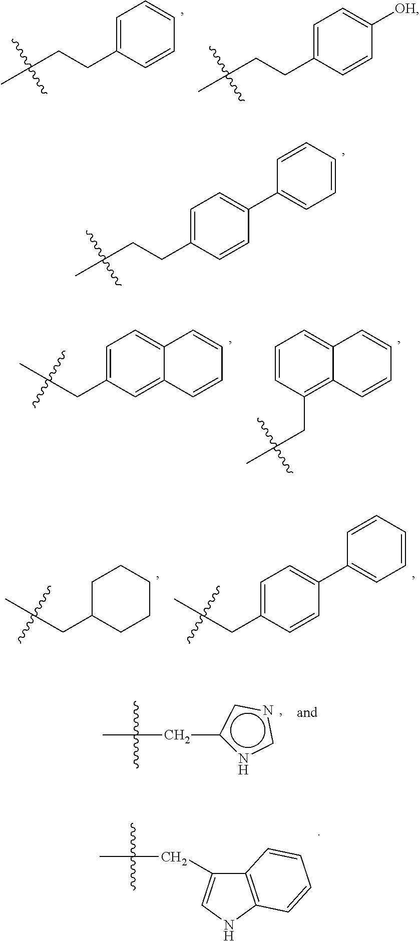

In some aspects, the subscript w is not 3.

In some aspects, each R¹⁹ is independently hydrogen, methyl, isopropyl, isobutyl, sec-butyl, —(CH₂)₃NH₂, or —(CH₂)₄NH₂. In some aspects, each R¹⁹ is independently hydrogen, isopropyl, or —(CH₂)₄NH₂.

Illustrative Peptide Releasable Linkers are represented by formulae (Pa), (Pb) and (Pc):

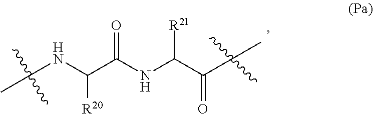

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 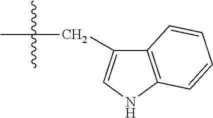 [SMILES: *Cc1c[nH]c2ccccc12] | (CH₂)₃NHCONH₂; |
| benzyl | methyl; and |
| benzyl | (CH₂)₃NHC(═NH)NH₂; |

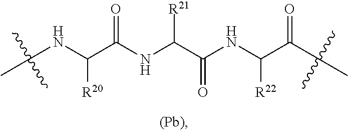

wherein R²⁰, R²¹ and R²² are as follows:

| R²⁰ | R²¹ | R²² |
|---|---|---|
| benzyl | benzyl | —(CH₂)₄NH₂ |
| isopropyl | benzyl | —(CH₂)₄NH₂ |
| H | Benzyl | —(CH₂)₄NH₂ |
| isopropyl | —(CH₂)₄NH₂ | —H |

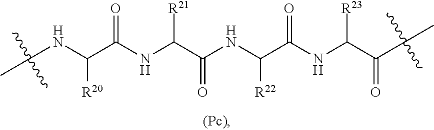

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl. |

In some embodiments, RL comprises a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala.

In other embodiments, RL comprises a peptide selected from the group consisting of gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, and val-lys-β-ala.

In still other embodiments, RL comprises a peptide selected from the group consisting of gly-gly-gly, val-gly-gly, val-cit-gly, val-gln-gly, val-glu-gly, phe-lys-gly, leu-lys-gly, val-lys-gly, val-lys-ala, val-lys-leu, leu-leu-gly and val-lys-β-ala.

In yet other embodiments, RL comprises a peptide selected from the group consisting of gly-gly-gly-gly, gly-val-lys-gly, val-lys-gly-gly, and gly-gly-phe-gly.

In other embodiments, RL is a peptide selected from the group consisting of val-gln-gly, val-glu-gly, phe-lys-gly, leu-lys-gly, val-lys-gly, val-lys-ala, val-lys-leu, leu-leu-gly and val-lys-β-ala.

In still other embodiments, RL is val-lys-gly.

In still other embodiments, RL is val-lys-β-ala.

### Glycoside Unit Releasable Linkers

In some embodiments, the Releasable Linker is a Glycoside (e.g., Glucuronide) Unit. In such embodiments, a self-immolation cascade is activated by operation of a glycosidase on a carbohydrate moiety of the Glycoside (e.g., Glucuronide) Unit. A number of sugars are useful in the embodiments described herein. Particular carbohydrate moieties include those of Galactose, Glucose, Mannose, Xylose, Arabinose, Mannose-6-phosphate, Fucose, Rhamnose, Gulose, Allose, 6-deoxy-glucose, Lactose, Maltose, Cellobiose, Gentiobiose, Maltotriose, GlcNAc, GalNAc and maltohexaose.

A Glycoside (e.g., Glucuronide) Unit typically comprises a sugar moiety (Su) linked via an oxygen glycosidic bond to a self-immolative spacer. Cleavage of the oxygen glycosidic bond initiates the self-immolation reaction sequence that result in release of free drug. In some embodiments, the self-immolation sequence is activated from cleavage by β-glucuronidase of a Glycoside (e.g., Glucuronide) Unit, which is an exemplary glycoside unit. The Glycoside (e.g., Glucuronide) Unit comprises an activation unit and a self-immolative Spacer Unit. The Glycoside (e.g., Glucuronide) Unit comprises a sugar moiety (Su) linked via an oxygen glycosidic bond to a self-immolative Spacer Unit.

In some embodiments, a Glycoside (e.g., Glucuronide) Unit comprises a sugar moiety (Su) linked via an oxygen glycoside bond (—O′—) to a Self-immolative Unit (SP) of the formula:

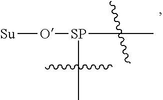

wherein the wavy lines indicate covalent attachment to the drug moiety or to a Spacer Unit that is attached to the drug moiety, and to the Stretcher Unit (Z) or its precursor (Z′), either directly or indirectly through the Connector Unit (A) or Parallel Connector Unit (B), Partitioning Agent (S*) or combinations of the Connector Unit and Parallel Connector Unit, as the case may be.

The oxygen glycosidic bond (—O′—) is typically a β-glucuronidase-cleavage site (i.e., Su is from glucuronide), such as a glycoside bond cleavable by human, lysosomal β-glucuronidase.

In some embodiments, the Glycoside (e.g., Glucuronide) Unit can be represented by formula Ga, Gb, or Gc:

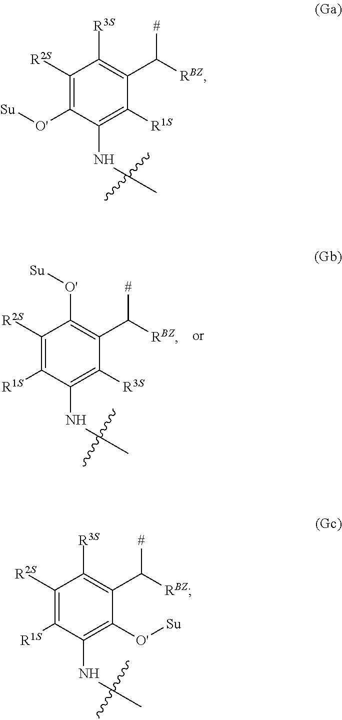

wherein Su is a Sugar moiety, —O′— represents an oxygen glycosidic bond; R^{1S}, R^{2S} and R^{3S} independently are hydrogen, a halogen, —CN, —NO₂, or other electron withdrawing group, or an electron donating group; RBZ is selected from the group consisting of C₁-C₆ alkyl, C₁-C₆ haloalkyl, a PEG unit, a cyclodextrin unit, a polyamide, a hydrophilic peptide, a polysaccharide, and a dendrimer; and wherein the wavy line indicates attachment to a Stretcher Unit (Z) (or its precursor (Z′), either directly or indirectly through a Connector Unit or Parallel Connector Unit or Connector unit and Parallel Connector Unit); and # indicates attachment to the drug moiety or to a Spacer (either directly or indirectly via an intervening functional group or other moiety).

In some embodiments, the Glycoside (e.g., Glucuronide) Unit can be represented by formula Ga*, Gb*, or Gc*:

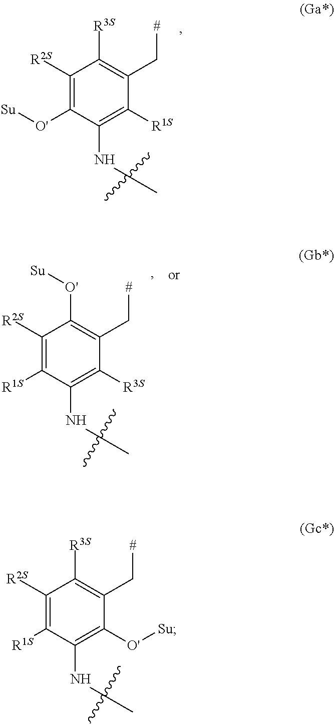

wherein Su is a Sugar moiety, —O′-represents an oxygen glycosidic bond; R^{1S}, R^{2S} and R^{3S} independently are hydrogen, a halogen, —CN, —NO₂, or other electron withdrawing group, or an electron donating group; wherein the wavy line indicates attachment to a Stretcher Unit (Z) (or its precursor (Z′), either directly or indirectly through a Connector Unit or Parallel Connector Unit or Connector unit and Parallel Connector Unit); and # indicates attachment to the drug moiety or to a Spacer (either directly or indirectly via an intervening functional group or other moiety).

In some embodiments, the Glycoside (e.g., Glucuronide) Unit can be represented by formula Ga**, Gb**, or Gc**:

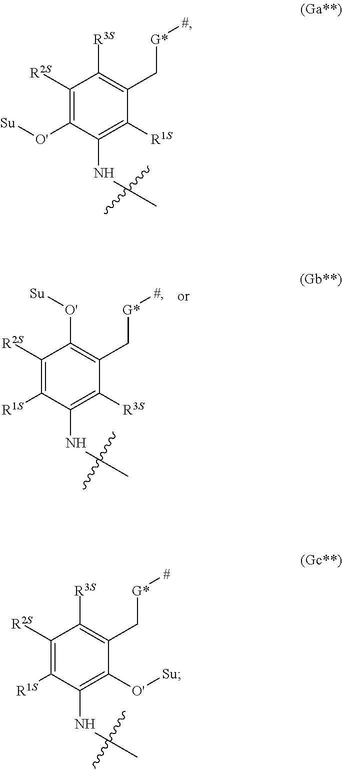

wherein Su is a Sugar moiety, —O′-represents an oxygen glycosidic bond; R^{1S}, R^{2S} and R^{3S} independently are hydrogen, a halogen, —CN, —NO₂, or other electron withdrawing group, or an electron donating group; wherein the wavy line indicates attachment to a Stretcher Unit (Z) (or its precursor (Z′), either directly or indirectly through a Connector Unit or Parallel Connector Unit or Connector unit and Parallel Connector Unit); # indicates attachment to the drug moiety, optionally through a Spacer Unit; and G* is an intervening moiety comprising a functional group that is capable of attachment to the Spacer Unit or the drug moiety. In some embodiments, the intervening moiety is-O—C(O)—.

In some embodiments, the Glycoside (e.g., Glucuronide) Unit can be represented by formula Ga***, Gb***, or Gc***:

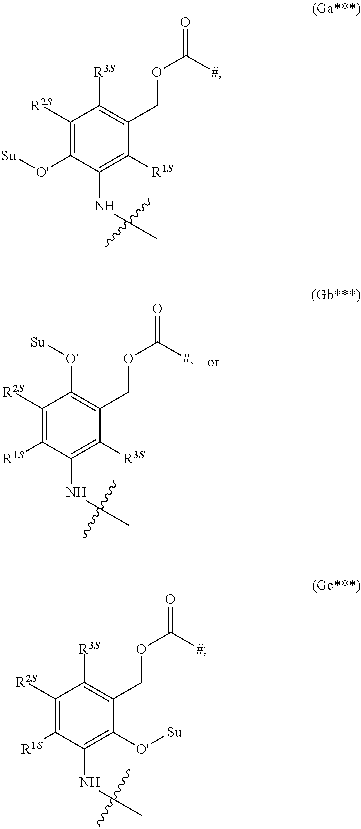

wherein Su is a Sugar moiety, —O′— represents an oxygen glycosidic bond; R^{1S}, R^{2S} and R^{3S} independently are hydrogen, a halogen, —CN, —NO₂, or other electron withdrawing group, or an electron donating group; wherein the wavy line indicates attachment to a Stretcher Unit (Z) (or its precursor (Z′), either directly or indirectly through a Connector Unit or Parallel Connector Unit or Connector unit and Parallel Connector Unit); and # indicates attachment to the drug moiety, optionally through a Spacer Unit.

In preferred embodiments R^{1S}, R^{2S} and R^{3S} are independently selected from hydrogen, halogen, —CN, or —NO₂. In other preferred embodiments, R^{1S}, R^{2S} and R^{3S} are each hydrogen. In other preferred embodiments R^{2S} is an electron withdrawing group, preferably NO₂, and R^{1S} and R^{3S} are each hydrogen.

In some such aspects the activatable self-immolative group capable of glycosidase cleavage to initiate the self-immolative reaction sequence is represented by the formula Gd:

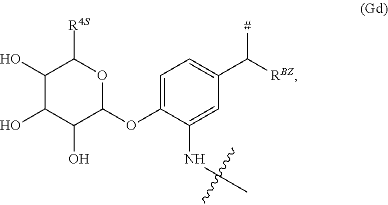

wherein R^{4S} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to the methylene carbamate unit.

In some such aspects the activatable self-immolative group capable of glycosidase cleavage to initiate the self-immolative reaction sequence is represented by the formula Gd*:

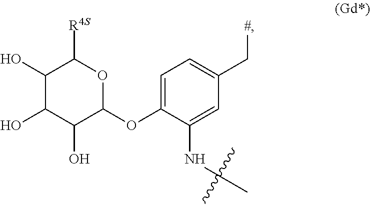

wherein R^{4S} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to a —OC(O)— unit that connects to a Spacer Unit or a drug moiety. In some embodiments, the —OC(O)— unit connects to a nitrogen atom of a Spacer Unit or a drug moiety to form a methylene carbamate moiety. In some embodiments, the —OC(O)— unit connects to an oxygen atom of a Spacer Unit or a drug moiety to form a methylene carbonate moiety.

In some such aspects the activatable self-immolative group capable of glycosidase cleavage to initiate the self-immolative reaction sequence is represented by the formula Gd**:

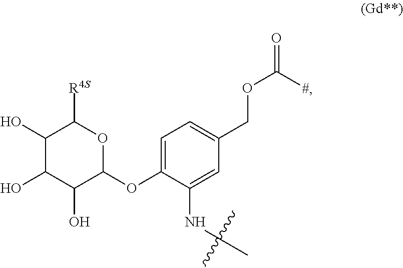

wherein R^{4S} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to a Spacer Unit or a drug moiety. In some embodiments, the —OC(O)— unit connects to a nitrogen atom of a Spacer Unit or a drug moiety to form a methylene carbamate moiety. In some embodiments, the —OC(O)— unit connects to an oxygen atom of a Spacer Unit or a drug moiety to form a methylene carbonate moiety.

In some embodiments wherein the activatable self-immolative moiety is comprised of a Glycoside (e.g., Glucuronide) Unit, it is represented by the following formula Ge:

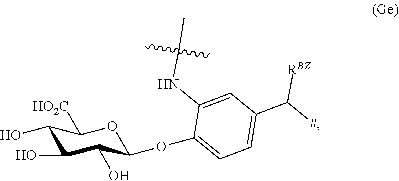

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark (#) indicates covalent attachment of the benzylic carbon of a Spacer or functional group attached to the drug. In some embodiments, the structure of formula Ge is attached to the drug moiety via a quaternized tertiary amine (N⁺), wherein the nitrogen atom is from a tertiary amine functional group on the unconjugated drug moiety.

In some embodiments wherein the activatable self-immolative moiety is comprised of a Glycoside (e.g., Glucuronide) Unit, it is represented by the following formula Ge*:

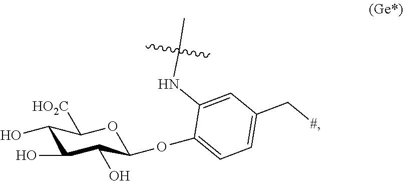

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark (#) indicates covalent attachment of the benzylic carbon of a Spacer or functional group attached to the drug. In some embodiments, the structure of formula Ge is attached to the drug moiety via a quaternized tertiary amine (N⁺), wherein the nitrogen atom is from a tertiary amine functional group on the unconjugated drug moiety.

Another type of Releasable Linker that provides a mechanism for separation of the drug from the cystiene engineered antibody and other components of the linker moeity through activation of a self-immolation cascade within the Linker Unit is comprised of a p-aminobenzyloxycarbonyl (PAB) moiety whose phenylene component is substituted with Jₘ wherein the subscript m indicating the number of substituents is an integer ranging from 0-4, and each J is independently-C₁-C₈ alkyl, —O—(C₁-C₈ alkyl), -halogen, -nitro or -cyano.

In some embodiments, R^{L} is a self-immolative group capable of releasing-D without the need for a separate hydrolysis step or subsequent self-immolative event. In some embodiments, -RL- is a PAB moiety that is linked to the carbonyl of —W— via the amino nitrogen atom of the PAB group, and connected directly to -D via a carbonate group. In related embodiments, -RL- is comprised of a PAB moiety that is linked to the carbonyl of -A-, —S*— or —B— via the amino nitrogen atom of the PAB group, and connected directly to -D via a carbonate group. Without being bound by any particular theory or mechanism, a possible mechanism of Drug release from RL comprised of a PAB moiety in which RL is attached directly to -D via a carbonate group is shown in Toki et al. (2002) J Org. Chem. 67:1866-1872.

In some embodiments, RL units containing a PAB moiety are represented by the formula:

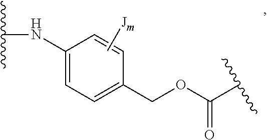

wherein subscript m is an integer ranging from 0-4, and each J is independently-C₁-C₈ alkyl, —O—(C₁-C₈ alkyl), -halogen, -nitro or -cyano.

Other examples of self-immolative groups include, but are not limited to, aromatic compounds that are electronically similar to the PAB moiety such as 2-aminoimidazol-5-methanol derivatives (Hay et al. (1999) Bioorg. Med. Chem. Lett. 9:2237) and ortho or para-aminobenzylacetals. Other RLs undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867).

In one embodiment, RL is a branched bis(hydroxymethyl) styrene (BHMS) unit.

In some embodiments, RL has the formula:

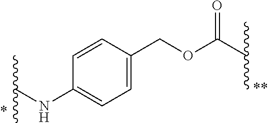 [SMILES: *Nc1ccc(COC(*)=O)cc1]

wherein the wavy line marked with ** indicates the site of attachment to D; and the wavy line marked with * indicates the point of attachment to additional linker components of Q.

In some embodiments, RL comprises a heterocyclic “self-immolating moiety” of Formulas I, II or III bound to the drug and incorporates an amide group that upon hydrolysis by an intracellular protease initiates a reaction that ultimately cleaves the self-immolative moiety from the drug such that the drug is released from the conjugate in an active form. The linker moiety further comprises a peptide sequence adjacent to the self-immolative moiety that is a substrate for an intracellular enzyme, for example an intracellular protease such as a cathepsin (e.g., cathepsin B), that cleaves the peptide at the amide bond shared with the self-immolative moiety.

In some embodiments, a heterocyclic self-immolating group (RL) is selected from Formulas I, II and III:

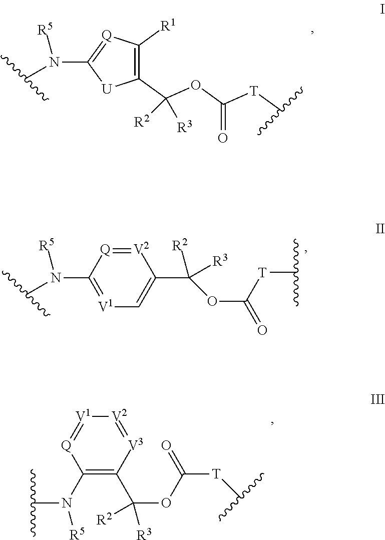

wherein the wavy lines indicate the covalent attachment sites to the cell-specific ligand and the D′ drug moiety, and wherein U is O, S or NR⁶; Q is CR⁴ or N; V¹, V² and V³ are independently CR⁴ or N provided that for formula II and III at least one of Q, V¹ and V² is N; T is a heteroatom of a drug moiety, wherein T and D′ together form a drug moiety; R¹, R², R³ and R⁴ are independently selected from the group consisting of H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and R⁵ and R⁶ are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; wherein C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ trifluoroalkyl, C₁-C₈alkyl, C₃-C₁₂ carbocycle, C₆-C₂₀ aryl, C₂-C₂₀ heterocycle, polyethyleneoxy, phosphonate, and phosphate.

The conjugate can be stable extracellularly, or in the absence of an enzyme capable of cleaving the amide bond of the self-immolative moiety. However, upon entry into a cell, or exposure to a suitable enzyme, an amide bond can be cleaved initiating a spontaneous self-immolative reaction resulting in the cleavage of the bond covalently linking the self-immolative moiety to the drug, to thereby effect release of the drug in its underivatized or pharmacologically active form.

The self-immolative moiety in conjugates of the invention can either incorporate one or more heteroatoms and thereby provide improved solubility, improve the rate of cleavage, and/or decrease propensity for aggregation of the conjugate. These improvements of the heterocyclic self-immolative linker constructs of the present invention over non-heterocyclic, PAB-type linkers can in some instances result in surprising and unexpected biological properties such as increased efficacy, decreased toxicity, and/or improvements in one or more desirable pharmacokinetic and/or pharmacodynamic properties.

Not to be limited by theory or any particular mechanism, the presence of electron-withdrawing groups on the heterocyclic ring of formula I, II or III linkers can moderate the rate of cleavage.

In one embodiment, the self-immolative moiety is the group of formula I in which Q is N, and U is O or S. Such a group has a non-linearity structural feature which can improve the solubility of the conjugates. In this context R can be H, methyl, nitro, or CF₃. In one embodiment, Q is N and U is O thereby forming an oxazole ring and R is H. In another embodiment, Q is N and U is S thereby forming a thiazole ring optionally substituted at R with an Me or CF₃ group.

In another exemplary embodiment, the self-immolative moiety is the group of formula II in which Q is N and V¹ and V² are independently N or CH. In another embodiment, Q, V¹ and V² are each N. In another embodiment, Q and V¹ are N while V² is CH. In another embodiment, Q and V² are N while V¹ is CH. In another embodiment, Q and V¹ are both CH and V² is N. In another embodiment, Q is N while V¹ and V² are both CH.

In another embodiment, the self-immolative moiety is the group of formula III in which Q, V¹, V² and V³ are each independently N or CH. In another embodiment Q is N while V¹, V² and V³ are each N. In another embodiment, Q V¹, and V² are each CH while V³ is N. In another embodiment Q, V² and V³ are each CH while V¹ is N. In another embodiment, Q, V¹ and V³ are each CH while V² is N. In another embodiment, Q and V² are both N while V¹ and V³ are both CH. In another embodiment Q and V² are both CH while V¹ and V³ are both N. In another embodiment, Q and V³ are both N while V¹ and V² are both CH.

### Partitioning Agent (S*):

The cysteine engineered ADCs described herein can also include a Partitioning Agent (S*). The Partitioning Agent portions are useful, for example, to mask the hydrophobicity of particular drug moieties or linker moiety components.

Representative Partitioning Agents include polyethylene glycol (PEG) units, cyclodextrin units, polyamides, hydrophilic peptides, polysaccharides and dendrimers.

When the polyethylene glycol (PEG) units, cyclodextrin units, polyamides, hydrophilic peptides, polysaccharides or dendrimers are included in Q, the groups may be present as an ‘in line’ component or as a side chain or branched component. For those embodiments in which a branched version is present, the linker moieties can include a lysine residue (or Parallel Connector Unit, B) that provides simple functional conjugation of, for example, the PEG unit, to the remainder of the linker moeity.

### Polyethylene Glycol Unit (PEG)

Polydisperse PEGs, monodisperse PEGs and discrete PEGs can be used as part of the Partitioning Agents in Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein can comprise one or multiple polyethylene glycol chains. A polyethylene glycol chain is composed of at least two ethylene oxide (CH₂CH₂O) subunits. In some embodiments the polyethylene glycol chains are linked together, for example, in a linear, branched or star shaped configuration. Typically, at least one of the PEG chains is derivitized at one end for covalent attachment to an appropriate site on a component of the Linker Unit (e.g. B) or can be used as an in-line (e.g., bifunctional) linking group within to covalently join two of the Linker Unit components (e.g., Z-A-S*-RL-, Z-A-S*-RL-Y—). Exemplary attachments within the Linker Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment within the linker moiety is by means of a non-conditionally cleavable linkage. In some embodiments, attachment within the linker moiety is not via an ester linkage, hydrazone linkage, oxime linkage, or disulfide linkage. In some embodiments, attachment within the linker moiety is not via a hydrazone linkage.

A conditionally cleavable linkage refers to a linkage that is not substantially sensitive to cleavage while circulating in the plasma but is sensitive to cleavage in an intracellular or intratumoral environment. A non-conditionally cleavable linkage is one that is not substantially sensitive to cleavage in any biological environment. Chemical hydrolysis of a hydrazone, reduction of a disulfide, and enzymatic cleavage of a peptide bond or glycosidic linkage are examples of conditionally cleavable linkages.

In some embodiments, the PEG Unit can be directly attached to a Parallel Connector Unit B. The other terminus (or termini) of the PEG Unit can be free and untethered and may take the form of a methoxy, carboxylic acid, alcohol or other suitable functional group. The methoxy, carboxylic acid, alcohol or other suitable functional group acts as a cap for the terminal PEG subunit of the PEG Unit. By untethered, it is meant that the PEG Unit will not be attached at that untethered site to a drug, to cysteine engineered antibody, or to another linking component. The skilled artisan will understand that the PEG Unit in addition to comprising repeating ethylene glycol subunits may also contain non-PEG material (e.g., to facilitate coupling of multiple PEG chains to each other). Non-PEG material refers to the atoms in the PEG Unit that are not part of the repeating —CH₂CH₂O— subunits. In some embodiments provided herein, the PEG Unit comprises two monomeric PEG chains attached to each other via non-PEG elements. In other embodiments provided herein, the PEG Unit comprises two linear PEG chains attached to a central core or Parallel Connector Unit (i.e., the PEG Unit itself is branched).

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) can also be useful as a reactive group for attaching PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, PEG molecules may be attached to amino groups using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG2-succinimidyl succinate (mPEG2-SS); mPEG-succinimidyl carbonate (mPEG-SC), mPEG2-succinimidyl carbonate (mPEG2-SC); mPEG-imidate, mPEG-para-nitrophenylcarbonate (mPEG-NPC), mPEG-imidate; mPEG2-para-nitrophenylcarbonate (mPEG2-NPC); mPEG-succinimidyl propionate (mPEG-SPA); mPEG2-succinimidyl propionate (mPEG2-SPA); mPEG-N-hydroxy-succinimide (mPEG-NHS); mPEG2-N-hydroxy-succinimide (mPEG2-NHS); mPEG-cyanuric chloride; mPEG2-cyanuric chloride; mPEG2-Lysinol-NPC, and mPEG2-Lys-NHS.

Generally, at least one of the PEG chains that make up the PEG Unit is functionalized so that it is capable of covalent attachment to other linker moiety components.

Functionalization includes, for example, via an amine, thiol, NHS ester, maleimide, alkyne, azide, carbonyl, or other functional group. In some embodiments, the PEG Unit further comprises non-PEG material (i.e., material not comprised of —CH₂CH₂O—) that provides coupling to other linker moiety components or to facilitate coupling of two or more PEG chains.

The presence of the PEG Unit (or other Partitioning Agent) in the Linker Unit can have two potential impacts upon the pharmacokinetics of the resulting cysteine engineered ADC. The desired impact is a decrease in clearance (and consequent increase in exposure) that arises from the reduction in non-specific interactions induced by the exposed hydrophobic elements of the cysteine engineered ADC or to the drug of the cysteine engineered ADC. The second impact is undesired and is a decrease in volume and rate of distribution that sometimes arises from the increase in the molecular weight of the cysteine engineered ADC.

Increasing the number of PEG subunits increases the hydrodynamic radius of a conjugate, typically resulting in decreased diffusivity. In turn, decreased diffusivity typically diminishes the ability of the cysteine engineered ADC to penetrate into a tumor (Schmidt and Wittrup, Mol Cancer Ther 2009; 8:2861-2871). Because of these two competing pharmacokinetic effects, it is desirable to use a PEG that is sufficiently large to decrease the cysteine engineered ADC clearance thus increasing plasma exposure, but not so large as to greatly diminish its diffusivity, to an extent that it interferes with the ability of the cysteine engineered ADC to reach the intended target cell population. See the examples (e.g., examples 1, 18, and 21) of US2016/0310612, which are incorporated by reference herein, for methodology for selecting an optimal PEG size for a particular drug-linker.

In one group of embodiments, the PEG Unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In preferred embodiments, the PEG Unit comprises a combined total of at least 4 subunits, at least 6 subunits, at least 8 subunits, at least 10 subunits, or at least 12 subunits. In some such embodiments, the PEG Unit comprises no more than a combined total of about 72 subunits, preferably no more than a combined total of about 36 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the Partitioning Agent S* is a linear PEG Unit comprising from 2 to 20, or from 2 to 12, or from 4 to 12, or 4, 8, or 12 —CH₂CH₂O— subunits. In some embodiments, the linear PEG Unit is connected at one end of the PEG Unit to the RL Unit and at the other end of the PEG Unit to the Stretcher/Connector Units (Z-A-). In some embodiments, the PEG Unit is connected to the RL Unit via a —CH₂CH₂C(O)— group that forms an amide bond with the RL Unit (e.g., —(CH₂CH₂O)ₙ—CH₂CH₂C(O)—RL) and to the Stretcher Unit/Connector Unit (Z-A-) via an —NH— group (e.g., Z-A-NH—(CH₂CH₂O)ₙ—) that forms an amide bond with the Z-A-portion.

Illustrative embodiments for PEG Units that are connected to the RL and Stretcher/Connector Units (Z-A-) are shown below:

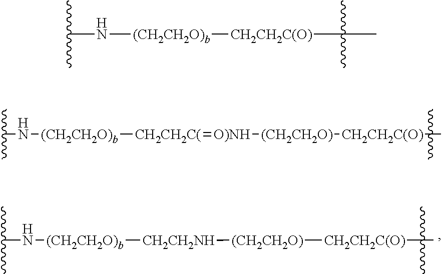

and in a particular embodiment, the PEG Unit is:

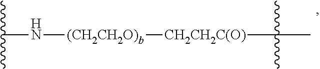

wherein the wavy line on the left indicates the site of attachment to Z-A-, the wavy line on the right indicates the site of attachment to RL, and each b is independently selected from 2 to 72, 4 to 72, 6 to 72, 8 to 72, 10 to 72, 12 to 72, 2 to 24, 4 to 24, 6 to 24, or 8 to 24, 2 to 12, 4 to 12, 6 to 12, and 8 to 12. In some embodiments, subscript b is 2, 4, 8, 12, or 24. In some embodiments, subscript b is 2. In some embodiments, subscript b is 4. In some embodiments, subscript b is 8. In some embodiments, subscript b is 12.

In some embodiments, the linear PEG Unit that is connected to the Parallel Connector Unit at one end and comprises a terminal cap at the other end. In some embodiments, the PEG Unit is connected to the Parallel Connector Unit via a carbonyl group that forms an amide bond with the Parallel Connector Unit lysine residue amino group (e.g., —(OCH₂CH₂)ₙ—C(O)—B—) and includes a PEG Unit terminal cap group selected from the group consisting of C₁₋₄alkyl and C₁₋₄alkyl-CO₂H. In some embodiments, the Partitioning Agent S* is a linear PEG Unit comprising 4, 8, or 12 —CH₂CH₂O-subunits and a terminal methyl cap.

Illustrative linear PEG Units that can be used in any of the embodiments provided herein are as follows:

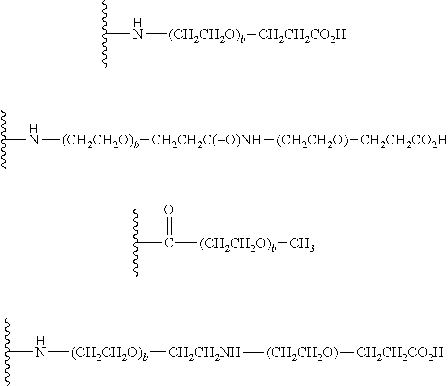

and in a particular embodiment, the PEG Unit is:

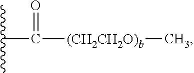

wherein the wavy line indicates site of attachment to the Parallel Connector Unit (B), and each n is independently selected from 4 to 72, 6 to 72, 8 to 72, 10 to 72, 12 to 72, 6 to 24, or 8 to 24. In some embodiments, subscript b is about 4, about 8, about 12, or about 24.

As used to herein, terms “PEG2”, “PEG4”, “PEG8”, and “PEG12” refers to specific embodiments of PEG Unit which comprises the number of PEG subunits (i.e., the number of subscription “b”). For example, “PEG2” refers to embodiments of PEG Unit that comprises 2 PEG subunits, “PEG4” refers to embodiments of PEG Unit that comprises 4 PEG subunits, “PEG8” refers to embodiments of PEG Unit that comprises 8 PEG subunits, and “PEG12” refers to embodiments of PEG Unit that comprises 12 PEG subunits.

As described herein, the PEG unit is selected such that it improves clearance of the resultant cysteine engineered ADC but does not significantly impact the ability of the Conjugate to penetrate into the tumor. In embodiments, the PEG unit to be selected for use will preferably have from 2 subunits to about 24 subunits, from 4 subunits to about 24 subunits, more preferably about 4 subunits to about 12 subunits.

In preferred embodiments of the present disclosure the PEG Unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG Unit has at least 6 subunits or at least 8, 10 or 12 subunits. In some such aspects, the PEG Unit has at least 6 subunits or at least 8, 10 or 12 subunits but no more than 72 subunits, preferably no more than 36 subunits.

It will be appreciated that when referring to PEG subunits, and depending on context, the number of subunits can represent an average number, e.g., when referring to a population of cysteine engineered ADCs or drug-linker compounds, and/or using polydisperse PEGs.

### Parallel Connector Unit (B):

In some embodiments, the cysteine engineered ADCs or drug-linker compounds will comprise a Parallel Connector Unit to provide a point of attachment to a Partitioning Agent (shown in the Linker Units as —B(S*)—). As a general embodiment, the PEG Unit can be attached to a Parallel Connector Unit such as lysine as shown below wherein the wavy line and asterisks indicate covalent linkage within the linker moiety of a cysteine engineered ADCs or drug-linker compounds:

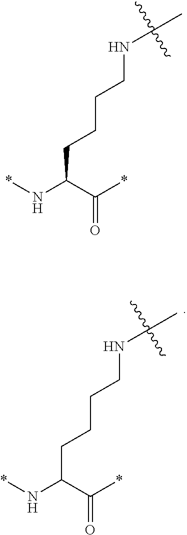 [SMILES: *NCCCC[C@H](N*)C(*)=O ; *NCCCCC(N*)C(*)=O]

In some embodiments, the Parallel Connector Unit (LP) and Partitioning Agent (S*) (together, —B(S*)—) have the structure of

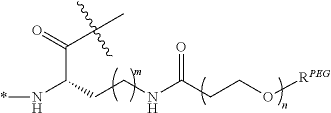

wherein m ranges from 0 to 6; n ranges from 2 to 24; R^{PEG} is a PEG Capping Unit, preferably H, —CH₃, or —CH₂CH₂CO₂H, the asterisk (*) indicates covalent attachment to a Connector Unit A corresponding in formula Za, Za′, Zb′ or Zc′ and the wavy line indicates covalent attachment to the Releasable Linker (RL). In some embodiments, the structure is attached to a Connector Unit A in formula Za or Za′. In some embodiments, n is 2, 4, 8, or 12. In instances such as those shown here, the shown PEG group is meant to be exemplary of a variety of Partitioning Agents including PEG groups of different lengths and other Partitioning Agents that can be directly attached or modified for attachment to the Parallel Connector Unit.

### Spacer Unit (Y):

In some embodiments, the cysteine engineered ADCs provided herein will have a Spacer (Y) between the Releasable Linker (RL) and the drug moiety. The Spacer Unit can be a functional group to facilitate attachment of RL to the drug moiety, or it can provide additional structural components to further facilitate release of the drug moiety from the remainder of the cysteine engineered ADC (e.g., a methylene carbamate unit or a self-immolative para-aminobenzyl (PAB) component).

In those embodiments to further facilitate release of the drug moiety as free drug, the Spacer Unit-drug moiety group (—Y-T*-D or —Y-D) is represented by one of the following formulae:

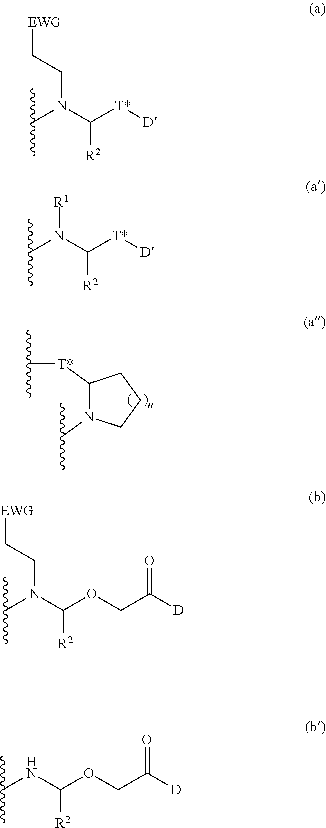

wherein R¹ and R² are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂₀ substituted heterocycle are independently optionally substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ trifluoroalkyl, C₁-C₈alkyl, C₃-C₁₂ carbocycle, C₆-C₂₀ aryl, C₂-C₂₀ heterocycle, polyethyleneoxy, phosphonate, and phosphate; subscript n is 1 or 2; wherein EWG represents an electron-withdrawing group; T* is the heteroatom of a drug moiety; D′ is the remainder of the drug moiety, wherein T* and D′ together form the drug moiety D; and the wavy line adjacent to the nitrogen atom is the point of covalent attachment to RL. In some embodiments, EWG is selected from the group consisting of —CN, —NO₂, —CX₃, —X, —C(═O)OR′, —C(═O)N(R′)₂, —C(═O)R′, —C(═O)X, —S(═O)₂R′, —S(═O)₂OR′, —S(═O)₂NHR′, —S(═O)₂N(R′) 2, —P(═O)(OR′)₂, —P(═O)(CH₃)NHR′, —NO, —N(R′)₃⁺, wherein X is —F, —Br, —Cl, or —I, and R′ is independently selected from the group consisting of hydrogen and C₁-C₆ alkyl.

There are a number of PEG attachment methods available to those skilled in the art, [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); PCT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal .alpha.-carbon of a peptide); Veronese et al., (1985) Appl. Biochem. Biotechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

In some embodiments, the Spacer Unit is represented by one of the following formulae:

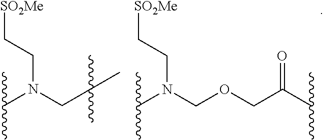 [SMILES: *CN(*)CCS(C)(=O)=O ; *C(=O)COCN(*)CCS(C)(=O)=O]

In still other embodiments, the Spacer Unit-drug moiety group (—Y-T*-D′ or —Y-D) comprises a methylene carbamate unit and is represented by one of the following the formulae:

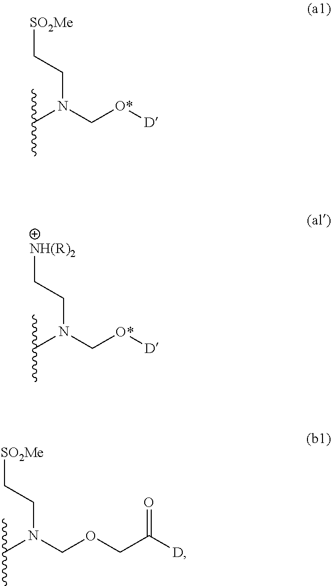

wherein formula (a1) and formula (a1′) in which each R is independently —H or C₁-C₄ alkyl represents Spacer Units in which O* is an oxygen atom from the drug moiety, and the wavy lines of formula (a 1), formula (a1′) and formula (b1) retain their previous meanings from formulae (a), (a′) and (b), respectively. In formula (a1′) the —CH₂CH₂N⁺(R)₂ moiety represents exemplary Basic Units in protonated form.

In some embodiments, the Spacer Unit-drug moiety group —Y-T*-D′ is represented by one of the following formulae:

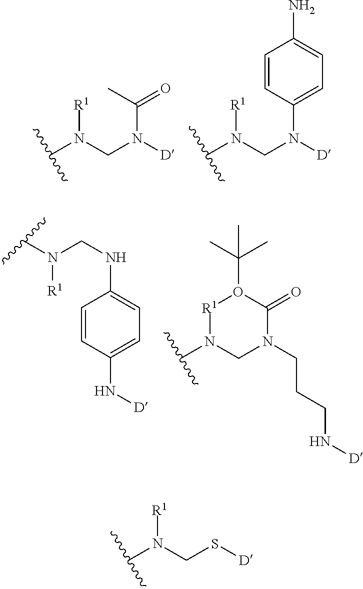

wherein R¹ is as defined for formula (a′), the wavy line adjacent to the nitrogen atom is the point of covalent attachment to RL, T* is as defined above, and D′ represents the remainder of the drug moiety, wherein T* and D′ together form a drug moiety D.

In some embodiments, the Spacer Unit-drug moiety group (—Y-T*-D) is represented by one of the following formulae:

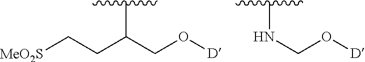

wherein the wavy line adjacent is the point of covalent attachment to RL, T* is as defined above, and D′ represents the remainder of the drug moiety, wherein T* and D′ together form the drug moiety D.

In some embodiments, the Spacer Unit-drug moiety group (—Y-T*-D) is represented by one of the following formulae:

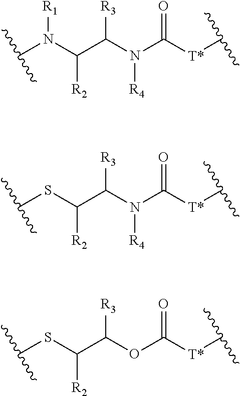

wherein R¹ and R⁴ are independently selected from H, C₁-C₈ alkyl, C₁-C₈ substituted alkyl, C₂-C₅ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate; R² is selected from the group consisting of H, C₁-C₈alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate, or is combined with R³ and the intervening atoms to form a 5-or 6-membered carbocyclo or heterocyclo; R³ is selected from the group consisting of H, C₁-C₈alkyl, C₁-C₈ substituted alkyl, C₂-C₈ alkenyl, C₂-C₈ substituted alkenyl, C₂-C₅ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; wherein the C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from the group consisting of F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O) N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate, or is combined with R² and the intervening atoms to form a 5-or 6-membered carbocyclo or heterocyclo; T* is the heteroatom of a drug moiety; D′ is the remainder of the drug moiety, wherein T* and D′ together form the drug moiety (i.e. T*+D′=D); D is the drug moiety; and the wavy line adjacent to the nitrogen atom is the point of covalent attachment to RL.

In some embodiments, the Spacer Unit-drug moiety (—Y-T*-D′) is represented by one of the following formulae:

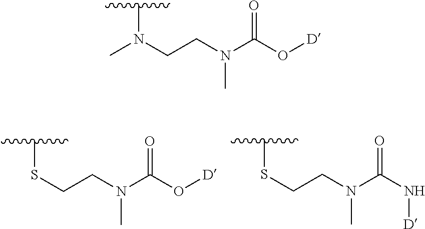

wherein the wavy line adjacent to the nitrogen atom is the point of covalent attachment to RL, T* is as defined above, and D′ represents the remainder of the drug moiety, wherein T* and D′ together form the drug moiety D.

In some embodiments, the Spacer Unit is represented by the formula:

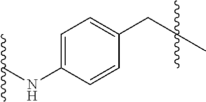 [SMILES: *Cc1ccc(N*)cc1]

wherein the wavy line adjacent to the nitrogen atom is the point of covalent attachment to RL, as defined above, and the wavy line next to the benzylic carbon atom connects to a drug moiety. In some embodiments, the drug moiety is attached to the benzylic carbon atom via a quaternized tertiary amine (N⁺) of D.

In still other embodiments, the Spacer Unit is represented by the formula:

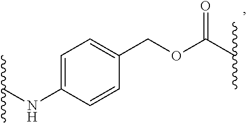 [SMILES: *Nc1ccc(COC(*)=O)cc1]

wherein the wavy line adjacent to the nitrogen atom is the point of covalent attachment to RL, as defined above, and the wavy line next to the —OC(O)— group connects to a drug moiety. In some embodiments, the drug moiety is attached via T*, wherein T* is the heteroatom of a drug moiety.

### 4. Drug Moiety

The cysteine engineered ADCs of the present invention may comprise a drug moiety. In some embodiments, the drug moiety is a free drug or a pharmaceutically acceptable salt thereof and may be useful for pharmaceutical treatment of hyperproliferative diseases and disorders. In some embodiments, the drug moiety is cytotoxic, cytostatic, immunosuppressive, immunostimulatory, or immunomodulatory drug. In some embodiments, the drug moiety is a cytotoxic agent, a growth inhibitory agent, or a chemotherapy.

Useful classes of cytotoxic agents include, for example, antitubulin agents (which may also be referred to as tubulin disrupting agents) and tubulin inhibitors. Exemplary agents include, for example, anthracyclines, auristatins (e.g., auristatin T, auristatin E, AFP, monomethyl auristatin F (MMAF), lipophilic monomethyl aurstatin F, monomethyl auristatin E (MMAE)), camptothecins, CC-1065 analogues, calicheamicin, analogues of dolastatin 10, duocarmycins, etoposides, maytansines and maytansinoids, melphalan, methotrexate, mitomycin C, taxanes (e.g., paclitaxel and docetaxel), nicotinamide phosphoribosyltranferase inhibitor (NAMPTi), tubulysin M, benzodiazepines and benzodiazepine containing drugs (e.g., pyrrolo[1,4]-benzodiazepines (PBDs), indolinobenzodiazepines, rhizoxin, paltoxin, and oxazolidinobenzodiazepines) and vinca alkaloids. Select benzodiazepine containing drugs are described in WO 2010/091150, WO 2012/112708, WO 2007/085930, and WO 2011/023883.

Particularly useful classes of cytotoxic agents include, for example tubulin disrupting agents. Other particularly useful cytotoxic agents include, for example, auristatins (e.g., auristatin T, auristatin E, AFP, monomethyl auristatin F (MMAF), lipophilic analogs of monomethyl auristatin F, monomethyl auristatin E (MMAE)) and camptothecins (e.g., camptothecin, irinotecan and topotecan).

In some embodiments, the drug moiety is a cytostatic or cytotoxic agent disclosed in U.S. Provisional Application No. 63/397,776, U.S. Provisional Application No. 63/321,105, U.S. Provisional Application No. 63/407,609, WO 2019/195665, WO 2019/236954, or WO 2021/067861.

The cytotoxic agent can also be an auristatin. The auristatin can be an auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatins include auristatin T, AFP, MMAF, and MMAE. The synthesis and structure of various auristatins are described in, for example, US 2005-0238649 and US2006-0074008.

In some embodiments, the drug is an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine), and auristatins (e.g., auristatin E, AFP, MMAF, MMAE, AEB, AEVB). Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be mytansine or a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et a1., 1992, Cancer Res.).

In some embodiments, D is a tubulin disrupting agent. In some embodiments, D is an auristatin or a tubulysin. In some embodiments, D is an auristatin. In some embodiments, D is a tubulysin.

In some embodiments, the drug moiety incorporates the structure of an anthracyclin compound. Without being bound by theory, the cytotoxicity of those compounds to some extent may also be due to topoisomerase inhibition. In some of those embodiments the anthracyclin compound has a structure disclosed in Minotti, G., et a1., “Anthracyclins: molecular advances and pharmacologic developments in antitumor activity and cardiotoxicity” Pharmacol Rev. (2004) 56 (2): 185-229. In some embodiments, the anthracyclin compound is doxorubicin, idarubicin, daunorubicin, doxorubicin propyloxazoline (DPO), morpholino-doxorubicin, or cyanomorpholino-doxorubicin.

In more preferred embodiments the auristatin drug compound incorporated into-D is monomethylauristatin E (MMAE) or monomethylauristatin F (MMAF).

In some embodiments, the drug moiety is a microtubule inhibitor. In some embodiments, the microtubule inhibitor is an auristatin or a tubulysin. In some embodiments, the drug moiety is selected from the group consisting of auristatin T, tubulysin M, tubulysin OEt, MMAE (monomethyl auristatin E), and MMAF (monomethyl auristatin F).

In some embodiments, the engineered ADC has low stability. In some embodiments, less than 100%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the drug moiety is conjugated to the antibody at day 7 after administration to a subject.

Also included are Cysteine engineered ADC compositions containing any of the Cysteine engineered ADC compounds listed above wherein p′ is replaced with p as described herein.

### C. Nucleic Acids, Vectors, and Host Cells

The invention further provides nucleic acids encoding a cysteine engineered antibody, such as any of the cysteine engineered antibodies described herein. Typically, the nucleic acids may also encode a signal peptide fused to the cysteine engineered antibody (e.g., fused to each of the heavy chains and/or each of the light chains of the cysteine engineered antibody). Coding sequences within the nucleic acids can be in operable linkage with regulatory sequences to ensure expression of the coding sequences, such as a promoter, enhancer, ribosome binding site, transcription termination signal and the like. The nucleic acids can be synthesized by for example, solid state synthesis or PCR of overlapping oligonucleotides.

In some embodiments, provided herein is a nucleic acid encoding a cysteine engineered antibody comprising cysteine amino acid substitutions. In some embodiments, the cysteine engineered antibody is a full-length antibody, a Fab, a Fab′, a (Fab′)₂, an Fv, or a single chain Fv (scFv). In some embodiments, the cysteine engineered antibody comprises one light chain and one heavy chain (e.g., a half antibody). In some embodiments, the cysteine amino acid substitution is at position 121 the light chain, according to Kabat numbering. In some embodiments, the cysteine amino acid substitution is at position 114 of the light chain, according to Kabat numbering. In some embodiments, the cysteine amino acid substitution is at position 400 of the heavy chain, according to EU numbering. In some embodiments, the cysteine amino acid substitution is at position 375 of the heavy chain, according to EU numbering. In some embodiments, the cysteine engineered antibody comprises two light chains and two heavy chains. In some embodiments, the cysteine engineered antibody is a full-length antibody. In some embodiments, the cysteine amino acid substitution is at position 121 of each of the light chains, according to Kabat numbering. In some embodiments, the cysteine amino acid substitution is at position 114 of each of the light chains, according to Kabat numbering. In some embodiments, the cysteine amino acid substitution is at position 400 of each of the heavy chains, according to EU numbering. In some embodiments, the cysteine amino acid substitution is at position 375 of each of the heavy chain, according to EU numbering.

In some embodiments, provided herein is a nucleic acid encoding a cysteine engineered antibody comprising two light chains and two heavy chains. In some embodiments, the cysteine engineered antibody comprises at least one cysteine amino acid substitution on each of the light chains. In some embodiments, the cysteine engineered antibody comprises at least one cysteine amino acid substitution on each of the heavy chains. In some embodiments, the cysteine engineered antibody comprises at least one cysteine amino acid substitution on each of the light chains and at least one cysteine amino acid substitution on each of the heavy chains. In some embodiments, the cysteine engineered antibody comprises one cysteine amino acid substitution on each of the light chains and one cysteine amino acid substitution on each of the heavy chains. In some embodiments, the cysteine engineered antibody comprises a different cysteine amino acid substitution on each of the light chains. In some embodiments, the cysteine engineered antibody comprises a different cysteine amino acid substitution on each of the heavy chains. In some embodiments, the cysteine engineered antibody comprises a different cysteine amino acid substitution on each of the light chains but the same cysteine amino acid substitution on each of the heavy chains. In some embodiments, the cysteine engineered antibody comprises a different cysteine amino acid substitution on each of the heavy chains but same cysteine amino acid substitution on each of the light chains. In some embodiments, the cysteine engineered antibody comprises the same cysteine amino acid substitution on each of the light chains. In some embodiments, the cysteine engineered antibody comprises the same cysteine amino acid substitution on each of the heavy chains. In some embodiments, the cysteine engineered antibody comprises the same cysteine amino acid substitution on each of the light chains the same cysteine amino acid substitution on each of the heavy chains. In some embodiments, the cysteine amino acid substitutions are at position 121 of each of the light chains and position 375 of each of the heavy chains. In some embodiments, the cysteine amino acid substitutions are at position 121 of each of the light chains and position 400 of each of the heavy chains. In some embodiments, the cysteine amino acid substitutions are at position 114 of each of the light chains and position 400 of each of the heavy chains. In some embodiments, the nucleic acid encodes a cysteine engineered antibody comprising cysteine amino acid substitutions at position 121 of each of the light chains and position 375 of each of the heavy chains. In some embodiments, the nucleic acid encodes a cysteine engineered antibody comprising cysteine amino acid substitutions at position 121 of each of the light chains and position 400 of each of the heavy chains. In some embodiments, the nucleic acid encodes a cysteine engineered antibody comprising cysteine amino acid substitutions at position 114 of each of the light chains and position 400 of each of the heavy chains. In some embodiments, the amino acid position numbering in the light chains are according to Kabat numbering and the amino acid position numbering in the heavy chains are according to EU numbering.

In some embodiments, provided herein is a set of nucleic acids, wherein a first nucleic acid of the set encodes a light chain of a cysteine engineered antibody and a second nucleic acid encodes a heavy chain of a cysteine engineered antibody. In some embodiments, the first nucleic acid encodes the light chain of a cysteine engineered antibody comprising a cysteine amino acid substitution at position 121. In some embodiments, the first nucleic acid encodes the light chain of a cysteine engineered antibody comprising a cysteine amino acid substitution at position 114. In some embodiments, the second nucleic acid encodes the heavy chain of a cysteine engineered antibody comprising a cysteine amino acid substitution at position 375. In some embodiments, the second nucleic acid encodes the heavy chain of a cysteine engineered antibody comprising a cysteine amino acid substitution at position 400. In some embodiments, the amino acid position numbering in the light chains are according to Kabat numbering and the amino acid position numbering in the heavy chains are according to EU numbering.

The nucleic acids encoding a cysteine engineered antibody can occur in isolated form (e.g., isolated nucleic acids), or can be cloned into one or more vectors. Thus, in some embodiments, provided herein are one or more vectors encoding a nucleic acid encoding a cysteine engineered antibody, such as any of the cysteine engineered antibodies described herein. Nucleic acids encoding a cysteine engineered antibody can be joined as one contiguous nucleic acid, e.g., within a single expression vector, or can be separate, e.g., each cloned into its own expression vector.

In some aspects, provided herein is a host cell comprising a nucleic acid encoding a cysteine engineered antibody, such as any of the cysteine engineered antibodies described herein. In other aspects, provided herein is a host cell comprising a vector encoding a nucleic acid encoding a cysteine engineered antibody, such as any of the cysteine engineered antibodies described herein. In some embodiments, the host cell is a mammalian host cell, e.g., a CHO cell.

### III. Pharmaceutical Compositions and Kits

The present disclosure provides pharmaceutical compositions comprising the cysteine engineered ADCs described herein and a pharmaceutically acceptable carrier. The cysteine engineered ADCs can be in any form that allows for the administration to a patient for the treatment of a disorder associated with expression of a target antigen to which the cysteine engineered antibody binds. For example, the cysteine engineered ADCs can be in the form of a liquid or solid. In some embodiments, the preferred route of administration is parenteral. Parenteral administration includes subcutaneous injections, intravenous, intramuscular, intrasternal injection or infusion techniques. In some embodiments, the pharmaceutical compositions are administered parenterally. In some embodiments, the pharmaceutical compositions are administered intravenously.

Pharmaceutical compositions can be formulated so as to allow the cysteine engineered ADC to be bioavailable upon administration of the pharmaceutical composition to a patient. Pharmaceutical compositions can take the form of one or more dosage units, where for example, a tablet (e.g., a tablet comprising the cysteine engineered ADCs of the present disclosure) can be a single dosage unit.

Materials used in preparing the pharmaceutical compositions can be non-toxic in the amounts used. It will be evident to those of ordinary skill in the art that the optimal dosage of the active ingredient(s) (e.g., cysteine engineered ADCs) in the pharmaceutical composition will depend on a variety of factors. Relevant factors include, without limitation, the type of animal (e.g., human), the particular form of the cysteine engineered ADC, the manner of administration, and the pharmaceutical composition employed.

The pharmaceutical composition can be, for example, in the form of a liquid. The liquid can be useful for delivery by injection. In a pharmaceutical composition for administration by injection, one or more of a surfactant, preservative, wetting agent, dispersing agent, suspending agent, buffer, stabilizer and isotonic agent can also be included.

The liquid pharmaceutical compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral pharmaceutical composition can be enclosed in ampoule, a disposable syringe or a multiple-dose vial made of glass, plastic or other material. Physiological saline is an exemplary adjuvant.

In some embodiments, the amount of the cysteine engineered ADC that is effective in the treatment of a particular disorder or condition (e.g., a cancer) will depend on the nature of the disorder or condition, and can be determined by standard clinical techniques. In addition, in vitro or in vivo assays can optionally be employed to help identify optimal dosage ranges. The precise dose to be employed in the pharmaceutical compositions will also depend on the route of administration, and the seriousness of the disease or disorder, and should be decided according to the judgment of the practitioner and each patient's circumstances.

In some embodiments, the pharmaceutical compositions comprise an effective amount of a drug moiety such that a suitable dosage will be obtained. Typically, this amount is at least about 0.01% of a cysteine engineered ADC by weight of the pharmaceutical composition.

For intravenous administration, the pharmaceutical composition can comprise from about 0.01 to about 100 mg of a cysteine engineered ADC per kg of the patient's body weight. In one aspect, the pharmaceutical composition can include from about 1 to about 100 mg of a cysteine engineered ADC per kg of the patient's body weight. In another aspect, the amount administered will be in the range from about 0.1 to about 25 mg/kg of body weight of the cysteine engineered ADC.

Generally, the dosage of a cysteine engineered ADC administered to a patient is typically about 0.01 mg/kg to about 100 mg/kg of the patient's body weight. In some embodiments, the dosage administered to a patient is between about 0.01 mg/kg to about 15 mg/kg of the patient's body weight. In some embodiments, the dosage administered to a patient is between about 0.1 mg/kg and about 15 mg/kg of the patient's body weight. In some embodiments, the dosage administered to a patient is between about 0.1 mg/kg and about 20 mg/kg of the patient's body weight. In some embodiments, the dosage administered is between about 0.1 mg/kg to about 5 mg/kg or about 0.1 mg/kg to about 10 mg/kg of the patient's body weight. In some embodiments, the dosage administered is between about 1 mg/kg to about 15 mg/kg of the patient's body weight. In some embodiments, the dosage administered is between about 1 mg/kg to about 10 mg/kg of the patient's body weight. In some embodiments, the dosage administered is between about 0.1 to 4 mg/kg, even more preferably 0.1 to 3.2 mg/kg, or even more preferably 0.1 to 2.7 mg/kg of the patient's body weight over a treatment cycle.

The cysteine engineered ADCs can be administered by any convenient route, for example by infusion or bolus injection, by absorption through epithelial or mucocutaneous linings (e.g., oral mucosa, rectal and intestinal mucosa). Administration can be systemic or local. Various delivery systems are known, e.g., encapsulation in liposomes, microparticles, microcapsules, capsules, and can be used to administer the cysteine engineered ADC. In certain embodiments, more than one compounds or composition is administered to a patient.

The term “carrier” refers to a diluent, adjuvant or excipient, with which a compound is administered. Such pharmaceutical carriers can be liquids, such as water and oils, including those of petroleum, animal, vegetable or synthetic origin, such as peanut oil, soybean oil, mineral oil, sesame oil. The carriers can be saline, gum acacia, gelatin, starch paste, talc, keratin, colloidal silica, urea. In addition, auxiliary, stabilizing, thickening, lubricating and coloring agents can be used. In one embodiment, when administered to a patient, the compound or compositions and pharmaceutically acceptable carriers are sterile. Water is an exemplary carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol. The present compositions, if desired, can also contain minor amounts of wetting or emulsifying agents, or pH buffering agents.

In some embodiments, the cysteine engineered ADCs are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the pharmaceutical compositions can also include a solubilizing agent. Pharmaceutical compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of cysteine engineered ADC. Where a cysteine engineered ADC is to be administered by infusion, it can be dispensed, for example, with an infusion bottle containing sterile pharmaceutical grade water or saline. Where the conjugate is administered by injection, an ampoule of sterile water for injection or saline can be provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

Pharmaceutical compositions of the present disclosure comprise the cysteine engineered ADCs of the present disclosure and a pharmaceutically acceptable carrier. In some preferred embodiments, all, or substantially all, or more than 50% of the cysteine engineered ADCs present in the pharmaceutical composition comprises a hydrolyzed thio-substituted succinimide. In some preferred embodiments, more than 55%, 60%, 65%, 70%, 75%, 80%, 85%, 90%, 91%, 92%, 93%, 94%, 95%, 96%, 97%, 98%, or 99% of the Cysteine engineered antibody Drug Conjugates present in the pharmaceutical composition comprises a hydrolyzed thio-substituted succinimide.

The present disclosure also provide for kits. In some embodiments, the kit comprises a cysteine engineered antibody, such as any of the cysteine engineered antibodies described herein. In some embodiments, the kit comprises: (a) a cysteine engineered antibody, such as any of the cysteine engineered antibodies described herein, and (b) one or more additional containers containing a cytotoxic agent, such as any of the cytotoxic described herein. In some embodiments, the kit comprises a container containing a cysteine engineered ADC, such as any of the cysteine engineered ADCs described herein. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### IV. Methods of Use

The cysteine engineered antibodies and/or cysteine engineered ADCs, or pharmaceutical compositions thereof, are useful for reducing toxicity of ADCs. Moreover, the cysteine engineered ADCs or pharmaceutical compositions thereof may be useful for treating a disease, such as a cancer, in a patient.

### A. Methods of Reducing Toxicity

In some aspects, the present disclosure provides methods of reducing toxicity of an ADC comprising an antibody moiety conjugated to a drug moiety. Reduced toxicity may be determined by the absence of conditions or symptoms, fewer conditions or symptoms, or reduced severity of conditions or symptoms in a patient, compared to a second cysteine engineered ADC that comprises a drug moiety conjugated to a cysteine amino acid at one or more other positions. In some embodiments, reduced toxicity is achieved by generating a cysteine engineered ADC comprising one or more cysteine amino acid substitutions. The cysteine engineered ADC may be any of the cysteine engineered ADCs described herein. In some embodiments, the generating comprises substituting an amino acid in the cysteine engineered antibody with a cysteine amino acid. In some embodiments, the generating comprises inserting a cysteine amino acid into the cysteine engineered antibody. In some embodiments, the toxicity of the cysteine engineered ADC is reduced compared to a second cysteine engineered ADC that comprises a drug moiety conjugated to a cysteine amino acid at one or more other positions, when administered to a patient.

In some embodiments, the cysteine engineered ADC comprises a cysteine engineered antibody, such as any of the cysteine engineered antibodies provided herein, conjugated to a drug moiety. In some embodiments, the cysteine engineered antibody comprises: a heavy chain and a light chain; and, ii) the cysteine amino acid substitutions are selected from the group consisting of: a) position 121 of the light chain and position 375 of the heavy chain; b) position 121 of the light chain and position 400 of the heavy chain; and, c) position 114 of the light chain and position 400 of the heavy chain, wherein the amino acid position numbering in the light chain is according to Kabat numbering and the amino acid position numbering in the heavy chain is according to EU numbering. In some embodiments, the cysteine engineered antibody is a full-length antibody, a Fab, a Fab′, a (Fab′)₂, an Fv, or a single chain Fv (scFv). In some embodiments, the cysteine engineered antibody comprises two heavy chains and two light chains, wherein the cysteine amino acid substitutions are selected from the group consisting of: a) position 121 of at least one the light chains and position 375 of at least one of the heavy chains; b) position 121 of at least one of the light chains and position 400 of at least one of the heavy chains; and, c) position 114 of at least one of the light chains and position 400 of each of at least one of the heavy chains, wherein the amino acid position numbering in the light chains are according to Kabat numbering and the amino acid position numbering in the heavy chains are according to EU numbering. In some embodiments, the cysteine engineered antibody comprises: i) two heavy chains and two light chains and ii) the cysteine amino acid substitutions selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

The risks of irreversible toxicities, such as ocular toxicity, bone marrow toxicity, and/or lung toxicity continue to exist for many currently available ADCs (e.g., both unmodified ADCs and current standard cysteine engineered ADCs). Newly developed ADCs are often abandoned by pharmaceutical companies or rejected by regulatory agencies because the damage or toxicity to non-target tissue exceeds the clinical and/or therapeutic benefit. In some embodiments, the methods provided herein generally result in reduced toxicity of ADCs (e.g., cysteine engineered ADCs) that otherwise would not be of clinical or therapeutic value due to toxicity when administered to a patient. In some embodiments, reduced toxicity can occur using the cysteine engineered antibodies, cysteine engineered ADCs, and/or pharmaceutical compositions thereof, including without limitation when: (1) the dosage administered to a patient is increased above current therapeutic dosages; (2) the dosage administered to a patient remains the same as current therapeutic dosages; or (3) the dosage administered to a patient is decreased below current therapeutic dosages. In some embodiments, the cysteine engineered antibodies, cysteine engineered ADCs, and/or pharmaceutical compositions thereof, including the specifically numbered scenarios in this paragraph can elicit improved or similar therapeutic effect as seen with the current therapeutic dosages with no worse, fewer, or no toxicities, when administered to a patient.

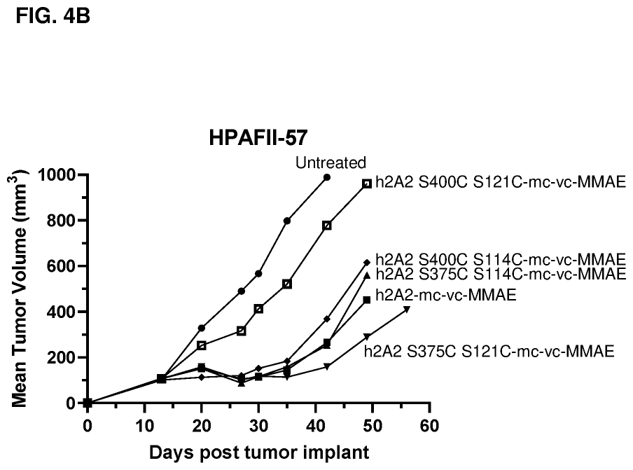

The plasma stability of a cysteine engineered ADC may serve as a measurement off-target drug release, and may therefore be predictive of the in vivo efficacy and safety of the cysteine engineered ADC. In some embodiments, one or more side effects in the patient is improved after administration of the cysteine engineered ADC as compared to administration of a second, comparison, cysteine engineered ADC. In some embodiments, the second cysteine engineered ADC is an unmodified or parental antibody conjugated to a drug moiety. In some embodiments, the second cysteine engineered ADC is a cysteine engineered antibody comprising a drug moiety conjugated to a cysteine amino acid substitution at a different position. In some embodiments, a least one drug moiety of the second cysteine engineered ADC is conjugated to a different cysteine amino acid substitution compared to the cysteine engineered ADC. In some embodiments, each drug moiety of the second cysteine engineered ADC is conjugated to a different cysteine amino acid substitution compared to the cysteine engineered ADC. In some embodiments, the second cysteine engineered ADC comprises the same drug moiety as the cysteine engineered ADC. In some embodiments, the second cysteine engineered ADC comprises the same number of drug moieties as the cysteine engineered ADC. In some embodiments, the second cysteine engineered ADC has about the same DAR as the cysteine engineered ADC. In some embodiments, the second cysteine engineered ADC has a DAR of about 3.5 to about 4. In some embodiments, the second cysteine engineered ADC has a DAR of about 3.5 to about 4 and each drug moiety is conjugated to a cysteine amino acid substitution at a different position compared to the cysteine engineered ADC. In some embodiments, the second cysteine engineered ADC has a DAR of about 3.5 to about 4 and 2 of the four drug moieties are conjugated to cysteine amino acid substitutions at a different position compared to the cysteine engineered ADC. Previously investigated cysteine engineered antibodies that may be used as a comparison ADC (e.g., a “second ADC”) are known in the are, with exemplary constructs shown in FIG. 4A, FIG. 4B, Table E2 and E3.

In some embodiments, the second ADC is comparably stable, or more stable, in plasma compared to a cysteine engineered ADC of the present disclosure. In some embodiments, a cysteine engineered ADC described herein is comparably stable, or less stable, than the second ADC. In some embodiments, a cysteine engineered ADC stability profile is determined for a cysteine engineered ADC. In some embodiments, the ADC stability profile is determined in vitro or in vivo. A cysteine engineered ADC stability profile may also be determined by measuring the percent of conjugated drug moiety that is reduced over time and comparing it to the percent of conjugated drug moiety of the second cysteine engineered ADC. In some embodiments, cysteine engineered ADC stability is determined by measuring the percent of conjugated drug moiety that is reduced over time and comparing it to the percent of conjugated drug moiety of the second engineered ADC at the time of administration and a time point after administration to the patient. For example, the time point after administration may be up to about 8 days, such as between about 5 minutes and about 7 days, between about 5 minutes and 3 days after administration, between about 1 day and about 7 days, between about 1 day and about 3 days, after administration to the patient. A cysteine engineered ADC stability profile may also be determined by measuring the DAR of the cysteine engineered ADC over time and comparing it to the DAR of the second cysteine engineered ADC.

In some embodiments, cysteine engineered ADC stability is determined by measuring the DAR of the cysteine engineered ADC over time after administration to a patient, and comparing it that of the second ADC over time after administration to a patient. For example, the DAR of both the cysteine engineered ADC and the second cysteine engineered ADC may be about 3.5, about 3.6, about 3.8, or about 4, at the time of administration to the patient. In some embodiments, following administration to the patient (e.g., about 1 day after administration to the patient), the DAR of the cysteine engineered ADC is lower than the DAR of the second cysteine engineered ADC. In some embodiments, the DAR of the cysteine engineered ADC decreases more rapidly over time following administration to the patient compared to the DAR of the second cysteine engineered ADC. For examples, in some embodiments, about 1 day after administration to the patient, the DAR of the cysteine engineered ADC is lower than about 3, and the DAR of the second cysteine engineered ADC is greater than about 3. In some embodiments, about 3 days after administration to the patient, the DAR of the cysteine engineered ADC is lower than about 2, and the DAR of the second cysteine engineered ADC is greater than about 3. In some embodiments, about 7 days after administration to the patient, the DAR of the cysteine engineered ADC is lower than about 1 to about 1.5, and the DAR of the second cysteine engineered ADC is greater than about 3. In some embodiments, about 7 days after administration to the patient, the DAR of the cysteine engineered ADC is lower than about 1 to about 1.5, and the DAR of the second cysteine engineered ADC is greater than about 3.

In some embodiments, the DAR of the cysteine engineered ADC is reduced compared to the second cysteine engineered ADC. In some embodiments, the DAR of the cysteine engineered ADC is reduced at least about 10%, at least about 15%, at least about 20%, at least about 25%, at least about 30%, at least about 35%, at least about 40%, at least about 45%, at least about 50%, at least about 60%, at least about 70%, or at least about 80%, compared to the DAR of the second cysteine engineered ADC at the same time point after administration to the patient. In some embodiments, about 1 day after administration to the patient, the DAR of the cysteine engineered ADC is reduced by about 25% to about 75% compared to the DAR of the second cysteine engineered ADC. In some embodiments, about 3 days after administration to the patient, the DAR of the cysteine engineered ADC is reduced by about 50% to about 75% compared to the DAR of the second cysteine engineered ADC. In some embodiments, about 7 days after administration to the patient, the DAR of the cysteine engineered ADC is reduced by about 50% to about 75% compared to the DAR of the second cysteine engineered ADC.

In some embodiments, the cysteine engineered ADC stability profile may be determined by measuring the concentration of the cysteine engineered ADC in blood plasma in vivo after administration to a patient, and comparing it to that of the second ADC after administration to a patient. In some embodiments, the cysteine engineered ADC stability profile is determined by measuring the concentration of the cysteine engineered ADC and the concentration of the second cysteine engineered ADC in blood plasma between about 5 minutes and about 28 days after administration to the patient. In some embodiments, the concentration of the cysteine engineered ADC and the concentration of the second cysteine engineered ADC in blood plasma is measured between about 5 minutes and about 48 hours, between about 24 hours and about 7 days, or between about 5 days and about 28 days, after administration to the patient. In some embodiments, the concentration of the cysteine engineered ADC and the concentration of the second cysteine engineered ADC in blood plasma is measured at any of about 5 minutes, 1 hour, 6 hours, 1 day, 2 days, 4 day, 7 days, 14 days, 21 days, or 28 days, after administration to the patient. In some embodiments, the second cysteine engineered ADC exhibits the same or greater concentration in blood plasma over time compared to the cysteine engineered ADC of the present disclosure.

In particular, the present disclosure relates to methods for reducing toxicity (e.g., lowering toxicity of the ADC in one or more organs or tissues,) of an ADC (e.g., a cysteine engineered ADC) by lowering or decreasing the stability of the cysteine engineered ADC. Thus, the combinations of cysteine amino acid substitutions in the cysteine engineered antibodies of the cysteine engineered ADCs of the present disclosure result in reduced exposure to the cytotoxic agent than the second cysteine engineered ADC that comprises a drug moiety conjugated to a cysteine amino acid at one or more other positions, when administered to a patient, and therefore have reduced toxicity. In some embodiments, the reduced toxicity is ocular toxicity, corneal toxicity, bone marrow toxicity, or lung toxicity.

In some embodiments, one or more side effects in the patient is improved after administration of the cysteine engineered ADC as compared to administration of a second cysteine engineered ADC that comprises a drug moiety conjugated to a cysteine amino acid at one or more other positions, when administered to a patient. In some embodiments, one or more side effects comprise ocular and/or corneal toxicity. In some embodiments, the ocular and/or corneal toxicity is determined based on the number mitotic and/or apoptotic figures in the eye. In some embodiments, one or more side effects comprise lung toxicity (e.g., pulmonary toxicity). In some embodiments, the lung toxicity is determined based on alveolar macrophage aggregation in the lung. In some embodiments, one or more side effects comprise bone marrow toxicity. In some embodiments, bone marrow toxicity is determined based on bone marrow suppression.

In some embodiments, ocular toxicity of the cysteine engineered ADC is reduced compared to the second cysteine engineered ADC. In some embodiments, corneal toxicity of the cysteine engineered ADC is reduced compared to the second cysteine engineered ADC. Ocular toxicity and/or corneal toxicity may present as conditions or symptoms such as but not limited to: an increase in mitotic and/or apoptotic figures within the eye and/or cornea, severe dry eye, lack of tear production, corneal haze, or corneal opacities. In some embodiments, the cysteine engineered ADC results in fewer mitotic and/or apoptotic figures within the eye and/or cornea compared to the second cysteine engineered ADC, when administered to a patient. In some embodiments, the cysteine engineered ADC does not result in dry eye or results in less severe dry eye compared to the second cysteine engineered ADC, when administered to a patient. In some embodiments, the cysteine engineered ADC does not result in a lack of tear production or results in less a severe lack of tear production compared to the second cysteine engineered ADC, when administered to a patient. In some embodiments, the cysteine engineered ADC does not result in corneal haze or results in less severe corneal haze compared to the second cysteine engineered ADC, when administered to a patient. In some embodiments, the cysteine engineered ADC does not result in corneal opacities or results in less severe corneal opacities compared to the second cysteine engineered ADC, when administered to a patient.

In some embodiments, bone marrow toxicity of the cysteine engineered ADC is reduced compared to the second cysteine engineered ADC. Several ADCs are known to suppress the bone marrow, thereby causing the marrow to make fewer red blood cells. The timing of this effect varies by the ADC used and the doses used for treatment. The risks for anemia, severe tiredness (fatigue), infection, bleeding, and bruising, among other symptoms may rise when the bone marrow is suppressed. Therefore, bone marrow toxicity may present as, but is not limited to, the conditions or symptoms relating to bone marrow suppression. In some embodiments, the cysteine engineered ADC does not result in bone marrow suppression or results in less severe bone marrow suppression compared to the second cysteine engineered ADC, when administered to a patient.

In some embodiments, lung toxicity of the cysteine engineered ADC is reduced compared to the second cysteine engineered ADC. Lung toxicity (e.g., pulmonary toxicity) may present as, but is not limited to: alveolar macrophage aggregation, pneumonitis, interstitial lung disease, and acute respiratory distress syndrome (ARDS), cough, and/or dyspnoea. In some embodiments, the cysteine engineered ADC does not result in alveolar macrophage aggregation or results in less severe alveolar macrophage aggregation compared to the second cysteine engineered ADC, when administered to a patient. In some embodiments, the cysteine engineered ADC does not result in pneumonitis or results in less severe pneumonitis compared to the second cysteine engineered ADC, when administered to a patient. In some embodiments, the cysteine engineered ADC does not result in interstitial lung disease or results in less severe interstitial lung disease compared to the second cysteine engineered ADC, when administered to a patient. In some embodiments, the cysteine engineered ADC does not result in ARDS or results in less severe ARDS compared to the second cysteine engineered ADC, when administered to a patient. In some embodiments, the cysteine engineered ADC does not result in coughing or results in less severe coughing compared to the second cysteine engineered ADC, when administered to a patient. In some embodiments, the cysteine engineered ADC does not result in dyspnoea or results in less severe dyspnoea compared to the second cysteine engineered ADC, when administered to a patient.

In some embodiments, the toxicity of cysteine engineered ADC is reduced compared to second cysteine engineered ADC between about 2 days and about 15 days after the administration, such as between about 2 days and about 10 days after the administration, between about 6 days and about 8 days after the administration, or between about 10 days and about 15 days after the administration. In some embodiments, the toxicity of cysteine engineered ADC is reduced compared to the second cysteine engineered ADC less than about 15 days after the administration, such as less than any of about 14, 13, 12, 11, 10, 9, 8, 7, 6, 5, 4, 3, 2, or fewer, days after the administration. In some embodiments, the toxicity of cysteine engineered ADC is reduced compared to the second cysteine engineered ADC greater than about 2 days after the administration, such as greater than any of about 3, 4, 5, 6, 7, 8, 9, 10, 11, 12, 13, 14, 15, or more, days after the administration. In some embodiments, the toxicity of cysteine engineered ADC is reduced compared to the second cysteine engineered ADC at about 7 days after the administration.

In some embodiments, the percentage of conjugated cytotoxic agent is reduced for the cysteine engineered ADC compared to a percentage of conjugated cytotoxic agent of the second cysteine engineered ADC between about 2 days and about 15 days after the administration, such as between about 2 days and about 10 days after the administration, between about 6 days and about 8 days after the administration, or between about 10 days and about 15 days after the administration. In some embodiments, the percentage of conjugated cytotoxic agent is reduced for the cysteine engineered ADC compared to a percentage of conjugated cytotoxic agent of the second cysteine engineered ADC less than about 15 days after the administration, such as less than any of about 14, 13, 12, 11, 10, 9, 8, 7, 6, 5, 4, 3, 2, or fewer, days after the administration. In some embodiments, the percentage of conjugated cytotoxic agent is reduced for the cysteine engineered ADC compared to a percentage of conjugated cytotoxic agent of the second cysteine engineered ADC greater than about 2 days after the administration, such as greater than any of about 3, 4, 5, 6, 7, 8, 9, 10, 11, 12, 13, 14, 15, or more, days after the administration. In some embodiments, the percentage of conjugated cytotoxic agent is reduced for the cysteine engineered ADC compared to a percentage of conjugated cytotoxic agent of the second cysteine engineered ADC at about 7 days after the administration.

In some embodiments, the percentage of conjugated cytotoxic agent is reduced between about 10% and about 80% for the cysteine engineered ADC compared to the percentage of conjugated cytotoxic agent of the second cysteine engineered ADC, such as between about 10% and about 30%, between about 20% and about 40%, between about 30% and about 50%, between about 40% and about 60%, between about 50% and about 70%, or between about 60% and about 80%, for the cysteine engineered ADC compared to the percentage of conjugated cytotoxic agent of the second cysteine engineered ADC. In some embodiments, the percentage of conjugated cytotoxic agent is reduced at least about 10% for the cysteine engineered ADC compared to the percentage of conjugated cytotoxic agent of the second cysteine engineered ADC, such as at least any of about 15%, 20%, 25%, 30%, 35%, 40%, 45%, 50%, 55%, 60%, 65%, 70%, 75%, 80%, or greater, for the cysteine engineered ADC compared to the percentage of conjugated cytotoxic agent of the second cysteine engineered ADC. In some embodiments, the percentage of conjugated cytotoxic agent is reduced less than about 80% for the cysteine engineered ADC compared to the percentage of conjugated cytotoxic agent of the second cysteine engineered ADC, such as less than any of about 75%, 70%, 65%, 60%, 55%, 50%, 45%, 40%, 35%, 30%, 25%, 20%, 15%, 10%, or less, for the cysteine engineered ADC compared to the percentage of conjugated cytotoxic agent of the second cysteine engineered ADC.

In some embodiments, the patient has reduced exposure to the cytotoxic agent for the cysteine engineered ADC compared to the exposure produced by the second cysteine engineered ADC.

### B. Methods of Treatment

In some embodiments, provided herein is a method of treating cancer in a patient, comprising administering an effective amount of a cysteine engineered ADC, such as any of the cysteine engineered ADCs described herein, or an effective amount of a pharmaceutical composition comprising any of the cysteine engineered ADCs described herein, to a patient. In some embodiments, the cysteine engineered ADC comprises a cysteine engineered antibody, such as any of the cysteine engineered antibodies provided herein, conjugated to a drug moiety. In some embodiments, the cysteine engineered antibody comprises: a heavy chain and a light chain; and, ii) the cysteine amino acid substitutions are selected from the group consisting of: a) position 121 of the light chain and position 375 of the heavy chain; b) position 121 of the light chain and position 400 of the heavy chain; and, c) position 114 of the light chain and position 400 of the heavy chain, wherein the amino acid position numbering in the light chain is according to Kabat numbering and the amino acid position numbering in the heavy chain is according to EU numbering. In some embodiments, the cysteine engineered antibody is a full-length antibody, a Fab, a Fab′, a (Fab′)₂, an Fv, or a single chain Fv (scFv). In some embodiments, the cysteine engineered antibody comprises two heavy chains and two light chains, wherein the cysteine amino acid substitutions are selected from the group consisting of: a) position 121 of at least one the light chains and position 375 of at least one of the heavy chains; b) position 121 of at least one of the light chains and position 400 of at least one of the heavy chains; and, c) position 114 of at least one of the light chains and position 400 of each of at least one of the heavy chains, wherein the amino acid position numbering in the light chains are according to Kabat numbering and the amino acid position numbering in the heavy chains are according to EU numbering. In some embodiments, the cysteine engineered antibody comprises: i) two heavy chains and two light chains and ii) the cysteine amino acid substitutions selected from the group consisting of: a) position 121 of each of the light chains and position 375 of each of the heavy chains; b) position 121 of each of the light chains and position 400 of each of the heavy chains; and, c) position 114 of each of the light chains and position 400 of each of the heavy chains, wherein the amino acid position numbering in the light chains are according to Kabat numbering and the amino acid position numbering in the heavy chains are according to EU numbering.

In some embodiments, one or more side effects in the patient is improved after administration of the cysteine engineered ADC as compared to administration of a second cysteine engineered ADC that comprises a drug moiety conjugated to a cysteine amino acid at one or more other positions, when administered to a patient. In some embodiments, the second cysteine engineered ADC is comparably stable or more stable in plasma compared to a cysteine engineered ADC of the present disclosure. In some embodiments, a cysteine engineered ADC described herein is comparably stable or less stable than the other ADC. In some embodiments, one or more side effects comprise ocular toxicity, corneal toxicity, lung toxicity, and/or bone marrow toxicity. In some embodiments, one or more side effects comprise ocular and/or corneal toxicity. In some embodiments, the side effect is ocular and/or corneal toxicity. In some embodiments, the ocular and/or corneal toxicity is determined based on the number mitotic and/or apoptotic figures in the eye. In some embodiments, one or more side effects comprise lung toxicity. In some embodiments, the side effect is lung toxicity. In some embodiments, the lung toxicity is determined based on alveolar macrophage aggregation in the lung. In some embodiments, one or more side effects comprise bone marrow toxicity. In some embodiments, the side effect is bone marrow toxicity. In some embodiments, bone marrow toxicity is determined by bone marrow suppression. In some embodiments, bone marrow suppression is determined by preservation of reticulocytes. In some embodiments, bone marrow suppression is determined by loss of preservation of reticulocytes. In some embodiments, bone marrow suppression is determined by loss of reticulocytes per microliter.

In some embodiments, bone marrow suppression is determined by degree of preservation of mononuclear cells. In some embodiments, bone marrow suppression is determined by the loss of preservation of mononuclear cells.

In some embodiments, the cancer a solid tumor, leukemia, or hematological cancer.

Administration can be parenteral, intravenous, oral, subcutaneous, intra-arterial, intracranial, intrathecal, intraperitoneal, topical, intranasal or intramuscular. Administration can also be localized directly into a tumor. Administration into the systemic circulation by intravenous or subcutaneous administration is preferred. Intravenous administration can be, for example, by infusion. In some embodiments, as described herein, the dosage of a cysteine engineered ADC or a pharmaceutical composition thereof administered to a patient is typically about 0.01 mg/kg to about 100 mg/kg of the patient's body weight.

The frequency of administration depends on the half-life of the cysteine engineered ADC or pharmaceutical composition thereof in the circulation, the condition of the patient, and the route of administration among other factors. The frequency can be daily, weekly, monthly, quarterly, or at irregular intervals in response to changes in the patient's condition or progression of the cancer being treated. An exemplary frequency for intravenous administration is between twice a week and quarterly over a continuous course of treatment, although more or less frequent dosing is also possible. Other exemplary frequencies for intravenous administration are between once weekly or once monthly over a continuous course of treatment, although more or less frequent dosing is also possible. For subcutaneous administration, an exemplary dosing frequency is daily to monthly, although more or less frequent dosing is also possible.

### 1. Treatment of Cancer

In some aspects, a cysteine engineered ADC, such as any of the cysteine engineered ADCs described herein, are useful for inhibiting the multiplication of a tumor cell or cancer cell, causing apoptosis in a tumor or cancer cell, or for treating cancer in a patient. The cysteine engineered antibodies and/or cysteine engineered ADCs can be used accordingly in a variety of settings for the treatment of cancers. The cysteine engineered ADCs can be used to deliver a cytotoxic agent to a tumor cell or cancer cell. Without being bound by theory, in one embodiment, the cysteine engineered antibody unit of a cysteine engineered ADC binds to or associates with a cancer-cell or a tumor-cell-associated antigen, and the cysteine engineered ADC can be taken up inside a tumor cell or cancer cell through receptor-mediated endocytosis. The antigen can be attached to a tumor cell or cancer cell or can be an extracellular matrix protein associated with the tumor cell or cancer cell. Once inside the cell, one or more specific peptide sequences within the linker unit are hydrolytically cleaved by one or more tumor-cell or cancer-cell-associated proteases, resulting in release of a cytotoxic agent. The released cytotoxic agent or cytotoxic agent-linker is then free to migrate within the cell and induce cytotoxic activities. In an alternative embodiment, the cytotoxic agent is cleaved from the cysteine engineered ADC outside the tumor cell or cancer cell, and the cytotoxic agent or cytotoxic agent-linker compound subsequently penetrates the cell.

In some embodiments, the cysteine engineered antibody unit binds to the tumor cell or cancer cell. In some embodiments, the cysteine engineered antibody unit binds to a tumor cell or cancer cell antigen which is on the surface of the tumor cell or cancer cell. In some embodiments, the cysteine engineered antibody unit binds to a tumor cell or cancer cell antigen which is an extracellular matrix protein associated with the tumor cell or cancer cell.

The specificity of the cysteine engineered antibody unit for a particular tumor cell or cancer cell can be important for determining those tumors or cancers that are most effectively treated. For example, cysteine engineered ADCs having a BR96 cysteine engineered antibody unit can be useful for treating antigen positive carcinomas including those of the lung, breast, colon, ovaries, and pancreas. Cysteine engineered ADCs having an anti-CD30 or an anti-CD40 cysteine engineered antibody unit can be useful for treating hematologic malignancies.

Other particular types of cancers that can be treated with cysteine engineered ADCs solid tumors or liquid tumors (e.g., blood tumors).

Examples of hematopoietic tumors that have the potential to give rise to solid tumors include, but are not limited to, diffuse large B-cell lymphomas (DLBCL), follicular lymphoma, myelodysplastic syndrome (MDS), a lymphoma, Hodgkin's disease, a malignant lymphoma, non-Hodgkin's lymphoma, Burkitt's lymphoma, multiple myeloma, Richter's Syndrome (Richter's Transformation) and the like.

In certain embodiments, the cancer is selected from, but not limited to, leukemia's such as acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AMOL).

In some embodiments, the cancer is another hematological cancer, including, but are not limited to, non-Hodgkin lymphoma (e.g., diffuse large B cell lymphoma, mantle cell lymphoma, B lymphoblastic lymphoma, peripheral T cell lymphoma and Burkitt's lymphoma), B-lymphoblastic lymphoma; B-cell chronic lymphocytic leukemia/small lymphocytic lymphoma; lymphoplasmacytic lymphoma; splenic marginal zone B-cell lymphoma (±villous lymphocytes); plasma cell myeloma/plasmacytoma; extranodal marginal zone B-cell lymphoma of the MALT type; nodal marginal zone B-cell lymphoma (±monocytoid B cells); follicular lymphoma; diffuse large B-cell lymphomas; Burkitt's lymphoma; precursor T-lymphoblastic lymphoma; T adult T-cell lymphoma (HTLV 1-positive); extranodal NK/T-cell lymphoma, nasal type; enteropathy-type T-cell lymphoma; hepatosplenic γ-δ T-cell lymphoma; subcutaneous panniculitis-like T-cell lymphoma; mycosis fungoides/sezary syndrome; anaplastic large cell lymphoma, T/null cell, primary cutaneous type; anaplastic large cell lymphoma, T-/null-cell, primary systemic type; peripheral T-cell lymphoma, not otherwise characterized; angioimmunoblastic T-cell lymphoma, multiple myeloma, polycythemia vera or myelofibrosis, cutaneous T-cell lymphoma, small lymphocytic lymphoma (SLL), marginal zone lymphoma, CNS lymphoma, immunoblastic large cell lymphoma, precursor B-lymphoblastic lymphoma and the like.

Exemplary solid tumors that can be treated include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell lung carcinoma (SCLC) or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer.

Cancers, including, but not limited to, a tumor, metastasis, or other disease or disorder characterized by uncontrolled cell growth, can be treated or prevented by administration of a cysteine engineered ADC or a pharmaceutical composition thereof to a patient. In some embodiments, one or more additional therapies (e.g., drugs) may administered to the patient along with the cysteine engineered ADC.

### Embodiments

Embodiment 1. A cysteine engineered antibody comprising cysteine amino acid substitutions, wherein:
i) the cysteine engineered antibody comprises a heavy chain and a light chain;
and,
ii) the cysteine amino acid substitutions are selected from the group consisting of:
   a) position 121 of the light chain and position 375 of the heavy chain;
   b) position 121 of the light chain and position 400 of the heavy chain; and
   c) position 114 of the light chain and position 400 of the heavy chain,
   wherein the amino acid position numbering in the light chain is according to Kabat numbering and the amino acid position numbering in the heavy chain is according to EU numbering.

Embodiment 2. The cysteine engineered antibody of embodiment 1, wherein the cysteine engineered antibody comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
wherein the amino acid position numbering in the light chains are according to Kabat numbering and the amino acid position numbering in the heavy chains are according to EU numbering.

Embodiment 3. The cysteine engineered antibody of embodiment 2, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

Embodiment 4. The cysteine engineered antibody of any one of embodiments 1-3, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

Embodiment 5. The cysteine engineered antibody of any one of embodiments 1-4, wherein the cysteine amino acid substitutions are of free cysteines.

Embodiment 6. The cysteine engineered antibody of any one of embodiments 1-5, wherein the cysteine engineered antibody is chimeric, human, or humanized.

Embodiment 7. The cysteine engineered antibody of any one of embodiments 1-6, wherein the cysteine engineered antibody is a full-length antibody.

Embodiment 8. The cysteine engineered antibody of embodiment 7, wherein the cysteine engineered antibody is IgA, IgD, IgE, IgG, or IgM.

Embodiment 9. The cysteine engineered antibody of any one of embodiments 1-8, wherein the cysteine engineered antibody is monospecific.

Embodiment 10. The cysteine engineered antibody of any one of embodiments 1-8, wherein the cysteine engineered antibody is multispecific.

Embodiment 11. The cysteine engineered antibody of any one of embodiments 1-10, wherein the cysteine engineered antibody specifically binds to an antigen selected from the group consisting of B7-H4, ITGB6, CD30, CD40, EpCAM, CEACAM5, and LIV1.

Embodiment 12. The cysteine engineered antibody of any one of embodiments 1-10, wherein the cysteine engineered antibody is selected from the group consisting of an anti-B7-H4 antibody, an anti-ITGB6 antibody, an anti-CD30 antibody, an anti-CD40 antibody, an anti-EpCAM antibody, an anti-CEACAM5 antibody, and an anti-LIV1 antibody.

Embodiment 13. The cysteine engineered antibody of any one of embodiments 1-10, wherein the cysteine engineered antibody comprises
a heavy chain variable domain (VH) comprising a heavy chain complementarity determining region (CDRH1) comprising the amino acid sequence of any one of SEQ ID NOs: 1, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, 360, 368, 376, 384, 392, 400, 408, 416, 424, 432, 440, 448, 456, 464, 474, 494, 502, 510, 518, 526, 534, 542, 550, 558, 566, 574, 582, 590, 598, 606, 614, 622, 630, 638, 646, 654, 662, 670, 678, 686, 694, 702, 710, 718, 726, 734, 742, 750, 758, 766, 774, 782, 794, 802, 810, 834, 842, 850, 858, 866, 874, 882, 900, 917, 931, 945, 959, 967, 975, 983, 991, 999, 1007, 1015, 1023, 1031, 1039, 1047, 1055, 1063, 1071, 1079, 1087, 1095, 1103, 1111, 1159, and 1178, a CDRH2 comprising the amino acid sequence of any one of SEQ ID Nos: 2, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, 337, 345, 353, 361, 369, 377, 385, 393, 401, 409, 417, 425, 433, 441, 449, 457, 465, 475, 495, 503, 511, 519, 527, 535, 543, 551, 559, 567, 575, 583, 591, 599, 607, 615, 623, 631, 639, 647, 655, 663, 671, 679, 687, 695, 703, 711, 719, 727, 735, 743, 751, 759, 767, 775, 783, 795, 803, 811, 835, 843, 851, 859, 867, 875, 883, 901, 918, 932, 946, 960, 968, 976, 984, 992, 1000, 1008, 1016, 1024, 1032, 1040, 1048, 1056, 1064, 1072, 1080, 1088, 1096, 1104, 1112, 1160, and 1179, a CDRH3 comprising the amino acid sequence DMG or any one of SEQ ID Nos: 3, 18, 26, 34, 42, 50, 58, 66, 74, 82, 90, 98, 114, 122, 130, 138, 146, 154, 162, 170, 178, 186, 194, 202, 210, 218, 226, 234, 242, 250, 258, 266, 274, 282, 290, 298, 306, 314, 322, 330, 338, 346, 354, 362, 370, 378, 386, 394, 402, 410, 418, 426, 434, 442, 450, 458, 466, 476, 496, 504, 512, 520, 528, 536, 544, 552, 560, 568, 576, 584, 592, 600, 608, 616, 624, 632, 640, 648, 656, 664, 672, 680, 688, 696, 704, 712, 720, 728, 736, 744, 752, 760, 768, 776, 784, 796, 804, 812, 836, 844, 852, 860, 868, 876, 884, 902, 919, 933, 947, 961, 969, 977, 985, 993, 1001, 1009, 1017, 1025, 1033, 1041, 1049, 1057, 1065, 1073, 1081, 1089, 1097, 1105, 1113, 1161, and 1180; and a light chain variable domain (VL) comprising a light chain complementarity determining region (CDRL1) comprising the amino acid sequence of any one of SEQ ID Nos: 4, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, 307, 315, 323, 331, 339, 347, 355, 363, 371, 379, 387, 395, 403, 411, 419, 427, 435, 443, 451, 459, 467, 477, 497, 505, 513, 521, 529, 537, 545, 553, 561, 569, 577, 585, 593, 601, 609, 617, 625, 633, 641, 649, 657, 665, 673, 681, 689, 697, 705, 713, 721, 729, 737, 745, 753, 761, 769, 777, 785, 797, 805, 813, 837, 845, 853, 861, 869, 877, 885, 903, 920, 934, 948, 962, 970, 978, 986, 994, 1002, 1010, 1018, 1026, 1034, 1042, 1050, 1058, 1066, 1074, 1082, 1090, 1098, 1106, 1114, 1162, and 1181, a CDRL2 comprising the amino acid sequence of AAS or any one of SEQ ID Nos: 5, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, 308, 316, 324, 332, 340, 348, 356, 364, 372, 380, 388, 396, 404, 412, 420, 428, 436, 444, 452, 460, 468, 478, 498, 506, 514, 522, 530, 538, 546, 554, 562, 570, 578, 586, 594, 602, 610, 618, 626, 634, 642, 650, 658, 666, 674, 682, 690, 698, 706, 714, 722, 730, 738, 746, 754, 762, 770, 778, 786, 798, 806, 814, 838, 846, 854, 862, 870, 878, 904, 921, 935, 949, 963, 971, 979, 987, 995, 1003, 1011, 1019, 1027, 1035, 1043, 1051, 1059, 1067, 1075, 1083, 1091, 1099, 1107, 1115, 1163, and 1182 and a CDRL3 comprising the amino acid sequence of any one of SEQ ID Nos: 6, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333, 341, 349, 357, 365, 373, 381, 389, 397, 405, 413, 421, 429, 437, 445, 453, 461, 469, 479, 499, 507, 515, 523, 531, 539, 547, 555, 563, 571, 579, 587, 595, 603, 611, 619, 627, 635, 643, 651, 659, 667, 675, 683, 691, 699, 707, 715, 723, 731, 739, 747, 755, 763, 771, 779, 787, 799, 807, 815, 839, 847, 855, 863, 871, 879, 887, 905, 922, 936, 950, 964, 972, 980, 988, 996, 1004, 1012, 1020, 1028, 1036, 1044, 1052, 1060, 1068, 1076, 1084, 1092, 1100, 1108, 1116, 1164, and 1183.

Embodiment 14. The cysteine engineered antibody of any one of embodiments 1-10, wherein the cysteine engineered antibody comprises
a V_{H} comprising an amino acid sequence of any one of SEQ ID Nos: 7, 12, 22, 30, 38, 46, 54, 66, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, 310, 318, 326, 334, 342, 350, 358, 366, 374, 382, 390, 398, 406, 414, 422, 430, 438, 446, 454, 462, 470, 472, 480, 482, 484, 500, 508, 516, 524, 532, 540, 548, 556, 564, 572, 580, 588, 596, 604, 612, 620, 628, 636, 644, 652, 660, 668, 676, 684, 692, 700, 708, 716, 724, 732, 740, 748, 756, 764, 772, 780, 788, 800, 808, 816, 840, 848, 856, 864, 872, 880, 888, 898, 906, 923, 937, 951, 965, 973, 981, 989, 997, 1005, 1013, 1021, 1029, 1037, 1045, 1053, 1061, 1069, 1077, 1085, 1093, 1101, 1109, 1117, and 1184, and a VL comprising the amino acid sequence of any one of SEQ ID Nos: 8, 13, 23, 31, 39, 47, 55, 67, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, 359, 367, 375, 383, 391, 399, 407, 415, 423, 431, 439, 447, 455, 463, 471, 473, 481, 483, 485, 501, 509, 517, 525, 533, 541, 549, 557, 565, 573, 581, 589, 597, 605, 613, 621, 629, 637, 645, 653, 661, 669, 677, 685, 693, 701, 709, 717, 725, 733, 741, 749, 757, 765, 773, 781, 789, 801, 809, 817, 841, 849, 857, 865, 873, 881, 889, 899, 907, 924, 938, 952, 966, 974, 982, 990, 998, 1006, 1014, 1022, 1030, 1038, 1046, 1054, 1062, 1070, 1078, 1086, 1094, 1102, 1110, 1118, and 1185.

Embodiment 15. A cysteine engineered antibody drug conjugate (ADC) comprising the cysteine engineered antibody of any one of embodiments 1-14 conjugated to a drug moiety.

Embodiment 16. The cysteine engineered ADC of embodiment 15, wherein the drug moiety is selected from the group consisting of a cytotoxic agent, growth inhibitory agent, and a chemotherapy agent.

Embodiment 17. The cysteine engineered ADC of embodiment 15 or embodiment 16, wherein the cysteine engineered antibody is conjugated to a cytotoxic agent.

Embodiment 18. The cysteine engineered ADC of any one of embodiments 15-17, wherein the cysteine engineered antibody is conjugated to the drug moiety via a linker moiety attached to a cysteine amino acid substitution.

Embodiment 19. The cysteine engineered ADC of any one of embodiments 15-18, wherein the cysteine engineered ADC comprises a drug moiety conjugated to each an individual cysteine amino acid substitution.

Embodiment 20. The cysteine engineered ADC of embodiment 15, wherein each of the drug moieties are the same drug moiety.

Embodiment 21. The cysteine engineered ADC of any one of embodiments 15-20, wherein the drug moiety is a microtubule inhibitor.

Embodiment 22. The cysteine engineered ADC of embodiment 21, wherein the drug moiety is an auristatin or a tubulysin.

Embodiment 23. The cysteine engineered ADC of any one of embodiments 15-22, wherein the drug moiety is selected from the group consisting of auristatin T, tubulysin M, tubulysin OEt, MMAE (monomethyl auristatin E), and MMAF (monomethyl auristatin F).

Embodiment 24. The cysteine engineered ADC of embodiment 23, wherein the drug moiety is MMAE.

Embodiment 25. The cysteine engineered ADC of any one of embodiments 15-24, wherein the cysteine engineered ADC has a drug antibody ratio (DAR) of about 3.5 to about 4.

Embodiment 26. The cysteine engineered ADC of any one of embodiments 15-25, wherein less than 50% of the drug moiety is conjugated to the antibody at day 7 after administration to a subject.

Embodiment 27. A nucleic acid encoding the cysteine engineered antibody of any one of embodiments 1-14.

Embodiment 28. A vector comprising the nucleic acid of embodiment 27.

Embodiment 29. A host cell comprising the nucleic acid of embodiment 27 or the vector of embodiment 28.

Embodiment 30. A pharmaceutical composition comprising the cysteine engineered antibody of any one of embodiments 1-14 or the cysteine engineered ADC of any one of embodiments 15-26, and a pharmaceutically acceptable carrier.

Embodiment 31. A method of reducing toxicity of an ADC comprising an antibody moiety conjugated to a drug moiety, the method comprising:
generating a cysteine engineered ADC comprising cysteine amino acid substitutions, wherein the cysteine engineered ADC comprises a cysteine engineered antibody comprising a heavy chain and a light chain, and wherein the cysteine amino acid substitutions are selected from the group consisting of:
   a) position 121 of the light chain and position 375 of the heavy chain;
   b) position 121 of the light chain and position 400 of the heavy chain;
   and,
   c) position 114 of the light chain and position 400 of the heavy chain,
   wherein the amino acid position numbering in the light chain is according to Kabat numbering and the amino acid position numbering in the heavy chain is according to EU numbering,
wherein the drug moiety is conjugated to each of the cysteine amino acid substitutions, thereby reducing toxicity of the cysteine engineered ADC compared to a second cysteine engineered ADC that comprises a drug moiety conjugated to a cysteine amino acid at one or more other positions, when administered to a patient.

Embodiment 32. The method of embodiment 31, wherein the cysteine engineered antibody moiety is a full-length antibody.

Embodiment 33. The method of embodiment 31 or 32, wherein the cysteine engineered antibody moiety comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
   wherein the amino acid position numbering in the light chains are according to Kabat numbering and the amino acid position numbering in the heavy chains are according to EU numbering.

Embodiment 34. The method of embodiment 33, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

Embodiment 35. The method of any one of embodiments 31-34, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

Embodiment 36. The method of any one of embodiments 31-35, wherein the generating comprises substituting an amino acid in the cysteine engineered antibody with a cysteine amino acid or inserting a cysteine amino acid into the cysteine engineered antibody.

Embodiment 37. The method of any one of embodiments 31-36, wherein ocular toxicity of the cysteine engineered ADC is reduced compared to the second cysteine engineered ADC.

Embodiment 38. The method of any one of embodiments 31-37, wherein corneal toxicity of the cysteine engineered ADC is reduced compared to the second cysteine engineered ADC.

Embodiment 39. The method of any one of embodiments 31-38, wherein bone marrow toxicity of the cysteine engineered ADC is reduced compared to the second cysteine engineered ADC.

Embodiment 40. The method of any one of embodiments 31-39, wherein lung toxicity of the cysteine engineered ADC is reduced compared to the second cysteine engineered ADC.

Embodiment 41. The method of any one of embodiments 31-40, wherein the toxicity of cysteine engineered ADC is reduced compared to the second cysteine engineered ADC at about 7 days after the administration.

Embodiment 42. The method of any one of embodiments 31-41, wherein the percentage of conjugated drug moiety is reduced for the cysteine engineered ADC compared to a percentage of conjugated drug moiety of the second cysteine engineered ADC at about 7 days after the administration.

Embodiment 43. The method of embodiment 42, wherein the percentage of conjugated drug moiety is reduced at least about 10%, at least about 15%, at least about 20%, at least about 25%, at least about 30%, at least about 35%, at least about 40%, at least about 45%, at least about 50%, at least about 60%, at least about 70%, or at least about 80% for the cysteine engineered ADC compared to the percentage of conjugated drug moiety of the second cysteine engineered ADC.

Embodiment 44. The method of any one of embodiments 31-43, wherein the patient has reduced exposure to the drug moiety for the cysteine engineered ADC compared to the exposure produced by the second cysteine engineered ADC.

Embodiment 45. A method of reducing toxicity of a cysteine engineered ADC when administered to a patient, wherein the cysteine engineered ADC comprises a cysteine engineered antibody and a drug moiety, the method comprising:
conjugating the drug moiety to a cysteine amino acid of the cysteine engineered antibody via a linker moiety, wherein the cysteine amino acid is at a position that reduces the stability of the cysteine engineered ADC compared to a second cysteine engineered ADC that comprises a drug moiety conjugated to a cysteine amino acid of a cysteine engineered antibody at one or more other positions.

Embodiment 46. The method of embodiment 45, wherein the cysteine amino acid is in a heavy chain of the antibody moiety.

Embodiment 47. The method of embodiment 45 or 46, wherein the cysteine amino acid is in a light chain of the antibody moiety.

Embodiment 48. The method of any one of embodiments 45-47, wherein the drug moiety is conjugated to each cysteine amino acid substitution of the cysteine engineered antibody.

Embodiment 49. The method of any one of embodiments 45-48, wherein:
i) the cysteine engineered antibody comprises a heavy chain and a light chain;
and,
ii) the cysteine amino acid substitutions are selected from the group consisting of:
   a) position 121 of the light chain and position 375 of the heavy chain;
   b) position 121 of the light chain and position 400 of the heavy chain;
   and,
   c) position 114 of the light chain and position 400 of the heavy chain,
   wherein the amino acid position numbering in the light chain is according to Kabat numbering and the amino acid position numbering in the heavy chain is according to EU numbering.

Embodiment 50. The method of embodiment 49, wherein the cysteine engineered antibody is a full-length antibody.

Embodiment 51. The method of embodiment 49 or 50, wherein the cysteine engineered antibody comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
wherein the amino acid position numbering in the light chains are according to Kabat numbering and the amino acid position numbering in the heavy chains are according to EU numbering.

Embodiment 52. The method of embodiment 51, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

Embodiment 53. The method of embodiment 52, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

Embodiment 54. A method of treating cancer in a patient, comprising administering an effective amount of the cysteine engineered ADC of any one of embodiments 15-26 or an effective amount of the pharmaceutical composition of embodiment 30 to the patient.

Embodiment 55. The method of embodiment 54, wherein the cancer is a solid tumor, leukemia, or hematological cancer.

Embodiment 56. The method of any one of embodiments 31-55, wherein administration of the cysteine-engineered ADC causes fewer side effects in the patient compared to the side effects that occur if a non-cysteine engineered ADC or a cysteine-engineered ADC with other cysteine substitutions is administered.

Embodiment 57. The method of embodiment 56, wherein the one or more side effects comprise ocular toxicity, corneal toxicity, lung toxicity, and/or bone marrow toxicity.

Embodiment 58. The method of embodiment 57, wherein:
i) the ocular and/or corneal toxicity is determined based on the number mitotic and/or apoptotic figures in the eye;
ii) the lung toxicity is determined based on alveolar macrophage aggregation in the lung; and/or,
iii) the bone marrow toxicity is determined based on suppression of blood cell production in the bone marrow.

Embodiment 59. The method of any one of embodiments 54-58, wherein less than 50% of the drug moiety is conjugated to the antibody at day 7 after administration to the patient.

Embodiment 60. A kit comprising:
i) the cysteine engineered antibody of any one of embodiments 1-14 or the cysteine engineered ADC of any one of embodiments 15-26; and,
ii) instructions for using the cysteine engineered antibody or the cysteine engineered ADC.

Embodiment 61. The cysteine-engineered antibody of any one of embodiments 1-12 or the cysteine engineered ADC of any one of embodiments 15-25, wherein the cysteine engineered antibody comprises
a heavy chain comprising the amino acid sequence set forth in SEQ ID NO: 896 and a light chain comprising the amino acid sequence set forth in SEQ ID NO:897;
a heavy chain comprising the amino acid sequence set forth in SEQ ID NO: 894 and a light chain comprising the amino acid sequence set forth in SEQ ID NO:895; or
a heavy chain comprising the amino acid sequence set forth in SEQ ID NO: 890 and a light chain comprising the amino acid sequence set forth in SEQ ID NO:891.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: EC4 ADCs Drug-Antibody Ratio Over Time Compared to Conventional Cysteine Conjugates

ADCs were assessed for stability by measuring drug loss during incubation in plasma.

### A. Methods

Antibody expression and purification: Sequences containing the IgG1 Fc and kappa engineered cysteine mutations were cloned into expression vectors using Gibson Assembly. Heavy and light chain cysteine mutant plasmids were transfected into Freestyle CHO—S cells and the resultant supernatants were harvested after 9 days. The antibodies containing the cysteine mutations were purified by Mab select protein A affinity columns.

Preparation of Antibody Drug Conjugates (ADC): Antibodies engineered with two residues mutated to cysteine (EC4) were fully reduced and re-oxidized to prepare for conjugation with a drug moiety. Briefly, EC4 antibodies were fully reduced by adding 10 equivalents of tris(2-carboxyethyl)phosphine (TCEP) and 2 mM EDTA and adjusting the pH to 7.4 using 1 M potassium phosphate buffer (pH 8). Following one hour incubation at 37° C., the reaction mixture was buffer exchanged into phosphate buffered saline (PBS) with 2 mM ethylenediaminetetraacetic acid (EDTA) using Sephadex G25 resin desalting columns. 7 equivalents of dehydroascorbic acid (DHA) were added to the reaction and incubated for 1 hr at room temperature, followed by an additional 10 equivalents of DHA and another 1 hr incubation at room temperature. The oxidation reaction resulted in the reformation of native disulfides, but left the engineered cysteines in the reduced state and available for conjugation.

Conjugation was carried out by the addition of 1.3 drug-linker equivalents per available thiol along with a total reaction co-solvent of 10% dimethyl Sulfoxide (DMSO). After 30 mins at ambient temperature, an excess of QuadraSil MPTM was added to the mixture to quench all unreacted maleimide groups. The resulting ADC was then purified, and buffer exchanged by desalting using Sephadex G25 resin into PBS buffer and kept at −80° C. until further use. The protein concentration of the resulting ADC composition was determined at 280 nm. The drug-antibody ratio (DAR) of the conjugate was determined by high performance liquid chromatography (HPLC) analysis on a PLRP column by integration of the heavy and light chain components.

Maleimide stability assays: The cysteine engineered antibodies were incubated in BALB/C mouse or Sprague Dawley rat plasma for 0, 0.25, 1, 3, and 7 days. ADCs were purified from plasma using anti-human capture resin, reduced with DTT, deglycosylated, and then analyzed using reverse phase (RP) UPLC-MS. Maleimide hydrolysis was identified by analyzing heavy and light chain containing drug and assessing a mass addition of 18 Daltons. Maleimide stability was assessed by comparing the DAR at each time point. Drug loss was measured as a loss of 1317 Daltons and calculated as a percentage of total drug remaining based on total drug at t=0.

### B. Results

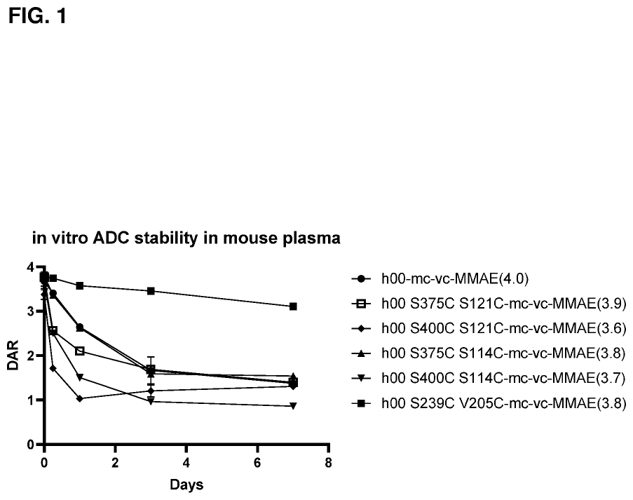

To assess maleimide hydrolysis and stability, conventional and EC4 ADCs conjugated with mc-vc-MMAE having a DAR of about 4 were prepared and incubated in BALB/C mouse plasma up to 7 days. Results indicate the MC linker conjugated to the various sites yielded different stability profiles (FIG. 1). Conjugation of the MC linker to the S239C/V²⁰⁵C combination yielded a highly stable conjugate over the course of the experiment. Conjugation of the MC linker to the S375C/S114C combination yielded a moderately stable conjugate similar to a native cysteine conjugate in vitro. Conjugation to combinations S375C/S121C, S400C/S121C, and S400C/S114C yielded less stable conjugates compared to native cysteine conjugate.

### Example 2: In Vivo Pharmacokinetic Assessment of EC4 ADCs Compared to Conventional ADCs

In this Example, various EC4 and conventional ADCs were evaluated for drug loss over time in vivo.

### A. Methods

Pharmacokinetic assessment: ADCs were prepared as described in Example 1 from reduced non-binding antibody and drug-linker and injected intravenously into female Sprague Dawley rats at various concentrations. Blood draws were taken at 5 min, 1 hr, 6 hrs, 1 day, 2 day, 4 day, 7 day, 14 day, 21 day, and 28 day timepoints post dose. Total antibody (TAb) concentration in plasma was measured using a generic ELISA method. To determine the concentration of antibody-conjugated drug, plasma was prepared by anti-human antibody capture, purification, and papain cleavage of val-cit linker, then released MMAE was quantified by LC/MS. The in vivo DAR was calculated by the ratio between the μM concentrations of antibody-conjugated drug and TAb.

### B. Results

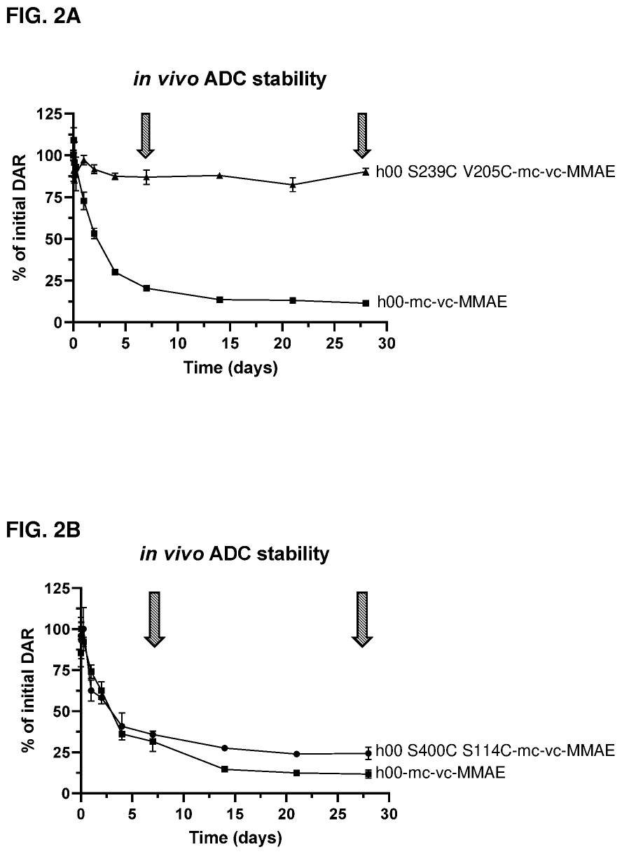

Conventional and EC4 ADCs conjugated with mc-vc-MMAE, having a DAR of about 4, were administered in Sprague Dawley rats to assess pharmacokinetic parameters and in vivo conjugated drug stability. All EC4-based ADCs had significantly greater total antibody and antibody-conjugated drug exposure than the native cysteine ADC comparator (Table E1). However, the degree of extended antibody-conjugated drug exposure varied depending on the stability of the MC linker and on the various engineered cysteine sites. The S375C/S114C and S239C/V205C EC combinations that had equivalent or greater stability profiles than the native conjugate were observed to have the highest antibody-conjugated drug exposure (Table E1). These combinations each had 1.7-fold increases in Area Under Curve for 0-7 days (AUC_{0-7d}) and a 2.6 and 3.1-fold increase in AUC_{0-28d}, respectively. The other EC combinations that were observed to be less stable in vitro compared to the native ADC comparator had a lesser degree of change in antibody-conjugated drug exposure. The S375C/S121C, S400C/S121C, and S400C/S114C had 1.1-1.4-fold increase in AUC_{0-7d} and a 1.8-2.2-fold increase in AUC_{0-28d}. The percent of conjugated drug between selected stable and unstable EC4 combinations aligned closely with in vitro findings (FIG. 1, FIG. 2A-2B).

TABLE E1
Antibody-conjugated drug pharmacokinetic parameters.

| | acMMAE | | | AUC_{0-7 d} | | AUC_{0-28 d} |
|---|---|---|---|---|---|---|
| | Equiv. | | AUC_{0-7 d} | Fold-change | AUC_{0-28 d} | Fold-change |
| Test Article | Dose | Cₘₐₓ | (day*μg/ | compared to | (day*μg/ | compared to |
| (4-load ADCs) | (mg/kg) | (μg/mL) | mL) | conventional | mL) | conventional |
| h00-mc-vc-MMAE | 0.282 | 8.44 | 9.8 | | 12.1 | |
| h00 S239C V205C-mc- | 0.268 | 8.34 | 16.4 | 1.7 | 37.5 | 3.1 |
| vc-MMAE | | | | | | |
| h00 S375C S114C-mc- | 0.268 | 8.15 | 16.2 | 1.7 | 31.3 | 2.6 |
| vc-MMAE | | | | | | |
| h00 S375C S121C-mc- | 0.281 | 8.27 | 13.1 | 1.3 | 26.9 | 2.2 |
| vc-MMAE | | | | | | |
| h00 S400C S121C-mc- | 0.281 | 7.83 | 10.8 | 1.1 | 22.1 | 1.8 |
| vc-MMAE | | | | | | |
| h00 S400C S114C-mc- | 0.281 | 8.83 | 13.9 | 1.4 | 24.4 | 2.0 |
| vc-MMAE | | | | | | |

### Example 3: Assessment of EC4 ADCs Plasma Aggregation In Vitro Compared to Conventional ADCs

In this example, EC4 ADCs were evaluated for high molecular weight formation in plasma compared to conventional ADCs.

### A. Methods

ADCs were prepared as described in Example 1. ADCs were labeled with Alexa Fluor 488 TFP ester (Molecular Probes), desalted, buffer exchanged into PBS (Gibco), and sterile filtered. The concentration and degree of labeling of the resulting ADC-AF488 conjugate was determined by UV absorbance prior to freezing at −80° C. On the day of experiment, AF488-ADC was diluted in plasma and incubated at 37° C. At the indicated time points, aliquots were analyzed by SEC-UPLC with fluorescence detection. The resulting chromatograms were analyzed to determine percent of high molecular weight species.

### B. Results

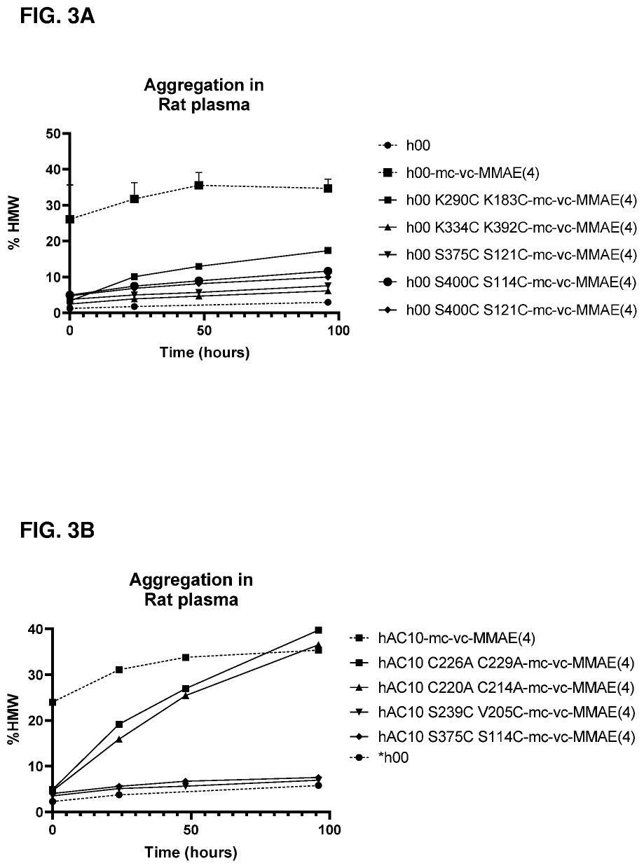

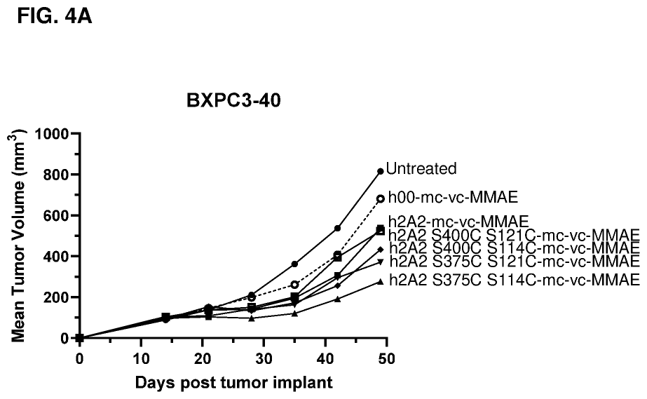

The aggregation of non-targeted (h00) and CD30-targeted (hAC10) ADCs after incubation in rat plasma for 96 hours was observed to be lower for EC4-mc-vc-MMAE ADCs than conventional mc-vc-MMAE ADCs (FIG. 3A-B). The observed aggregation of hAC10 S375C/S114C ADC after incubation in rat plasma was observed to be about the same compared to hAC10 S239C/V205C but significantly lower than conventional and other engineered 4-load ADCs (FIG. 3B). H00 S400C/S121C, H00 S400C/S114C, H00 S375C/S121C had a higher percentage of aggregate formation compared to other EC4 ADCs by about 95 hours, suggesting their stability in plasma was lower.

### Example 4: EC4-Based ADCs are Efficacious in Reducing Tumor Size in Xenograft Mouse Models

In this Example, ADCs were evaluated for clinical benefit by assessing their ability to reduce tumor volumes in xenograft tumor mouse models.

### A. Methods

Preparation of xenograft mouse models: Cells from a cancer cell line (either BXPC3-40 or HPAFII) were implanted into nude mice. After the tumor reached a volume of 100 mm³, various EC4 ADCs were administered via an intraperitoneal injection. Each EC4-based ADC was administered at the same subcurative dose (1 mg/kg at 7 day intervals for 3 times for BXPC3 or 3 mg/kg at 7 day intervals for 3 times for HPAFII) which was previously determined for the conventional ADC comparator in order to clearly distinguish efficacy differences. Tumor size was then measured twice a week until the end of the study.

### B. Results

The targeted h2A2 (anti-ITGB6) EC4-mc-vc-MMAE ADCs showed similar efficacy in suppressing tumor volume in BXPC3-40 xenograft mouse models compared to a h2A2 conventional 4-load comparator ADC (e.g. an ADC without engineered cysteines) (FIG. 4A). S375C/S121C and S400C/S114C exhibited similar clinical benefit at all assay time points (FIG. 4A). In HPAFII tumor bearing mice, h2A2 S375C/S121C was most efficacious at suppressing tumor volume (FIG. 4B). In both mouse models, all tested EC4 ADCs reduced tumor volume at a greater degree than both targeted and untargeted conventional antibodies (FIG. 4A-B). Therefore, ADC tumor killing efficacy was retained in ADCs having different drug conjugation sites.

### Example 5: Evaluation of Bone Marrow Toxicity Induced by Treatment with EC4-Based ADCs

In this Example, differences in in vivo toxicity against normal bone marrow tissue was explored by using a non-binding control (h00) antibody.

### A. Methods

Treatment and analysis of reticulocytes: EC4 based ADCs were prepared as described in Example 1. Reduced non-binding EC4 antibody was reacted with drug-linker to provide a non-binding control ADC. Each of these non-targeting antibodies were reacted with mc-vc-MMAE and the resulting ADCs were then administered intravenously at 15 mg/kg to female Sprague Dawley rats, whose blood were analyzed at day 4 post-administration for reticulocyte counts as a proxy for bone marrow toxicity compared to vehicle treated animals.

Whole blood for routine clinical pathology evaluation was collected in K2-EDTA-containing tubes from the jugular or caudal vena cava under isoflurane anesthesia and analyzed with the Advia 2120i.

Animals were euthanized and sternum and/or femur were collected and fixed in 10% neutral-buffered formalin. Bones were then decalcified, sectioned, processed to glass slides, and bone marrow evaluated for toxicity by a board certified veterinary anatomic pathologist.

### B. Results

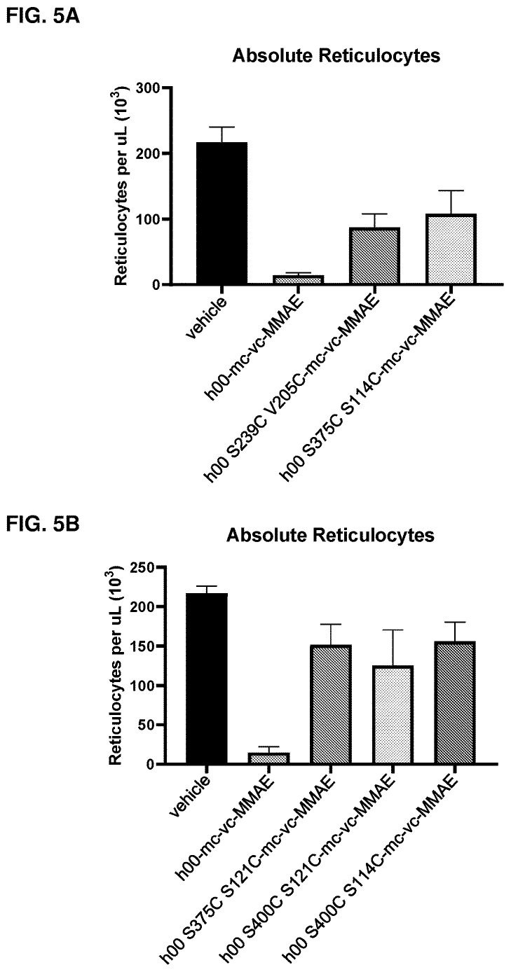

EC4-based h00 conjugates with cysteines located at S375C/S121C, S400C/S121C, and S400C/S114C showed improved preservation of reticulocyte counts in comparison to the conventional comparator conjugate. At the 15 mg/kg dose level tested, all EC4 conjugates had significantly higher levels of reticulocytes compared to rats treated with conventional ADC. (FIG. 5A-B).

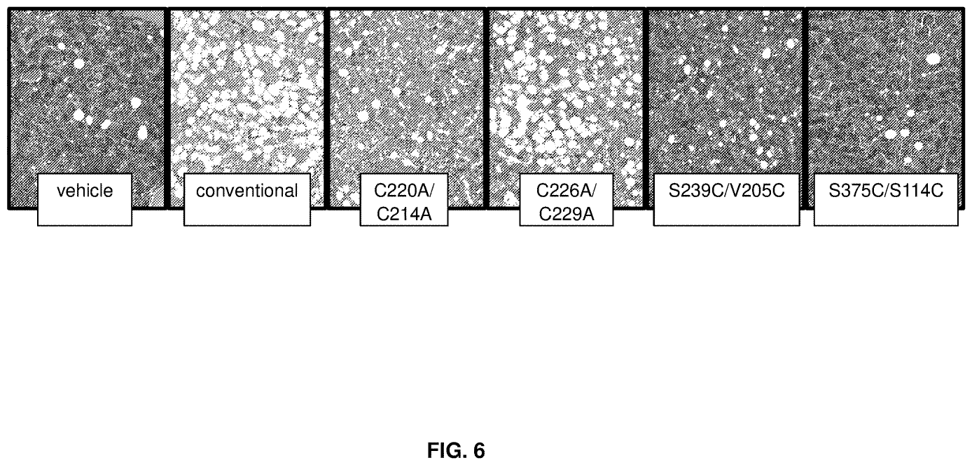

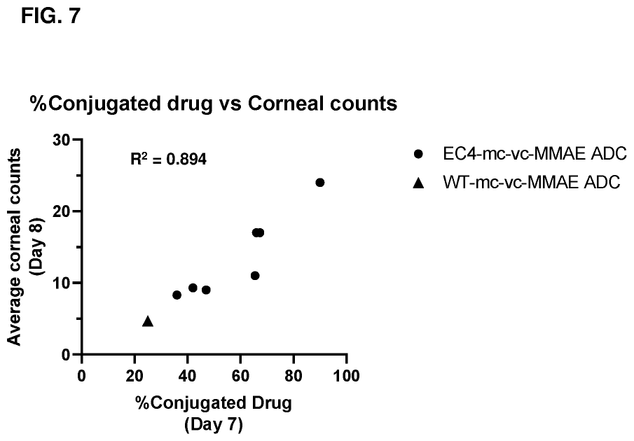

Histopathology of the bone marrow with IHC for mononuclear cells confirmed the preservation of mononuclear bone marrow cells by the EC4-based conjugates compared with administration of the conventional ADC comparator (FIG. 6). EC4-mc-vc-MMAE treated rats had minimal depletion compared to vehicle treated rats at this dose level (FIG. 6).

### Example 6: Evaluation of Ocular/Corneal Toxicity Induced by Treatment with EC4-Based ADCs

In this Example, the effect of cysteine substitutions on the degree of ocular/corneal toxicity was explored by using a non-binding control (h00) antibody.

### A. Methods

ADC preparation, rat injection and euthanization methods were performed as described in Examples 1 and 5. Each of the non-targeting antibodies were reacted with mc-vc-MMAE and the resulting ADCs were then administered at 15 mg/kg to rats, and the rats underwent scheduled necropsies at days 4 and 8 post-administration to collect corneal tissue. EC4 ADCs used in the experiment are described in Table E2. The degree of corneal toxicity was measured by counting the number of mitotic and apoptotic figures in corneal tissue samples. Animals were euthanized and eyes were collected and fixed in in Modified Davidson's Fixative (MDF). Eyes were then sectioned, processed to glass slides, and evaluated for toxicity by a board certified veterinary anatomic pathologist.

### B. Results

Histopathology of the cornea indicated that only EC4 combinations of S375C/S121C, S400C/S121C, and S400C/S114C maintained corneal counts similar to vehicle treated rats (Table E2). EC4-based ADCs with higher conjugated drug stability elevated the number of mitotic and apoptotic figures observed in the cornea on day 8 compared to vehicle treated rats (FIG. 1, 2A, 2B, 7). The degree of corneal and ocular toxicity was also observed to be correlated to ADC stability in vitro and in vivo (FIG. 1, 2A, 2B, 3, 7). Notably, S239C/V205C was observed to exhibit less drug loss and form less aggregates than S400C/S114C, S400C/S121C, S375C/S121C in multiple stability assays (FIG. 1, 2A-2B, 3, 7) but induced about 3-fold more corneal counts in comparison. This Example demonstrates that ADC stability varies based upon cysteine location and that the engineered cysteines at positions that lower ADC stability also result in reduced toxicity.

TABLE E2
Number of mitotic and apoptotic figures (corneal counts)
observed in corneal tissue for various ADCs.

| | Dose | Avg. Corneal |
|---|---|---|
| ADC | (mg/kg) | counts (Day 8) |
| h00-mc-vc-MMAE | 15 | 10 |
| h00 S239C V205C-mc-vc-MMAE | 15 | 24 |
| h00 S375C S114C-mc-vc-MMAE | 15 | 17 |
| h00 S375C S121C-mc-vc-MMAE | 15 | 9.3 |
| h00 S400C S121C-mc-vc-MMAE | 15 | 9 |
| h00 S400C S114C-mc-vc-MMAE | 15 | 8.3 |

### Example 7: EC4-Based ADCs Assessed for Comparative Potency to Conventional In Vitro Cytotoxicity Assays

Using the cysteine sites identified in the ADCs described above, EC4 ADCs are prepared and tested for efficacy in cytotoxicity assays.

Preparation of ADCs: Antigen ITGB6 is upregulated in various solid tumors including pancreatic, head and neck, lung, and esophageal tumors. Anti-ITGB6-humanized EC4 antibodies were used to prepare antibody drug conjugates having a drug antibody ratio (DAR) of about 4. ADCs are prepared according to Example 1.

In vitro cytotoxicity assay: The cytotoxic effect of an EC4-based ADC was measured by a cell proliferation assay employing the protocol in the Promega Corp. Technical Bulletin TB288; and Mendoza et a1., 2002, Cancer Res. 62:5485-5488), the methods of which are specifically incorporated by reference herein. Briefly, an aliquot of 40 μl of cell culture containing about 400 cells in medium was deposited in each well of a 384-well, opaque-walled plate. A 10 μL aliquot of free cytotoxic agent or ADC was added to the experimental wells and incubated for 96 hr and then incubated at room temperature for approximately 30 mins whereupon a volume of CellTiter-Glo™ reagent equal to the volume of cell culture medium present in each well was added. The contents were mixed for 2 mins on an orbital shaker to induce cell lysis and the plate was incubated at room temperature for 10 mins to stabilize the luminescence signal for recordation.

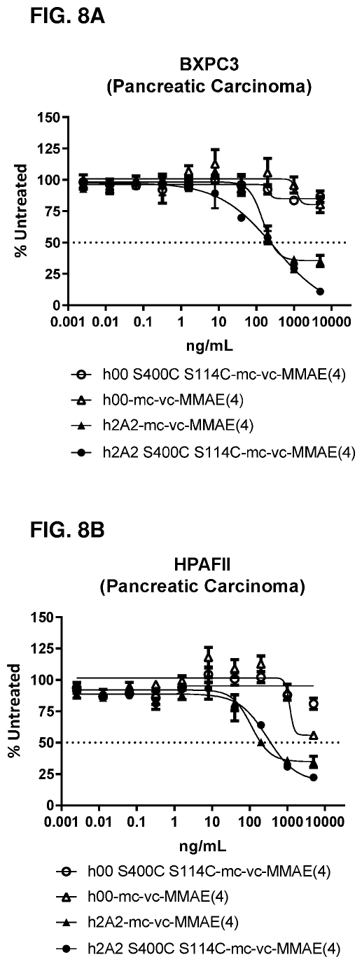

Anti-ITGB6 ADCs were tested for potency. The antibodies were engineered with the cysteine pairs disclosed above in the previous Examples. The efficacy of anti-ITGB6 EC4 ADCs were also tested against conventional anti-ITGB6 ADCs. The cells were treated with conventional or EC4 ADCs, then the remaining cells were counted to assess the cytotoxic effects of each EC4 variant compared to conventional ADCs. Treatment with up to approximately 500 ng/ml of conventional non-targeting antibody conjugated to MMAE (h00-mc-vc-MMAE) or EC4 non-targeting antibody conjugated to MMAE (h00 S400C S114C-mc-vc-MMAE) resulted in 100% cell viability in BxPC-3 cells (FIG. 8A) and HPAFII cells (FIG. 8B). Cytotoxicity in BxPC-3 cells increased when the cells were treated with greater than 50 ng/mL of the positive control, conventional anti-ITGB6 ADCs (h2A2-mc-vc-MMAE). A similar concentration of anti-ITGB6 EC4 ADC (h2A2 S400C S114C-mc-vc-MMAE) was needed to cause cytotoxicity, demonstrating equal potency of the EC4 counterpart for anti-ITGB6 ADCs (FIG. 8A). In HPAFII cells, there was no cytotoxicity when the cells were treated with up to approximately 100 ng/ml h00-mc-vc-MMAE or h00 S400C S114C-mc-vc-MMAE. Both h2A2-mc-vc-MMAE and h2A2 S400C S114C-mc-vc-MMAE were cytotoxic to HPAFII cells when greater than 10 ng/mL ADC were used on the cells. Overall, both EC4 and conventional ADCs showed consistent cytotoxicity (FIG. 8A, FIG. 8B, and Table E3).

TABLE E3
in vitro cytotoxicity of ITGB6-targeted and non-targeted ADCs.

| | BXPC3 | | HPAFII | |
|---|---|---|---|---|
| | | % viability | | % viability |
| | x50 | at top | x50 | at top |
| ADC/Drug | (ng/mL) | concentration | (ng/mL) | concentration |
| h2A2 S400C | 236 | NA | 360 | 19 |
| S114C-mc-vc- | | | | |
| MMAE(4) | | | | |
| h2A2-mc-vc- | 251 | 36 | 199 | 35 |
| MMAE(4) | | | | |
| h00-mc-vc- | >5K | 80 | >5K | 56 |
| MMAE(4) | | | | |
| h00 S400C | >5K | 85 | >5K | 90 |
| S114C-mc-vc- | | | | |
| MMAE(4) | | | | |

### Example 8: Stability and Toxicity of EC4s ADCs were Assessed in Rat Models

In this experiment, a panel of additional EC4 combinations (Table E4) were introduced into a non-targeting antibody. The EC4 ADCs bearing various engineered cysteine pairs were assessed for drug loss over time. ADCs were administered intravenously at 15 mg/kg into rats and the percent of initial DAR was determined. This experiment was performed using a conventional ADC comparator to compare relative ADC stability.

TABLE E4
Additional EC4 combinations

| | Engineered |
|---|---|
| | Cysteine (EC) |
| mAb | sites |
| h00 | S375C S114C |
| h00 | S239C V205C |
| h00 | S375C R142C |
| h00 | E258C E165C |
| h00 | P171C R142C |
| h00 | A118C V205C |
| h00 | A140C V205C |
| h00 | A118C R142C |

EC4 ADCs were tested for in vivo stability as described in Example 2, and for bone marrow and ocular/corneal toxicity as described in Examples 5 and 6. The EC4 ADCs in Table E4 were evaluated for bone marrow toxicity by investigating the degree of bone marrow suppression and preservation of reticulocytes in rats.

The panel of EC4 combinations (Table E4) was also expressed on a non-targeting antibody and injected into rats at 15 mg/kg. Corneal tissue was analyzed for mitotic and apoptotic figures. This data was compared to vehicle-treated rats.

This data was used to identify the relationship between the stability of engineered cysteine antibodies and in vivo toxicity.

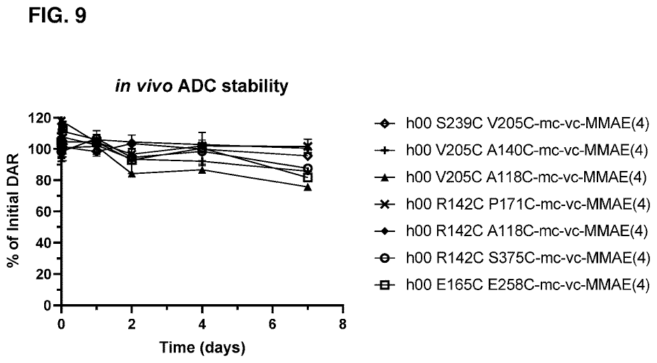

EC4 combinations previously reported in the art were conjugated with about 4 copies of mc-vc-MMAE per antibody and administered in Sprague Dawley rats to assess pharmacokinetic parameters and in vivo conjugated drug stability. All EC4-based ADCs had significantly greater total antibody and antibody-conjugated drug exposure than h00-mc-vc-MMAE, the native cysteine ADC comparator from Example 2 (Table E5). Each of the EC4 ADCs from Table E4 were highly stable in vivo with greater than 75% conjugated drug remaining on the antibody through day 7 (FIG. 9).

TABLE E5
Antibody-conjugated drug pharmacokinetic parameters.

| | acMMAE | | | % of |
|---|---|---|---|---|
| | Equiv. | | | initial |
| Test Article | Dose | Cₘₐₓ | AUC_{0-7 d} | DAR |
| (4-load ADCs) | (mg/kg) | (μg/mL) | (day*μg/mL) | (day 7) |
| h00 V205C A140C-mc-vc- | 0.282 | 7.4 | 13.4 | 85.7 |
| MMAE(4) | | | | |
| h00 V205C A118C- mc-vc- | 0.282 | 7.78 | 17.1 | 75.8 |
| MMAE(4) | | | | |
| h00 R142C P171C- mc-vc- | 0.276 | 7.37 | 18.8 | 102 |
| MMAE(4) | | | | |
| h00 R142C A118C- mc-vc- | 0.283 | 7.74 | 18 | 100 |
| MMAE(4) | | | | |
| h00 R142C S375C- mc-vc- | 0.282 | 7.43 | 16.9 | 87.7 |
| MMAE(4) | | | | |
| h00 E165C E258C- mc-vc- | 0.276 | 8.07 | 15.8 | 81.8 |
| MMAE(4) | | | | |

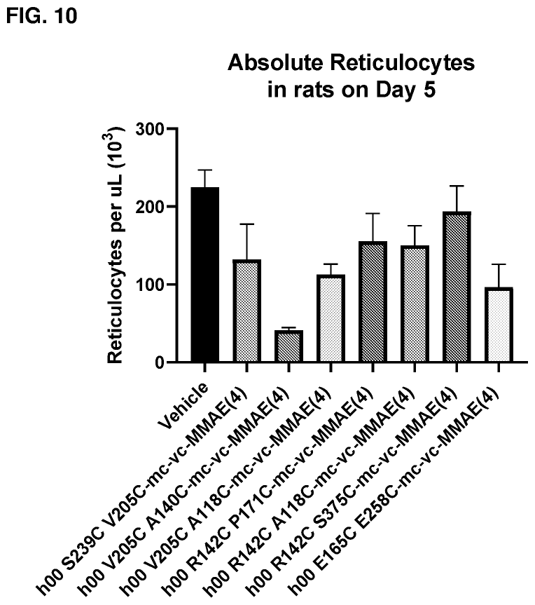

EC4-based h00 conjugates listed in Table E4 showed improved preservation of reticulocyte counts in comparison to h00-mc-vc-MMAE, the native cysteine comparator from Example 5. At the 15 mg/kg dose level tested, EC4 conjugates tended to have significantly higher levels of reticulocytes compared to rats treated with conventional ADC (FIG. 10).

Histopathology of the bone marrow with IHC for mononuclear cells confirmed the preservation of mononuclear bone marrow cells by the EC4-based conjugates. EC4-mc-vc-MMAE treated rats had minimal to mild depletion compared to vehicle treated rats at this dose level (Table E6).

TABLE E6
Histopathology severity scoring for rats treated with EC4-based ADCs.

| | Dose | Bone Marrow, Femur | | | | | Eye | | | | |
|---|---|---|---|---|---|---|---|---|---|---|---|
| Treatment | (mg/kg) | Day 8 | | | | | Day 8 | | | | |
| Vehicle | 15 | — | — | — | [symbol] | | — | — | — | [symbol] | |
| h00 S239C V205C- mc-vc- | 15 | 1 | — | — | — | 1 | 1 | 1 | — | 1 | 1 |
| MMAE(4) | | | | | | | | | | | |
| h00 V205C A140C- mc-vc- | 15 | 2 | 2 | 2 | 2 | 2 | 1 | 1 | 1 | 1 | 1 |
| MMAE(4) | | | | | | | | | | | |
| h00 V205C A118C- mc-vc- | 15 | 2 | 2 | 2 | 1 | 1 | — | — | — | 2 | 1 |
| MMAE(4) | | | | | | | | | | | |
| h00 R142C P171C- mc-vc- | 15 | — | — | — | 2 | 1 | 2 | 2 | 2 | 2 | — |
| MMAE(4) | | | | | | | | | | | |
| h00 R142C A118C- mc-vc- | 15 | 2 | 1 | 2 | 1 | — | 1 | 1 | 2 | 2 | — |
| MMAE(4) | | | | | | | | | | | |
| h00 R142C S375C- mc-vc- | 15 | 1 | 2 | 1 | 1 | — | — | 1 | 1 | — | 2 |
| MMAE(4) | | | | | | | | | | | |
| h00 E165C E258C- mc-vc- | 15 | 1 | — | 1 | 2 | 1 | 1 | 2 | 2 | 3 | 3 |
| MMAE(4) | | | | | | | | | | | |

(—) = no findings;
1 = minimal;
2 = mild;
3 = moderate;
4 = severe

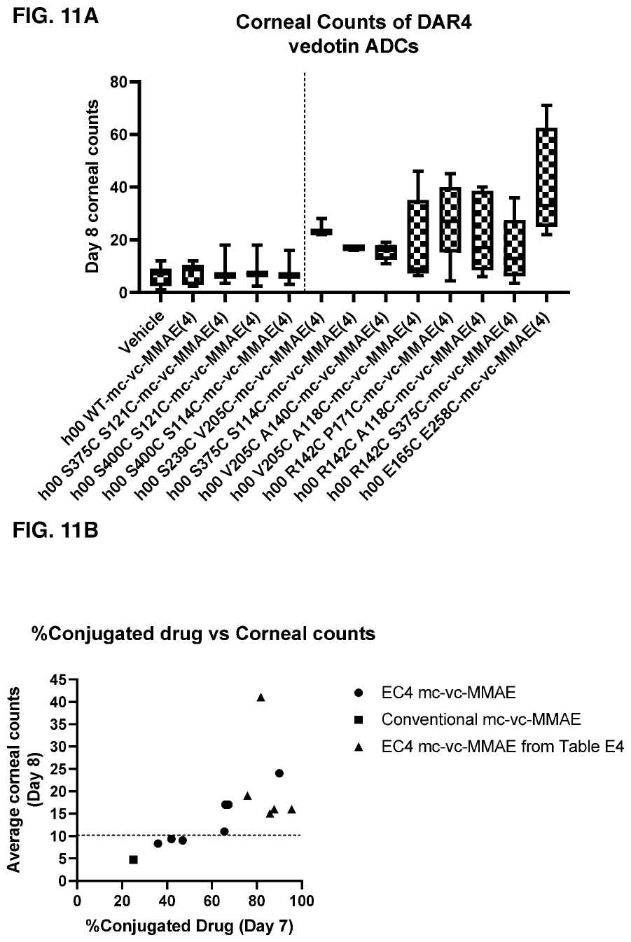

Histopathology of the cornea indicated that EC4 combinations from Table E4 tended to have elevated the number of mitotic and apoptotic figures observed in the cornea compared to vehicle treated rats, as well as previously disclosed low stability variants from Example 6 (FIG. 11A). This is consistent with findings from Example 6 where EC4-based ADCs with higher conjugated drug stability elevated the number of corneal counts on Day 8 compared to vehicle treated rats (FIG. 11B).

### Example 9: Stability and Ocular Toxicity of EC4 ADCs Conjugated to Various Drug Moieties

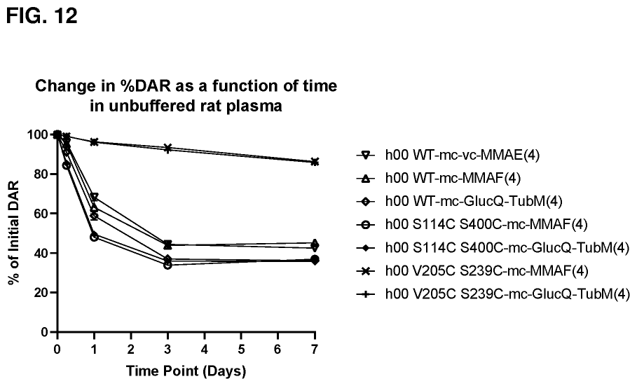

A panel of alternative drug classes were tested in combination with stable and unstable EC4 combinations (Table E7) to determine the impact on corneal toxicity. ADCs with various drug moieties were assessed in plasma stability to determine the relative stability on various EC combinations. To assess maleimide stability, S400C/S114C and S239C/V205C antibodies conjugated with various drug moieties (mc-GlucQ-TubM or mc-MMAF) having a DAR of about 4 were prepared and incubated in unbuffered Sprague Dawley rat plasma up to 7 days. Results indicated the MC linker conjugated to the S400C S114C sites across all drug moieties had consistently lower stability profiles compared to various drug moieties conjugated to S239C V205C (FIG. 12).

The EC4 ADCs conjugated with MC-GlucQ-TubM were administered intravenously at 10 and 15 mg/kg into rats and corneal tissue was examined for number of corneal counts, which was compared to relative ADC stability.

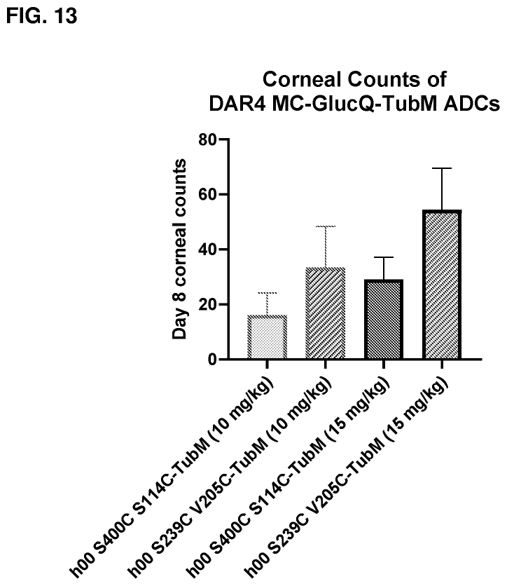

The degree of corneal toxicity in rats was evaluated for MC-GlucQ-Tubulysin M ADCs as described in Example 6. Histopathology of the cornea on Day 8 indicated that h00 S400C S114C conjugated with MC-GlucQ-TubM, which had a lower stability profile, had less severe corneal toxicity compared to the stable h00 S239C V205C conjugated with MC-GlucQ-TubM. Stable EC4-based ADC with higher conjugated drug stability elevated the number of mitotic and apoptotic figures observed in the cornea on Day 8 compared to unstable EC4-based ADC treated rats (FIG. 13).

TABLE E7
List of EC4 ADCs with alternative payloads
evaluated for stability across EC sites.

| mAb | EC sites | Drug-linker | Relative stability |
|---|---|---|---|
| h00 | HC S239C LC V205C | mc-GlucQ-TubM | High |
| h00 | HC S400C LC S114C | mc-GlucQ-TubM | Low |
| h00 | HC S239C LC V205C | mc-MMAF | High |
| h00 | HC S400C LC S114C | mc-MMAF | Low |

### Example 10: In Vitro Stability Profiles of EC4 Antibodies with Different Targets

The engineered cysteine combination S400C/S114C was applied to various IgG1 antibody backbones that target various antigens (Table E8) as described in Example 1. EC4 with various antibody backbones were compared to identify variations in ADC stability profile. The panel of S400C/S114C EC4 antibodies targeting different antigens was evaluated in in vitro using mouse plasma as described in Example 1.

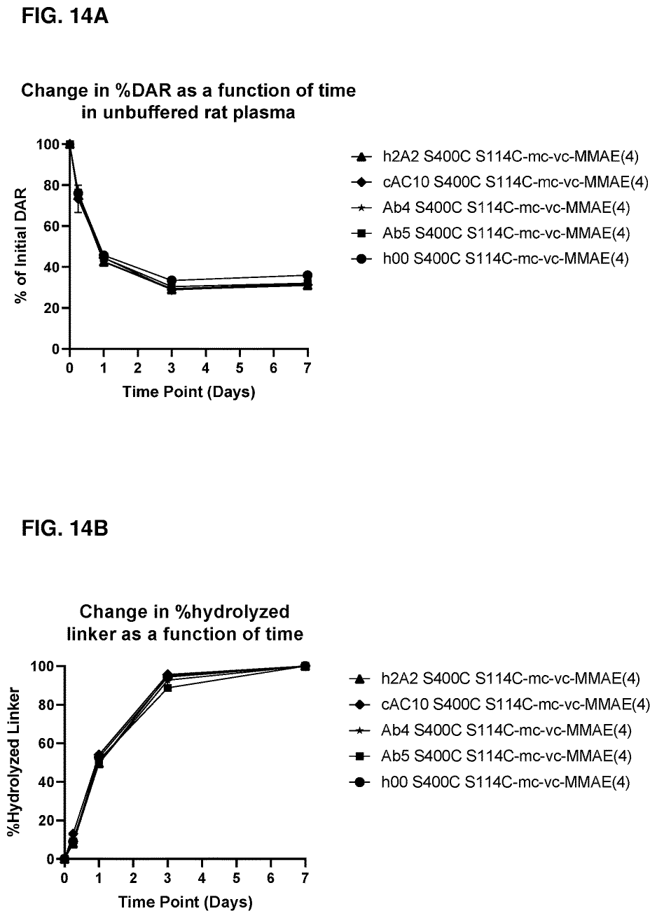

To assess maleimide hydrolysis and stability, the panel of S400C/S114C antibodies conjugated with mc-vc-MMAE having a DAR of about 4 were prepared and incubated in unbuffered Sprague Dawley rat plasma for up to 7 days. Results indicated the MC linker conjugated to the S400C S114C sites across the various antibody backbones had consistent stability profiles (FIG. 14A). Additionally, the change in percent hydrolyzed maleimide remained consistent across all antibody backbones over the course of the 7 days in unbuffered Sprague Dawley rat plasma (FIG. 14B).

TABLE E8
List of antibody backbones with alternative targets
that will be engineered with the S400C/S114C combination
and screened for in vitro plasma stability.

| mAb | Antigen |
|---|---|
| h2A2 | ITGB6 |
| cAC10 | CD30 |
| Ab4 | CEACAM5 |
| Ab5 | B7-H4 |

SEQUENCE LISTING

| SEQ ID | | |
|---|---|---|
| NO | Description | Sequence |
| 1 | cAC10 and hAC10 CDR- | DYYIT |
| | H1 | |
| 2 | cAC10 and hAC10 CDR- | WIYPGSGNTKYNEKFKG |
| | H2 | |
| 3 | cAC10 and hAC10 CDR- | YGNYWFAY |
| | H3 | |
| 4 | cAC10 and hAC10 CDR-L1 | KASQSVDFDGDSYMN |
| 5 | cAC10 and hAC10 CDR-L2 | AASNLES |
| 6 | cAC10 and hAC10 CDR-L3 | QQSNEDPWT |
| 7 | cAC10 VH | QIQLQQSGPEVVKPGASVKISCKASGYTFTDYYIT |
| | | WVKQKPGQGLEWIGWIYPGSGNTKY |
| | | NEKFKGKATLTVDTSSSTAFMQLSSLTSEDTAVYF |
| | | CANYGNYWFAYWGQGTQVTVSA |
| 8 | cAC10 VL | DIVLTQSPASLAVSLGQRATISCKASQSVDFDGDS |
| | | YMNWYQQKPGQPPKVLIYAASNLES |
| | | GIPARFSGSGSGTDFTLNIHPVEEEDAATYYCQQS |
| | | NEDPWTFGGGTKLEIK |
| 9 | cAC10 HC | QIQLQQSGPEVVKPGASVKISCKASGYTFTDYYIT |
| | | WVKQKPGQGLEWIGWIYPGSGNTKY |
| | | NEKFKGKATLTVDTSSSTAFMQLSSLTSEDTAVYF |
| | | CANYGNYWFAYWGQGTQVTVSAAST |
| | | KGPSVFPLAPSSKSTSGGTAALGCLVKDYFPEPVT |
| | | VSWNSGALTSGVHTFPAVLQSS |
| | | GLYSLSSVVTVPSSSLGTQTYICNVNHKPSNTKVD |
| | | KKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYN |
| | | STYRVVSVLTVLHQDWLNGKEYKCKVSNKALPA |
| | | PIEKTISKAKGQPREPQVYTLPPSRDE |
| | | LTKNQVSLTCLVKGFYPSDIAVEWESNGQPENNY |
| | | KTTPPVLDSDGSFFLYSKLTVDKSRW |
| | | QQGNVFSCSVMHEALHNHYTQKSLSLSPGK |
| 10 | cAC10 HC v2 | QIQLQQSGPEVVKPGASVKISCKASGYTFTDYYIT |
| | | WVKQKPGQGLEWIGWIYPGSGNTKY |
| | | NEKFKGKATLTVDTSSSTAFMQLSSLTSEDTAVYF |
| | | CANYGNYWFAYWGQGTQVTVSAAST |
| | | KGPSVFPLAPSSKSTSGGTAALGCLVKDYFPEPVT |
| | | VSWNSGALTSGVHTFPAVLQSS |
| | | GLYSLSSVVTVPSSSLGTQTYICNVNHKPSNTKVD |
| | | KKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYN |
| | | STYRVVSVLTVLHQDWLNGKEYKCKVSNKALPA |
| | | PIEKTISKAKGQPREPQVYTLPPSRDE |
| | | LTKNQVSLTCLVKGFYPSDIAVEWESNGQPENNY |
| | | KTTPPVLDSDGSFFLYSKLTVDKSRW |
| | | QQGNVFSCSVMHEALHNHYTQKSLSLSPG |
| 11 | cAC10 LC | DIVLTQSPASLAVSLGQRATISCKASQSVDFDGDS |
| | | YMNWYQQKPGQPPKVLIYAASNLES |
| | | GIPARFSGSGSGTDFTLNIHPVEEEDAATYYCQQS |
| | | NEDPWTFGGGTKLEIKR |
| | | TVAAPSVFIFPPSDEQLKSGTASVVCLLNNFYPRE |
| | | AKVQWKVDNALQSGNSQESVTEQDS |
| | | KDSTYSLSSTLTLSKADYEKHKVYACEVTHQGLS |
| | | SPVTKSFNRGEC |
| 12 | h1F6 VH | QVQLVQSGAEVKKPGASVKVSCKASGYTFTNYG |
| | | MNWVRQAPGQGLKWMGWINTYTGEPTY |
| | | ADAFKGRVTMTRDTSISTAYMELSRLRSDDTAVY |
| | | YCARDYGDYGMDYWGQGTTVTVSS |
| 13 | h1F6 VL | DIVMTQSPDSLAVSLGERATINCRASKSVSTSGYS |
| | | FMHWYQQKPGQPPKLLIYLASNLES |
| | | GVPDRFSGSGSGTDFTLTISSLQAEDVAVYYCQHS |
| | | REVPWTFGQGTKVEIK |
| 14 | h1F6 HC | QVQLVQSGAEVKKPGASVKVSCKASGYTFTNYG |
| | | MNWVRQAPGQGLKWMGWINTYTGEPTY |
| | | ADAFKGRVTMTRDTSISTAYMELSRLRSDDTAVY |
| | | YCARDYGDYGMDYWGQGTTVTVSSAS |
| | | TKGPSVFPLAPSSKSTSGGTAALGCLVKDYFPEPV |
| | | TVSWNSGALTSGVHTFPAVLQSSGL |
| | | YSLSSVVTVPSSSLGTQTYICNVNHKPSNTKVDKK |
| | | VEPKSCDKTHTCPPCPAPELLGGPS |
| | | VFLFPPKPKDTLMISRTPEVTCVVVDVSHEDPEVK |
| | | FNWYVDGVEVHNAKTKPREEQYNST |
| | | YRVVSVLTVLHQDWLNGKEYKCKVSNKALPAPI |
| | | EKTISKAKGQPREPQVYTLPPSRDELT |
| | | KNQVSLTCLVKGFYPSDIAVEWESNGQPENNYKT |
| | | TPPVLDSDGSFFLYSKLTVDKSRWQQ |
| | | GNVFSCSVMHEALHNHYTQKSLSLSPGK |
| 15 | h1F6 LC | DIVMTQSPDSLAVSLGERATINCRASKSVSTSGYS |
| | | FMHWYQQKPGQPPKLLIYLASNLES |
| | | GVPDRFSGSGSGTDFTLTISSLQAEDVAVYYCQHS |
| | | REVPWTFGQGTKVEIKRTVAAPSVF |
| | | IFPPSDEQLKSGTASVVCLLNNFYPREAKVQWKV |
| | | DNALQSGNSQESVTEQDSKDSTYSLS |
| | | STLTLSKADYEKHKVYACEVTHQGLSSPVTKSFN |
| | | RGEC |
| 16 | TROP2 CDR-H1 | NYGMN |
| 17 | TROP2 CDR-H2 | WINTYTGEPTYTDDFKG |
| 18 | TROP2 CDR-H3 | GGFGSSYWYFDV |
| 19 | TROP2 CDR-L1 | KASQDVSIAVA |
| 20 | TROP2 CDR-L2 | SASYRYT |
| 21 | TROP2 CDR-L3 | QQHYITPLT |
| 22 | TROP2 VH | QVQLQQSGSELKKPGASVKVSCKASGYTFTNYG |
| | | MNWVKQAPGQGLKWMGWINTYTGEPT |
| | | YTDDFKGRFAFSLDTSVSTAYLQISSLKADDTAVY |
| | | FCARGGFGSSYWYFDVWGQGSLVTVSS |
| 23 | TROP2 VL | DIQLTQSPSSLSASVGDRVSITCKASQDVSIAVAW |
| | | YQQKPGKAPKLLIYSASYRYTGVP |
| | | DRFSGSGSGTDFTLTISSLQPEDFAVYYCQQHYITP |
| | | LTFGAGTKVEIK |
| 24 | TROP2 CDR-H1 | TAGMQ |
| 25 | TROP2 CDR-H2 | WINTHSGVPKYAEDFKG |
| 26 | TROP2 CDR-H3 | SGFGSSYWYFDV |
| 27 | TROP2 CDR-L1 | KASQDVSTAVA |
| 28 | TROP2 CDR-L2 | SASYRYT |
| 29 | TROP2 CDR-L3 | QQHYITPLT |
| 30 | TROP2 VH | QVQLVQSGAEVKKPGASVKVSCKASGYTFTTAG |
| | | MQWVRQAPGQGLEWMGWINTHSGVPKYAEDFK |
| | | GRVTISADTSTSTAYLQLSSLKSEDTAVYYCARSG |
| | | FGSSYWYFDVWGQGTLVTVSS |
| 31 | TROP2 VL | DIQMTQSPSSLSASVGDRVTITCKASQDVSTAVA |
| | | WYQQKPGKAPKLLIYSASYRYTGVPSRFSGSGSG |
| | | TDFTLTISSLQPEDFAVYYCQQHYITPLTFGQGTK |
| | | LEIK |
| 32 | MICA CDR-H1 | SQNIY |
| 33 | MICA CDR-H2 | YIEPYNVVPMYNPKFKG |
| 34 | MICA CDR-H3 | SGSSNFDY |
| 35 | MICA CDR-L1 | SASSSISSHYLH |
| 36 | MICA CDR-L2 | RTSNLAS |
| 37 | MICA CDR-L3 | QQGSSLPLT |
| 38 | MICA VH | EIQLVQSGAEVKKPGASVKVSCKASGYAFTSQNI |
| | | YWVRQAPGQGLEWIGYIEPYNVVPMYNPKFKGR |
| | | ATLTVDKSTSTAYLELSSLRSEDTAVYYCARSGSS |
| | | NFDYWGQGTLVTVSS |
| 39 | MICA VL | DIQLTQSPSSLSASVGDRVTITCSASSSISSHYLHW |
| | | YQQKPGKSPKLLIYRTSNLASGVPSRFSGSGSGTD |
| | | YTLTISSLQPEDFATYYCQQGSSLPLTFGQGTKVEI |
| | | K |
| 40 | MICA CDR-H1 | NYAMH |
| 41 | MICA CDR-H2 | LIWYDGSNKFYGDSVKG |
| 42 | MICA CDR-H3 | EGSGHY |
| 43 | MICA CDR-L1 | RASQGISSALA |
| 44 | MICA CDR-L2 | DASSLES |
| 45 | MICA CDR-L3 | QQFNSYPIT |
| 46 | MICA VH | QVQLVESGGGVVQPGRSLRLSCAASGFTFSNYAM |
| | | HWVRQAPGEGLEWVALIWYDGSNKFYGDSVKG |
| | | RFTISRDNSKNTLYLQMNSLSAEDTAVYYCAREG |
| | | SGHYWGQGTLVTVSS |
| 47 | MICA VL | AIQLTQSPSSLSASVGDRVTITCRASQGISSALAWY |
| | | QQKPGKVPKSLIYDASSLESGVPSRFSGSGSGTDF |
| | | TLTISSLQPEDFATYYCQQFNSYPITFGQGTRLEIK |
| 48 | MICA CDR-H1 | NYAMS |
| 49 | MICA CDR-H2 | YISPGGDYIYYADSVKG |
| 50 | MICA CDR-H3 | DRRHYGSYAMDY |
| 51 | MICA CDR-L1 | RSSKSLLHSNLNTYLY |
| 52 | MICA CDR-L2 | RMSNLAS |
| 53 | MICA CDR-L3 | MQHLEYPFT |
| 54 | MICA VH | QVQLVESGGGLVKPGGSLRLSCAASGFTFSNYAM |
| | | SWIRQAPGKGLEWVSYISPGGDYIYYADSVKGRF |
| | | TISRDNAKNSLYLQMNSLRAEDTAVYYCTTDRRH |
| | | YGSYAMDYWGQGTLVTVSS |
| 55 | MICA VL | DIVMTQSPLSLPVTPGEPASISCRSSKSLLHSNLNT |
| | | YLYWFLQKPGQSPQILIYRMSNLASGVPDRFSGSG |
| | | SGTAFTLKISRVEAEDVGVYYCMQHLEYPFTFGP |
| | | GTKLEIK |
| 56 | MICA CDR-H1 | TYAFH |
| 57 | MICA CDR-H2 | GIVPIFGTLKYAQKFQD |
| 58 | MICA CDR-H3 | AIQLEGRPFDH |
| 59 | MICA CDR-L1 | RASQGITSYLA |
| 60 | MICA CDR-L2 | AASALQS |
| 61 | MICA CDR-L3 | QQVNRGAAIT |
| 62 | MICA VH | QVQLVQSGAEVKKPGSSVRVSCRASGGSSTTYAF |
| | | HWVRQAPGQGLEWMGGIVPIFGTLKYAQKFQDR |
| | | VTLTADKSTGTAYMELNSLRLDDTAVYYCARAIQ |
| | | LEGRPFDHWGQGTQVTVSA |
| 63 | MICA VL | DIQLTQSPSFLSASVGDRVTITCRASQGITSYLAW |
| | | YQQKPGKAPKLLIYAASALQSGVPSRFSGRGSGT |
| | | EFTLTISSLQPEDFATYYCQQVNRGAAITFGHGTR |
| | | LDIK |
| 64 | CD24 CDR-H1 | TYAFH |
| 65 | CD24 CDR-H2 | GIVPIFGTLKYAQKFQD |
| 66 | CD24 CDR-H3 | AIQLEGRPFDH |
| 67 | CD24 CDR-L1 | RASQGITSYLA |
| 68 | CD24 CDR-L2 | AASALQS |
| 69 | CD24 CDR-L3 | QQVNRGAAIT |
| 70 | CD24 VH | QVQLVQSGAEVKKPGSSVRVSCRASGGSSTTYAF |
| | | HWVRQAPGQGLEWMGGIVPIFGTLKYAQKFQDR |
| | | VTLTADKSTGTAYMELNSLRLDDTAVYYCARAIQ |
| | | LEGRPFDHWGQGTQVTVSA |
| 71 | CD24 VL | DIQLTQSPSFLSASVGDRVTITCRASQGITSYLAW |
| | | YQQKPGKAPKLLIYAASALQSGVPS |
| | | RFSGRGSGTEFTLTISSLQPEDFATYYCQQVNRGA |
| | | AITFGHGTRLDIK |
| 72 | ITGav/CD51 CDR-H1 | RYTMH |
| 73 | ITGav/CD51 CDR-H2 | VISFDGSNKYYVDSVKG |
| 74 | ITGav/CD51 CDR-H3 | EARGSYAFDI |
| 75 | ITGav/CD51 CDR-L1 | RASQSVSSYLA |
| 76 | ITGav/CD51 CDR-L2 | DASNRAT |
| 77 | ITGav/CD51 CDR-L3 | QQRSNWPPFT |
| 78 | ITGav/CD51 VH | QVQLVESGGGVVQPGRSRRLSCAASGFTFSRYTM |
| | | HWVRQAPGKGLEWVAVISEDGSNKYYVDSVKGR |
| | | FTISRDNSENTLYLQVNILRAEDTAVYYCAREARG |
| | | SYAFDIWGQGTMVTVSS |
| 79 | ITGav/CD51 VL | EIVLTQSPATLSLSPGERATLSCRASQSVSSYLAW |
| | | YQQKPGQAPRLLIYDASNRATGIPARFSGSGSGTD |
| | | FTLTISSLEPEDFAVYYCQQRSNWPPFTFGPGTKV |
| | | DIK |
| 80 | ITGav/CD51 CDR-H1 | SFWMH |
| 81 | ITGav/CD51 CDR-H2 | YINPRSGYTEYNEIFRD |
| 82 | ITGav/CD51 CDR-H3 | FLGRGAMDY |
| 83 | ITGav/CD51 CDR-L1 | RASQDISNYLA |
| 84 | ITGav/CD51 CDR-L2 | YTSKIHS |
| 85 | ITGav/CD51 CDR-L3 | QQGNTFPYT |
| 86 | ITGav/CD51 VH | QVQLQQSGGELAKPGASVKVSCKASGYTFSSFW |
| | | MHWVRQAPGQGLEWIGYINPRSGYTEYNEIFRDK |
| | | ATMTTDTSTSTAYMELSSLRSEDTAVYYCASFLG |
| | | RGAMDYWGQGTTVTVSS |
| 87 | ITGav/CD51 VL | DIQMTQSPSSLSASVGDRVTITCRASQDISNYLAW |
| | | YQQKPGKAPKLLIYYTSKIHSGVPSRFSGSGSGTD |
| | | YTFTISSLQPEDIATYYCQQGNTFPYTFGQGTKVEI |
| | | K |
| 88 | gpA33 CDR-H1 | TSSYYWG |
| 89 | gpA33 CDR-H2 | TIYYNGSTYYSPSLKS |
| 90 | gpA33 CDR-H3 | QGYDIKINIDV |
| 91 | gpA33 CDR-L1 | RASQSVSSYLA |
| 92 | gpA33 CDR-L2 | VASNRAT |
| 93 | gpA33 CDR-L3 | QQRSNWPLT |
| 94 | gpA33 VH | QLQLQESGPGLVKPSETLSLTCTVSGGSISTSSYY |
| | | WGWIRQPPGKGLEWIGTIYYNGSTYYSPSLKSRV |
| | | SISVDTSKNQFSLKLSSVTAADTSVYYCARQGYDI |
| | | KINIDVWGQGTTVTVSS |
| 95 | gpA33 VL | EIVLTQSPATLSLSPGERATLSCRASQSVSSYLAW |
| | | YQQKPGQAPRLLIYVASNRATGIPARFSGSGSGTD |
| | | FTLTISSLEPEDFAVYYCQQRSNWPLTFGGGTKVE |
| | | IK |
| 96 | IL1Rap CDR-H1 | SSWMN |
| 97 | IL1Rap CDR-H2 | RIYPGDGNTHYAQKFQG |
| 98 | IL1Rap CDR-H3 | GYLDPMDY |
| 99 | IL1Rap CDR-L1 | QASQGINNYLN |
| 100 | IL1Rap CDR-L2 | YTSGLHA |
| 101 | IL1Rap CDR-L3 | QQYSILPWT |
| 102 | IL1Rap VH | QVQLVQSGAEVKKPGSSVKVSCKASGYAFTSSW |
| | | MNWVRQAPGQGLEWMGRIYPGDGNTHYAQKFQ |
| | | GRVTLTADKSTSTAYMELSSLRSEDTAVYYCGEG |
| | | YLDPMDYWGQGTLVTVSS |
| 103 | IL1Rap VL | DIQMTQSPSSLSASVGDRVTITCQASQGINNYLNW |
| | | YQQKPGKAPKLLIHYTSGLHAGVPSRFSGSGSGT |
| | | DYTLTISSLEPEDVATYYCQQYSILPWTFGGGTKV |
| | | EIK |
| 104 | EpCAM CDR-H1 | SYGMH |
| 105 | EpCAM CDR-H2 | VISYDGSNKYYADSVKG |
| 106 | EpCAM CDR-H3 | DMGWGSGWRPYYYYGMDV |
| 107 | EpCAM CDR-L1 | RTSQSISSYLN |
| 108 | EpCAM CDR-L2 | WASTRES |
| 109 | EpCAM CDR-L3 | QQSYDIPYT |
| 110 | EpCAM VH | EVQLLESGGGVVQPGRSLRLSCAASGFTFSSYGM |
| | | HWVRQAPGKGLEWVAVISYDGSNKYYADSVKG |
| | | RFTISRDNSKNTLYLQMNSLRAEDTAVYYCAKD |
| | | MGWGSGWRPYYYYGMDVWGQGTTVTVSS |
| 111 | EpCAM VL | ELQMTQSPSSLSASVGDRVTITCRTSQSISSYLNW |
| | | YQQKPGQPPKLLIYWASTRESGVPDRFSGSGSGT |
| | | DFTLTISSLQPEDSATYYCQQSYDIPYTFGQGTKLE |
| | | IK |
| 112 | EpCAM CDR-H1 | NYWMS |
| 113 | EpCAM CDR-H2 | NIKQDGSEKFYADSVKG |
| 114 | EpCAM CDR-H3 | VGPSWEQDY |
| 115 | EpCAM CDR-L1 | TGSSSNIGSYYGVH |
| 116 | EpCAM CDR-L2 | SDTNRPS |
| 117 | EpCAM CDR-L3 | QSYDKGFGHRV |
| 118 | EpCAM VH | EVQLVESGGGLVQPGGSLRLSCAASGFTFSNYWM |
| | | SWVRQAPGKGLEWVANIKQDGSEKFYADSVKGR |
| | | FTISRDNAKNSLYLQMNSLRAEDTAVYYCARVGP |
| | | SWEQDYWGQGTLVTVSA |
| 119 | EpCAM VL | QSVLTQPPSVSGAPGQRVTISCTGSSSNIGSYYGV |
| | | HWYQQLPGTAPKLLIYSDTNRPSGVPDRFSGSKS |
| | | GTSASLAITGLQAEDEADYYCQSYDKGFGHRVFG |
| | | GGTKLTVL |
| 120 | EpCAM CDR-H1 | SYAIS |
| 121 | EpCAM CDR-H2 | GIIPIFGTANYAQKFQG |
| 122 | EpCAM CDR-H3 | GLLWNY |
| 123 | EpCAM CDR-L1 | RASQSVSSNLA |
| 124 | EpCAM CDR-L2 | GASTTAS |
| 125 | EpCAM CDR-L3 | QQYNNWPPAYT |
| 126 | EpCAM VH | QVQLVQSGAEVKKPGSSVKVSCKASGGTFSSYAI |
| | | SWVRQAPGQGLEWMGGIIPIFGTANYAQKFQGRV |
| | | TITADESTSTAYMELSSLRSEDTAVYYCARGLLW |
| | | NYWGQGTLVTVSS |
| 127 | EpCAM VL | EIVMTQSPATLSVSPGERATLSCRASQSVSSNLAW |
| | | YQQKPGQAPRLIIYGASTTASGIPARFSASGSGTDF |
| | | TLTISSLQSEDFAVYYCQQYNNWPPAYTFGQGTK |
| | | LEIK |
| 128 | EpCAM CDR-H1 | NYGMN |
| 129 | EpCAM CDR-H2 | WINTYTGEPTYGEDFKG |
| 130 | EpCAM CDR-H3 | FGNYVDY |
| 131 | EpCAM CDR-L1 | RSSKNLLHSNGITYLY |
| 132 | EpCAM CDR-L2 | QMSNLAS |
| 133 | EpCAM CDR-L3 | AQNLEIPRT |
| 134 | EpCAM VH | QVQLVQSGPEVKKPGASVKVSCKASGYTFTNYG |
| | | MNWVRQAPGQGLEWMGWINTYTGEPTYGEDFK |
| | | GRFAFSLDTSASTAYMELSSLRSEDTAVYFCARFG |
| | | NYVDYWGQGSLVTVSS |
| 135 | EpCAM VL | DIVMTQSPLSLPVTPGEPASISCRSSKNLLHSNGIT |
| | | YLYWYLQKPGQSPQLLIYQMSNLASGVPDRFSSS |
| | | GSGTDFTLKISRVEAEDVGVYYCAQNLEIPRTFGQ |
| | | GTKVEIK |
| 136 | EpCAM CDR-H1 | KYGMN |
| 137 | EpCAM CDR-H2 | WINTYTEEPTYGDDFKG |
| 138 | EpCAM CDR-H3 | FGSAVDY |
| 139 | EpCAM CDR-L1 | RSSKSLLHSNGITYLY |
| 140 | EpCAM CDR-L2 | QMSNRAS |
| 141 | EpCAM CDR-L3 | AQNLELPRT |
| 142 | EpCAM VH | QIQLVQSGPEVKKPGESVKISCKASGYTFTKYGM |
| | | NWVKQAPGQGLKWMGWINTYTEEPTYGDDFKG |
| | | RFTFTLDTSTSTAYLEISSLRSEDTATYFCARFGSA |
| | | VDYWGQGTLVTVSS |
| 143 | EpCAM VL | DIVMTQSALSNPVTLGESGSISCRSSKSLLHSNGIT |
| | | YLYWYLQKPGQSPQLLIYQMSNRASGVPDRFSSS |
| | | GSGTDFTLKISRVEAEDVGVYYCAQNLELPRTFG |
| | | QGTKLEMKR |
| 144 | EpCAM CDR-H1 | DYSMH |
| 145 | EpCAM CDR-H2 | WINTETGEPTYADDFKG |
| 146 | EpCAM CDR-H3 | TAVY |
| 147 | EpCAM CDR-L1 | RASQEISVSLS |
| 148 | EpCAM CDR-L2 | ATSTLDS |
| 149 | EpCAM CDR-L3 | LQYASYPWT |
| 150 | EpCAM VH | QVKLQESGPELKKPGETVKISCKASGYTFTDYSM |
| | | HWVKQAPGKGLKWMGWINTETGEPTYADDFKG |
| | | RFAFSLETSASTAYLQINNLKNEDTATYFCARTAV |
| | | YWGQGTTVTVSS |
| 151 | EpCAM VL | DIQMTQSPSSLSASLGERVSLTCRASQEISVSLSWL |
| | | QQEPDGTIKRLIYATSTLDSGVPKRFSGSRSGSDY |
| | | SLTISSLESEDFVDYYCLQYASYPWTFGGGTKLEI |
| | | KR |
| 152 | CD352 CDR-H1 | NYGMN |
| 153 | CD352 CDR-H2 | WINTYSGEPRYADDFKG |
| 154 | CD352 CDR-H3 | DYGRWYFDV |
| 155 | CD352 CDR-L1 | RASSSVSHMH |
| 156 | CD352 CDR-L2 | ATSNLAS |
| 157 | CD352 CDR-L3 | QQWSSTPRT |
| 158 | CD352 VH | QIQLVQSGSELKKPGASVKVSCKASGYTFTNYGM |
| | | NWVRQAPGQDLKWMGWINTYSGEPRYADDFKG |
| | | RFVFSLDKSVNTAYLQISSLKAEDTAVYYCARDY |
| | | GRWYFDVWGQGTTVTVSS |
| 159 | CD352 VL | QIVLSQSPATLSLSPGERATMSCRASSSVSHMHW |
| | | YQQKPGQAPRPWIYATSNLASGVPARFSGSGSGT |
| | | DYTLTISSLEPEDFAVYYCQQWSSTPRTFGGGTKV |
| | | EIKR |
| 160 | CS1/SLAMF7 CDR-H1 | RYWMS |
| 161 | CS1/SLAMF7 CDR-H2 | EINPDSSTINYAPSLKD |
| 162 | CS1/SLAMF7 CDR-H3 | PDGNYWYFDV |
| 163 | CS1/SLAMF7 CDR-L1 | KASQDVGIAVA |
| 164 | CS1/SLAMF7 CDR-L2 | WASTRHT |
| 165 | CS1/SLAMF7 CDR-L3 | QQYSSYPYT |
| 166 | CS1/SLAMF7 VH | EVQLVESGGGLVQPGGSLRLSCAASGFDFSRYW |
| | | MSWVRQAPGKGLEWIGEINPDSSTINYAPSLKDK |
| | | FIISRDNAKNSLYLQMNSLRAEDTAVYYCARPDG |
| | | NYWYFDVWGQGTLVTVSS |
| 167 | CS1/SLAMF7 VL | DIQMTQSPSSLSASVGDRVTITCKASQDVGIAVA |
| | | WYQQKPGKVPKLLIYWASTRHTGVPDRFSGSGSG |
| | | TDFTLTISSLQPEDVATYYCQQYSSYPYTFGQGTK |
| | | VEIKR |
| 168 | CD38 CDR-H1 | SFAMS |
| 169 | CD38 CDR-H2 | AISGSGGGTYYADSVKG |
| 170 | CD38 CDR-H3 | DKILWFGEPVFDY |
| 171 | CD38 CDR-L1 | RASQSVSSYLA |
| 172 | CD38 CDR-L2 | DASNRAT |
| 173 | CD38 CDR-L3 | QQRSNWPPT |
| 174 | CD38 VH | EVQLLESGGGLVQPGGSLRLSCAVSGFTFNSFAM |
| | | SWVRQAPGKGLEWVSAISGSGGGTYYADSVKGR |
| | | FTISRDNSKNTLYLQMNSLRAEDTAVYFCAKDKI |
| | | LWFGEPVFDYWGQGTLVTVSS |
| 175 | CD38 VL | EIVLTQSPATLSLSPGERATLSCRASQSVSSYLAW |
| | | YQQKPGQAPRLLIYDASNRATGIPARFSGSGSGTD |
| | | FTLTISSLEPEDFAVYYCQQRSNWPPTFGQGTKVE |
| | | IKR |
| 176 | CD25 CDR-H1 | SYRMH |
| 177 | CD25 CDR-H2 | YINPSTGYTEYNQKFKD |
| 178 | CD25 CDR-H3 | GGGVFDY |
| 179 | CD25 CDR-L1 | SASSSISYMH |
| 180 | CD25 CDR-L2 | TTSNLAS |
| 181 | CD25 CDR-L3 | HQRSTYPLT |
| 182 | CD25 VH | QVQLVQSGAEVKKPGSSVKVSCKASGYTFTSYR |
| | | MHWVRQAPGQGLEWIGYINPSTGYTEYNQKFKD |
| | | KATITADESTNTAYMELSSLRSEDTAVYYCARGG |
| | | GVFDYWGQGTLVTVSS |
| 183 | CD25 VL | DIQMTQSPSTLSASVGDRVTITCSASSSISYMHWY |
| | | QQKPGKAPKLLIYTTSNLASGVPARFSGSGSGTEF |
| | | TLTISSLQPDDFATYYCHQRSTYPLTFGQGTKVEV |
| | | K |
| 184 | ADAM9 CDR-H1 | SYWMH |
| 185 | ADAM9 CDR-H2 | EIIPINGHTNYNEKFKS |
| 186 | ADAM9 CDR-H3 | GGYYYYGSRDYFDY |
| 187 | ADAM9 CDR-L1 | KASQSVDYDGDSYMN |
| 188 | ADAM9 CDR-L2 | AASDLES |
| 189 | ADAM9 CDR-L3 | QQSHEDPFT |
| 190 | ADAM9 VH | QVQLQQPGAELVKPGASVKLSCKASGYTFTSYW |
| | | MHWVKQRPGQGLEWIGEIIPINGHTNYNEKFKSK |
| | | ATLTLDKSSSTAYMQLSSLASEDSAVYYCARGGY |
| | | YYYGSRDYFDYWGQGTTLTVSS |
| 191 | ADAM9 VL | DIVLTQSPASLAVSLGQRATISCKASQSVDYDGDS |
| | | YMNWYQQIPGQPPKLLIYAASDLESGIPARFSGSG |
| | | SGTDFTLNIHPVEEEDAATYYCQQSHEDPFTFGGG |
| | | TKLEIK |
| 192 | ADAM9 CDR-H1 | SYWMH |
| 193 | ADAM9 CDR-H2 | EIIPIFGHTNYNEKFKS |
| 194 | ADAM9 CDR-H3 | GGYYYYPRQGFLDY |
| 195 | ADAM9 CDR-L1 | KASQSVDYSGDSYMN |
| 196 | ADAM9 CDR-L2 | AASDLES |
| 197 | ADAM9 CDR-L3 | QQSHEDPFT |
| 198 | ADAM9 VH | EVQLVESGGGLVKPGGSLRLSCAASGFTFSSYWM |
| | | HWVRQAPGKGLEWVGEIIPIFGHTNYNEKFKSRF |
| | | TISLDNSKNTLYLQMGSLRAEDTAVYYCARGGY |
| | | YYYPRQGFLDYWGQGTTVTVSS |
| 199 | ADAM9 VL | DIVMTQSPDSLAVSLGERATISCKASQSVDYSGDS |
| | | YMNWYQQKPGQPPKLLIYAASDLESGIPARFSGS |
| | | GSGTDFTLTISSLEPEDFATYYCQQSHEDPFTFGQ |
| | | GTKLEIK |
| 200 | CD59 CDR-H1 | YGMN |
| 201 | CD59 CDR-H2 | YISSSSSTIYYADSVKG |
| 202 | CD59 CDR-H3 | GPGMDV |
| 203 | CD59 CDR-L1 | KSSQSVLYSSNNKNYLA |
| 204 | CD59 CDR-L2 | WASTRES |
| 205 | CD59 CDR-L3 | QQYYSTPQLT |
| 206 | CD59 VH | QVQLQQSGGGVVQPGRSLGLSCAASGFTFSSYGM |
| | | NWVRQAPGKGLEWVSYISSSSSTIYYADSVKGRF |
| | | TISRDNSKNTLYLQMNSLRAEDTAVYYCARGPG |
| | | MDVWGQGTTVTVS |
| 207 | CD59 VL | DIVLTQSPDSLAVSLGERATINCKSSQSVLYSSNN |
| | | KNYLAWYQQKPGQPPKLLIYWASTRESGVPDRFS |
| | | GSGSGTDFTPAISSLQAEDVAVYYCQQYYSTPQLT |
| | | FGGGTKVDIK |
| 208 | CD19 (hBU12) CDR-H1 | TSGMGVG |
| 209 | CD19 (hBU12) CDR-H2 | HIWWDDDKRYNPALKS |
| 210 | CD19 (hBU12) CDR-H3 | MELWSYYFDY |
| 211 | CD19 (hBU12) CDR-L1 | SASSSVSYMH |
| 212 | CD19 (hBU12) CDR-L2 | DTSKLAS |
| 213 | CD19 (hBU12) CDR-L3 | FQGSVYPFT |
| 214 | CD19 (hBU12) VH | QVQLQESGPGLVKPSQTLSLTCTVSGGSISTSGMG |
| | | VGWIRQHPGKGLEWIGHIWWDDDKRYNPALKSR |
| | | VTISVDTSKNQFSLKLSSVTAADTAVYYCARMEL |
| | | WSYYFDYWGQGTLVTVSS |
| 215 | CD19 (hBU12) VL | EIVLTQSPATLSLSPGERATLSCSASSSVSYMHWY |
| | | QQKPGQAPRLLIYDTSKLASGIPARFSGSGSGTDF |
| | | TLTISSLEPEDVAVYYCFQGSVYPFTFGQGTKLEIK |
| | | R |
| 216 | CD70 CDR-H1 | NYGMN |
| 217 | CD70 CDR-H2 | WINTYTGEPTYADAFKG |
| 218 | CD70 CDR-H3 | DYGDYGMDY |
| 219 | CD70 CDR-L1 | RASKSVSTSGYSFMH |
| 220 | CD70 CDR-L2 | LASNLES |
| 221 | CD70 CDR-L3 | QHSREVPWT |
| 222 | CD70 VH | QVQLVQSGAEVKKPGASVKVSCKASGYTFTNYG |
| | | MNWVRQAPGQGLKWMGWINTYTGEPTYADAFK |
| | | GRVTMTRDTSISTAYMELSRLRSDDTAVYYCARD |
| | | YGDYGMDYWGQGTTVTVSS |
| 223 | CD70 VL | DIVMTQSPDSLAVSLGERATINCRASKSVSTSGYS |
| | | FMHWYQQKPGQPPKLLIYLASNLESGVPDRFSGS |
| | | GSGTDFTLTISSLQAEDVAVYYCQHSREVPWTFG |
| | | QGTKVEIK |
| 224 | B7H4 CDR-H1 | SGYSWH |
| 225 | B7H4 CDR-H2 | YIHSSGSTNYNPSLKS |
| 226 | B7H4 CDR-H3 | YDDYFEY |
| 227 | B7H4 CDR-L1 | KASQNVGFNVA |
| 228 | B7H4 CDR-L2 | SASYRYS |
| 229 | B7H4 CDR-L3 | QQYNWYPFT |
| 230 | B7H4 VH | EVQLQESGPGLVKPSETLSLTCAVTGYSITSGYSW |
| | | HWIRQFPGNGLEWMGYIHSSGSTNYNPSLKSRISI |
| | | SRDTSKNQFFLKLSSVTAADTAVYYCAGYDDYFE |
| | | YWGQGTTVTVSS |
| 231 | B7H4 VL | DIQMTQSPSSLSASVGDRVTITCKASQNVGFNVA |
| | | WYQQKPGKSPKALIYSASYRYSGVPSRFSGSGSG |
| | | TDFTLTISSLQPEDFAEYFCQQYNWYPFTFGQGTK |
| | | LEIK |
| 232 | CD138 CDR-H1 | NYWIE |
| 233 | CD138 CDR-H2 | EILPGTGRTIYNEKFKG |
| 234 | CD138 CDR-H3 | RDYYGNFYYAMDY |
| 235 | CD138 CDR-L1 | SASQGINNYLN |
| 236 | CD138 CDR-L2 | YTSTLQS |
| 237 | CD138 CDR-L3 | QQYSKLPRT |
| 238 | CD138 VH | QVQLQQSGSELMMPGASVKISCKATGYTFSNYWI |
| | | EWVKQRPGHGLEWIGEILPGTGRTIY |
| | | NEKFKGKATFTADISSNTVQMQLSSLTSEDSAVY |
| | | YCARRDYYGNFYYAMDYWGQGTSVTVSS |
| 239 | CD138 VL | DIQMTQSTSSLSASLGDRVTISCSASQGINNYLNW |
| | | YQQKPDGTVELLIYYTSTLQSGVP |
| | | SRFSGSGSGTDYSLTISNLEPEDIGTYYCQQYSKLP |
| | | RTFGGGTKLEIK |
| 240 | CD166 CDR-H1 | TYGMGVG |
| 241 | CD166 CDR-H2 | NIWWSEDKHYSPSLKS |
| 242 | CD166 CDR-H3 | IDYGNDYAFTY |
| 243 | CD166 CDR-L1 | RSSKSLLHSNGITYLY |
| 244 | CD166 CDR-L2 | QMSNLAS |
| 245 | CD166 CDR-L3 | AQNLELPYT |
| 246 | CD166 VH | QITLKESGPTLVKPTQTLTLTCTFSGFSLSTYGMG |
| | | VGWIRQPPGKALEWLANIWWSEDKHYSPSLKSRL |
| | | TITKDTSKNQVVLTITNVDPVDTATYYCVQIDYG |
| | | NDYAFTYWGQGTLVTVSS |
| 247 | CD166 VL | DIVMTQSPLSLPVTPGEPASISCRSSKSLLHSNGIT |
| | | YLYWYLQKPGQSPQLLIYQMSNLASGVPDRFSGS |
| | | GSGTDFTLKISRVEAEDVGVYYCAQNLELPYTFG |
| | | QGTKLEIK |
| 248 | CD51 CDR-H1 | RYTMH |
| 249 | CD51 CDR-H2 | VISFDGSNKYYVDSVKG |
| 250 | CD51 CDR-H3 | EARGSYAFDI |
| 251 | CD51 CDR-L1 | RASQSVSSYLA |
| 252 | CD51 CDR-L2 | DASNRAT |
| 253 | CD51 CDR-L3 | QQRSNWPPFT |
| 254 | CD51 VH | QVQLVESGGGVVQPGRSRRLSCAASGFTFSRYTM |
| | | HWVRQAPGKGLEWVAVISFDGSNKYYVDSVKGR |
| | | FTISRDNSENTLYLQVNILRAEDTAVYYCAREARG |
| | | SYAFDIWGQGTMVTVSS |
| 255 | CD51 VL | EIVLTQSPATLSLSPGERATLSCRASQSVSSYLAW |
| | | YQQKPGQAPRLLIYDASNRATGIPARFSGSGSGTD |
| | | FTLTISSLEPEDFAVYYCQQRSNWPPFTFGPGTKV |
| | | DIK |
| 256 | CD56 CDR-H1 | SFGMH |
| 257 | CD56 CDR-H2 | YISSGSFTIYYADSVKG |
| 258 | CD56 CDR-H3 | MRKGYAMDY |
| 259 | CD56 CDR-L1 | RSSQIIIHSDGNTYLE |
| 260 | CD56 CDR-L2 | KVSNRFS |
| 261 | CD56 CDR-L3 | FQGSHVPHT |
| 262 | CD56 VH | QVQLVESGGGVVQPGRSLRLSCAASGFTFSSFGM |
| | | HWVRQAPGKGLEWVAYISSGSFTIYYADSVKGRF |
| | | TISRDNSKNTLYLQMNSLRAEDTAVYYCARMRK |
| | | GYAMDYWGQGTLVTVSS |
| 263 | CD56 VL | DVVMTQSPLSLPVTLGQPASISCRSSQIIIHSDGNT |
| | | YLEWFQQRPGQSPRRLIYKVSNRFSGVPDRFSGSG |
| | | SGTDFTLKISRVEAEDVGVYYCFQGSHVPHTFGQ |
| | | GTKVEIK |
| 264 | CD74 CDR-H1 | NYGVN |
| 265 | CD74 CDR-H2 | WINPNTGEPTFDDDFKG |
| 266 | CD74 CDR-H3 | SRGKNEAWFAY |
| 267 | CD74 CDR-L1 | RSSQSLVHRNGNTYLH |
| 268 | CD74 CDR-L2 | TVSNRFS |
| 269 | CD74 CDR-L3 | SQSSHVPPT |
| 270 | CD74 VH | QVQLQQSGSELKKPGASVKVSCKASGYTFTNYG |
| | | VNWIKQAPGQGLQWMGWINPNTGEPTFDDDFKG |
| | | RFAFSLDTSVSTAYLQISSLKADDTAVYFCSRSRG |
| | | KNEAWFAYWGQGTLVTVSS |
| 271 | CD74 VL | DIQLTQSPLSLPVTLGQPASISCRSSQSLVHRNGNT |
| | | YLHWFQQRPGQSPRLLIYTVSNRFSGVPDRFSGSG |
| | | SGTDFTLKISRVEAEDVGVYFCSQSSHVPPTFGAG |
| | | TRLEIK |
| 272 | CEACAM5 CDR-H1 | TYWMS |
| 273 | CEACAM5 CDR-H2 | EIHPDSSTINYAPSLKD |
| 274 | CEACAM5 CDR-H3 | LYFGFPWFAY |
| 275 | CEACAM5 CDR-L1 | KASQDVGTSVA |
| 276 | CEACAM5 CDR-L2 | WTSTRHT |
| 277 | CEACAM5 CDR-L3 | QQYSLYRS |
| 278 | CEACAM5 VH | EVQLVESGGGVVQPGRSLRLSCSASGFDFTTYWM |
| | | SWVRQAPCKGLEWIGEIHPDSSTINYAPSLKDRFT |
| | | ISRDNAKNTLFLQMDSLRPEDTGVYFCASLYFGFP |
| | | WFAYWGQGTPVTVSS |
| 279 | CEACAM5 VL | DIQLTQSPSSLSASVGDRVTITCKASQDVGTSVAW |
| | | YQQKPGKAPKLLIYWTSTRHTGVPSRFSGSGSGT |
| | | DFTFTISSLQPEDIATYYCQQYSLYRSFGQGTKVEI |
| | | K |
| 280 | CanAg CDR-H1 | YYGMN |
| 281 | CanAg CDR-H2 | WIDTTTGEPTYAQKFQG |
| 282 | CanAg CDR-H3 | RGPYNWYFDV |
| 283 | CanAg CDR-L1 | RSSKSLLHSNGNTYLY |
| 284 | CanAg CDR-L2 | RMSNLVS |
| 285 | CanAg CDR-L3 | LQHLEYPFT |
| 286 | CanAg VH | QVQLVQSGAEVKKPGETVKISCKASDYTFTYYG |
| | | MNWVKQAPGQGLKWMGWIDTTTGEPTYAQKFQ |
| | | GRIAFSLETSASTAYLQIKSLKSEDTATYFCARRGP |
| | | YNWYFDVWGQGTTVTVSS |
| 287 | CanAg VL | DIVMTQSPLSVPVTPGEPVSISCRSSKSLLHSNGNT |
| | | YLYWFLQRPGQSPQLLIYRMSNLVSGVPDRFSGS |
| | | GSGTAFTLRISRVEAEDVGVYYCLQHLEYPFTFGP |
| | | GTKLELK |
| 288 | DLL-3 CDR-H1 | NYGMN |
| 289 | DLL-3 CDR-H2 | WINTYTGEPTYADDFKG |
| 290 | DLL-3 CDR-H3 | IGDSSPSDY |
| 291 | DLL-3 CDR-L1 | KASQSVSNDVV |
| 292 | DLL-3 CDR-L2 | YASNRYT |
| 293 | DLL-3 CDR-L3 | QQDYTSPWT |
| 294 | DLL-3 VH | QVQLVQSGAEVKKPGASVKVSCKASGYTFTNYG |
| | | MNWVRQAPGQGLEWMGWINTYTGEPTY |
| | | ADDFKGRVTMTTDTSTSTAYMELRSLRSDDTAV |
| | | YYCARIGDSSPSDYWGQGTLVTVSS |
| 295 | DLL-3 VL | EIVMTQSPATLSVSPGERATLSCKASQSVSNDVV |
| | | WYQQKPGQAPRLLIYYASNRYTGIPA |
| | | RFSGSGSGTEFTLTISSLQSEDFAVYYCQQDYTSP |
| | | WTFGQGTKLEIK |
| 296 | DPEP-3 CDR-H1 | SYWIE |
| 297 | DPEP-3 CDR-H2 | EILPGSGNTYYNERFKD |
| 298 | DPEP-3 CDR-H3 | RAAAYYSNPEWFAY |
| 299 | DPEP-3 CDR-L1 | TASSSVNSFYLH |
| 300 | DPEP-3 CDR-L2 | STSNLAS |
| 301 | DPEP-3 CDR-L3 | HQYHRSPYT |
| 302 | DPEP-3 VH | QVQLVQSGAEVKKPGSSVKVSCKASGGTFSSYWI |
| | | EWVRQAPGQGLEWMGEILPGSGNTYYNERFKDR |
| | | VTITADESTSTAYMELSSLRSEDTAVYYCARRAA |
| | | AYYSNPEWFAYWGQGTLVTVSS |
| 303 | DPEP-3 VL | EIVLTQSPATLSLSPGERATLSCTASSSVNSFYLHW |
| | | YQQKPGLAPRLLIYSTSNLASGIPDRFSGSGSGTDF |
| | | TLTISRLEPEDFAVYYCHQYHRSPYTFGQGTKLEI |
| | | K |
| 304 | EGFR CDR-H1 | SYWMQ |
| 305 | EGFR CDR-H2 | TIYPGDGDTTYTQKFQG |
| 306 | EGFR CDR-H3 | YDAPGYAMDY |
| 307 | EGFR CDR-L1 | RASQDINNYLA |
| 308 | EGFR CDR-L2 | YTSTLHP |
| 309 | EGFR CDR-L3 | LQYDNLLYT |
| 310 | EGFR VH | QVQLVQSGAEVAKPGASVKLSCKASGYTFTSYW |
| | | MQWVKQRPGQGLECIGTIYPGDGDTTYTQKFQG |
| | | KATLTADKSSSTAYMQLSSLRSEDSAVYYCARYD |
| | | APGYAMDYWGQGTLVTVSS |
| 311 | EGFR VL | DIQMTQSPSSLSASVGDRVTITCRASQDINNYLAW |
| | | YQHKPGKGPKLLIHYTSTLHPGIPSRFSGSGSGRD |
| | | YSFSISSLEPEDIATYYCLQYDNLLYTFGQGTKLEI |
| | | K |
| 312 | EGFR CDR-H1 | RDFAWN |
| 313 | EGFR CDR-H2 | YISYNGNTRYQPSLKS |
| 314 | EGFR CDR-H3 | ASRGFPY |
| 315 | EGFR CDR-L1 | HSSQDINSNIG |
| 316 | EGFR CDR-L2 | HGTNLDD |
| 317 | EGFR CDR-L3 | VQYAQFPWT |
| 318 | EGFR VH | EVQLQESGPGLVKPSQTLSLTCTVSGYSISRDFAW |
| | | NWIRQPPGKGLEWMGYISYNGNTRYQPSLKSRITI |
| | | SRDTSKNQFFLKLNSVTAADTATYYCVTASRGFP |
| | | YWGQGTLVTVSS |
| 319 | EGFR VL | DIQMTQSPSSMSVSVGDRVTITCHSSQDINSNIGW |
| | | LQQKPGKSFKGLIYHGTNLDDGVPSRFSGSGSGT |
| | | DYTLTISSLQPEDFATYYCVQYAQFPWTFGGGTK |
| | | LEIK |
| 320 | EGFR CDR-H1 | RDFAWN |
| 321 | EGFR CDR-H2 | YISYNGNTRYQPSLKS |
| 322 | EGFR CDR-H3 | ASRGFPY |
| 323 | EGFR CDR-L1 | HSSQDINSNIG |
| 324 | EGFR CDR-L2 | HGTNLDD |
| 325 | EGFR CDR-L3 | VQYAQFPWT |
| 326 | EGFR VH | EVQLQESGPGLVKPSQTLSLTCTVSGYSISRDFAW |
| | | NWIRQPPGKGLEWMGYISYNGNTRYQPSLKSRITI |
| | | SRDTSKNQFFLKLNSVTAADTATYYCVTASRGFP |
| | | YWGQGTLVTVSS |
| 327 | EGFR VL | DIQMTQSPSSMSVSVGDRVTITCHSSQDINSNIGW |
| | | LQQKPGKSFKGLIYHGTNLDDGVPSRFSGSGSGT |
| | | DYTLTISSLQPEDFATYYCVQYAQFPWTFGGGTK |
| | | LEIK |
| 328 | EGFR CDR-H1 | NYGVH |
| 329 | EGFR CDR-H2 | VIWSGGNTDYNTPFTS |
| 330 | EGFR CDR-H3 | ALTYYDYEFAY |
| 331 | EGFR CDR-L1 | RASQSIGTNIH |
| 332 | EGFR CDR-L2 | YASESIS |
| 333 | EGFR CDR-L3 | QQNNNWPTT |
| 334 | EGFR VH | QVQLKQSGPGLVQPSQSLSITCTVSGFSLTNYGVH |
| | | WVRQSPGKGLEWLGVIWSGGNTDYNTPFTSRLSI |
| | | NKDNSKSQVFFKMNSLQSNDTAIYYCARALTYY |
| | | DYEFAYWGQGTLVTVSA |
| 335 | EGFR VL | DILLTQSPVILSVSPGERVSFSCRASQSIGTNIHWY |
| | | QQRTNGSPRLLIKYASESISGIPSRFSGSGSGTDFTL |
| | | SINSVESEDIADYYCQQNNNWPTTFGAGTKLELK |
| 336 | FRa CDR-H1 | GYFMN |
| 337 | FRa CDR-H2 | RIHPYDGDTFYNQKFQG |
| 338 | FRa CDR-H3 | YDGSRAMDY |
| 339 | FRa CDR-L1 | KASQSVSFAGTSLMH |
| 340 | FRa CDR-L2 | RASNLEA |
| 341 | FRa CDR-L3 | QQSREYPYT |
| 342 | FRa VH | QVQLVQSGAEVVKPGASVKISCKASGYTFTGYFM |
| | | NWVKQSPGQSLEWIGRIHPYDGDTFYNQKFQGK |
| | | ATLTVDKSSNTAHMELLSLTSEDFAVYYCTRYDG |
| | | SRAMDYWGQGTTVTVSS |
| 343 | FRa VL | DIVLTQSPLSLAVSLGQPAIISCKASQSVSFAGTSL |
| | | MHWYHQKPGQQPRLLIYRASNLEAGVPDRFSGS |
| | | GSKTDFTLTISPVEAEDAATYYCQQSREYPYTFGG |
| | | GTKLEIK |
| 344 | FRa CDR-H1 | GYGLS |
| 345 | FRa CDR-H2 | MISSGGSYTYYADSVKG |
| 346 | FRa CDR-H3 | HGDDPAWFAY |
| 347 | FRa CDR-L1 | SVSSSISSNNLH |
| 348 | FRa CDR-L2 | GTSNLAS |
| 349 | FRa CDR-L3 | QQWSSYPYMYT |
| 350 | FRa VH | EVQLVESGGGVVQPGRSLRLSCSASGFTFSGYGLS |
| | | WVRQAPGKGLEWVAMISSGGSYTYY |
| | | ADSVKGRFAISRDNAKNTLFLQMDSLRPEDTGVY |
| | | FCARHGDDPAWFAYWGQGTPVTVSS |
| 351 | FRa VL | DIQLTQSPSSLSASVGDRVTITCSVSSSISSNNLHW |
| | | YQQKPGKAPKPWIYGTSNLASGVPSRFSGSGSGT |
| | | DYTFTISSLQPEDIATYYCQQWSSYPYMYTFGQGT |
| | | KVEIK |
| 352 | MUC-1 CDR-H1 | NYWMN |
| 353 | MUC-1 CDR-H2 | EIRLKSNNYTTHYAESVKG |
| 354 | MUC-1 CDR-H3 | HYYFDY |
| 355 | MUC-1 CDR-L1 | RSSKSLLHSNGITYFF |
| 356 | MUC-1 CDR-L2 | QMSNLAS |
| 357 | MUC-1 CDR-L3 | AQNLELPPT |
| 358 | MUC-1 VH | EVQLVESGGGLVQPGGSMRLSCVASGFPFSNYW |
| | | MNWVRQAPGKGLEWVGEIRLKSNNYTTHYAESV |
| | | KGRFTISRDDSKNSLYLQMNSLKTEDTAVYYCTR |
| | | HYYFDYWGQGTLVTVSS |
| 359 | MUC-1 VL | DIVMTQSPLSNPVTPGEPASISCRSSKSLLHSNGIT |
| | | YFFWYLQKPGQSPQLLIYQMSNLASGVPDRFSGS |
| | | GSGTDFTLRISRVEAEDVGVYYCAQNLELPPTFGQ |
| | | GTKVEIK |
| 360 | Mesothelin CDR-H1 | SYWIG |
| 361 | Mesothelin CDR-H2 | IIDPGDSRTRYSPSFQG |
| 362 | Mesothelin CDR-H3 | GQLYGGTYMDG |
| 363 | Mesothelin CDR-L1 | TGTSSDIGGYNSVS |
| 364 | Mesothelin CDR-L2 | GVNNRPS |
| 365 | Mesothelin CDR-L3 | SSYDIESATPV |
| 366 | Mesothelin VH | QVELVQSGAEVKKPGESLKISCKGSGYSFTSYWIG |
| | | WVRQAPGKGLEWMGIIDPGDSRTRYSPSFQGQVT |
| | | ISADKSISTAYLQWSSLKASDTAMYYCARGQLYG |
| | | GTYMDGWGQGTLVTVSS |
| 367 | Mesothelin VL | DIALTQPASVSGSPGQSITISCTGTSSDIGGYNSVS |
| | | WYQQHPGKAPKLMIYGVNNRPSGV |
| | | SNRFSGSKSGNTASLTISGLQAEDEADYYCSSYDI |
| | | ESATPVFGGGTKLTVL |
| 368 | ROR-1 CDR-H1 | AYNIH |
| 369 | ROR-1 CDR-H2 | SFDPYDGGSSYNQKFKD |
| 370 | ROR-1 CDR-H3 | GWYYFDY |
| 371 | ROR-1 CDR-L1 | RASKSISKYLA |
| 372 | ROR-1 CDR-L2 | SGSTLQS |
| 373 | ROR-1 CDR-L3 | QQHDESPYT |
| 374 | ROR-1 VH | QVQLQESGPGLVKPSQTLSLTCTVSGYAFTAYNIH |
| | | WVRQAPGQGLEWMGSFDPYDGGSSYNQKFKDR |
| | | LTISKDTSKNQVVLTMTNMDPVDTATYYCARGW |
| | | YYFDYWGHGTLVTVSS |
| 375 | ROR-1 VL | DIVMTQTPLSLPVTPGEPASISCRASKSISKYLAWY |
| | | QQKPGQAPRLLIYSGSTLQSGIPPRFSGSGYGTDFT |
| | | LTINNIESEDAAYYFCQQHDESPYTFGEGTKVEIK |
| 376 | B7H4 CDR-H1 | GSIKSGSYYWG |
| 377 | B7H4 CDR-H2 | NIYYSGSTYYNPSLRS |
| 378 | B7H4 CDR-H3 | AREGSYPNQFDP |
| 379 | B7H4 CDR-L1 | RASQSVSSNLA |
| 380 | B7H4 CDR-L2 | GASTRAT |
| 381 | B7H4 CDR-L3 | QQYHSFPFT |
| 382 | B7H4 VH | QLQLQESGPGLVKPSETLSLTCTVSGGSIKSGSYY |
| | | WGWIRQPPGKGLEWIGNIYYSGSTY |
| | | YNPSLRSRVTISVDTSKNQFSLKLSSVTAADTAVY |
| | | YCAREGSYPNQFDPWGQGTLVTVSS |
| 383 | B7H4 VL | EIVMTQSPATLSVSPGERATLSCRASQSVSSNLAW |
| | | YQQKPGQAPRLLIYGASTRATGIPA |
| | | RFSGSGSGTEFTLTISSLQSEDFAVYYCQQYHSFPF |
| | | TFGGGTKVEIK |
| 384 | B7-H3 CDR-H1 | SFGMH |
| 385 | B7-H3 CDR-H2 | YISSDSSAIYYADTVKG |
| 386 | B7-H3 CDR-H3 | GRENIYYGSRLDY |
| 387 | B7-H3 CDR-L1 | KASQNVDTNVA |
| 388 | B7-H3 CDR-L2 | SASYRYS |
| 389 | B7-H3 CDR-L3 | QQYNNYPFT |
| 390 | B7-H3 VH | DVQLVESGGGLVQPGGSRKLSCAASGFTFSSFGM |
| | | HWVRQAPEKGLEWVAYISSDSSAIYYADTVKGRF |
| | | TISRDNPKNTLFLQMTSLRSEDTAMYYCGRGRENI |
| | | YYGSRLDYWGQGTTLTVSS |
| 391 | B7-H3 VL | DIAMTQSQKFMSTSVGDRVSVTCKASQNVDTNV |
| | | AWYQQKPGQSPKALIYSASYRYSGVPDRFTGSGS |
| | | GTDFTLTINNVQSEDLAEYFCQQYNNYPFTFGSGT |
| | | KLEIK |
| 392 | B7-H3 CDR-H1 | SYWMQWVRQA |
| 393 | B7-H3 CDR-H2 | TIYPGDGDTRY |
| 394 | B7-H3 CDR-H3 | RGIPRLWYFDVM |
| 395 | B7-H3 CDR-L1 | ITCRASQDIS |
| 396 | B7-H3 CDR-L2 | YTSRLHSGVPS |
| 397 | B7-H3 CDR-L3 | QQGNTLPPFTGG |
| 398 | B7-H3 VH | DVQLVESGGGLVQPGGSRKLSCAASGFTFSSFGM |
| | | HWVRQAPEKGLEWVAYISSDSSAIYY |
| | | ADTVKGRFTISRDNPKNTLFLQMTSLRSEDTAMY |
| | | YCGRGRENIYYGSRLDYWGQGTTLTVSS |
| 399 | B7-H3 VL | DIAMTQSQKFMSTSVGDRVSVTCKASQNVDTNV |
| | | AWYQQKPGQSPKALIYSASYRYSGVPD |
| | | RFTGSGSGTDFTLTINNVQSEDLAEYFCQQYNNYP |
| | | FTFGSGTKLEIK |
| 400 | B7-H3 CDR-H1 | SYGMS |
| 401 | B7-H3 CDR-H2 | TINSGGSNTYYPDSLKG |
| 402 | B7-H3 CDR-H3 | HDGGAMDY |
| 403 | B7-H3 CDR-L1 | RASESIYSYLA |
| 404 | B7-H3 CDR-L2 | NTKTLPE |
| 405 | B7-H3 CDR-L3 | QHHYGTPPWT |
| 406 | B7-H3 VH | EVQLVESGGGLVKPGGSLRLSCAASGFTFSSYGM |
| | | SWVRQAPGKGLEWVATINSGGSNTYY |
| | | PDSLKGRFTISRDNAKNSLYLQMNSLRAEDTAVY |
| | | YCARHDGGAMDYWGQGTTVTVSS |
| 407 | B7-H3 VL | DIQMTQSPSSLSASVGDRVTITCRASESIYSYLAW |
| | | YQQKPGKAPKLLVYNTKTLPEGVPSRFSGSGSGT |
| | | DFTLTISSLQPEDFATYYCQHHYGTPPWTFGQGTR |
| | | LEIK |
| 408 | B7-H3 CDR-H1 | SFGMH |
| 409 | B7-H3 CDR-H2 | YISSGSGTIYYADTVKG |
| 410 | B7-H3 CDR-H3 | HGYRYEGFDY |
| 411 | B7-H3 CDR-L1 | KASQNVDTNVA |
| 412 | B7-H3 CDR-L2 | SASYRYS |
| 413 | B7-H3 CDR-L3 | QQYNNYPFT |
| 414 | B7-H3 VH | EVQLVESGGGLVQPGGSLRLSCAASGFTFSSFGM |
| | | HWVRQAPGKGLEWVAYISSGSGTIYYADTVKGR |
| | | FTISRDNAKNSLYLQMNSLRAEDTAVYYCARHG |
| | | YRYEGFDYWGQGTTVTVSS |
| 415 | B7-H3 VL | DIQMTQSPSFLSASVGDRVTITCKASQNVDTNVA |
| | | WYQQKPGKAPKALIYSASYRYSGVPSRFSGSGSG |
| | | TDFTLTISSLQPEDFAEYFCQQYNNYPFTFGQGTK |
| | | LEIK |
| 416 | B7-H3 CDR-H1 | NYVMH |
| 417 | B7-H3 CDR-H2 | YINPYNDDVKYNEKFKG |
| 418 | B7-H3 CDR-H3 | WGYYGSPLYYFDY |
| 419 | B7-H3 CDR-L1 | RASSRLIYMH |
| 420 | B7-H3 CDR-L2 | ATSNLAS |
| 421 | B7-H3 CDR-L3 | QQWNSNPPT |
| 422 | B7-H3 VH | EVQLQQSGPELVKPGASVKMSCKASGYTFTNYV |
| | | MHWVKQKPGQGLEWIGYINPYNDDVKYNEKFK |
| | | GKATQTSDKSSSTAYMELSSLTSEDSAVYYCARW |
| | | GYYGSPLYYFDYWGQGTTLTVSS |
| 423 | B7-H3 VL | QIVLSQSPTILSASPGEKVTMTCRASSRLIYMHWY |
| | | QQKPGSSPKPWIYATSNLASGVPAR |
| | | FSGSGSGTSYSLTISRVEAEDAATYYCQQWNSNPP |
| | | TFGTGTKLELK |
| 424 | B7-H3 CDR-H1 | NYVMH |
| 425 | B7-H3 CDR-H2 | YINPYNDDVKYNEKFKG |
| 426 | B7-H3 CDR-H3 | WGYYGSPLYYFDY |
| 427 | B7-H3 CDR-L1 | RASSRLIYMH |
| 428 | B7-H3 CDR-L2 | ATSNLAS |
| 429 | B7-H3 CDR-L3 | QQWNSNPPT |
| 430 | B7-H3 VH | QVQLVQSGAEVKKPGSSVKVSCKASGYTFTNYV |
| | | MHWVRQAPGQGLEWMGYINPYNDDVKYNE |
| | | KFKGRVTITADESTSTAYMELSSLRSEDTAVYYC |
| | | ARWGYYGSPLYYFDYWGQGTLVTVSS |
| 431 | B7-H3 VL | EIVLTQSPATLSLSPGERATLSCRASSRLIYMHWY |
| | | QQKPGQAPRPLIYATSNLASGIPARFSGSGSGTDF |
| | | TLTISSLEPEDFAVYYCQQWNSNPPTFGQGTKVEI |
| | | K |
| 432 | B7-H3 CDR-H1 | SYTIH |
| 433 | B7-H3 CDR-H2 | YINPNSRNTDYAQKFQG |
| 434 | B7-H3 CDR-H3 | YSGSTPYWYFDV |
| 435 | B7-H3 CDR-L1 | RASSSVSYMN |
| 436 | B7-H3 CDR-L2 | ATSNLAS |
| 437 | B7-H3 CDR-L3 | QQWSSNPLT |
| 438 | B7-H3 VH | EVQLVQSGAEVKKPGSSVKVSCKASGYSFTSYTI |
| | | HWVRQAPGQGLEWMGYINPNSRNTDYAQKFQG |
| | | RVTLTADKSTSTAYMELSSLRSEDTAVYYCARYS |
| | | GSTPYWYFDVWGQGTTVTVSS |
| 439 | B7-H3 VL | DIQLTQSPSFLSASVGDRVTITCRASSSVSYMNWY |
| | | QQKPGKSPKPWIYATSNLASGVPSRFSVSVSGTEH |
| | | TLTISSLQPEDFATYYCQQWSSNPLTFGQGTKLEI |
| | | K |
| 440 | B7-H3 CDR-H1 | SYWMH |
| 441 | B7-H3 CDR-H2 | LIHPDSGSTNYNEMFKN |
| 442 | B7-H3 CDR-H3 | GGRLYFD |
| 443 | B7-H3 CDR-L1 | RSSQSLVHSNGDTYLR |
| 444 | B7-H3 CDR-L2 | KVSNRFS |
| 445 | B7-H3 CDR-L3 | SQSTHVPYT |
| 446 | B7-H3 VH | EVQLVQSGAEVKKPGSSVKVSCKASGYTFSSYW |
| | | MHWVRQAPGQGLEWIGLIHPDSGSTNYNEMFKN |
| | | RATLTVDRSTSTAYVELSSLRSEDTAVYFCAGGG |
| | | RLYFDYWGQGTTVTVSS |
| 447 | B7-H3 VL | DVVMTQSPLSLPVTPGEPASISCRSSQSLVHSNGD |
| | | TYLRWYLQKPGQSPQLLIYKVSNRFSGVPDRFSG |
| | | SGSGTDFTLKISRVEAEDVGVYYCSQSTHVPYTFG |
| | | GGTKVEIK |
| 448 | B7-H3 CDR-H1 | SYWMH |
| 449 | B7-H3 CDR-H2 | LIHPESGSTNYNEMFKN |
| 450 | B7-H3 CDR-H3 | GGRLYFDY |
| 451 | B7-H3 CDR-L1 | RSSQSLVHSNQDTYLR |
| 452 | B7-H3 CDR-L2 | KVSNRFS |
| 453 | B7-H3 CDR-L3 | SQSTHVPYT |
| 454 | B7-H3 VH | EVQLVQSGAEVKKPGSSVKVSCKASGYTFSSYW |
| | | MHWVRQAPGQGLEWIGLIHPESGSTNYNEMFKN |
| | | RATLTVDRSTSTAYMELSSLRSEDTAVYYCAGGG |
| | | RLYFDYWGQGTTVTVSS |
| 455 | B7-H3 VL | DIVMTQSPLSLPVTPGEPASISCRSSQSLVHSNQDT |
| | | YLRWYLQKPGQSPQLLIYKVSNRF |
| | | SGVPDRFSGSGSGTDFTLKKISRVEAEDVGVYYCS |
| | | QSTHVPYTFGGGTKVEIK |
| 456 | B7-H3 CDR-H1 | SGYSWH |
| 457 | B7-H3 CDR-H2 | YIHSSGSTNYNPSLKS |
| 458 | B7-H3 CDR-H3 | YDDYFEY |
| 459 | B7-H3 CDR-L1 | KASQNVGENVA |
| 460 | B7-H3 CDR-L2 | SASYRYS |
| 461 | B7-H3 CDR-L3 | QQYNWYPFT |
| 462 | B7-H3 VH | EVQLQESGPGLVKPSETLSLTCAVTGYSITSGYSW |
| | | HWIRQFPGNGLEWMGYIHSSGSTNY |
| | | NPSLKSRISISRDTSKNQFFLKLSSVTAADTAVYY |
| | | CAGYDDYFEYWGQGTTVTVSS |
| 463 | B7-H3 VL | DIQMTQSPSSLSASVGDRVTITCKASQNVGFNVA |
| | | WYQQKPGKSPKALIYSASYRYSGVPSRFSGSGSG |
| | | TDFTLTISSLQPEDFAEYFCQQYNWYPFTFGQGTK |
| | | LEIK |
| 464 | B7-H3 CDR-H1 | NYDIN |
| 465 | B7-H3 CDR-H2 | WIFPGDDSTQYNEKFKG |
| 466 | B7-H3 CDR-H3 | QTTGTWFAY |
| 467 | B7-H3 CDR-L1 | RASQSISDYLY |
| 468 | B7-H3 CDR-L2 | YASQSIS |
| 469 | B7-H3 CDR-L3 | QNGHSFPLT |
| 470 | B7-H3 VH | QVQLVQSGAEVVKPGASVKLSCKTSGYTFTNYDI |
| | | NWVRQRPGQGLEWIGWIFPGDDSTQY |
| | | NEKFKGKATLTTDTSTSTAYMELSSLRSEDTAVY |
| | | FCARQTTGTWFAYWGQGTLVTVSS |
| 471 | B7-H3 VL | EIVMTQSPATLSVSPGERVTLSCRASQSISDYLYW |
| | | YQQKSHESPRLLIKYASQSISGIPA |
| | | RFSGSGSGSEFTLTINSVEPEDVGVYYCQNGHSFP |
| | | LTFGQGTKLELK |
| 472 | B7-H3 VH | QVQLQQSGAEVKKPGSSVKVSCKASGGTFSSYAI |
| | | SWVRQAPGQGLEWMGGIIPILGIAN |
| | | YAQKFQGRVTITADESTSTAYMELSSLRSEDTAV |
| | | YYCARGGSGSYHMDVWGKGTTVTVSS |
| 473 | B7-H3 VL | EIVLTQSPATLSLSPGERATLSCRASQSVSSYLAW |
| | | YQQKPGQAPRLLIYDASNRATGIP |
| | | ARFSGSGSGTDFTLTISSLEPEDFAVYYCQQRSNW |
| | | PPRITFGQGTRLEIK |
| 474 | B7-H3 CDR-H1 | IYNVH |
| 475 | B7-H3 CDR-H2 | TIFPGNGDTSYNQKFKD |
| 476 | B7-H3 CDR-H3 | WDDGNVGFAH |
| 477 | B7-H3 CDR-L1 | RASENINNYLT |
| 478 | B7-H3 CDR-L2 | HAKTLAE |
| 479 | B7-H3 CDR-L3 | QHHYGTPPT |
| 480 | B7-H3 VH | QVQLQQPGAELVKPGASVKMSCKASGYTFTIYNV |
| | | HWIKQTPGQGLEWMGTIFPGNGDTSY |
| | | NQKFKDKATLTTDKSSKTAYMQLNSLTSEDSAVY |
| | | YCARWDDGNVGFAHWGQGTLVTVSA |
| 481 | B7-H3 VL | DIQMTQSPASLSASVGETVTITCRASENINNYLTW |
| | | FQQKQGKSPQLLVYHAKTLAEGVPS |
| | | RFSGSGSGTQFSLKINSLQPEDFGSYYCQHHYGTP |
| | | PTFGGGTKLEIK |
| 482 | B7-H3 VH | EVQLVQSGAEVKKPGASVKVSCKASGYTFTIYNV |
| | | HWVRQAPGQGLEWMGTIFPGNGDTS |
| | | YNQKFKDKVTMTTDTSTSTAYMELSSLRSEDTAV |
| | | YYCARWDDGNVGFAHWGQGTLVTVSS |
| 483 | B7-H3 VL | DIQMTQSPSSLSASVGDRVTITCRASENINNYLTW |
| | | FQQKQGKSPQLLIYHAKTLAEGVP |
| | | SRFSGSGSGTDFTLTISSLQPEDFATYYCQHHYGT |
| | | PPTFGGGTKVEIK |
| 484 | B7-H3 VH | EVQLVQSGAEVKKPGASVKVSCKASGYTFTIYNV |
| | | HWIRQAPGQGLEWMGTIFPGNGDTSY |
| | | NQKFKDRATLTTDKSTKTAYMELRSLRSDDTAV |
| | | YYCARWDDGNVGFAHWGQGTLVTVSS |
| 485 | B7-H3 VL | DIQMTQSPSSLSASVGDRVTITCRASENINNYLTW |
| | | FQQKPGKAPKLLVYHAKTLAEGVPS |
| | | RFSGSGSGTQFTLTISSLQPEDFATYYCQHHYGTP |
| | | PTFGQGTKLEIK |
| 486 | HER3 H | QVQLQQWGAGLLKPSETLSLTCAVYGGSFSGYY |
| | | WSWIRQPPGKGLEWIGEINHSGSTNYN |
| | | PSLKSRVTISVETSKNQFSLKLSSVTAADTAVYYC |
| | | ARDKWTWYFDLWGRGTLVTVSSAST |
| | | KGPSVFPLAPSSKSTSGGTAALGCLVKDYFPEPVT |
| | | VSWNSGALTSGVHTFPAVLQSSGL |
| | | YSLSSVVTVPSSSLGTQTYICNVNHKPSNTKVDKR |
| | | VEPKSCDKTHTCPPCPAPELLGGPSVFLFPPKPKD |
| | | TLMISRTPEVTCVVVDVSHEDPEVKFNWYVDGVE |
| | | VHNAKTKPREEQYNSTYRVVSVLTVLHQDWLNG |
| | | KEYKCKVSNKALPAPIEKTISKAKGQPREPQVYTL |
| | | PPSREEMTKNQVSLTCLVKGFYPSDIAVEWESNG |
| | | QPENNYKTTPPVLDSDGSFFLYSKLTVDKSRWQQ |
| | | GNVFSCSVMHEALHNHYTQKSLSLSPGK |
| 487 | HER3 L | DIEMTQSPDSLAVSLGERATINCRSSQSVLYSSSN |
| | | RNYLAWYQQNPGQPPKLLIYWASTRESGVPDRFS |
| | | GSGSGTDFTLTISSLQAEDVAVYYCQQYYSTPRTF |
| | | GQGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| 488 | HER3 H | EVQLLESGGGLVQPGGSLRLSCAASGFTFSHYVM |
| | | AWVRQAPGKGLEWVSSISSSGGWTLY |
| | | ADSVKGRFTISRDNSKNTLYLQMNSLRAEDTAVY |
| | | YCTRGLKMATIFDYWGQGTLVTVSSA |
| | | STKGPSVFPLAPCSRSTSESTAALGCLVKDYFPEP |
| | | VTVSWNSGALTSGVHTFPAVLQSSG |
| | | LYSLSSVVTVPSSNFGTQTYTCNVDHKPSNTKVD |
| | | KTVERKCCVECPPCPAPPVAGPSVFL |
| | | FPPKPKDTLMISRTPEVTCVVVDVSHEDPEVQFN |
| | | WYVDGVEVHNAKTKPREEQFNSTFRV |
| | | VSVLTVVHQDWLNGKEYKCKVSNKGLPAPIEKTI |
| | | SKTKGQPREPQVYTLPPSRFEMTKNQ |
| | | VSLTCLVKGFYPSDIAVEWESNGQPENNYKTTPP |
| | | MLDSDGSFFLYSKLTVDKSRWQQGNV |
| | | FSCSVMHEALHNHYTQKSLSLSPGK |
| 489 | HER3 L | QSALTQPASVSGSPGQSITISCTGTSSDVGSYNVVS |
| | | WYQQHPGKAPKLIIYEVSQRPSGVSNRFSGSKSG |
| | | NTASLTISGLQTEDEADYYCCSYAGSSIFVIFGGGT |
| | | KVTVLGQPKAAPSVTLFPPSSEELQANKATLVCL |
| | | VSDFYPGAVTVAWKADGSPVKVGVETTKPSKQS |
| | | NNKYAASSYLSLTPEQWKSHRSYSCRVTHEGSTV |
| | | EKTVAPAECS |
| 490 | HER3 H | EVQLLESGGGLVQPGGSLRLSCAASGFTFSSYAM |
| | | SWVRQAPGKGLEWVSAINSQGKSTYYADSVKGR |
| | | FTISRDNSKNTLYLQMNSLRAEDTAVYYCARWG |
| | | DEGFDIWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSL |
| | | SPGK |
| 491 | HER3 L | DIQMTQSPSSLSASVGDRVTITCRASQGISNWLAW |
| | | YQQKPGKAPKLLIYGASSLQSGVPSRFSGSGSGTD |
| | | FTLTISSLQPEDFATYYCQQYSSFPTTFGQGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 492 | HER3 H | QVQLVQSGAEVKKPGASVKVSCKASGYTFRSSYI |
| | | SWVRQAPGQGLEWMGWIYAGTGSPSYNQKLQG |
| | | RVTMTTDTSTSTAYMELRSLRSDDTAVYYCARH |
| | | RDYYSNSLTYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTI |
| | | SKAKGQPREPQVYTLPPSRDELTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPG |
| 493 | HER3 L | DIVMTQSPDSLAVSLGERATINCKSSQSVLNSGNQ |
| | | KNYLTWYQQKPGQPPKLLIYWASTRESGVPDRFS |
| | | GSGSGTDFTLTISSLQAEDVAVYYCQSDYSYPYTF |
| | | GQGTKLEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| 494 | PTK7 CDR-H1 | TSNMGVG |
| 495 | PTK7 CDR-H2 | HIWWDDDKYYSPSLKS |
| 496 | PTK7 CDR-H3 | SNYGYAWFAY |
| 497 | PTK7 CDR-L1 | KASQDIYPYLN |
| 498 | PTK7 CDR-L2 | RTNRLLD |
| 499 | PTK7 CDR-L3 | LQYDEFPLT |
| 500 | PTK7 VH | QITLKESGPTLVKPTQTLTLTCTFSGFSLSTSNMGV |
| | | GWIRQPPGKALEWLAHIWWDDDKYYSPSLKSRL |
| | | TITKDTSKNQVVLTMTNMDPVDTATYYCVRSNY |
| | | GYAWFAYWGQGTLVTVSS |
| 501 | PTK7 VL | DIQMTQSPSSLSASVGDRVTITCKASQDIYPYLNW |
| | | FQQKPGKAPKTLIYRTNRLLDGVPS |
| | | RFSGSGSGTDFTFTISSLQPEDIATYYCLQYDEFPL |
| | | TFGAGTKLEIK |
| 502 | PTK7 CDR-H1 | DYAVH |
| 503 | PTK7 CDR-H2 | VISTYNDYTYNNQDFKG |
| 504 | PTK7 CDR-H3 | GNSYFYALDY |
| 505 | PTK7 CDR-L1 | RASESVDSYGKSFMH |
| 506 | PTK7 CDR-L2 | RASNLES |
| 507 | PTK7 CDR-L3 | QQSNEDPWT |
| 508 | PTK7 VH | QVQLVQSGPEVKKPGASVKVSCKASGYTFTDYA |
| | | VHWVRQAPGKRLEWIGVISTYNDYTY |
| | | NNQDFKGRVTMTRDTSASTAYMELSRLRSEDTA |
| | | VYYCARGNSYFYALDYWGQGTSVTVSS |
| 509 | PTK7 VL | EIVLTQSPATLSLSPGERATLSCRASESVDSYGKSF |
| | | MHWYQQKPGQAPRLLIYRASNLES |
| | | GIPARFSGSGSGTDFTLTISSLEPEDFAVYYCQQSN |
| | | EDPWTFGGGTKLEIK |
| 510 | PTK7 CDR-H1 | RYWMS |
| 511 | PTK7 CDR-H2 | DLNPDSSAINYVDSVKG |
| 512 | PTK7 CDR-H3 | ITTLVPYTMDF |
| 513 | PTK7 CDR-L1 | ITNTDIDDDMN |
| 514 | PTK7 CDR-L2 | EGNGLRP |
| 515 | PTK7 CDR-L3 | LQSDNLPLT |
| 516 | PTK7 VH | EVQLVESGGGLVQPGGSLRLSCAASGFDFSRYW |
| | | MSWVRQAPGKGLEWIGDLNPDSSAINY |
| | | VDSVKGRFTISRDNAKNSLYLQMNSLRAEDTAVY |
| | | YCTLITTLVPYTMDFWGQGTSVTVSS |
| 517 | PTK7 VL | ETTLTQSPAFMSATPGDKVNISCITNTDIDDDMNW |
| | | YQQKPGEAAILLISEGNGLRPGIPPRFSGSGYGTDF |
| | | TLTINNIESEDAAYYFCLQSDNLPLTFGSGTKLEIK |
| 518 | hLIV22/LIV1 CDR-H1 | DYYMH |
| 519 | hLIV22/LIV1 CDR-H2 | WIDPENGDTEYGPKFQG |
| 520 | hLIV22/LIV1 CDR-H3 | HNAHYGTWFAY |
| 521 | hLIV22/LIV1 CDR-L1 | RSSQSLLHSSGNTYLE |
| 522 | hLIV22/LIV1 CDR-L2 | KISTRFS |
| 523 | hLIV22/LIV1 CDR-L3 | FQGSHVPYT |
| 524 | hLIV22/LIV1 VH | QVQLVQSGAEVKKPGASVKVSCKASGLTIEDYY |
| | | MHWVRQAPGQGLEWMGWIDPENGDTEY |
| | | GPKFQGRVTMTRDTSINTAYMELSRLRSDDTAVY |
| | | YCAVHNAHYGTWFAYWGQGTLVTVSS |
| 525 | hLIV22/LIV1 VL | DVVMTQSPLSLPVTLGQPASISCRSSQSLLHSSGN |
| | | TYLEWYQQRPGQSPRPLIYKISTRFSGVPDRFSGS |
| | | GSGTDFTLKISRVEAEDVGVYYCFQGSHVPYTFG |
| | | GGTKVEIK |
| 526 | avb6 CDR-H1 | DYNVN |
| 527 | avb6 CDR-H2 | VINPKYGTTRYNQKFKG |
| 528 | avb6 CDR-H3 | GLNAWDY |
| 529 | avb6 CDR-L1 | GASENIYGALN |
| 530 | avb6 CDR-L2 | GATNLED |
| 531 | avb6 CDR-L3 | QNVLTTPYT |
| 532 | avb6 VH | QFQLVQSGAEVKKPGASVKVSCKASGYSFTDYN |
| | | VNWVRQAPGQGLEWIGVINPKYGTTRY |
| | | NQKFKGRATLTVDKSTSTAYMELSSLRSEDTAVY |
| | | YCTRGLNAWDYWGQGTLVTVSS |
| 533 | avb6 VL | DIQMTQSPSSLSASVGDRVTITCGASENIYGALNW |
| | | YQQKPGKAPKLLIYGATNLEDGVPS |
| | | RFSGSGSGRDYTFTISSLQPEDIATYYCQNVLTTPY |
| | | TFGQGTKLEIK |
| 534 | h15H3/avb6 CDR-H1 | GYFMN |
| 535 | h15H3/avb6 CDR-H2 | LINPYNGDSFYNQKFKG |
| 536 | h15H3/avb6 CDR-H3 | GLRRDFDY |
| 537 | h15H3/avb6 CDR-L1 | KSSQSLLDSDGKTYLN |
| 538 | h15H3/avb6 CDR-L2 | LVSELDS |
| 539 | h15H3/avb6 CDR-L3 | WQGTHFPRT |
| 540 | h15H3/avb6 VH | QVQLVQSGAEVKKPGASVKVSCKASGYSFSGYF |
| | | MNWVRQAPGQGLEWMGLINPYNGDSFY |
| | | NQKFKGRVTMTRQTSTSTVYMELSSLRSEDTAVY |
| | | YCVRGLRRDFDYWGQGTLVTVSS |
| 541 | h15H3/avb6 VL | DVVMTQSPLSLPVTLGQPASISCKSSQSLLDSDGK |
| | | TYLNWLFQRPGQSPRRLIYLVSELD |
| | | SGVPDRFSGSGSGTDFTLKISRVEAEDVGVYYCW |
| | | QGTHFPRTFGGGTKLEIK |
| 542 | CD48 CDR-H1 | DFGMN |
| 543 | CD48 CDR-H2 | WINTFTGEPSYGNVFKG |
| 544 | CD48 CDR-H3 | RHGNGNVFDS |
| 545 | CD48 CDR-L1 | RASQSIGSNIH |
| 546 | CD48 CDR-L2 | YTSESIS |
| 547 | CD48 CDR-L3 | QQSNSWPLT |
| 548 | CD48 VH | QVQLVQSGSELKKPGASVKVSCKASGYTFTDFG |
| | | MNWVRQAPGQGLEWMGWINTFTGEPSYGNVFK |
| | | GRFVFSLDTSVSTAYLQISSLKAEDTAVYYCARRH |
| | | GNGNVFDSWGQGTLVTVSS |
| 549 | CD48 VL | EIVLTQSPDFQSVTPKEKVTITCRASQSIGSNIHWY |
| | | QQKPDQSPKLLIKYTSESISGVPSRFSGSGSGTDFT |
| | | LTINSLEAEDAATYYCQQSNSWPLTFGGGTKVEIK |
| | | R |
| 550 | SG-559-01/PD-L1 CDR-H1 | TAAIS |
| 551 | SG-559-01/PD-L1 CDR-H2 | GIIPIFGKAHYAQKFQG |
| 552 | SG-559-01/PD-L1 CDR-H3 | KFHFVSGSPFGMDV |
| 553 | SG-559-01/PD-L1 CDR-L1 | RASQSVSSYLA |
| 554 | SG-559-01/PD-L1 CDR-L2 | DASNRAT |
| 555 | SG-559-01/PD-L1 CDR-L3 | QQRSNWPT |
| 556 | SG-559-01/PD-L1 VH | QVQLVQSGAEVKKPGSSVKVSCKTSGDTFSTAAI |
| | | SWVRQAPGQGLEWMGGIIPIFGKAHYAQKFQGR |
| | | VTITADESTSTAYMELSSLRSEDTAVYFCARKFHF |
| | | VSGSPFGMDVWGQGTTVTVSS |
| 557 | SG-559-01/PD-L1 VL | EIVLTQSPATLSLSPGERATLSCRASQSVSSYLAW |
| | | YQQKPGQAPRLLIYDASNRATGIPA |
| | | RFSGSGSGTDFTLTISSLEPEDFAVYYCQQRSNWP |
| | | TFGQGTKVEIK |
| 558 | IGF-1R CDR-H1 | SYAIS |
| 559 | IGF-1R CDR-H2 | GIIPIFGTANYAQKFQG |
| 560 | IGF-1R CDR-H3 | APLRFLEWSTQDHYYYYYMDV |
| 561 | IGF-1R CDR-L1 | QGDSLRSYYAT |
| 562 | IGF-1R CDR-L2 | GENKRPS |
| 563 | IGF-1R CDR-L3 | KSRDGSGQHLV |
| 564 | IGF-1R VH | EVQLVQSGAEVKKPGSSVKVSCKASGGTFSSYAI |
| | | SWVRQAPGQGLEWMGGIIPIFGTANY |
| | | AQKFQGRVTITADKSTSTAYMELSSLRSEDTAVY |
| | | YCARAPLRFLEWSTQDHYYYYYMDVWGKGTTV |
| | | TVSS |
| 565 | IGF-1R VL | SSELTQDPAVSVALGQTVRITCQGDSLRSYYATW |
| | | YQQKPGQAPILVIYGENKRPSGIPDR |
| | | FSGSSSGNTASLTITGAQAEDEADYYCKSRDGSG |
| | | QHLVFGGGTKLTVL |
| 566 | Claudin-18.2 CDR-H1 | SYWIN |
| 567 | Claudin-18.2 CDR-H2 | NIYPSDSYTNYNQKFKD |
| 568 | Claudin-18.2 CDR-H3 | SWRGNSFDY |
| 569 | Claudin-18.2 CDR-L1 | KSSQSLLNSGNQKNYLT |
| 570 | Claudin-18.2 CDR-L2 | WASTRES |
| 571 | Claudin-18.2 CDR-L3 | QNDYSYPFT |
| 572 | Claudin-18.2 VH | QVQLQQPGAELVRPGASVKLSCKASGYTFTSYWI |
| | | NWVKQRPGQGLEWIGNIYPSDSYTN |
| | | YNQKFKDKATLTVDKSSSTAYMQLSSPTSEDSAV |
| | | YYCTRSWRGNSFDYWGQGTTLTVSS |
| 573 | Claudin-18.2 VL | DIVMTQSPSSLTVTAGEKVTMSCKSSQSLLNSGN |
| | | QKNYLTWYQQKPGQPPKLLIYWASTR |
| | | ESGVPDRFTGSGSGTDFTLTISSVQAEDLAVYYCQ |
| | | NDYSYPFTFGSGTKLEIK |
| 574 | Claudin-18.2 CDR-H1 | NYGMN |
| 575 | Claudin-18.2 CDR-H2 | WINTNTGEPTYAEEFKG |
| 576 | Claudin-18.2 CDR-H3 | LGFGNAMDY |
| 577 | Claudin-18.2 CDR-L1 | KSSQSLLNSGNQKNYLT |
| 578 | Claudin-18.2 CDR-L2 | WASTRES |
| 579 | Claudin-18.2 CDR-L3 | QNDYSYPLT |
| 580 | Claudin-18.2 VH | QIQLVQSGPELKKPGETVKISCKASGYTFTNYGM |
| | | NWVKQAPGKGLKWMGWINTNTGEPTY |
| | | AEEFKGRFAFSLETSASTAYLQINNLKNEDTATYF |
| | | CARLGFGNAMDYWGQGTSVTVSS |
| 581 | Claudin-18.2 VL | DIVMTQSPSSLTVTAGEKVTMSCKSSQSLLNSGN |
| | | QKNYLTWYQQKPGQPPKLLIYWASTR |
| | | ESGVPDRFTGSGSGTDFTLTISSVQAEDLAVYYCQ |
| | | NDYSYPLTFGAGTKLELK |
| 582 | Nectin-4 CDR-H1 | SYNMN |
| 583 | Nectin-4 CDR-H2 | YISSSSSTIYYADSVKG |
| 584 | Nectin-4 CDR-H3 | AYYYGMDV |
| 585 | Nectin-4 CDR-L1 | RASQGISGWLA |
| 586 | Nectin-4 CDR-L2 | AASTLQS |
| 587 | Nectin-4 CDR-L3 | QQANSFPPT |
| 588 | Nectin-4 VH | EVQLVESGGGLVQPGGSLRLSCAASGFTFSSYNM |
| | | NWVRQAPGKGLEWYSYISSSSSTIYY |
| | | ADSVKGRFTISRDNAKNSLSLQMNSLRDEDTAVY |
| | | YCARAYYYGMDVWGQGTTVTVSS |
| 589 | Nectin-4 VL | DIQMTQSPSSVSASVGDRVTITCRASQGISGWLA |
| | | WYQQKPGKAPKFLIYAASTLQSGVPS |
| | | RFSGSGSGTDFTLTISSLQPEDFATYYCQQANSFPP |
| | | TFGGGTKVEIK |
| 590 | SLTRK6 CDR-H1 | SYGMH |
| 591 | SLTRK6 CDR-H2 | VIWYDGSNQYYADSVKG |
| 592 | SLTRK6 CDR-H3 | GLTSGRYGMDV |
| 593 | SLTRK6 CDR-L1 | RSSQSLLLSHGFNYLD |
| 594 | SLTRK6 CDR-L2 | LGSSRAS |
| 595 | SLTRK6 CDR-L3 | MQPLQIPWT |
| 596 | SLTRK6 VH | QVQLVESGGGVVQPGRSLRLSCAASGFTFSSYGM |
| | | HWVRQAPGKGLEWVAVIWYDGSNQYY |
| | | ADSVKGRFTISRDNSKNTLFLQMHSLRAEDTAVY |
| | | YCARGLTSGRYGMDVWGQGTTVTVSS |
| 597 | SLTRK6 VL | DIVMTQSPLSLPVTPGEPASISCRSSQSLLLSHGFN |
| | | YLDWYLQKPGQSPQLLIYLGSSRASGVPDRFSGS |
| | | GSGTDFTLKISRVEAEDVGLYYCMQPLQIPWTFG |
| | | QGTKVEIK |
| 598 | CD228 CDR-H1 | SGYWN |
| 599 | CD228 CDR-H2 | YISDSGITYYNPSLKS |
| 600 | CD228 CDR-H3 | RTLATYYAMDY |
| 601 | CD228 CDR-L1 | RASQSLVHSDGNTYLH |
| 602 | CD228 CDR-L2 | RVSNRFS |
| 603 | CD228 CDR-L3 | SQSTHVPPT |
| 604 | CD228 VH | QVQLQESGPGLVKPSETLSLTCTVSGDSITSGYWN |
| | | WIRQPPGKGLEYIGYISDSGITYYN |
| | | PSLKSRVTISRDTSKNQYSLKLSSVTAADTAVYYC |
| | | ARRTLATYYAMDYWGQGTLVTVSS |
| 605 | CD228 VL | DFVMTQSPLSLPVTLGQPASISCRASQSLVHSDGN |
| | | TYLHWYQQRPGQSPRLLIYRVSNRFSGVPDRFSG |
| | | SGSGTDFTLKISRVEAEDVGVYYCSQSTHVPPTFG |
| | | QGTKLEIKR |
| 606 | CD142 (TF) CDR-H1 | NYAMS |
| 607 | CD142 (TF) CDR-H2 | SISGSGDYTYYTDSVKG |
| 608 | CD142 (TF) CDR-H3 | SPWGYYLDS |
| 609 | CD142 (TF) CDR-L1 | RASQGISSRLA |
| 610 | CD142 (TF) CDR-L2 | AASSLQS |
| 611 | CD142 (TF) CDR-L3 | QQYNSYPYT |
| 612 | CD142 (TF) VH | EVQLLESGGGLVQPGGSLRLSCAASGFTFSNYAM |
| | | SWVRQAPGKGLEWVSSISGSGDYTY |
| | | YTDSVKGRFTISRDNSKNTLYLQMNSLRAEDTAV |
| | | YYCARSPWGYYLDSWGQGTLVTVSS |
| 613 | CD142 (TF) VL | DIQMTQSPPSLSASAGDRVTITCRASQGISSRLAW |
| | | YQQKPEKAPKSLIYAASSLQSGVPS |
| | | RFSGSGSGTDFTLTISSLQPEDFATYYCQQYNSYP |
| | | YTFGQGTKLEIK |
| 614 | h2G12/STn CDR-H1 | DHAIH |
| 615 | h2G12/STn CDR-H2 | YFSPGNDDIKYNEKFRG |
| 616 | h2G12/STn CDR-H3 | SLSTPY |
| 617 | h2G12/STn CDR-L1 | KSSQSLLNRGNHKNYLT |
| 618 | h2G12/STn CDR-L2 | WASTRES |
| 619 | h2G12/STn CDR-L3 | QNDYTYPYT |
| 620 | h2G12/STn VH | EVQLVQSGAEVKKPGASVKVSCKASGYTFTDHAI |
| | | HWVRQAPGQGLEWMGYFSPGNDDIKY |
| | | NEKFRGRVTMTADKSSSTAYMELRSLRSDDTAV |
| | | YFCKRSLSTPYWGQGTLVTVSS |
| 621 | h2G12/STn VL | DIVMTQSPDSLAVSLGERATINCKSSQSLLNRGNH |
| | | KNYLTWYQQKPGQPPKLLIYWAST |
| | | RESGVPDRFSGSGSGTDFTLTISSLQAEDVAVYYC |
| | | QNDYTYPYTFGQGTKVEIK |
| 622 | CD20 CDR-H1 | SYNMH |
| 623 | CD20 CDR-H2 | AIYPGNGDTSYNQKFKG |
| 624 | CD20 CDR-H3 | STYYGGDWYFNV |
| 625 | CD20 CDR-L1 | RASSSVSYIH |
| 626 | CD20 CDR-L2 | ATSNLAS |
| 627 | CD20 CDR-L3 | QQWTSNPPT |
| 628 | CD20 VH | QVQLQQPGAELVKPGASVKMSCKASGYTFTSYN |
| | | MHWVKQTPGRGLEWIGAIYPGNGDTSY |
| | | NQKFKGKATLTADKSSSTAYMQLSSLTSEDSAVY |
| | | YCARSTYYGGDWYFNVWGAGTTVTVSA |
| 629 | CD20 VL | QIVLSQSPAILSASPGEKVTMTCRASSSVSYIHWF |
| | | QQKPGSSPKPWIYATSNLASGVPVR |
| | | FSGSGSGTSYSLTISRVEAEDAATYYCQQWTSNPP |
| | | TFGGGTKLEIK |
| 630 | HER2 CDR-H1 | DTYIH |
| 631 | HER2 CDR-H2 | RIYPTNGYTRYADSVKG |
| 632 | HER2 CDR-H3 | WGGDGFYAMDY |
| 633 | HER2 CDR-L1 | RASQDVNTAVA |
| 634 | HER2 CDR-L2 | SASFLYS |
| 635 | HER2 CDR-L3 | QQHYTTPPT |
| 636 | HER2 VH | EVQLVESGGGLVQPGGSLRLSCAASGFNIKDTYIH |
| | | WVRQAPGKGLEWVARIYPTNGYTRY |
| | | ADSVKGRFTISADTSKNTAYLQMNSLRAEDTAVY |
| | | YCSRWGGDGFYAMDYWGQGTLVTVSS |
| 637 | HER2 VL | DIQMTQSPSSLSASVGDRVTITCRASQDVNTAVA |
| | | WYQQKPGKAPKLLIYSASFLYSGVPS |
| | | RFSGSRSGTDFTLTISSLQPEDFATYYCQQHYTTPP |
| | | TFGQGTKVEIK |
| 638 | CD79b CDR-H1 | SYWIE |
| 639 | CD79b CDR-H2 | EILPGGGDTNYNEIFKG |
| 640 | CD79b CDR-H3 | RVPIRLDY |
| 641 | CD79b CDR-L1 | KASQSVDYEGDSFLN |
| 642 | CD79b CDR-L2 | AASNLES |
| 643 | CD79b CDR-L3 | QQSNEDPLT |
| 644 | CD79b VH | EVQLVESGGGLVQPGGSLRLSCAASGYTESSYWI |
| | | EWVRQAPGKGLEWIGEILPGGGDTNYNEIFKGRA |
| | | TFSADTSKNTAYLQMNSLRAEDTAVYYCTRRVPI |
| | | RLDYWGQGTLVTVSS |
| 645 | CD79b VL | DIQLTQSPSSLSASVGDRVTITCKASQSVDYEGDS |
| | | FLNWYQQKPGKAPKLLIYAASNLES |
| | | GVPSRFSGSGSGTDFTLTISSLQPEDFATYYCQQS |
| | | NEDPLTFGQGTKVEIK |
| 646 | NaPi2B CDR-H1 | DFAMS |
| 647 | NaPi2B CDR-H2 | TIGRVAFHTYYPDSMKG |
| 648 | NaPi2B CDR-H3 | HRGFDVGHFDF |
| 649 | NaPi2B CDR-L1 | RSSETLVHSSGNTYLE |
| 650 | NaPi2B CDR-L2 | RVSNRFS |
| 651 | NaPi2B CDR-L3 | FQGSFNPLT |
| 652 | NaPi2B VH | EVQLVESGGGLVQPGGSLRLSCAASGFSFSDFAM |
| | | SWVRQAPGKGLEWVATIGRVAFHTYY |
| | | PDSMKGRFTISRDNSKNTLYLQMNSLRAEDTAVY |
| | | YCARHRGFDVGHFDFWGQGTLVTVSS |
| 653 | NaPi2B VL | DIQMTQSPSSLSASVGDRVTITCRSSETLVHSSGNT |
| | | YLEWYQQKPGKAPKLLIYRVSNRF |
| | | SGVPSRFSGSGSGTDFTLTISSLQPEDFATYYCFQG |
| | | SFNPLTFGQGTKVEIK |
| 654 | Muc16 CDR-H1 | NDYAWN |
| 655 | Muc16 CDR-H2 | YISYSGYTTYNPSLKS |
| 656 | Muc16 CDR-H3 | WTSGLDY |
| 657 | Muc16 CDR-L1 | KASDLIHNWLA |
| 658 | Muc16 CDR-L2 | GATSLET |
| 659 | Muc16 CDR-L3 | QQYWTTPFT |
| 660 | Muc16 VH | EVQLVESGGGLVQPGGSLRLSCAASGYSITNDYA |
| | | WNWVRQAPGKGLEWVGYISYSGYTTY |
| | | NPSLKSRFTISRDTSKNTLYLQMNSLRAEDTAVYY |
| | | CARWTSGLDYWGQGTLVTVSS |
| 661 | Muc16 VL | DIQMTQSPSSLSASVGDRVTITCKASDLIHNWLA |
| | | WYQQKPGKAPKLLIYGATSLETGVPSRFSGSGSG |
| | | TDFTLTISSLQPEDFATYYCQQYWTTPFTFGQGTK |
| | | VEIK |
| 662 | STEAP1 CDR-H1 | SDYAWN |
| 663 | STEAP1 CDR-H2 | YISNSGSTSYNPSLKS |
| 664 | STEAP1 CDR-H3 | ERNYDYDDYYYAMDY |
| 665 | STEAP1 CDR-L1 | KSSQSLLYRSNQKNYLA |
| 666 | STEAP1 CDR-L2 | WASTRES |
| 667 | STEAP1 CDR-L3 | QQYYNYPRT |
| 668 | STEAP1 VH | EVQLVESGGGLVQPGGSLRLSCAVSGYSITSDYA |
| | | WNWVRQAPGKGLEWVGYISNSGSTSYNPSLKSR |
| | | FTISRDTSKNTLYLQMNSLRAEDTAVYYCARERN |
| | | YDYDDYYYAMDYWGQGTLVTVSS |
| 669 | STEAP1 VL | DIQMTQSPSSLSASVGDRVTITCKSSQSLLYRSNQ |
| | | KNYLAWYQQKPGKAPKLLIYWASTRESGVPSRFS |
| | | GSGSGTDFTLTISSLQPEDFATYYCQQYYNYPRTF |
| | | GQGTKVEIK |
| 670 | BCMA CDR-H1 | NYWMH |
| 671 | BCMA CDR-H2 | ATYRGHSDTYYNQKFKG |
| 672 | BCMA CDR-H3 | GAIYDGYDVLDN |
| 673 | BCMA CDR-L1 | SASQDISNYLN |
| 674 | BCMA CDR-L2 | YTSNLHS |
| 675 | BCMA CDR-L3 | QQYRKLPWT |
| 676 | BCMA VH | QVQLVQSGAEVKKPGSSVKVSCKASGGTFSNYW |
| | | MHWVRQAPGQGLEWMGATYRGHSDTYYNQKF |
| | | KGRVTITADKSTSTAYMELSSLRSEDTAVYYCAR |
| | | GAIYDGYDVLDNWGQGTLVTVSS |
| 677 | BCMA VL | DIQMTQSPSSLSASVGDRVTITCSASQDISNYLNW |
| | | YQQKPGKAPKLLIYYTSNLHSGVPSRFSGSGSGTD |
| | | FTLTISSLQPEDFATYYCQQYRKLPWTFGQGTKLE |
| | | IK |
| 678 | c-Met CDR-H1 | AYTMH |
| 679 | c-Met CDR-H2 | WIKPNNGLANYAQKFQG |
| 680 | c-Met CDR-H3 | SEITTEFDY |
| 681 | c-Met CDR-L1 | KSSESVDSYANSFLH |
| 682 | c-Met CDR-L2 | RASTRES |
| 683 | c-Met CDR-L3 | QQSKEDPLT |
| 684 | c-Met VH | QVQLVQSGAEVKKPGASVKVSCKASGYIFTAYT |
| | | MHWVRQAPGQGLEWMGWIKPNNGLAN |
| | | YAQKFQGRVTMTRDTSISTAYMELSRLRSDDTAV |
| | | YYCARSEITTEFDYWGQGTLVTVSS |
| 685 | c-Met VL | DIVMTQSPDSLAVSLGERATINCKSSESVDSYANS |
| | | FLHWYQQKPGQPPKLLIYRASTRE |
| | | SGVPDRFSGSGSGTDFTLTISSLQAEDVAVYYCQQ |
| | | SKEDPLTFGGGTKVEIK |
| 686 | EGFR CDR-H1 | SDFAWN |
| 687 | EGFR CDR-H2 | YISYSGNTRYQPSLKS |
| 688 | EGFR CDR-H3 | AGRGFPY |
| 689 | EGFR CDR-L1 | HSSQDINSNIG |
| 690 | EGFR CDR-L2 | HGTNLDD |
| 691 | EGFR CDR-L3 | VQYAQFPWT |
| 692 | EGFR VH | QVQLQESGPGLVKPSQTLSLTCTVSGYSISSDFAW |
| | | NWIRQPPGKGLEWMGYISYSGNTRY |
| | | QPSLKSRITISRDTSKNQFFLKLNSVTAADTATYY |
| | | CVTAGRGFPYWGQGTLVTVSS |
| 693 | EGFR VL | DIQMTQSPSSMSVSVGDRVTITCHSSQDINSNIGW |
| | | LQQKPGKSFKGLIYHGTNLDDGVPS |
| | | RFSGSGSGTDYTLTISSLQPEDFATYYCVQYAQFP |
| | | WTFGGGTKLEIK |
| 694 | SLAMF7 CDR-H1 | DYYMA |
| 695 | SLAMF7 CDR-H2 | SINYDGSSTYYVDSVKG |
| 696 | SLAMF7 CDR-H3 | DRGYYFDY |
| 697 | SLAMF7 CDR-L1 | RSSQSLVHSNGNTYLH |
| 698 | SLAMF7 CDR-L2 | KVSNRFS |
| 699 | SLAMF7 CDR-L3 | SQSTHVPPFT |
| 700 | SLAMF7 VH | EVQLVESGGGLVQPGGSLRLSCAASGFTFSDYYM |
| | | AWVRQAPGKGLEWVASINYDGSSTY |
| | | YVDSVKGRFTISRDNAKNSLYLQMNSLRAEDTAV |
| | | YYCARDRGYYFDYWGQGTTVTVSS |
| 701 | SLAMF7 VL | DVVMTQTPLSLSVTPGQPASISCRSSQSLVHSNGN |
| | | TYLHWYLQKPGQSPQLLIYKVSNRF |
| | | SGVPDRFSGSGSGTDFTLKISRVEAEDVGVYFCSQ |
| | | STHVPPFTFGGGTKVEIK |
| 702 | SLITRK6 CDR-H1 | SYGMH |
| 703 | SLITRK6 CDR-H2 | VIWYDGSNQYYADSVKG |
| 704 | SLITRK6 CDR-H3 | GLTSGRYGMDV |
| 705 | SLITRK6 CDR-L1 | RSSQSLLLSHGFNYLD |
| 706 | SLITRK6 CDR-L2 | LGSSRAS |
| 707 | SLITRK6 CDR-L3 | MQPLQIPWT |
| 708 | SLITRK6 VH | QVQLVESGGGVVQPGRSLRLSCAASGFTFSSYGM |
| | | HWVRQAPGKGLEWVAVIWYDGSNQYY |
| | | ADSVKGRFTISRDNSKNTLFLQMHSLRAEDTAVY |
| | | YCARGLTSGRYGMDVWGQGTTVTVSS |
| 709 | SLITRK6 VL | DIVMTQSPLSLPVTPGEPASISCRSSQSLLLSHGFN |
| | | YLDWYLQKPGQSPQLLIYLGSSRA |
| | | SGVPDRFSGSGSGTDFTLKISRVEAEDVGLYYCM |
| | | QPLQIPWTFGQGTKVEIK |
| 710 | C4.4a CDR-H1 | NAWMS |
| 711 | C4.4a CDR-H2 | YISSSGSTIYYADSVKG |
| 712 | C4.4a CDR-H3 | EGLWAFDY |
| 713 | C4.4a CDR-L1 | TGSSSNIGAGYVVH |
| 714 | C4.4a CDR-L2 | DNNKRPS |
| 715 | C4.4a CDR-L3 | AAWDDRLNGPV |
| 716 | C4.4a VH | EVQLLESGGGLVQPGGSLRLSCAASGFTFSNAWM |
| | | SWVRQAPGKGLEWVSYISSSGSTIYY |
| | | ADSVKGRFTISRDNSKNTLYLQMNSLRAEDTAVY |
| | | YCAREGLWAFDYWGQGTLVTVSS |
| 717 | C4.4a VL | ESVLTQPPSVSGAPGQRVTISCTGSSSNIGAGYVV |
| | | HWYQQLPGTAPKLLIYDNNKRPSGV |
| | | PDRFSGSKSGTSASLAISGLRSEDEADYYCAAWD |
| | | DRLNGPVFGGGTKLTVL |
| 718 | GCC CDR-H1 | GYYWS |
| 719 | GCC CDR-H2 | EINHRGNTNDNPSLKS |
| 720 | GCC CDR-H3 | ERGYTYGNFDH |
| 721 | GCC CDR-L1 | RASQSVSRNLA |
| 722 | GCC CDR-L2 | GASTRAT |
| 723 | GCC CDR-L3 | QQYKTWPRT |
| 724 | GCC VH | QVQLQQWGAGLLKPSETLSLTCAVFGGSFSGYY |
| | | WSWIRQPPGKGLEWIGEINHRGNTNDN |
| | | PSLKSRVTISVDTSKNQFALKLSSVTAADTAVYYC |
| | | ARERGYTYGNFDHWGQGTLVTVSS |
| 725 | GCC VL | EIVMTQSPATLSVSPGERATLSCRASQSVSRNLAW |
| | | YQQKPGQAPRLLIYGASTRATGIP |
| | | ARFSGSGSGTEFTLTIGSLQSEDFAVYYCQQYKT |
| | | WPRTFGQGTNVEIK |
| 726 | Axl CDR-H1 | SYAMN |
| 727 | Axl CDR-H2 | TTSGSGASTYYADSVKG |
| 728 | Axl CDR-H3 | IWIAFDI |
| 729 | Axl CDR-L1 | RASQSVSSSYLA |
| 730 | Axl CDR-L2 | GASSRAT |
| 731 | Axl CDR-L3 | QQYGSSPYT |
| 732 | Axl VH | EVQLLESGGGLVQPGGSLRLSCAASGFTFSSYAM |
| | | NWVRQAPGKGLEWVSTTSGSGASTYY |
| | | ADSVKGRFTISRDNSKNTLYLQMNSLRAEDTAVY |
| | | YCAKIWIAFDIWGQGTMVTVSS |
| 733 | Axl VL | EIVLTQSPGTLSLSPGERATLSCRASQSVSSSYLA |
| | | WYQQKPGQAPRLLIYGASSRATGIP |
| | | DRFSGSGSGTDFTLTISRLEPEDFAVYYCQQYGSS |
| | | PYTFGQGTKLEIK |
| 734 | CR011/gpNMB CDR-H1 | SFNYYWS |
| 735 | CR011/gpNMB CDR-H2 | YIYYSGSTYSNPSLKS |
| 736 | CR011/gpNMB CDR-H3 | GYNWNYFDY |
| 737 | CR011/gpNMB CDR-L1 | RASQSVDNNLV |
| 738 | CR011/gpNMB CDR-L2 | GASTRAT |
| 739 | CR011/gpNMB CDR-L3 | QQYNNWPPWT |
| 740 | CR011/gpNMB VH | QVQLQESGPGLVKPSQTLSLTCTVSGGSISSFNYY |
| | | WSWIRHHPGKGLEWIGYIYYSGSTY |
| | | SNPSLKSRVTISVDTSKNQFSLTLSSVTAADTAVY |
| | | YCARGYNWNYFDYWGQGTLVTVSS |
| 741 | CR011/gpNMB VL | EIVMTQSPATLSVSPGERATLSCRASQSVDNNLV |
| | | WYQQKPGQAPRLLIYGASTRATGIPA |
| | | RFSGSGSGTEFTLTISSLQSEDFAVYYCQQYNNWP |
| | | PWTFGQGTKVEIK |
| 742 | Prolactin receptor CDR-H1 | TYWMH |
| 743 | Prolactin receptor CDR-H2 | EIDPSDSYSNYNQKFKD |
| 744 | Prolactin receptor CDR-H3 | NGGLGPAWFSY |
| 745 | Prolactin receptor CDR-L1 | KASQYVGTAVA |
| 746 | Prolactin receptor CDR-L2 | SASNRYT |
| 747 | Prolactin receptor CDR-L3 | QQYSSYPWT |
| 748 | Prolactin receptor VH | EVQLVQSGAEVKKPGSSVKVSCKASGYTFTTYW |
| | | MHWVRQAPGQGLEWIGEIDPSDSYSNY |
| | | NQKFKDRATLTVDKSTSTAYMELSSLRSEDTAVY |
| | | YCARNGGLGPAWFSYWGQGTLVTVSS |
| 749 | Prolactin receptor VL | DIQMTQSPSSVSASVGDRVTITCKASQYVGTAVA |
| | | WYQQKPGKSPKLLIYSASNRYTGVPS |
| | | RFSDSGSGTDFTLTISSLQPEDFATYFCQQYSSYP |
| | | WTFGGGTKVEIK |
| 750 | FGFR2 CDR-H1 | SYAMS |
| 751 | FGFR2 CDR-H2 | AISGSGTSTYYADSVKG |
| 752 | FGFR2 CDR-H3 | VRYNWNHGDWFDP |
| 753 | FGFR2 CDR-L1 | SGSSSNIGNNYVS |
| 754 | FGFR2 CDR-L2 | ENYNRPA |
| 755 | FGFR2 CDR-L3 | SSWDDSLNYWV |
| 756 | FGFR2 VH | EVQLLESGGGLVQPGGSLRLSCAASGFTFSSYAM |
| | | SWVRQAPGKGLEWVSAISGSGTSTYYADSVKGRF |
| | | TISRDNSKNTLYLQMNSLRAEDTAVYYCARVRY |
| | | NWNHGDWFDPWGQGTLVTVSS |
| 757 | FGFR2 VL | QSVLTQPPSASGTPGQRVTISCSGSSSNIGNNYVS |
| | | WYQQLPGTAPKLLIYENYNRPAGVP |
| | | DRFSGSKSGTSASLAISGLRSEDEADYYCSSWDDS |
| | | LNYWVFGGGTKLTVL |
| 758 | CDCP1 CDR-H1 | SYGMS |
| 759 | CDCP1 CDR-H2 | TISSGGSYKYYVDSVKG |
| 760 | CDCP1 CDR-H3 | HPDYDGVWFAY |
| 761 | CDCP1 CDR-L1 | SVSSSVFYVH |
| 762 | CDCP1 CDR-L2 | DTSKLAS |
| 763 | CDCP1 CDR-L3 | QQWNSNPPT |
| 764 | CDCP1 VH | EVQLVESGGGLVQPGGSLRLSCAASGFTFNSYGM |
| | | SWVRQAPGKGLEWVATISSGGSYKYY |
| | | VDSVKGRFTISRDNAKNSLYLQMNSLRAEDTAVY |
| | | YCARHPDYDGVWFAYWGQGTLVTVSS |
| 765 | CDCP1 VL | DIQMTQSPSSLSASVGDRVTITCSVSSSVFYVHWY |
| | | QQKPGKAPKLLIYDTSKLASSGVPS |
| | | RFSGSGSGTDFTFTISSLQPEDIATYYCQQWNSNPP |
| | | TFGGGTKVEIK |
| 766 | CDCP1 CDR-H1 | SYGMS |
| 767 | CDCP1 CDR-H2 | TISSGGSYTYYPDSVKG |
| 768 | CDCP1 CDR-H3 | HPDYDGVWFAY |
| 769 | CDCP1 CDR-L1 | SVSSSVFYVH |
| 770 | CDCP1 CDR-L2 | DTSKLAS |
| 771 | CDCP1 CDR-L3 | QQWNSNPPT |
| 772 | CDCP1 VH | EVQLVESGGDLVKPGGSLKLSCAASGFTFNSYGM |
| | | SWVRQTPDKRLEWVATISSGGSYTYY |
| | | PDSVKGRFTISRDNAKNTLYLQMSSLKSEDTAMY |
| | | YCARHPDYDGVWFAYWGQGTLVTVSA |
| 773 | CDCP1 VL | QIVLTQSPAIMASPGEKVTMTCSVSSSVFYVHWY |
| | | QQKSGTSPKRWIYDTSKLASGVPARF |
| | | SGSGSGTSYSLTISSMEAEDAATYYCQQWNSNPP |
| | | TFGGGTKLEIK |
| 774 | CDCP1 CDR-H1 | SYYMH |
| 775 | CDCP1 CDR-H2 | IINPSGGSTSYAQKFQG |
| 776 | CDCP1 CDR-H3 | DGVLRYFDWLLDYYYY |
| 777 | CDCP1 CDR-L1 | RASQSVGSYLA |
| 778 | CDCP1 CDR-L2 | DASNRAT |
| 779 | CDCP1 CDR-L3 | QQRANVFT |
| 780 | CDCP1 VH | EVQLVQSGAEVKKPGASVKVSCKASGYTFTSYY |
| | | MHWVRQAPGQGLEWMGIINPSGGSTSY |
| | | AQKFQGRVTMTRDTSTSTVYMELSSLRSEDTAVY |
| | | YCARDGVLRYFDWLLDYYYYMDVWGKG |
| | | TTVTVSS |
| 781 | CDCP1 VL | EIVLTQSPATLSLSPGERATLSCRASQSVGSYLAW |
| | | YQQRPGQAPRLLIYDASNRATGIPA |
| | | RFSGSGSGTDFTLTISSLEPEDFAVYYCQQRANVF |
| | | TFGQGTKVEIK |
| 782 | CDCP1 CDR-H1 | SYYMH |
| 783 | CDCP1 CDR-H2 | IINPSGGSTSYAQKFQG |
| 784 | CDCP1 CDR-H3 | DAELRHFDHLLDYHYYMDV |
| 785 | CDCP1 CDR-L1 | RASQSVGSYLA |
| 786 | CDCP1 CDR-L2 | DASNRAT |
| 787 | CDCP1 CDR-L3 | QQRAQEFT |
| 788 | CDCP1 VH | EVQLVQSGAEVKKPGASVKVSCKASGYTFTSYY |
| | | MHWVRQAPGQGLEWMGIINPSGGSTSYAQKFQG |
| | | RVTMTRDTSTSTVYMELSSLRSEDTAVYYCARDA |
| | | ELRHFDHLLDYHYYMDVWGQGTTVTVSS |
| 789 | CDCP1 VL | EIVMTQSPATLSLSPGERATLSCRASQSVGSYLAW |
| | | YQQKPGQAPRLLIYDASNRATGIPA |
| | | RFSGSGSGTDFTLTISSLQPEDFAVYYCQQRAQEF |
| | | TFGQGTKVEIK |
| 790 | ASCT2 VH | QVQLVQSGSELKKPGAPVKVSCKASGYTFSTFGM |
| | | SWVRQAPGQGLKWMGWIHTYAGVPIYGDDFKG |
| | | RFVFSLDTSVSTAYLQISSLKAEDTAVYFCARRSD |
| | | NYRYFFDYWGQGTTVTVSS |
| 791 | ASCT2 VL | DIQMTQSPSSLSASLGDRVTITCRASQDIRNYLNW |
| | | YQQKPGKAPKLLIYYTSRLHSGVPSRFSGSGSGTD |
| | | YTLTISSLQPEDFATYFCQQGHTLPPTFGQGTKLEI |
| | | K |
| 792 | ASCT2 VH | QIQLVQSGPELKKPGAPVKISCKASGYTFTTFGMS |
| | | WVKQAPGQGLKWMGWIHTYAGVPIYGDDFKGR |
| | | FVFSLDTSVSTAYLQISSVKAEDTATYFCARRSDN |
| | | YRYFFDYWGQGTTLTVSS |
| 793 | ASCT2 VL | DIQMTQSPSSLSASLGDRVTITCRASQDIRNYLNW |
| | | YQQKPGKAPKLLIYYTSRLHSGVPS |
| | | RFSGSGSGTDYTLTISSLQPEDFATYFCQQGHTLPP |
| | | TFGQGTKLEIK |
| 794 | ASCT2 CDR-H1 | NYYMA |
| 795 | ASCT2 CDR-H2 | SITKGGGNTYYRDSVKG |
| 796 | ASCT2 CDR-H3 | QVTIAAVSTSYFDS |
| 797 | ASCT2 CDR-L1 | KTNQKVDYYGNSYVY |
| 798 | ASCT2 CDR-L2 | LASNLAS |
| 799 | ASCT2 CDR-L3 | QQSRNLPYT |
| 800 | ASCT2 VH | EVQLVESGGGLVQSGRSIRLSCAASGFSFSNYYM |
| | | AWVRQAPSKGLEWVASITKGGGNTYYRDSVKGR |
| | | FTFSRDNAKSTLYLQMDSLRSEDTATYYCARQVT |
| | | IAAVSTSYFDSWGQGVMVTVSS |
| 801 | ASCT2 VL | DIVLTQSPALAVSLGQRATISCKTNQKVDYYGNS |
| | | YVYWYQQKPGQQPKLLIYLASNLASGIPARFSGR |
| | | GSGTDFTLTIDPVEADDTATYYCQQSRNLPYTFG |
| | | AGTKLELK |
| 802 | CD123 CDR-H1 | DYYMK |
| 803 | CD123 CDR-H2 | DIIPSNGATFYNQKFKG |
| 804 | CD123 CDR-H3 | SHLLRASWFAY |
| 805 | CD123 CDR-L1 | KSSQSLLNSGNQKNYLT |
| 806 | CD123 CDR-L2 | WASTRES |
| 807 | CD123 CDR-L3 | QNDYSYPYT |
| 808 | CD123 VH | QVQLVQSGAEVKKPGASVKMSCKASGYTFTDYY |
| | | MKWVKQAPGQGLEWIGDIIPSNGATFYNQKFKG |
| | | KATLTVDRSISTAYMHLNRLRSDDTAVYYCTRSH |
| | | LLRASWFAYWGQGTLVTVSS |
| 809 | CD123 VL | DFVMTQSPDSLAVSLGERATINCKSSQSLLNSGNQ |
| | | KNYLTWYLQKPGQPPKLLIYWASTRESGVPDRFS |
| | | GSGSGTDFTLTISSLQAEDVAVYYCQNDYSYPYTF |
| | | GQGTKLEIK |
| 810 | GPC3 CDR-H1 | DYEMH |
| 811 | GPC3 CDR-H2 | GIDPETGGTAYNQKFKG |
| 812 | GPC3 CDR-H3 | YYSFAY |
| 813 | GPC3 CDR-L1 | RSSQSIVHSNANTYLQ |
| 814 | GPC3 CDR-L2 | KVSNRFS |
| 815 | GPC3 CDR-L3 | FQVSHVPYT |
| 816 | GPC3 VH | EVQLVQSGAEVKKPGATVKISCKVSGYTFTDYEM |
| | | HWVQQAPGKGLEWMGGIDPETGGTAYNQKFKG |
| | | RVTLTADKSTDTAYMELSSLRSEDTAVYYCGRYY |
| | | SFAYWGQGTLVTVSS |
| 817 | GPC3 VL | DVVMTQSPLSLPVTLGQPASISCRSSQSIVHSNAN |
| | | TYLQWFQQRPGQSPRLLIYKVSNRFSGVPDRFSGS |
| | | GSGTDFTLKISRVEAEDVGVYYCFQVSHVPYTFG |
| | | QGTKLEIK |
| 834 | PD-L1 CDR-H1 | TAAIS |
| 835 | PD-L1 CDR-H2 | GIIPIFGKAHYAQKFQG |
| 836 | PD-L1 CDR-H3 | KFHFVSGSPFGMDV |
| 837 | PD-L1 CDR-L1 | RASQSVSSYLA |
| 838 | PD-L1 CDR-L2 | DASNRAT |
| 839 | PD-L1 CDR-L3 | QQRSNWPT |
| 840 | PD-L1 VH | QVQLVQSGAEVKKPGSSVKVSCKTSGDTESTAAI |
| | | SWVRQAPGQGLEWMGGIIPIFGKAHYAQKFQGR |
| | | VTITADESTSTAYMELSSLRSEDTAVYFCARKFHF |
| | | VSGSPFGMDVWGQGTTVTVSS |
| 841 | PD-L1 VL | EIVLTQSPATLSLSPGERATLSCRASQSVSSYLAW |
| | | YQQKPGQAPRLLIYDASNRATGIPARFSGSGSGTD |
| | | FTLTISSLEPEDFAVYYCQQRSNWPTFGQGTKVEI |
| | | K |
| 842 | TIGIT CDR-H1 | GTFSSYAIS |
| 843 | TIGIT CDR-H2 | SIIPIFGTANYAQKFQG |
| 844 | TIGIT CDR-H3 | GPSEVGAILGYVWFDP |
| 845 | TIGIT CDR-L1 | RSSQSLLHSNGYNYLD |
| 846 | TIGIT CDR-L2 | LGSNRAS |
| 847 | TIGIT CDR-L3 | MQARRIPIT |
| 848 | TIGIT VH | QVQLVQSGAEVKKPGSSVKVSCKASGGTFSSYAI |
| | | SWVRQAPGQGLEWMGSIIPIFGTANYAQKFQGRV |
| | | TITADESTSTAYMELSSLRSEDTAVYYCARGPSEV |
| | | GAILGYVWFDPWGQGTLVTVSS |
| 849 | TIGIT VL | DIVMTQSPLSLPVTPGEPASISCRSSQSLLHSNGYN |
| | | YLDWYLQKPGQSPQLLIYLGSNRASGVPDRFSGS |
| | | GSGTDFTLKISRVEAEDVGVYYCMQARRIPITFGG |
| | | GTKVEIK |
| 850 | STN CDR-H1 | GYTFTDHAIHWV |
| 851 | STN CDR-H2 | FSPGNDDIKY |
| 852 | STN CDR-H3 | KRSLSTPY |
| 853 | STN CDR-L1 | QSLLNRGNHKNY |
| 854 | STN CDR-L2 | WASTRES |
| 855 | STN CDR-L3 | QNDYTYPYT |
| 856 | STN VH | EVQLVQSGAEVKKPGASVKVSCKASGYTFTDHAI |
| | | HWVRQAPGQGLEWMGYFSPGNDDIKYNEKFRGR |
| | | VTMTADKSSSTAYMELRSLRSDDTAVYFCKRSLS |
| | | TPYWGQGTLVTVSS |
| 857 | STN VL | DIVMTQSPDSLAVSLGERATINCKSSQSLLNRGNH |
| | | KNYLTWYQQKPGQPPKLLIYWASTRESGVPDRFS |
| | | GSGSGTDFTLTISSLQAEDVAVYYCONDYTYPYT |
| | | FGQGTKVEIK |
| 858 | CD33 CDR-H1 | NYDIN |
| 859 | CD33 CDR-H2 | WIYPGDGSTKYNEKFKA |
| 860 | CD33 CDR-H3 | GYEDAMDY |
| 861 | CD33 CDR-L1 | KASQDINSYLS |
| 862 | CD33 CDR-L2 | RANRLVD |
| 863 | CD33 CDR-L3 | LQYDEFPLT |
| 864 | CD33 VH | QVQLVQSGAE VKKPGASVKV SCKASGYTFT |
| | | NYDINWVRQA PGQGLEWIGW IYPGDGSTKY |
| | | NEKFKAKATL TADTSTSTAY MELRSLRSDD |
| | | TAVYYCASGY EDAMDYWGQG TTVTVSS |
| 865 | CD33 VL | DIQMTQSPSSLSASVGDRVTINCKASQDINSYLSW |
| | | FQQKPGKAPKTLIYRANRLVDGVPSRFSGSGSGQ |
| | | DYTLTISSLQPEDFATYYCLQYDEFPLTFGGGTKV |
| | | EIK |
| 866 | NTBA CDR-H1 | NYGMN |
| 867 | NTBA CDR-H2 | WINTYSGEPRYADDFKG |
| 868 | NTBA CDR-H3 | DYGRWYFDV |
| 869 | NTBA CDR-L1 | RASSSVSHMH |
| 870 | NTBA CDR-L2 | ATSNLAS |
| 871 | NTBA CDR-L3 | QQWSSTPRT |
| 872 | NTBA VH | QIQLVQSGSELKKPGASVKVSCKASGYTFTNYGM |
| | | NWVRQAPGQDLKWMGWINTYSGEPRYADDFKG |
| | | RFVFSLDKSVNTAYLQISSLKAEDTAVYYCARDY |
| | | GRWYFDVWGQGTTVTVSS |
| 873 | NTBA VL | QIVLSQSPATLSLSPGERATMSCRASSSVSHMHW |
| | | YQQKPGQAPRPWIYATSNLASGVPARFSGSGSGT |
| | | DYTLTISSLEPEDFAVYYCQQWSSTPRTFGGGTKV |
| | | EIK |
| 874 | BCMA CDR-H1 | DYYIH |
| 875 | BCMA CDR-H2 | YINPNSGYTNYAQKFQG |
| 876 | BCMA CDR-H3 | YMWERVTGFFDF |
| 877 | BCMA CDR-L1 | LASEDISDDLA |
| 878 | BCMA CDR-L2 | TTSSLQS |
| 879 | BCMA CDR-L3 | QQTYKFPPT |
| 880 | BCMA VH | QVQLVQSGAEVKKPGASVKLSCKASGYTFTDYYI |
| | | HWVRQAPGQGLEWIGYINPNSGYTNYAQKFQGR |
| | | ATMTADKSINTAYVELSRLRSDDTAVYFCTRYM |
| | | WERVTGFFDFWGQGTMVTVSS |
| 881 | BCMA VL | DIQMTQSPSSVSASVGDRVTITCLASEDISDDLAW |
| | | YQQKPGKAPKVLVYTTSSLQSGVPSRFSGSGSGT |
| | | DFTLTISSLQPEDFATYFCQQTYKFPPTFGGGTKV |
| | | EIK |
| 882 | TF CDR-H1 | GFTFSNYA |
| 883 | TF CDR-H2 | ISGSGDYT |
| 884 | TF CDR-H3 | ARSPWGYYLDS |
| 885 | TF CDR-L1 | QGISSR |
| 886 | TF CDR-L2 | AAS |
| 887 | TF CDR-L3 | QQYNSYPYT |
| 888 | TF VH | EVQLLESGGGLVQPGGSLRLSCAASGFTFSNYAM |
| | | SWVRQAPGKGLEWVSSISGSGDYTYYTDSVKGRF |
| | | TISRDNSKNTLYLQMNSLRAEDTAVYYCARSPWG |
| | | YYLDSWGQGTLVTVSS |
| 889 | TF VL | DIQMTQSPPSLSASAGDRVTITCRASQGISSRLAW |
| | | YQQKPEKAPKSLIYAASSLQSGVPSRFSGSGSGTD |
| | | FTLTISSLQPEDFATYYCQQYNSYPYTFGQGTKLEI |
| | | K |
| 890 | AvB6 EC4 Heavy chain full | QFQLVQSGAEVKKPGASVKVSCKASGYSFTDYN |
| | sequence | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSSastkgpsvfplapsskstsggtaalgc |
| | | lvkdyfpepvtvswnsgaltsgvhtfpavlqssglyslssvvtvpssslgtqt |
| | | yicnvnhkpsntkvdkkvepkscdkthtcppcpapellggpsvflfppkp |
| | | kdtlmisrtpevtcvvvdvshedpevkfnwyvdgvevhnaktkpreeqy |
| | | nstyrvvsvltvlhqdwlngkeykckvsnkalpapiektiskakgqprepq |
| | | vytlppsrdeltknqvsltclvkgfypsdiavewesngqpennykttppvld |
| | | cdgsfflyskltvdksrwqqgnvfscsvmhealhnhytqkslslspgk |
| | | (constant domain shown in lowercase) |
| 891 | AvB6 EC4 Light chain full | DIQMTQSPSSLSASVGDRVTITCGASENIYGALNW |
| | sequence | YQQKPGKAPKLLIYGATNLEDGVPSRFSGSGSGR |
| | | DYTFTISSLQPEDIATYYCQNVLTTPYTFGQGTKL |
| | | EIKrtvaapcvfifppsdeqlksgtasvvcllnnfypreakvqwkvdnalq |
| | | sgnsqesvteqdskdstyslsstltlskadyekhkvyacevthqglsspvtks |
| | | fnrgec (constant domain shown in lowercase) |
| 892 | CH3 SH400C | Gqprepqvytlppsrdeltknqvsltclvkgfypsdiavewesngqpenn |
| | | ykttppvldcdgsfflyskltvdksrwqqgnvfscsvmhealhnhytqksl |
| | | slspgk |
| 893 | Kappa SL114C | Rtvaapcvfifppsdeqlksgtasvvcllnnfypreakvqwkvdnalqsgn |
| | | sqesvteqdskdstyslsstltlskadyekhkvyacevthqglsspvtksfnr |
| | | gec |
| 894 | cAC10 ec4 heavy chain full | QIQLQQSGPEVVKPGASVKISCKASGYTFTDYYIT |
| | sequence | WVKQKPGQGLEWIGWIYPGSGNTKYNEKFKGKA |
| | | TLTVDTSSSTAFMQLSSLTSEDTAVYFCANYGNY |
| | | WFAYWGQGTQVTVSAastkgpsvfplapsskstsggtaalgcl |
| | | vkdyfpepvtvswnsgaltsgvhtfpavlqssglyslssvvtvpssslgtqty |
| | | icnvnhkpsntkvdkkvepkscdkthtcppcpapellggpsvflfppkpk |
| | | dtlmisrtpevtcvvvdvshedpevkfnwyvdgvevhnaktkpreeqyn |
| | | styrvvsvltvlhqdwlngkeykckvsnkalpapiektiskakgqprepqv |
| | | ytlppsrdeltknqvsltclvkgfypsdiavewesngqpennykttppvldc |
| | | dgsfflyskltvdksrwqqgnvfscsvmhealhnhytqkslslspgk |
| | | (constant domain shown in lowercase) |
| 895 | cAC10 ec4 light chain full | DIVLTQSPASLAVSLGQRATISCKASQSVDFDGDS |
| | sequence | YMNWYQQKPGQPPKVLIYAASNLESGIPARFSGS |
| | | GSGTDFTLNIHPVEEEDAATYYCQQSNEDPWTFG |
| | | GGTKLEIKrtvaapcvfifppsdeqlksgtasvvcllnnfypreakvq |
| | | wkvdnalqsgnsqesvteqdskdstyslsstltlskadyekhkvyacevth |
| | | qglsspvtksfnrgec (constant domain shown in lowercase) |
| 896 | hAC10 ec4 heavy chain full | QVQLVQSGAEVKKPGASVKVSCKASGYTFTDYYI |
| | sequence | TWVRQAPGQGLEWMGWIYPGSGNTKYNEKFKG |
| | | RVTMTRDTSISTAYMELSRLRSDDTAVYYCANYG |
| | | NYWFAYWGQGTLVTVSSastkgpsvfplapsskstsggtaalg |
| | | clvkdyfpepvtvswnsgaltsgvhtfpavlqssglyslssvvtvpssslgtq |
| | | tyicnvnhkpsntkvdkkvepkscdkthtcppcpapellggpsvflfppkp |
| | | kdtlmisrtpevtcvvvdvshedpevkfnwyvdgvevhnaktkpreeqy |
| | | nstyrvvsvltvlhqdwlngkeykckvsnkalpapiektiskakgqprepq |
| | | vytlppsrdeltknqvsltclvkgfypsdiavewesngqpennykttppvld |
| | | cdgsfflyskltvdksrwqqgnvfscsvmhealhnhytqkslslspgk |
| | | (constant domain shown in lowercase) |
| 897 | hAC10 ec4 light chain full | DIVMTQSPDSLAVSLGERATINCKASQSVDFDGDS |
| | sequence | YMNWYQQKPGQPPKLLIYAASNLESGVPDRFSGS |
| | | GSGTDFTLTISSLQAEDVAVYYCQQSNEDPWTFG |
| | | QGTKVEIKrtvaapcvfifppsdeqlksgtasvvcllnnfypreakvq |
| | | wkvdnalqsgnsqesvteqdskdstyslsstltlskadyekhkvyacevth |
| | | qglsspvtksfnrgec (constant domain shown in lowercase) |
| 898 | hAC10 VH | QVQLVQSGAEVKKPGASVKVSCKASGYTFTDYYI |
| | | TWVRQAPGQGLEWMGWIYPGSGNTKYNEKFKG |
| | | RVTMTRDTSISTAYMELSRLRSDDTAVYYCANYG |
| | | NYWFAYWGQGTLVTVSS |
| 899 | hAC10 VL | DIVMTQSPDSLAVSLGERATINCKASQSVDFDGDS |
| | | YMNWYQQKPGQPPKLLIYAASNLESGVPDRFSGS |
| | | GSGTDFTLTISSLQAEDVAVYYCQQSNEDPWTFG |
| | | QGTKVEIK |
| 900 | h1C1 CDR-H1 | HYMMA |
| 901 | h1C1 CDR-H2 | RIGPSGGPTHYADSVKG |
| 902 | h1C1 CDR-H3 | YDSGYDYVAVAGPAEYFQH |
| 903 | h1C1 CDR-L1 | RASQSISTWLA |
| 904 | h1C1 CDR-L2 | KASNLHT |
| 905 | h1C1 CDR-L3 | QQYNSYSRT |
| 906 | h1C1 VH | EVQLLESGGGLVQPGGSLRLSCAASGFTFSHYMM |
| | | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSS |
| 907 | h1C1 VL | DIQMTQSPSSLSASVGDRVTITCRASQSISTWLAW |
| | | YQQKPGKAPKLLIYKASNLHTGVPSRFSGSGSGTE |
| | | FSLTISGLQPDDFATYYCQQYNSYSRTFGQGTKVE |
| | | IK |
| 908 | h1C1 HC | EVQLLESGGGLVQPGGSLRLSCAASGFTFSHYMM |
| | | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSASTKGP |
| | | SVFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTCP |
| | | PCPAPELLGGPSVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALP |
| | | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| 909 | h1C1 HC v2 | EVQLLESGGGLVQPGGSLRLSCAASGFTFSHYMM |
| | | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSASTKGP |
| | | SVFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTCP |
| | | PCPAPELLGGPSVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALP |
| | | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPG |
| 910 | h1C1 LC | DIQMTQSPSSLSASVGDRVTITCRASQSISTWLAW |
| | | YQQKPGKAPKLLIYKASNLHTGVPSRFSGSGSGTE |
| | | FSLTISGLQPDDFATYYCQQYNSYSRTFGQGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 911 | h1C1 mIgG2a HC | EVQLLESGGGLVQPGGSLRLSCAASGFTFSHYMM |
| | | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSAKTTAP |
| | | SVYPLAPVCGDTTGSSVTLGCLVKGYFPEPVTLT |
| | | WNSGSLSSGVHTFPAVLQSDLYTLSSSVTVTSST |
| | | WPSQSITCNVAHPASSTKVDKKIEPRGPTIKPCPPC |
| | | KCPAPNLLGGPSVFIFPPKIKDVLMISLSPIVTCVV |
| | | VDVSEDDPDVQISWFVNNVEVHTAQTQTHREDY |
| | | NSTLRVVSALPIQHQDWMSGKEFKCKVNNKDLP |
| | | APIERTISKPKGSVRAPQVYVLPPPEEEMTKKQVT |
| | | LTCMVTDFMPEDIYVEWTNNGKTELNYKNTEPV |
| | | LDSDGSYFMYSKLRVEKKNWVERNSYSCSVVHE |
| | | GLHNHHTTKSFSRTPGK |
| 912 | h1C1 mIgG2a HC v2 | EVQLLESGGGLVQPGGSLRLSCAASGFTFSHYMM |
| | | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSAKTTAP |
| | | SVYPLAPVCGDTTGSSVTLGCLVKGYFPEPVTLT |
| | | WNSGSLSSGVHTFPAVLQSDLYTLSSSVTVTSST |
| | | WPSQSITCNVAHPASSTKVDKKIEPRGPTIKPCPPC |
| | | KCPAPNLLGGPSVFIFPPKIKDVLMISLSPIVTCVV |
| | | VDVSEDDPDVQISWFVNNVEVHTAQTQTHREDY |
| | | NSTLRVVSALPIQHQDWMSGKEFKCKVNNKDLP |
| | | APIERTISKPKGSVRAPQVYVLPPPEEEMTKKQVT |
| | | LTCMVTDFMPEDIYVEWTNNGKTELNYKNTEPV |
| | | LDSDGSYFMYSKLRVEKKNWVERNSYSCSVVHE |
| | | GLHNHHTTKSFSRTPG |
| 913 | h1C1 mk LC | DIQMTQSPSSLSASVGDRVTITCRASQSISTWLAW |
| | | YQQKPGKAPKLLIYKASNLHTGVPSRFSGSGSGTE |
| | | FSLTISGLQPDDFATYYCQQYNSYSRTFGQGTKVE |
| | | IKRADAAPTVSIFPPSSEQLTSGGASVVCFLNNFYP |
| | | KDINVKWKIDGSERQNGVLNSWTDQDSKDSTYS |
| | | MSSTLTLTKDEYERHNSYTCEATHKTSTSPIVKSF |
| | | NRNEC |
| 914 | h1C1 mIgG2a LALAPG | EVQLLESGGGLVQPGGSLRLSCAASGFTFSHYMM |
| | HC | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSAKTTAP |
| | | SVYPLAPVCGDTTGSSVTLGCLVKGYFPEPVTLT |
| | | WNSGSLSSGVHTFPAVLQSDLYTLSSSVTVTSST |
| | | WPSQSITCNVAHPASSTKVDKKIEPRGPTIKPCPPC |
| | | KCPAPNAAGGPSVFIFPPKIKDVLMISLSPIVTCVV |
| | | VDVSEDDPDVQISWFVNNVEVHTAQTQTHREDY |
| | | NSTLRVVSALPIQHQDWMSGKEFKCKVNNKDLG |
| | | APIERTISKPKGSVRAPQVYVLPPPEEEMTKKQVT |
| | | LTCMVTDFMPEDIYVEWTNNGKTELNYKNTEPV |
| | | LDSDGSYFMYSKLRVEKKNWVERNSYSCSVVHE |
| | | GLHNHHTTKSFSRTPGK |
| 915 | h1C1 mIgG2a LALAPG | EVQLLESGGGLVQPGGSLRLSCAASGFTFSHYMM |
| | HC v2 | AWVRQAPGKGLEWVSRIGPSGGPTHYADSVKGR |
| | | FTISRDNSKNTLYLQMNSLRAEDTAVYYCAGYDS |
| | | GYDYVAVAGPAEYFQHWGQGTLVTVSSAKTTAP |
| | | SVYPLAPVCGDTTGSSVTLGCLVKGYFPEPVTLT |
| | | WNSGSLSSGVHTFPAVLQSDLYTLSSSVTVTSST |
| | | WPSQSITCNVAHPASSTKVDKKIEPRGPTIKPCPPC |
| | | KCPAPNAAGGPSVFIFPPKIKDVLMISLSPIVTCVV |
| | | VDVSEDDPDVQISWFVNNVEVHTAQTQTHREDY |
| | | NSTLRVVSALPIQHQDWMSGKEFKCKVNNKDLG |
| | | APIERTISKPKGSVRAPQVYVLPPPEEEMTKKQVT |
| | | LTCMVTDFMPEDIYVEWTNNGKTELNYKNTEPV |
| | | LDSDGSYFMYSKLRVEKKNWVERNSYSCSVVHE |
| | | GLHNHHTTKSFSRTPG |
| 916 | h1C1 LALAPG mk LC | DIQMTQSPSSLSASVGDRVTITCRASQSISTWLAW |
| | | YQQKPGKAPKLLIYKASNLHTGVPSRFSGSGSGTE |
| | | FSLTISGLQPDDFATYYCQQYNSYSRTFGQGTKVE |
| | | IKRADAAPTVSIFPPSSEQLTSGGASVVCFLNNFYP |
| | | KDINVKWKIDGSERQNGVLNSWTDQDSKDSTYS |
| | | MSSTLTLTKDEYERHNSYTCEATHKTSTSPIVKSF |
| | | NRNEC |
| 917 | hL49 HA CDR-H1 | SGYWN |
| 918 | hL49 HA CDR-H2 | YISDSGITYYNPSLKS |
| 919 | hL49 HA CDR-H3 | RTLATYYAMDY |
| 920 | hL49 LC CDR-L1 | RASQSLVHSDGNTYLH |
| 921 | hL49 LC CDR-L2 | RVSNRFS |
| 922 | hL49 LC CDR-L3 | SQSTHVPPT |
| 923 | hL49 HA VH | QVQLQESGPGLVKPSETLSLTCTVSGDSITSGYWN |
| | | WIRQPPGKGLEYIGYISDSGITYYNPSLKSRVTISR |
| | | DTSKNQYSLKLSSVTAADTAVYYCARRTLATYY |
| | | AMDYWGQGTLVTVSS |
| 924 | hL49 LC VL | DFVMTQSPLSLPVTLGQPASISCRASQSLVHSDGN |
| | | TYLHWYQQRPGQSPRLLIYRVSNRFSGVPDRFSG |
| | | SGSGTDFTLKISRVEAEDVGVYYCSQSTHVPPTFG |
| | | QGTKLEIK |
| 925 | hL49 HA HC | QVQLQESGPGLVKPSETLSLTCTVSGDSITSGYWN |
| | | WIRQPPGKGLEYIGYISDSGITYYNPSLKSRVTISR |
| | | DTSKNQYSLKLSSVTAADTAVYYCARRTLATYY |
| | | AMDYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 926 | hL49 HA HC v2 | QVQLQESGPGLVKPSETLSLTCTVSGDSITSGYWN |
| | | WIRQPPGKGLEYIGYISDSGITYYNPSLKSRVTISR |
| | | DTSKNQYSLKLSSVTAADTAVYYCARRTLATYY |
| | | AMDYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| 927 | hL49 LC LC | DFVMTQSPLSLPVTLGQPASISCRASQSLVHSDGN |
| | | TYLHWYQQRPGQSPRLLIYRVSNRFSGVPDRFSG |
| | | SGSGTDFTLKISRVEAEDVGVYYCSQSTHVPPTFG |
| | | QGTKLEIKRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| 928 | hL49 HA LALAKA HC | QVQLQESGPGLVKPSETLSLTCTVSGDSITSGYWN |
| | | WIRQPPGKGLEYIGYISDSGITYYNPSLKSRVTISR |
| | | DTSKNQYSLKLSSVTAADTAVYYCARRTLATYY |
| | | AMDYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCAVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 929 | hL49 HA LALAKA HC v2 | QVQLQESGPGLVKPSETLSLTCTVSGDSITSGYWN |
| | | WIRQPPGKGLEYIGYISDSGITYYNPSLKSRVTISR |
| | | DTSKNQYSLKLSSVTAADTAVYYCARRTLATYY |
| | | AMDYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCAVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| 930 | hL49 LC LALAKA LC | DFVMTQSPLSLPVTLGQPASISCRASQSLVHSDGN |
| | | TYLHWYQQRPGQSPRLLIYRVSNRFSGVPDRFSG |
| | | SGSGTDFTLKISRVEAEDVGVYYCSQSTHVPPTFG |
| | | QGTKLEIKRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| 931 | H2A2 HC CDR-H1 | DYNVN |
| 932 | H2A2 HC CDR-H2 | VINPKYGTTRYNQKFKG |
| 933 | H2A2 HC CDR-H3 | GLNAWDY |
| 934 | H2A2 LG CDR-L1 | GASENIYGALN |
| 935 | H2A2 LG CDR-L2 | GATNLED |
| 936 | H2A2 LG CDR-L3 | QNVLTTPYT |
| 937 | h2A2 HC VH | QFQLVQSGAEVKKPGASVKVSCKASGYSFTDYN |
| | | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSS |
| 938 | h2A2 LG VL | DIQMTQSPSSLSASVGDRVTITCGASENIYGALNW |
| | | YQQKPGKAPKLLIYGATNLEDGVPSRFSGSGSGR |
| | | DYTFTISSLQPEDIATYYCQNVLTTPYTFGQGTKL |
| | | EIK |
| 939 | h2A2 HC HC | QFQLVQSGAEVKKPGASVKVSCKASGYSFTDYN |
| | | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 940 | h2A2 HC HC v2 | QFQLVQSGAEVKKPGASVKVSCKASGYSFTDYN |
| | | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| 941 | h2A2 LG LC | DIQMTQSPSSLSASVGDRVTITCGASENIYGALNW |
| | | YQQKPGKAPKLLIYGATNLEDGVPSRFSGSGSGR |
| | | DYTFTISSLQPEDIATYYCQNVLTTPYTFGQGTKL |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 942 | h2A2 HC LALAKA HC | QFQLVQSGAEVKKPGASVKVSCKASGYSFTDYN |
| | | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPEAAGGP |
| | | SVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCAVSNKALPAPIEKTISKAK |
| | | GQPREPQVYTLPPSRDELTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSL |
| | | SPGK |
| 943 | h2A2 HC LALAKA HC v2 | QFQLVQSGAEVKKPGASVKVSCKASGYSFTDYN |
| | | VNWVRQAPGQGLEWIGVINPKYGTTRYNQKFKG |
| | | RATLTVDKSTSTAYMELSSLRSEDTAVYYCTRGL |
| | | NAWDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPEAAGGP |
| | | SVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCAVSNKALPAPIEKTISKAK |
| | | GQPREPQVYTLPPSRDELTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSL |
| | | SPG |
| 944 | h2A2 LG LALAKA LC | DIQMTQSPSSLSASVGDRVTITCGASENIYGALNW |
| | | YQQKPGKAPKLLIYGATNLEDGVPSRFSGSGSGR |
| | | DYTFTISSLQPEDIATYYCQNVLTTPYTFGQGTKL |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 945 | B7H41001 CDR-H1 | SGSYYWG |
| 946 | B7H41001 CDR-H2 | NIYYSGSTYYNPSLRS |
| 947 | B7H41001 CDR-H3 | EGSYPNQFDP |
| 948 | B7H41001 CDR-L1 | RASQSVSSNLA |
| 949 | B7H41001 CDR-L2 | GASTRAT |
| 950 | B7H41001 CDR-L3 | QQYHSFPFT |
| 951 | B7H41001 VH | QLQLQESGPGLVKPSETLSLTCTVSGGSIKSGSYY |
| | | WGWIRQPPGKGLEWIGNIYYSGSTYYNPSLRSRV |
| | | TISVDTSKNQFSLKLSSVTAADTAVYYCAREGSYP |
| | | NQFDPWGQGTLVTVSS |
| 952 | B7H41001 VL | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | K |
| 953 | B7H41001 HC | QLQLQESGPGLVKPSETLSLTCTVSGGSIKSGSYY |
| | | WGWIRQPPGKGLEWIGNIYYSGSTYYNPSLRSRV |
| | | TISVDTSKNQFSLKLSSVTAADTAVYYCAREGSYP |
| | | NQFDPWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 954 | B7H41001 HC v2 | QLQLQESGPGLVKPSETLSLTCTVSGGSIKSGSYY |
| | | WGWIRQPPGKGLEWIGNIYYSGSTYYNPSLRSRV |
| | | TISVDTSKNQFSLKLSSVTAADTAVYYCAREGSYP |
| | | NQFDPWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| 955 | B7H41001 LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 956 | B7H41001 LALAKA HC | QLQLQESGPGLVKPSETLSLTCTVSGGSIKSGSYY |
| | | WGWIRQPPGKGLEWIGNIYYSGSTYYNPSLRSRV |
| | | TISVDTSKNQFSLKLSSVTAADTAVYYCAREGSYP |
| | | NQFDPWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCAVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 957 | B7H41001 LALAKA HC | QLQLQESGPGLVKPSETLSLTCTVSGGSIKSGSYY |
| | v2 | WGWIRQPPGKGLEWIGNIYYSGSTYYNPSLRSRV |
| | | TISVDTSKNQFSLKLSSVTAADTAVYYCAREGSYP |
| | | NQFDPWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCAVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| 958 | B7H41001 LALAKA LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 959 | B7H4-15461 CDR-H1 | GSISSSSYYWG |
| 960 | B7H4-15461 CDR-H2 | NIYYSGSTYYNPSLKS |
| 961 | B7H4-15461 CDR-H3 | AREGSYPNWFDP |
| 962 | B7H4-15461 CDR-L1 | RASQSVSSNLA |
| 963 | B7H4-15461 CDR-L2 | GASTRAT |
| 964 | B7H4-15461 CDR-L3 | QQYHSFPFT |
| 965 | B7H4-15461 VH | QLQLQESGPGLVKPSETLSLTCTVSGGSISSSSYY |
| | | WGWIRQPPGKGLEWIGNIYYSGSTYYNPSLKSRV |
| | | TISVDTSKNQFSLKLSSVTAADTAVYYCAREGSYP |
| | | NWFDPWGQGTLVTVSS |
| 966 | B7H4-15461 VL | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | K |
| 967 | B7H4-20500 CDR-H1 | GSIKSGSHYWG |
| 968 | B7H4-20500 CDR-H2 | NIYYSGSTYYNPSLRS |
| 969 | B7H4-20500 CDR-H3 | AREGSYPNWFDP |
| 970 | B7H4-20500 CDR-L1 | RASQSVSSNLA |
| 971 | B7H4-20500 CDR-L2 | GASTRAT |
| 972 | B7H4-20500 CDR-L3 | QQYHSFPFT |
| 973 | B7H4-20500 VH | QLQLQESGPGLVKPSETLSLTCTVSGGSIKSGSHY |
| | | WGWIRQPPGKGLEWIGNIYYSGSTYYNPSLRSRV |
| | | TISVDTSKNQFSLKLSSVTAADTAVYYCAREGSYP |
| | | NWFDPWGQGTLVTVSS |
| 974 | B7H4-20500 VL | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | K |
| 975 | B7H4-20501 CDR-H1 | GSIKSGSHYWG |
| 976 | B7H4-20501 CDR-H2 | NIYYSGSTYYNPSLKS |
| 977 | B7H4-20501 CDR-H3 | AREGSYPNWLDP |
| 978 | B7H4-20501 CDR-L1 | RASQSVSSNLA |
| 979 | B7H4-20501 CDR-L2 | GASTRAT |
| 980 | B7H4-20501 CDR-L3 | QQYHSFPFT |
| 981 | B7H4-20501 VH | QLQLQESGPGLVKPSETLSLTCTVSGGSIKSGSHY |
| | | WGWIRQPPGKGLEWIGNIYYSGSTYYNPSLKSRV |
| | | TISVDTSKNQFSLKLSSVTAADTAVYYCAREGSYP |
| | | NWLDPWGQGTLVTVSS |
| 982 | B7H4-20501 VL | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | K |
| 983 | B7H4-20502.1 CDR-H1 | GSIKSGSYYWG |
| 984 | B7H4-20502.1 CDR-H2 | NIYYSGSTYYNPSLKS |
| 985 | B7H4-20502.1 CDR-H3 | AREGSYPNQFDP |
| 986 | B7H4-20502.1 CDR-L1 | RASQSVSSNLA |
| 987 | B7H4-20502.1 CDR-L2 | GASTRAT |
| 988 | B7H4-20502.1 CDR-L3 | QQYHSFPFT |
| 989 | B7H4-20502.1 VH | QLQLQESGPGLVKPSETLSLTCTVSGGSIKSGSYY |
| | | WGWIRQPPGKGLEWIGNIYYSGSTYYNPSLKSRV |
| | | TISVDTSKNQFSLKLSSVTAADTAVYYCAREGSYP |
| | | NQFDPWGQGILVTVSS |
| 990 | B7H4-20502.1 VL | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | K |
| 991 | B7H4-22208 CDR-H1 | GSIKSGSHYWG |
| 992 | B7H4-22208 CDR-H2 | NIYYSGSTYYNPSLKS |
| 993 | B7H4-22208 CDR-H3 | AREGSYPNWFDP |
| 994 | B7H4-22208 CDR-L1 | RASQSVSTNLA |
| 995 | B7H4-22208 CDR-L2 | DASARVT |
| 996 | B7H4-22208 CDR-L3 | QQYHSFPFT |
| 997 | B7H4-22208 VH | QLQLQESGPGLVKPSETLSLTCTVSGGSIKSGSHY |
| | | WGWIRQPPGKGLEWIGNIYYSGSTYYNPSLKSRV |
| | | TMSVDTSKNQFSLKLSSVTAADTAVYYCAREGSY |
| | | PNWFDPWGQGTLVTVSS |
| 998 | B7H4-22208 VL | EIVMTQSPATLSVSPGERATLSCRASQSVSTNLAW |
| | | YQQKPGQAPRLLIYDASARVTGIPARFSGSGSGTE |
| | | FTLTISSLQSEDFAVYYCQQYHSFPFTFGGGTKVEI |
| | | K |
| 999 | B7H4-15462 CDR-H1 | GSISSSSYYWG |
| 1000 | B7H4-15462 CDR-H2 | NIYYSGSTYYNPSLKS |
| 1001 | B7H4-15462 CDR-H3 | AREGSYTTVLNV |
| 1002 | B7H4-15462 CDR-L1 | RASQSVSSSYLA |
| 1003 | B7H4-15462 CDR-L2 | GASSRAT |
| 1004 | B7H4-15462 CDR-L3 | QQAASYPLT |
| 1005 | B7H4-15462 VH | QLQLQESGPGLVKPSETLSLTCTVSGGSISSSSYY |
| | | WGWIRQPPGKGLEWIGNIYYSGSTYYNPSLKSRV |
| | | TISVDTSKNQFSLKLSSVTAADTAVYYCAREGSYT |
| | | TVLNVWGQGTMVTVSS |
| 1006 | B7H4-15462 VL | EIVLTQSPGTLSLSPGERATLSCRASQSVSSSYLA |
| | | WYQQKPGQAPRLLIYGASSRATGIPDRFSGSGSGT |
| | | DFTLTISRLEPEDFAVYYCQQAASYPLTFGGGTKV |
| | | EIK |
| 1007 | B7H4-22213 CDR-H1 | GSIGRGSYYWG |
| 1008 | B7H4-22213 CDR-H2 | NIYYSGSTYYNPSLKS |
| 1009 | B7H4-22213 CDR-H3 | AREGSYTTVLNV |
| 1010 | B7H4-22213 CDR-L1 | RASQSVASSHLA |
| 1011 | B7H4-22213 CDR-L2 | DAVSRAT |
| 1012 | B7H4-22213 CDR-L3 | QQAASYPLT |
| 1013 | B7H4-22213 VH | QLQLQESGPGLVKPSETLSLTCTVSGGSIGRGSYY |
| | | WGWIRQPPGKGLEWIGNIYYSGSTYYNPSLKSRV |
| | | TISVDTSKNQFSLKLSSVTAADTAVYYCAREGSYT |
| | | TVLNVWGQGTMVTVSS |
| 1014 | B7H4-22213 VL | EIVLTQSPGTLSLSPGERATLSCRASQSVASSHLA |
| | | WYQQKPGQAPRLLIYDAVSRATGIPDRFSGSGSG |
| | | TDFTLTISRLEPEDFAVYYCQQAASYPLTFGGGTK |
| | | VEIK |
| 1015 | B7H4-15465 CDR-H1 | GSISSGGYYWS |
| 1016 | B7H4-15465 CDR-H2 | NIYYSGSTYYNPSLKS |
| 1017 | B7H4-15465 CDR-H3 | ARESSTISADFDL |
| 1018 | B7H4-15465 CDR-L1 | RASQGISRWLA |
| 1019 | B7H4-15465 CDR-L2 | AASSLQS |
| 1020 | B7H4-15465 CDR-L3 | QQAHTFPYT |
| 1021 | B7H4-15465 VH | QVQLQESGPGLVKPSQTLSLTCTVSGGSISSGGYY |
| | | WSWIRQHPGKGLEWIGNIYYSGSTYYNPSLKSRV |
| | | TISVDTSKNQFSLKLSSVTAADTAVYYCARESSTIS |
| | | ADFDLWGRGTLVTVSS |
| 1022 | B7H4-15465 VL | DIQMTQSPSSVSASVGDRVTITCRASQGISRWLAW |
| | | YQQKPGKAPKLLIYAASSLQSGVPSRFSGSGSGTD |
| | | FTLTISSLQPEDFATYYCQQAHTFPYTFGGGTKVEI |
| | | K |
| 1023 | B7H4-20506 CDR-H1 | GSISHGGYYWS |
| 1024 | B7H4-20506 CDR-H2 | NIYYSGSTYYNPSLKS |
| 1025 | B7H4-20506 CDR-H3 | ARESSTISADFDL |
| 1026 | B7H4-20506 CDR-L1 | RASQGISRWLA |
| 1027 | B7H4-20506 CDR-L2 | AASSLQS |
| 1028 | B7H4-20506 CDR-L3 | QQAHTFPYT |
| 1029 | B7H4-20506 VH | QLQLQESGPGLVKPSETLSLTCTASGGSISHGGYY |
| | | WSWIRQHPGKGLEWIGNIYYSGSTYYNPSLKSRV |
| | | TMSVDTSKNQFSLKLSSVTAADTAVYYCARESSTI |
| | | SADFDLWGRGTLVTVSS |
| 1030 | B7H4-20506 VL | DIQMTQSPSSVSASVGDRVTITCRASQGISRWLAW |
| | | YQQKPGKAPKLLIYAASSLQSGVPSRFSGSGSGTD |
| | | FTLTISSLQPEDFATYYCQQAHTFPYTFGGGTKVEI |
| | | K |
| 1031 | B7H4-15483 CDR-H1 | GSISSGGYYWS |
| 1032 | B7H4-15483 CDR-H2 | NIYYSGSTYYNPSLKS |
| 1033 | B7H4-15483 CDR-H3 | ARGLSTIDEAFDP |
| 1034 | B7H4-15483 CDR-L1 | RASQSISSWLA |
| 1035 | B7H4-15483 CDR-L2 | KASSLES |
| 1036 | B7H4-15483 CDR-L3 | QQDNSYPYT |
| 1037 | B7H4-15483 VH | QVQLQESGPGLVKPSQTLSLTCTVSGGSISSGGYY |
| | | WSWIRQHPGKGLEWIGNIYYSGSTYYNPSLKSRV |
| | | TISVDTSKNQFSLKLSSVTAADTAVYYCARGLSTI |
| | | DEAFDPWGQGTLVTVSS |
| 1038 | B7H4-15483 VL | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSYPYTFGGGTKVE |
| | | IK |
| 1039 | B7H4-20513 CDR-H1 | GSISDGSYYWS |
| 1040 | B7H4-20513 CDR-H2 | NIYYSGSTYYNPSLRS |
| 1041 | B7H4-20513 CDR-H3 | ARGLSTIDEAFDP |
| 1042 | B7H4-20513 CDR-L1 | RASQSISSWLA |
| 1043 | B7H4-20513 CDR-L2 | KASSLES |
| 1044 | B7H4-20513 CDR-L3 | QQDNSYPYT |
| 1045 | B7H4-20513 VH | QLQLQESGPGLVKPSETLSLTCTVSGGSISDGSYY |
| | | WSWIRQHPGKGLEWIGNIYYSGSTYYNPSLRSRV |
| | | TMSVDTSKNQFSLKLSSVTAADTAVYYCARGLST |
| | | IDEAFDPWGQGTLVTVSS |
| 1046 | B7H4-20513 VL | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSYPYTFGGGTKVE |
| | | IK |
| 1047 | B7H4-22216 CDR-H1 | GSISDGSYYWS |
| 1048 | B7H4-22216 CDR-H2 | NIYYSGSTYYNPSLRS |
| 1049 | B7H4-22216 CDR-H3 | ARGLSTIDEAFDP |
| 1050 | B7H4-22216 CDR-L1 | RASKSISSWLA |
| 1051 | B7H4-22216 CDR-L2 | EASSLHS |
| 1052 | B7H4-22216 CDR-L3 | QQDNSYPYT |
| 1053 | B7H4-22216 VH | QVQLQESGPGLVKPSQTLSLTCTVSGGSISDGSYY |
| | | WSWIRQHPGKGLEWIGNIYYSGSTYYNPSLRSRV |
| | | TMSVDTSKNQFSLKLSSVTAADTAVYYCARGLST |
| | | IDEAFDPWGQGTLVTVSS |
| 1054 | B7H4-22216 VL | DIQMTQSPSTLSASVGDRVTITCRASKSISSWLAW |
| | | YQQKPGKAPKLLIYEASSLHSGVPSRFSGSGSGTE |
| | | FTLTISSLQPDDFATYYCQQDNSYPYTFGGGTKVE |
| | | IK |
| 1055 | B7H4-15489 CDR-H1 | GSISSYYWS |
| 1056 | B7H4-15489 CDR-H2 | YIYSSGSTNYNPSLKS |
| 1057 | B7H4-15489 CDR-H3 | ARGSGQYAAPDYGMD |
| 1058 | B7H4-15489 CDR-L1 | RASQSISSWLA |
| 1059 | B7H4-15489 CDR-L2 | KASSLES |
| 1060 | B7H4-15489 CDR-L3 | QQDNSFPFT |
| 1061 | B7H4-15489 VH | QVQLQESGPGLVKPSETLSLTCTVSGGSISSYYWS |
| | | WIRQPPGKGLEWIGYIYSSGSTNYNPSLKSRVTISV |
| | | DTSKNQFSLKLSSVTAADTAVYYCARGSGQYAAP |
| | | DYGMDVWGQGTTVTVSS |
| 1062 | B7H4-15489 VL | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSFPFTFGGGTKVEI |
| | | K |
| 1063 | B7H4-20516 CDR-H1 | GSIISYYWG |
| 1064 | B7H4-20516 CDR-H2 | YIYSSGSTSYNPSLKS |
| 1065 | B7H4-20516 CDR-H3 | ARGSGLYAAPDYGLDV |
| 1066 | B7H4-20516 CDR-L1 | RASQSISSWLA |
| 1067 | B7H4-20516 CDR-L2 | KASSLES |
| 1068 | B7H4-20516 CDR-L3 | QQDNSFPFT |
| 1069 | B7H4-20516 VH | QVQLQESGPGLVKPSETLSLTCTVSGGSIISYYWG |
| | | WIRQPPGKGLEWIGYIYSSGSTSYNPSLKSRVTISV |
| | | DTSKNQFSLKLSSVTAADTAVYYCARGSGLYAAP |
| | | DYGLDVWGQGTTVTVSS |
| 1070 | B7H4-20516 VL | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSFPFTFGGGTKVEI |
| | | K |
| 1071 | B7H4-15472 CDR-H1 | FTFSSYAMS |
| 1072 | B7H4-15472 CDR-H2 | TISGSGGSTYYADSVKG |
| 1073 | B7H4-15472 CDR-H3 | ARGAGHYDLVGRY |
| 1074 | B7H4-15472 CDR-L1 | RASQSISSYLN |
| 1075 | B7H4-15472 CDR-L2 | AASSLQS |
| 1076 | B7H4-15472 CDR-L3 | QQLYSLPPT |
| 1077 | B7H4-15472 VH | EVQLLESGGGLVQPGGSLRLSCAASGFTFSSYAM |
| | | SWVRQAPGKGLEWVSTISGSGGSTYYADSVKGRF |
| | | TISRDNSKNTLYLQMNSLRAEDTAVYYCARGAG |
| | | HYDLVGRYWGQGTLVTVSS |
| 1078 | B7H4-15472 VL | DIQMTQSPSSLSASVGDRVTITCRASQSISSYLNW |
| | | YQQKPGKAPKLLIYAASSLQSGVPSRFSGSGSGTD |
| | | FTLTISSLQPEDFATYYCQQLYSLPPTFGGGTKVEI |
| | | K |
| 1079 | B7H4-15503 CDR-H1 | FTFSSYAMS |
| 1080 | B7H4-15503 CDR-H2 | AISGSGGSTYYADSVKG |
| 1081 | B7H4-15503 CDR-H3 | ARVGFRALNY |
| 1082 | B7H4-15503 CDR-L1 | RASQDISSWLA |
| 1083 | B7H4-15503 CDR-L2 | AASSLQS |
| 1084 | B7H4-15503 CDR-L3 | QQATSYPPWT |
| 1085 | B7H4-15503 VH | EVQLLESGGGLVQPGGSLRLSCAASGFTFSSYAM |
| | | SWVRQAPGKGLEWVSAISGSGGSTYYADSVKGR |
| | | FTISRDNSKNTLYLQMNSLRAEDTAVYYCARVGF |
| | | RALNYWGQGTTVTVSS |
| 1086 | B7H4-15503 VL | DIQLTQSPSSVSASVGDRVTITCRASQDISSWLAW |
| | | YQQKPGKAPKLLIYAASSLQSGVPSRFSGSGSGTD |
| | | FTLTISSLQPEDFATYYCQQATSYPPWTFGGGTKV |
| | | EIK |
| 1087 | B7H4-15495 CDR-H1 | GTFSSYAIS |
| 1088 | B7H4-15495 CDR-H2 | GIIPIFGTASYAQKFQG |
| 1089 | B7H4-15495 CDR-H3 | ARQQYDGRRYFGL |
| 1090 | B7H4-15495 CDR-L1 | RASQSVSSNLA |
| 1091 | B7H4-15495 CDR-L2 | SASTRAT |
| 1092 | B7H4-15495 CDR-L3 | QQVNVWPPT |
| 1093 | B7H4-15495 VH | QVQLVQSGAEVKKPGSSVKVSCKASGGTFSSYAI |
| | | SWVRQAPGQGLEWMGGIIPIFGTASYAQKFQGRV |
| | | TITADESTSTAYMELSSLRSEDTAVYYCARQQYD |
| | | GRRYFGLWGRGTLVTVSS |
| 1094 | B7H4-15495 VL | EIVMTQSPATLSVSPGERATLSCRASQSVSSNLAW |
| | | YQQKPGQAPRLLIYSASTRATGIPARFSGSGSGTE |
| | | FTLTISSLQSEDFAVYYCQQVNVWPPTFGGGTKV |
| | | EIK |
| 1095 | B7H4-15478 CDR-H1 | GTFSSYAIS |
| 1096 | B7H4-15478 CDR-H2 | GIIPIFGTANYAQKFQG |
| 1097 | B7H4-15478 CDR-H3 | ARGGPWFDP |
| 1098 | B7H4-15478 CDR-L1 | RASQSISSWLA |
| 1099 | B7H4-15478 CDR-L2 | KASSLES |
| 1100 | B7H4-15478 CDR-L3 | QQYNSYPPFT |
| 1101 | B7H4-15478 VH | QVQLVQSGAEVKKPGSSVKVSCKASGGTFSSYAI |
| | | SWVRQAPGQGLEWMGGIIPIFGTANYAQKFQGRV |
| | | TITADESTSTAYMELSSLRSEDTAVYYCARGGPW |
| | | FDPWGQGTLVTVSS |
| 1102 | B7H4-15478 VL | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQYNSYPPFTFGGGTKV |
| | | EIK |
| 1103 | B7H4-15441 CDR-H1 | FTFSSYAMS |
| 1104 | B7H4-15441 CDR-H2 | AISGSGGSTSYADSVKG |
| 1105 | B7H4-15441 CDR-H3 | AKPSLATMLAFDI |
| 1106 | B7H4-15441 CDR-L1 | RASQSISSWLA |
| 1107 | B7H4-15441 CDR-L2 | DASSLES |
| 1108 | B7H4-15441 CDR-L3 | QQSKSYPRT |
| 1109 | B7H4-15441 VH | EVQLLESGGGLVQPGGSLRLSCAASGFTFSSYAM |
| | | SWVRQAPGKGLEWVSAISGSGGSTSYADSVKGRF |
| | | TISRDNSKNTLYLQMNSLRAEDTAVYYCAKPSLA |
| | | TMLAFDIWGQGTMVTVSS |
| 1110 | B7H4-15441 VL | DIQMTQSPSTLSASVGDRVTITCRASQSISSWLAW |
| | | YQQKPGKAPKLLIYDASSLESGVPSRFSGSGSGTE |
| | | FTLTISSLQPDDFATYYCQQSKSYPRTFGGGTKVEI |
| | | K |
| 1111 | B7H4-20496 CDR-H1 | GSISSSVYYWS |
| 1112 | B7H4-20496 CDR-H2 | SILVSGSTYYNPSLKS |
| 1113 | B7H4-20496 CDR-H3 | ARAVSFLDV |
| 1114 | B7H4-20496 CDR-L1 | RASQSISSYLN |
| 1115 | B7H4-20496 CDR-L2 | GASSLQS |
| 1116 | B7H4-20496 CDR-L3 | QQSYDPPWT |
| 1117 | B7H4-20496 VH | QLQLQESGPGLVKPSETLSLTCTVSGGSISSSVYY |
| | | WSWIRQPPGKGLEWIGSILVSGSTYYNPSLKSRVT |
| | | ISVDTSKNQFSLKLSSVTAADTAVYYCARAVSFL |
| | | DVWGQGTMVIVSS |
| 1118 | B7H4-20496 VL | DIQMTQSPSSLSASVGDRVTITCRASQSISSYLNW |
| | | YQQKPGKAPKLLIYGASSLQSGVPSRFSGSGSGTD |
| | | FTLTISSLQPEDFATYYCQQSYDPPWTFGGGTKVE |
| | | IK |
| 1119 | B7H4-15461 HC | QLQLQESGPGLVKPSETLSLTCTVSGGSISSSSYY |
| | | WGWIRQPPGKGLEWIGNIYYSGSTYYNPSLKSRV |
| | | TISVDTSKNQFSLKLSSVTAADTAVYYCAREGSYP |
| | | NWFDPWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1120 | B7H4-15461 LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1121 | B7H4-20500 HC | QLQLQESGPGLVKPSETLSLTCTVSGGSIKSGSHY |
| | | WGWIRQPPGKGLEWIGNIYYSGSTYYNPSLRSRV |
| | | TISVDTSKNQFSLKLSSVTAADTAVYYCAREGSYP |
| | | NWFDPWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1122 | B7H4-20500 LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1123 | B7H4-20501 HC | QLQLQESGPGLVKPSETLSLTCTVSGGSIKSGSHY |
| | | WGWIRQPPGKGLEWIGNIYYSGSTYYNPSLKSRV |
| | | TISVDTSKNQFSLKLSSVTAADTAVYYCAREGSYP |
| | | NWLDPWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1124 | B7H4-20501 LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1125 | B7H4-20502.1 HC | QLQLQESGPGLVKPSETLSLTCTVSGGSIKSGSYY |
| | | WGWIRQPPGKGLEWIGNIYYSGSTYYNPSLKSRV |
| | | TISVDTSKNQFSLKLSSVTAADTAVYYCAREGSYP |
| | | NQFDPWGQGILVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 1126 | B7H4-20502.1 LC | EIVMTQSPATLSVSPGERATLSCRASQSVSSNLAW |
| | | YQQKPGQAPRLLIYGASTRATGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1127 | B7H4-22208 HC | QLQLQESGPGLVKPSETLSLTCTVSGGSIKSGSHY |
| | | WGWIRQPPGKGLEWIGNIYYSGSTYYNPSLKSRV |
| | | TMSVDTSKNQFSLKLSSVTAADTAVYYCAREGSY |
| | | PNWFDPWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1128 | B7H4-22208 LC | EIVMTQSPATLSVSPGERATLSCRASQSVSTNLAW |
| | | YQQKPGQAPRLLIYDASARVTGIPARFSGSGSGTE |
| | | FTLTISSLQSEDFAVYYCQQYHSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1129 | B7H4-15462 HC | QLQLQESGPGLVKPSETLSLTCTVSGGSISSSSYY |
| | | WGWIRQPPGKGLEWIGNIYYSGSTYYNPSLKSRV |
| | | TISVDTSKNQFSLKLSSVTAADTAVYYCAREGSYT |
| | | TVLNVWGQGTMVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1130 | B7H4-15462 LC | EIVLTQSPGTLSLSPGERATLSCRASQSVSSSYLA |
| | | WYQQKPGQAPRLLIYGASSRATGIPDRFSGSGSGT |
| | | DFTLTISRLEPEDFAVYYCQQAASYPLTFGGGTKV |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 1131 | B7H4-22213 HC | QLQLQESGPGLVKPSETLSLTCTVSGGSIGRGSYY |
| | | WGWIRQPPGKGLEWIGNIYYSGSTYYNPSLKSRV |
| | | TISVDTSKNQFSLKLSSVTAADTAVYYCAREGSYT |
| | | TVLNVWGQGTMVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1132 | B7H4-22213 LC | EIVLTQSPGTLSLSPGERATLSCRASQSVASSHLA |
| | | WYQQKPGQAPRLLIYDAVSRATGIPDRFSGSGSG |
| | | TDFTLTISRLEPEDFAVYYCQQAASYPLTFGGGTK |
| | | VEIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 1133 | B7H4-15465 HC | QVQLQESGPGLVKPSQTLSLTCTVSGGSISSGGYY |
| | | WSWIRQHPGKGLEWIGNIYYSGSTYYNPSLKSRV |
| | | TISVDTSKNQFSLKLSSVTAADTAVYYCARESSTIS |
| | | ADFDLWGRGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 1134 | B7H4-15465 LC | DIQMTQSPSSVSASVGDRVTITCRASQGISRWLAW |
| | | YQQKPGKAPKLLIYAASSLQSGVPSRFSGSGSGTD |
| | | FTLTISSLQPEDFATYYCQQAHTFPYTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1135 | B7H4-20506 HC | QLQLQESGPGLVKPSETLSLTCTASGGSISHGGYY |
| | | WSWIRQHPGKGLEWIGNIYYSGSTYYNPSLKSRV |
| | | TMSVDTSKNQFSLKLSSVTAADTAVYYCARESSTI |
| | | SADFDLWGRGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1136 | B7H4-20506 LC | DIQMTQSPSSVSASVGDRVTITCRASQGISRWLAW |
| | | YQQKPGKAPKLLIYAASSLQSGVPSRFSGSGSGTD |
| | | FTLTISSLQPEDFATYYCQQAHTFPYTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1137 | B7H4-15483 HC | QVQLQESGPGLVKPSQTLSLTCTVSGGSISSGGYY |
| | | WSWIRQHPGKGLEWIGNIYYSGSTYYNPSLKSRV |
| | | TISVDTSKNQFSLKLSSVTAADTAVYYCARGLSTI |
| | | DEAFDPWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPELLGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 1138 | B7H4-15483 LC | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSYPYTFGGGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1139 | B7H4-20513 HC | QLQLQESGPGLVKPSETLSLTCTVSGGSISDGSYY |
| | | WSWIRQHPGKGLEWIGNIYYSGSTYYNPSLRSRV |
| | | TMSVDTSKNQFSLKLSSVTAADTAVYYCARGLST |
| | | IDEAFDPWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1140 | B7H4-20513 LC | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSYPYTFGGGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1141 | B7H4-22216 HC | QVQLQESGPGLVKPSQTLSLTCTVSGGSISDGSYY |
| | | WSWIRQHPGKGLEWIGNIYYSGSTYYNPSLRSRV |
| | | TMSVDTSKNQFSLKLSSVTAADTAVYYCARGLST |
| | | IDEAFDPWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1142 | B7H4-22216 LC | DIQMTQSPSTLSASVGDRVTITCRASKSISSWLAW |
| | | YQQKPGKAPKLLIYEASSLHSGVPSRFSGSGSGTE |
| | | FTLTISSLQPDDFATYYCQQDNSYPYTFGGGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1143 | B7H4-15489 HC | QVQLQESGPGLVKPSETLSLTCTVSGGSISSYYWS |
| | | WIRQPPGKGLEWIGYIYSSGSTNYNPSLKSRVTISV |
| | | DTSKNQFSLKLSSVTAADTAVYYCARGSGQYAAP |
| | | DYGMDVWGQGTTVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1144 | B7H4-15489 LC | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1145 | B7H4-20516 HC | QVQLQESGPGLVKPSETLSLTCTVSGGSIISYYWG |
| | | WIRQPPGKGLEWIGYIYSSGSTSYNPSLKSRVTISV |
| | | DTSKNQFSLKLSSVTAADTAVYYCARGSGLYAAP |
| | | DYGLDVWGQGTTVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1146 | B7H4-20516 LC | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQDNSFPFTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1147 | B7H4-15472 HC | EVQLLESGGGLVQPGGSLRLSCAASGFTFSSYAM |
| | | SWVRQAPGKGLEWVSTISGSGGSTYYADSVKGRF |
| | | TISRDNSKNTLYLQMNSLRAEDTAVYYCARGAG |
| | | HYDLVGRYWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSRDELTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPGK |
| 1148 | B7H4-15472 LC | DIQMTQSPSSLSASVGDRVTITCRASQSISSYLNW |
| | | YQQKPGKAPKLLIYAASSLQSGVPSRFSGSGSGTD |
| | | FTLTISSLQPEDFATYYCQQLYSLPPTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1149 | B7H4-15503 HC | EVQLLESGGGLVQPGGSLRLSCAASGFTFSSYAM |
| | | SWVRQAPGKGLEWVSAISGSGGSTYYADSVKGR |
| | | FTISRDNSKNTLYLQMNSLRAEDTAVYYCARVGF |
| | | RALNYWGQGTTVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 1150 | B7H4-15503 LC | DIQLTQSPSSVSASVGDRVTITCRASQDISSWLAW |
| | | YQQKPGKAPKLLIYAASSLQSGVPSRFSGSGSGTD |
| | | FTLTISSLQPEDFATYYCQQATSYPPWTFGGGTKV |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 1151 | B7H4-15495 HC | QVQLVQSGAEVKKPGSSVKVSCKASGGTFSSYAI |
| | | SWVRQAPGQGLEWMGGIIPIFGTASYAQKFQGRV |
| | | TITADESTSTAYMELSSLRSEDTAVYYCARQQYD |
| | | GRRYFGLWGRGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSV |
| | | LTVLHQDWLNGKEYKCKVSNKALPAPIEKTISKA |
| | | KGQPREPQVYTLPPSRDELTKNQVSLTCLVKGFY |
| | | PSDIAVEWESNGQPENNYKTTPPVLDSDGSFFLYS |
| | | KLTVDKSRWQQGNVFSCSVMHEALHNHYTQKSL |
| | | SLSPGK |
| 1152 | B7H4-15495 LC | EIVMTQSPATLSVSPGERATLSCRASQSVSSNLAW |
| | | YQQKPGQAPRLLIYSASTRATGIPARFSGSGSGTE |
| | | FTLTISSLQSEDFAVYYCQQVNVWPPTFGGGTKV |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 1153 | B7H4-15478 HC | QVQLVQSGAEVKKPGSSVKVSCKASGGTFSSYAI |
| | | SWVRQAPGQGLEWMGGIIPIFGTANYAQKFQGRV |
| | | TITADESTSTAYMELSSLRSEDTAVYYCARGGPW |
| | | FDPWGQGTLVTVSSASTKGPSVFPLAPSSKSTSGG |
| | | TAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALPAPIEKTISKAKGQP |
| | | REPQVYTLPPSRDELTKNQVSLTCLVKGFYPSDIA |
| | | VEWESNGQPENNYKTTPPVLDSDGSFFLYSKLTV |
| | | DKSRWQQGNVFSCSVMHEALHNHYTQKSLSLSP |
| | | GK |
| 1154 | B7H4-15478 LC | DIQMTQSPSTLSASVGDRVTITCRASQSISSWLAW |
| | | YQQKPGKAPKLLIYKASSLESGVPSRFSGSGSGTE |
| | | FTLTISSLQPDDFATYYCQQYNSYPPFTFGGGTKV |
| | | EIKRTVAAPSVFIFPPSDEQLKSGTASVVCLLNNFY |
| | | PREAKVQWKVDNALQSGNSQESVTEQDSKDSTY |
| | | SLSSTLTLSKADYEKHKVYACEVTHQGLSSPVTK |
| | | SFNRGEC |
| 1155 | B7H4-15441 HC | EVQLLESGGGLVQPGGSLRLSCAASGFTFSSYAM |
| | | SWVRQAPGKGLEWVSAISGSGGSTSYADSVKGRF |
| | | TISRDNSKNTLYLQMNSLRAEDTAVYYCAKPSLA |
| | | TMLAFDIWGQGTMVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPELLG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALPAPIEKTISK |
| | | AKGQPREPQVYTLPPSRDELTKNQVSLTCLVKGF |
| | | YPSDIAVEWESNGQPENNYKTTPPVLDSDGSFFLY |
| | | SKLTVDKSRWQQGNVFSCSVMHEALHNHYTQKS |
| | | LSLSPGK |
| 1156 | B7H4-15441 LC | DIQMTQSPSTLSASVGDRVTITCRASQSISSWLAW |
| | | YQQKPGKAPKLLIYDASSLESGVPSRFSGSGSGTE |
| | | FTLTISSLQPDDFATYYCQQSKSYPRTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1157 | B7H4-20496 HC | QLQLQESGPGLVKPSETLSLTCTVSGGSISSSVYY |
| | | WSWIRQPPGKGLEWIGSILVSGSTYYNPSLKSRVT |
| | | ISVDTSKNQFSLKLSSVTAADTAVYYCARAVSFL |
| | | DVWGQGTMVIVSSASTKGPSVFPLAPSSKSTSGG |
| | | TAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALPAPIEKTISKAKGQP |
| | | REPQVYTLPPSRDELTKNQVSLTCLVKGFYPSDIA |
| | | VEWESNGQPENNYKTTPPVLDSDGSFFLYSKLTV |
| | | DKSRWQQGNVFSCSVMHEALHNHYTQKSLSLSP |
| | | GK |
| 1158 | B7H4-20496 LC | DIQMTQSPSSLSASVGDRVTITCRASQSISSYLNW |
| | | YQQKPGKAPKLLIYGASSLQSGVPSRFSGSGSGTD |
| | | FTLTISSLQPEDFATYYCQQSYDPPWTFGGGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| 1159 | h1F6 CDR-H1 | NYGMN |
| 1160 | h1F6 CDR-H2 | WINTYTGEPTYADAFKG |
| 1161 | h1F6 CDR-H3 | DYGDYGMDY |
| 1162 | h1F6 CDR-L1 | RASKSVSTSGYSFMH |
| 1163 | h1F6 CDR-L2 | LASNLES |
| 1164 | h1F6 CDR-L3 | QHSREVPWT |
| 1165 | CD19 (hBU12) HC | QVQLQESGPGLVKPSQTLSLTCTVSGGSISTSGMG |
| | | VGWIRQHPGKGLEWIGHIWWDDDKRYNPALKSR |
| | | VTISVDTSKNQFSLKLSSVTAADTAVYYCARMEL |
| | | WSYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPELLG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALPAPIEKTISK |
| | | AKGQPREPQVYTLPPSRDELTKNQVSLTCLVKGF |
| | | YPSDIAVEWESNGQPENNYKTTPPVLDSDGSFFLY |
| | | SKLTVDKSRWQQGNVFSCSVMHEALHNHYTQKS |
| | | LSLSPGK |
| 1166 | CD19 (hBU12) LC | EIVLTQSPATLSLSPGERATLSCSASSSVSYMHWY |
| | | QQKPGQAPRLLIYDTSKLASGIPARFSGSGSGTDF |
| | | TLTISSLEPEDVAVYYCFQGSVYPFTFGQGTKLEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| 1167 | hLIV22/LIV1 HC | QVQLVQSGAEVKKPGASVKVSCKASGLTIEDYY |
| | | MHWVRQAPGQGLEWMGWIDPENGDTEYGPKFQ |
| | | GRVTMTRDTSINTAYMELSRLRSDDTAVYYCAV |
| | | HNAHYGTWFAYWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PELLGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALPAPI |
| | | EKTISKAKGQPREPQVYTLPPSRDELTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPG |
| 1168 | hLIV22/LIV1 LC | DVVMTQSPLSLPVTLGQPASISCRSSQSLLHSSGN |
| | | TYLEWYQQRPGQSPRPLIYKISTRFSGVPDRFSGS |
| | | GSGTDFTLKISRVEAEDVGVYYCFQGSHVPYTFG |
| | | GGTKVEIKRTVAAPSVFIFPPSDEQLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| 1169 | CR011/gpNMB HC | QVQLQESGPGLVKPSQTLSLTCTVSGGSISSFNYY |
| | | WSWIRHHPGKGLEWIGYIYYSGSTYSNPSLKSRV |
| | | TISVDTSKNQFSLTLSSVTAADTAVYYCARGYNW |
| | | NYFDYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRDELTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 1170 | CR011/gpNMB LC | EIVMTQSPATLSVSPGERATLSCRASQSVDNNLV |
| | | WYQQKPGQAPRLLIYGASTRATGIPARFSGSGSGT |
| | | EFTLTISSLQSEDFAVYYCQQYNNWPPWTFGQGT |
| | | KVEIKRTVAAPSVFIFPPSDEQLKSGTASVVCLLN |
| | | NFYPREAKVQWKVDNALQSGNSQESVTEQDSKD |
| | | STYSLSSTLTLSKADYEKHKVYACEVTHQGLSSP |
| | | VTKSFNRGEC |
| 1171 | <Q13433; protein | MARKLSVILI LTFALSVTNP LHELKAAAFP |
| | | QTTEKISPNW ESGINVDLAI STRQYHLQQL |
| | | FYRYGENNSL SVEGFRKLLQ NIGIDKIKRI |
| | | HIHHDHDHHS DHEHHSDHER HSDHEHHSEH |
| | | EHHSDHDHHS HHNHAASGKN KRKALCPDHD |
| | | SDSSGKDPRN SQGKGAHRPE HASGRRNVKD |
| | | SVSASEVTST VYNTVSEGTH FLETIETPRP |
| | | GKLFPKDVSS STPPSVTSKS RVSRLAGRKT |
| | | NESVSEPRKG FMYSRNTNEN PQECFNASKL |
| | | LTSHGMGIQV PLNATEFNYL CPAIINQIDA |
| | | RSCLIHTSEK KAEIPPKTYS LQIAWVGGFI |
| | | AISIISFLSL LGVILVPLMN RVFFKFLLSF |
| | | LVALAVGTLS GDAFLHLLPH SHASHHHSHS |
| | | HEEPAMEMKR GPLFSHLSSQ NIEESAYFDS |
| | | TWKGLTALGG LYFMFLVEHV LTLIKQFKDK |
| | | KKKNQKKPEN DDDVEIKKQL SKYESQLSTN |
| | | EEKVDTDDRT EGYLRADSQE PSHFDSQQPA |
| | | VLEEEEVMIA HAHPQEVYNE YVPRGCKNKC |
| | | HSHFHDTLGQ SDDLIHHHHD YHHILHHHHH |
| | | QNHHPHSHSQ RYSREELKDA GVATLAWMVI |
| | | MGDGLHNFSD GLAIGAAFTE GLSSGLSTSV |
| | | AVFCHELPHE LGDFAVLLKA GMTVKQAVLY |
| | | NALSAMLAYL GMATGIFIGH YAENVSMWIF |
| | | ALTAGLFMYV ALVDMVPEML HNDASDHGCS |
| | | RWGYFFLQNA GMLLGFGIML LISIFEHKIV FRINF |
| 1172 | hLIV22 epitope | KGAHRPEH |
| 1173 | RL peptide | GGGG |
| 1174 | RL peptide | GVKG |
| 1175 | RL peptide | VKGG |
| 1176 | RL peptide | GGFG |
| 1177 | RL peptide | GGFGG |
| 1178 | RC48 CDR-H1 | DYYIH |
| 1179 | RC48 CDR-H2 | RVNPDHGDSYYNQKFKD |
| 1180 | RC48 CDR-H3 | NYLFDH |
| 1181 | RC48 CDR-L1 | KASQDVGTAVA |
| 1182 | RC48 CDR-L2 | WASIRHT |
| 1183 | RC48 CDR-L3 | HQFATYT |
| 1184 | RC48 VH | EVQLVQSGAEVKKPGATVKISCKVSGYTFTDYYI |
| | | HWVQQAPGKGLEWMGRVNPDHGDSYYNQKFK |
| | | DKATITADKSTDTAYMELSSLRSEDTAVYFCARN |
| | | YLFDHWGQGTLVTVSS |
| 1185 | RC48 VL | DIQMTQSPSSVSASVGDRVTITCKASQDVGTAVA |
| | | WYQQKPGKAPKLLIYWASIRHTGVPSRFSGSGSG |
| | | TDFTLTISSLQPEDFATYYCHQFATYTFGGGTKVE |
| | | IK |
| 1186 | RC48 HC | EVQLVQSGAEVKKPGATVKISCKVSGYTFTDYYI |
| | | HWVQQAPGKGLEWMGRVNPDHGDSYYNQKFK |
| | | DKATITADKSTDTAYMELSSLRSEDTAVYFCARN |
| | | YLFDHWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRFEMTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGK |
| 1187 | RC48 HC v2 | EVQLVQSGAEVKKPGATVKISCKVSGYTFTDYYI |
| | | HWVQQAPGKGLEWMGRVNPDHGDSYYNQKFK |
| | | DKATITADKSTDTAYMELSSLRSEDTAVYFCARN |
| | | YLFDHWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLTV |
| | | LHQDWLNGKEYKCKVSNKALPAPIEKTISKAKGQ |
| | | PREPQVYTLPPSRFEMTKNQVSLTCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| 1188 | RC48 LC | DIQMTQSPSSVSASVGDRVTITCKASQDVGTAVA |
| | | WYQQKPGKAPKLLIYWASIRHTGVPSRFSGSGSG |
| | | TDFTLTISSLQPEDFATYYCHQFATYTFGGGTKVE |
| | | IKRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |

1. A cysteine engineered antibody comprising cysteine amino acid substitutions, wherein:
i) the cysteine engineered antibody comprises a heavy chain and a light chain;
and,
ii) the cysteine amino acid substitutions are selected from the group consisting of:
a) position 121 of the light chain and position 375 of the heavy chain;
b) position 121 of the light chain and position 400 of the heavy chain; and
c) position 114 of the light chain and position 400 of the heavy chain,
wherein the amino acid position numbering in the light chain is according to Kabat numbering and the amino acid position numbering in the heavy chain is according to EU numbering.

2. The cysteine engineered antibody of claim 1, wherein the cysteine engineered antibody comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
wherein the amino acid position numbering in the light chains are according to Kabat numbering and the amino acid position numbering in the heavy chains are according to EU numbering.

3. The cysteine engineered antibody of claim 2, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

4. The cysteine engineered antibody of any one of claims 1-3, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

5. The cysteine engineered antibody of any one of claims 1-4, wherein the cysteine amino acid substitutions are of free cysteines.

6. The cysteine engineered antibody of any one of claims 1-5, wherein the cysteine engineered antibody is chimeric, human, or humanized.

7. The cysteine engineered antibody of any one of claims 1-6, wherein the cysteine engineered antibody is a full-length antibody.

8. The cysteine engineered antibody of claim 7, wherein the cysteine engineered antibody is IgA, IgD, IgE, IgG, or IgM.

9. The cysteine engineered antibody of any one of claims 1-8, wherein the cysteine engineered antibody is monospecific.

10. The cysteine engineered antibody of any one of claims 1-8, wherein the cysteine engineered antibody is multispecific.

11. The cysteine engineered antibody of any one of claims 1-10, wherein the cysteine engineered antibody specifically binds to an antigen selected from the group consisting of B7-H4, ITGB6, CD30, CD40, EpCAM, CEACAM5, and LIV1.

12. The cysteine engineered antibody of any one of claims 1-10, wherein the cysteine engineered antibody is selected from the group consisting of an anti-B7-H4 antibody, an anti-ITGB6 antibody, an anti-CD30 antibody, an anti-CD40 antibody, an anti-EpCAM antibody, an anti-CEACAM5 antibody, and an anti-LIV1 antibody.

13. The cysteine engineered antibody of any one of claims 1-10, wherein the cysteine engineered antibody comprises
a heavy chain variable domain (V_{H}) comprising a heavy chain complementarity determining region (CDRH1) comprising the amino acid sequence of any one of SEQ ID NOs: 1, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, 360, 368, 376, 384, 392, 400, 408, 416, 424, 432, 440, 448, 456, 464, 474, 494, 502, 510, 518, 526, 534, 542, 550, 558, 566, 574, 582, 590, 598, 606, 614, 622, 630, 638, 646, 654, 662, 670, 678, 686, 694, 702, 710, 718, 726, 734, 742, 750, 758, 766, 774, 782, 794, 802, 810, 834, 842, 850, 858, 866, 874, 882, 900, 917, 931, 945, 959, 967, 975, 983, 991, 999, 1007, 1015, 1023, 1031, 1039, 1047, 1055, 1063, 1071, 1079, 1087, 1095, 1103, 1111, 1159, and 1178, a CDRH2 comprising the amino acid sequence of any one of SEQ ID Nos: 2, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, 337, 345, 353, 361, 369, 377, 385, 393, 401, 409, 417, 425, 433, 441, 449, 457, 465, 475, 495, 503, 511, 519, 527, 535, 543, 551, 559, 567, 575, 583, 591, 599, 607, 615, 623, 631, 639, 647, 655, 663, 671, 679, 687, 695, 703, 711, 719, 727, 735, 743, 751, 759, 767, 775, 783, 795, 803, 811, 835, 843, 851, 859, 867, 875, 883, 901, 918, 932, 946, 960, 968, 976, 984, 992, 1000, 1008, 1016, 1024, 1032, 1040, 1048, 1056, 1064, 1072, 1080, 1088, 1096, 1104, 1112, 1160, and 1179, a CDRH3 comprising the amino acid sequence DMG or any one of SEQ ID Nos: 3, 18, 26, 34, 42, 50, 58, 66, 74, 82, 90, 98, 114, 122, 130, 138, 146, 154, 162, 170, 178, 186, 194, 202, 210, 218, 226, 234, 242, 250, 258, 266, 274, 282, 290, 298, 306, 314, 322, 330, 338, 346, 354, 362, 370, 378, 386, 394, 402, 410, 418, 426, 434, 442, 450, 458, 466, 476, 496, 504, 512, 520, 528, 536, 544, 552, 560, 568, 576, 584, 592, 600, 608, 616, 624, 632, 640, 648, 656, 664, 672, 680, 688, 696, 704, 712, 720, 728, 736, 744, 752, 760, 768, 776, 784, 796, 804, 812, 836, 844, 852, 860, 868, 876, 884, 902, 919, 933, 947, 961, 969, 977, 985, 993, 1001, 1009, 1017, 1025, 1033, 1041, 1049, 1057, 1065, 1073, 1081, 1089, 1097, 1105, 1113, 1161, and 1180; and a light chain variable domain (VL) comprising a light chain complementarity determining region (CDRL1) comprising the amino acid sequence of any one of SEQ ID Nos: 4, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, 307, 315, 323, 331, 339, 347, 355, 363, 371, 379, 387, 395, 403, 411, 419, 427, 435, 443, 451, 459, 467, 477, 497, 505, 513, 521, 529, 537, 545, 553, 561, 569, 577, 585, 593, 601, 609, 617, 625, 633, 641, 649, 657, 665, 673, 681, 689, 697, 705, 713, 721, 729, 737, 745, 753, 761, 769, 777, 785, 797, 805, 813, 837, 845, 853, 861, 869, 877, 885, 903, 920, 934, 948, 962, 970, 978, 986, 994, 1002, 1010, 1018, 1026, 1034, 1042, 1050, 1058, 1066, 1074, 1082, 1090, 1098, 1106, 1114, 1162, and 1181, a CDRL2 comprising the amino acid sequence of AAS or any one of SEQ ID Nos: 5, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, 308, 316, 324, 332, 340, 348, 356, 364, 372, 380, 388, 396, 404, 412, 420, 428, 436, 444, 452, 460, 468, 478, 498, 506, 514, 522, 530, 538, 546, 554, 562, 570, 578, 586, 594, 602, 610, 618, 626, 634, 642, 650, 658, 666, 674, 682, 690, 698, 706, 714, 722, 730, 738, 746, 754, 762, 770, 778, 786, 798, 806, 814, 838, 846, 854, 862, 870, 878, 904, 921, 935, 949, 963, 971, 979, 987, 995, 1003, 1011, 1019, 1027, 1035, 1043, 1051, 1059, 1067, 1075, 1083, 1091, 1099, 1107, 1115, 1163, and 1182 and a CDRL3 comprising the amino acid sequence of any one of SEQ ID Nos: 6, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333, 341, 349, 357, 365, 373, 381, 389, 397, 405, 413, 421, 429, 437, 445, 453, 461, 469, 479, 499, 507, 515, 523, 531, 539, 547, 555, 563, 571, 579, 587, 595, 603, 611, 619, 627, 635, 643, 651, 659, 667, 675, 683, 691, 699, 707, 715, 723, 731, 739, 747, 755, 763, 771, 779, 787, 799, 807, 815, 839, 847, 855, 863, 871, 879, 887, 905, 922, 936, 950, 964, 972, 980, 988, 996, 1004, 1012, 1020, 1028, 1036, 1044, 1052, 1060, 1068, 1076, 1084, 1092, 1100, 1108, 1116, 1164, and 1183.

14. The cysteine engineered antibody of any one of claims 1-10, wherein the cysteine engineered antibody comprises
a V_{H} comprising an amino acid sequence of any one of SEQ ID Nos: 7, 12, 22, 30, 38, 46, 54, 66, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, 310, 318, 326, 334, 342, 350, 358, 366, 374, 382, 390, 398, 406, 414, 422, 430, 438, 446, 454, 462, 470, 472, 480, 482, 484, 500, 508, 516, 524, 532, 540, 548, 556, 564, 572, 580, 588, 596, 604, 612, 620, 628, 636, 644, 652, 660, 668, 676, 684, 692, 700, 708, 716, 724, 732, 740, 748, 756, 764, 772, 780, 788, 800, 808, 816, 840, 848, 856, 864, 872, 880, 888, 898, 906, 923, 937, 951, 965, 973, 981, 989, 997, 1005, 1013, 1021, 1029, 1037, 1045, 1053, 1061, 1069, 1077, 1085, 1093, 1101, 1109, 1117, and 1184, and a VL comprising the amino acid sequence of any one of SEQ ID Nos: 8, 13, 23, 31, 39, 47, 55, 67, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, 359, 367, 375, 383, 391, 399, 407, 415, 423, 431, 439, 447, 455, 463, 471, 473, 481, 483, 485, 501, 509, 517, 525, 533, 541, 549, 557, 565, 573, 581, 589, 597, 605, 613, 621, 629, 637, 645, 653, 661, 669, 677, 685, 693, 701, 709, 717, 725, 733, 741, 749, 757, 765, 773, 781, 789, 801, 809, 817, 841, 849, 857, 865, 873, 881, 889, 899, 907, 924, 938, 952, 966, 974, 982, 990, 998, 1006, 1014, 1022, 1030, 1038, 1046, 1054, 1062, 1070, 1078, 1086, 1094, 1102, 1110, 1118, and 1185.

15. A cysteine engineered antibody drug conjugate (ADC) comprising the cysteine engineered antibody of any one of claims 1-14 conjugated to a drug moiety.

16. The cysteine engineered ADC of claim 15, wherein the drug moiety is selected from the group consisting of a cytotoxic agent, growth inhibitory agent, and a chemotherapy agent.

17. The cysteine engineered ADC of claim 15 or claim 16, wherein the cysteine engineered antibody is conjugated to a cytotoxic agent.

18. The cysteine engineered ADC of any one of claims 15-17, wherein the cysteine engineered antibody is conjugated to the drug moiety via a linker moiety attached to a cysteine amino acid substitution.

19. The cysteine engineered ADC of any one of claims 15-18, wherein the cysteine engineered ADC comprises a drug moiety conjugated to each an individual cysteine amino acid substitution.

20. The cysteine engineered ADC of claim 15, wherein each of the drug moieties are the same drug moiety.

21. The cysteine engineered ADC of any one of claims 15-20, wherein the drug moiety is a microtubule inhibitor.

22. The cysteine engineered ADC of claim 21, wherein the drug moiety is an auristatin or a tubulysin.

23. The cysteine engineered ADC of any one of claims 15-22, wherein the drug moiety is selected from the group consisting of auristatin T, tubulysin M, tubulysin OEt, MMAE (monomethyl auristatin E), and MMAF (monomethyl auristatin F).

24. The cysteine engineered ADC of claim 23, wherein the drug moiety is MMAE.

25. The cysteine engineered ADC of any one of claims 15-24, wherein the cysteine engineered ADC has a drug antibody ratio (DAR) of about 3.5 to about 4.

26. The cysteine engineered ADC of any one of claims 15-25, wherein less than 50% of the drug moiety is conjugated to the antibody at day 7 after administration to a subject.

27. A nucleic acid encoding the cysteine engineered antibody of any one of claims 1-14.

28. A vector comprising the nucleic acid of claim 27.

29. A host cell comprising the nucleic acid of claim 27 or the vector of claim 28.

30. A pharmaceutical composition comprising the cysteine engineered antibody of any one of claims 1-14 or the cysteine engineered ADC of any one of claims 15-26, and a pharmaceutically acceptable carrier.

31. A method of reducing toxicity of an ADC comprising an antibody moiety conjugated to a drug moiety, the method comprising:
generating a cysteine engineered ADC comprising cysteine amino acid substitutions, wherein the cysteine engineered ADC comprises a cysteine engineered antibody comprising a heavy chain and a light chain, and wherein the cysteine amino acid substitutions are selected from the group consisting of:
a) position 121 of the light chain and position 375 of the heavy chain;
b) position 121 of the light chain and position 400 of the heavy chain;
and,
c) position 114 of the light chain and position 400 of the heavy chain,
wherein the amino acid position numbering in the light chain is according to Kabat numbering and the amino acid position numbering in the heavy chain is according to EU numbering,
wherein the drug moiety is conjugated to each of the cysteine amino acid substitutions, thereby reducing toxicity of the cysteine engineered ADC compared to a second cysteine engineered ADC that comprises a drug moiety conjugated to a cysteine amino acid at one or more other positions, when administered to a patient.

32. The method of claim 31, wherein the cysteine engineered antibody moiety is a full-length antibody.

33. The method of claim 31 or 32, wherein the cysteine engineered antibody moiety comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
wherein the amino acid position numbering in the light chains are according to Kabat numbering and the amino acid position numbering in the heavy chains are according to EU numbering.

34. The method of claim 33, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

35. The method of any one of claims 31-34, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

36. The method of any one of claims 31-35, wherein the generating comprises substituting an amino acid in the cysteine engineered antibody with a cysteine amino acid or inserting a cysteine amino acid into the cysteine engineered antibody.

37. The method of any one of claims 31-36, wherein ocular toxicity of the cysteine engineered ADC is reduced compared to the second cysteine engineered ADC.

38. The method of any one of claims 31-37, wherein corneal toxicity of the cysteine engineered ADC is reduced compared to the second cysteine engineered ADC.

39. The method of any one of claims 31-38, wherein bone marrow toxicity of the cysteine engineered ADC is reduced compared to the second cysteine engineered ADC.

40. The method of any one of claims 31-39, wherein lung toxicity of the cysteine engineered ADC is reduced compared to the second cysteine engineered ADC.

41. The method of any one of claims 31-40, wherein the toxicity of cysteine engineered ADC is reduced compared to the second cysteine engineered ADC at about 7 days after the administration.

42. The method of any one of claims 31-41, wherein the percentage of conjugated drug moiety is reduced for the cysteine engineered ADC compared to a percentage of conjugated drug moiety of the second cysteine engineered ADC at about 7 days after the administration.

43. The method of claim 42, wherein the percentage of conjugated drug moiety is reduced at least about 10%, at least about 15%, at least about 20%, at least about 25%, at least about 30%, at least about 35%, at least about 40%, at least about 45%, at least about 50%, at least about 60%, at least about 70%, or at least about 80% for the cysteine engineered ADC compared to the percentage of conjugated drug moiety of the second cysteine engineered ADC.

44. The method of any one of claims 31-43, wherein the patient has reduced exposure to the drug moiety for the cysteine engineered ADC compared to the exposure produced by the second cysteine engineered ADC.

45. A method of reducing toxicity of a cysteine engineered ADC when administered to a patient, wherein the cysteine engineered ADC comprises a cysteine engineered antibody and a drug moiety, the method comprising:
conjugating the drug moiety to a cysteine amino acid of the cysteine engineered antibody via a linker moiety, wherein the cysteine amino acid is at a position that reduces the stability of the cysteine engineered ADC compared to a second cysteine engineered ADC that comprises a drug moiety conjugated to a cysteine amino acid of a cysteine engineered antibody at one or more other positions.

46. The method of claim 45, wherein the cysteine amino acid is in a heavy chain of the antibody moiety.

47. The method of claim 45 or 46, wherein the cysteine amino acid is in a light chain of the antibody moiety.

48. The method of any one of claims 45-47, wherein the drug moiety is conjugated to each cysteine amino acid substitution of the cysteine engineered antibody.

49. The method of any one of claims 45-48, wherein:
i) the cysteine engineered antibody comprises a heavy chain and a light chain;
and,
ii) the cysteine amino acid substitutions are selected from the group consisting of:
a) position 121 of the light chain and position 375 of the heavy chain;
b) position 121 of the light chain and position 400 of the heavy chain;
and,
c) position 114 of the light chain and position 400 of the heavy chain,
wherein the amino acid position numbering in the light chain is according to Kabat numbering and the amino acid position numbering in the heavy chain is according to EU numbering.

50. The method of claim 49, wherein the cysteine engineered antibody is a full-length antibody.

51. The method of claim 49 or 50, wherein the cysteine engineered antibody comprises two heavy chains and two light chains, wherein the cysteine amino acid substitutions are selected from the group consisting of:
a) position 121 of at least one the light chains and position 375 of at least one of the heavy chains;
b) position 121 of at least one of the light chains and position 400 of at least one of the heavy chains; and,
c) position 114 of at least one of the light chains and position 400 of each of at least one of the heavy chains,
wherein the amino acid position numbering in the light chains are according to Kabat numbering and the amino acid position numbering in the heavy chains are according to EU numbering.

52. The method of claim 51, wherein the cysteine amino acid substitutions are selected from the group consisting of:
a) position 121 of each of the light chains and position 375 of each of the heavy chains;
b) position 121 of each of the light chains and position 400 of each of the heavy chains; and,
c) position 114 of each of the light chains and position 400 of each of the heavy chains,
wherein the amino acid position numbering in the light chains are according to Kabat numbering and the amino acid position numbering in the heavy chains are according to EU numbering.

53. The method of claim 52, wherein the cysteine amino acid substitutions are position 114 of the light chain and position 400 of the heavy chain, wherein the amino acid position number in the light chains is according to Kabat numbering and the amino acid position numbering in the heavy chains is according to EU numbering.

54. A method of treating cancer in a patient, comprising administering an effective amount of the cysteine engineered ADC of any one of claims 15-26 or an effective amount of the pharmaceutical composition of claim 30 to the patient.

55. The method of claim 54, wherein the cancer is a solid tumor, leukemia, or hematological cancer.

56. The method of any one of claims 31-55, wherein administration of the cysteine-engineered ADC causes fewer side effects in the patient compared to the side effects that occur if a non-cysteine engineered ADC or a cysteine-engineered ADC with other cysteine substitutions is administered.

57. The method of claim 56, wherein the one or more side effects comprise ocular toxicity, corneal toxicity, lung toxicity, and/or bone marrow toxicity.

58. The method of claim 57, wherein:
i) the ocular and/or corneal toxicity is determined based on the number mitotic and/or apoptotic figures in the eye;
ii) the lung toxicity is determined based on alveolar macrophage aggregation in the lung; and/or,
iii) the bone marrow toxicity is determined based on suppression of blood cell production in the bone marrow.

59. The method of any one of claims 54-58, wherein less than 50% of the drug moiety is conjugated to the antibody at day 7 after administration to the patient.

60. A kit comprising:
i) the cysteine engineered antibody of any one of claims 1-14 or the cysteine engineered ADC of any one of claims 15-26; and,
ii) instructions for using the cysteine engineered antibody or the cysteine engineered ADC.

61. The cysteine-engineered antibody of any one of claims 1-12 or the cysteine engineered ADC of any one of claims 15-25, wherein the cysteine engineered antibody comprises
a heavy chain comprising the amino acid sequence set forth in SEQ ID NO: 896 and a light chain comprising the amino acid sequence set forth in SEQ ID NO:897;
a heavy chain comprising the amino acid sequence set forth in SEQ ID NO: 894 and a light chain comprising the amino acid sequence set forth in SEQ ID NO:895; or
a heavy chain comprising the amino acid sequence set forth in SEQ ID NO: 890 and a light chain comprising the amino acid sequence set forth in SEQ ID NO:891.
